(19) United States
(12) Patent Application Publication
(10) Pub. No.: US 2026/0317745 A1
(43) Pub. Date: Oct. 15, 2026

(54) Beta-hexosaminidase vectors

(71) Applicant: Universitat Autònoma de Barcelona, Cerdanyola del Vallès (ES)

(72) Inventors: Maria Fàtima Bosch Tubert, Cerdanyola del Valles (ES); Sara Marcó Costa, Salt (ES); Gemma Elias Bosch, Santa Coloma de Farners (ES); Miguel García Martínez, Terrassa (ES)

(73) Assignee: Universitat Autònoma de Barcelona, Cerdanyola del Vallès (ES)

(21) Appl. No.: 19/104,240

(22) PCT Filed: Sep. 6, 2023
(86) PCT No.: PCT/EP2023/074474

§ 371 Date: Feb. 17, 2025

(30) Foreign Application Priority Data
Sep. 7, 2022 (EP) 22382832.8

(51) Int. Cl. C12N 9/24; A61K 48/00; C12N 15/86

(52) U.S. Cl. CPC C12N 9/2402; A61K 48/0075; C12N 15/86; C12N 2750/14143; C12Y 302/01052

(57) ABSTRACT

Described herein is a gene construct comprising a nucleotide sequence encoding an alpha subunit of a beta-hexosaminidase, a nucleotide sequence encoding a peptide linker, and a nucleotide sequence encoding a beta subunit of a beta-hexosaminidase. Aspects and embodiments described herein may be used in the treatment of GM2 gangliosidoses, including Tay-Sachs and Sandhoff disease.

### FIELD

Aspects and embodiments described herein relate to the field of medicine, in particular to beta-hexosaminidase gene therapy for the treatment of GM2 gangliosidoses in mammals, particularly in humans.

### BACKGROUND

GM2 gangliosidosis is a group of three neurodegenerative lysosomal storage diseases (LSD) with an autosomal recessive inheritance caused by beta-hexosaminidase deficiency (Okada & O'Brien, 1969). This enzyme is a glycoprotein synthesized in the ER lumen, processed in the Golgi, and transported via the mannose-6-phosphate receptor to the lysosome (Sun et al, 2021). Beta-hexosaminidase enzyme is composed of two subunits, alpha and beta, in which dimer formation is required for catalytic activity since both subunits possess an active site (Sun et al, 2021). In humans, subunits alpha and beta are encoded by HEXA and HEXB genes, respectively. There are three isoforms of this enzyme: hexosaminidase A (HexA), a heterodimer composed of alpha and beta subunits; hexosaminidase B (HexB), a homodimer formed by two beta subunits; and hexosaminidase S (HexS), a homodimer composed of two alpha subunits (Cachon-Gonzalez et al, 2018). Mutations in HEXA lead to Tays-Sachs disease (OMIM 272800), where the isoform A and S are deficient; mutations in HEXB lead to Sandhoff disease (OMIM 268800), where HexA and HexB isoforms are involved; and mutations in GM2 activator protein lead to GM2 activator deficiency (OMIM 272750) (Cachon-Gonzalez et al, 2018). These three genes are located on chromosome 15q23, 5q13.3, and 5q33.1, respectively (Sun et al, 2021).

Gangliosides are a group of glycosphingolipids composed of a ceramide linked to a glycan with at least one sialic acid (Schnaar, 2019). They are mainly located in caveolae-rich microdomains of the plasma membrane (Yu et al, 2011). Gangliosides are responsible for several pivotal biological functions for the correct functioning of the central nervous system (CNS), such as membrane organization, neuronal differentiation, cell adhesion, cell-cell recognition, signal transduction, inflammation, and neurite outgrowth, among others (Schnaar, 2010; Zeller & Marchase, 1992; Lopez & Báez, 2018; Sonnino et al, 2018; Rubovitch et al, 2017; Regina Todeschini & Hakomori, 2008). About 5% of all brain gangliosides are GM2 gangliosides, a specific type of ganglioside (Schnaar, 2019). In association with the GM2 activator protein, GM2 gangliosides interact exclusively with the beta-hexosaminidase HexA isoform to remove the terminal N-acetylgalactosamine residue that allows GM2 ganglioside degradation (Cachon-Gonzalez et al, 2018; Sandhoff & Harzer, 2013). In Sandhoff and Tay-Sachs diseases, the beta-hexosaminidase deficiency leads to an impairment of the catabolism of GM2 ganglioside which, in turn, results in its accumulation in the endo-lysosomal compartment.

Clinically, both Tay-Sachs and Sandhoff disease are severe, progressive neurodegenerative disorders with clinically indistinguishable phenotypes, but for subtle visceral (organomegalies) and skeletal features in Sandhoff patients (Jain et al, 2010; Venugopalan & Joshi, 2002). The main characteristics are developmental regression, neurological deterioration, motor deficits such as progressive weakness leading to hypotonia, ataxia, spasticity, seizures and vision deterioration or blindness with macular cherry-red spot (Masingue et al, 2020). There is a broad spectrum of clinical presentations and severity in GM2 gangliosidoses mainly due to the residual enzyme activity (Sun et al, 2021; Cachon-Gonzalez et al, 2018). Based on the time of onset, the GM2 gangliosidoses are divided into three clinical subtypes: infantile, juvenile, and adult forms (Cachon-Gonzalez et al, 2018). In general, the later the disease occurs, the more slowly it progresses. The severe infantile form begins in the first year of life with rapidly progressive diffuse neurological deterioration and a premature death within the first 5 years of life (Cachon-Gonzalez et al, 2018; Sun et al, 2021; Leal et al, 2020). On the other hand, juvenile form has an onset between 2 and 10 years, while adult type show an onset after age 10. Both the juvenile and adult form are more slowly progressive neurological disorders in which the clinical manifestations depend on which parts of the central nervous system are affected. These patients usually die around the second decade of life or, in some cases of adult chronic forms, can survive until 60-80 years of age (Leal et al, 2020; Cachon-Gonzalez et al, 2018; Sun et al, 2021). The diagnosis for these diseases begins with recognition of the clinical signs and symptoms, followed by the measurement of beta-hexosaminidase activity, the gold-standard method for diagnosis (Zhang et al, 2019; Hall et al, 2014; Lowden et al, 1973). This diagnosis could be further confirmed by a DNA-based test to identify the underlying mutation (Leal et al, 2020).

Both the understanding of physiopathology and the development of new therapeutic approaches for GM2 gangliosidoses have been studied in several animal models available for these disorders. These animal models include mice, cats, sheep, rabbits and flamingos, and mimic some of the biochemical and physiological characteristics of GM2 gangliosidoses (Lawson & Martin, 2016; Zeng et al, 2008; Sango et al, 1995; Phaneuf et al, 1996; Torres et al, 2010; Rahman et al, 2012; Sanders et al, 2013; Seyrantepe et al, 2018).

To date, there is no cure for Tay-Sachs and Sandhoff disease and existing treatments are aimed at controlling the symptoms to improve the quality of life of patients and, in case of the late-onset form, just delaying the progression of the disease. Currently, there are several therapeutic approaches under pre-clinical investigation in different animal models, including substrate reduction therapy, enzyme replacement therapy, bone marrow transplantation, hematopoietic stem cell transplantation, pharmacological chaperones, and gene therapy to restore expression of the dysfunctional protein (Leal et al, 2020; Solovyeva et al, 2018). Among all therapeutic approaches, two of them based on adeno-associated viral vectors (AAV)-mediated gene therapy are being tested in clinical trials (www.clinicaltrials.gov, NCT04798235 and NCT04669535) (Flotte et al, 2022). Both treatments deliver into the CSF or thalamus either two AAVrh8 vectors comprising HEXA or HEXB (NCT04669535) or one single AAV9 vector comprising both HEXA and HEXB fused with the self-cleaving linker P2A (NCT04798235). Preliminary data from the clinical trial using the AAVrh8-HEXA/HEXB vectors (NCT04669535) demonstrated the safety of the approach after intrathecal and bilateral thalamic delivery accompanied by a modest increase in CSF HexA activity (Flotte et al, 2022).

### SUMMARY

Because beta-hexosaminidase isoform A (HexA) is an alpha beta (aβ) heterodimeric protein, efficient expression of both subunits in the same cell is important to achieve functional protein and a good therapeutic response. In that respect, a single AAV vector comprising both subunits may have an advantage over two-vector approaches, because an advantageous 1:1 ratio of genes encoding the alpha and beta subunits is obtained automatically in transduced cells. Single AAV vectors comprising both subunits also allow decreasing vector dose (because no double transduction of the same cell is required), which in turn results in reduced risk of capsid-triggered immunity or other toxicities. From a regulatory point of view, the use of a single AAV vector comprising both subunits will also greatly facilitate the development of the treatment. Moreover, the use of a single AAV vector comprising both subunits will allow for a dramatic reduction in the cost of manufacturing of AAV vectors.

To date, there are different approaches to generate AAV vectors expressing both alpha and beta subunits and comparable strategies such as engineered hybrid subunits in a single AAV vector, such as:
(i) IRES sequence, the most used element to generate bicistronic vectors, which is an internal entry site of the ribosome to allow production of two proteins from a single mRNA (Arfi et al, 2005; Batista et al, 2010);
(ii) bidirectional promoter, another alternative used to drive expression of two genes (HEXA and HEXB) from a single construct (the direction of expression comes from the center of the vector genome toward the inverted terminal repeats (ITR) in opposite directions) (Lahey et al, 2020); and
(iii) P2A element, a self-cleaving linker to produce alpha and beta subunits from a single protein where the final product has, in the end, some amino acid residues added to the C terminal of beta subunit and a single proline in the N terminal of alpha subunit (Ornaghi et al, 2020; Woodley et al, 2019; Shaimardanova et al, 2022).

A similar strategy aiming to overcome the above-mentioned “heterodimer” challenge of achieving an appropriate subunit ratio involves HexM, a hybrid beta-hexosaminidase subunit which is a new variant of the human HexA alpha-subunit, incorporating critical sequences from the beta-subunit that produce a stable homodimer and promote functional interactions with the GM2 activator protein and GM2 gangliosides (Karumuthil-Melethil et al, 2016b; Tropak et al, 2016; Osmon et al, 2016; Karumuthil-Melethil et al, 2016a; Ou et al, 2020; Kot et al, 2021).

However, most of these existing vectors and comparable strategies such as engineered hybrid subunits are associated with important drawbacks which hamper their therapeutic applicability. For example, it has been reported that the therapeutic efficacy of HexM constructs in Sandhoff and Tay-Sachs mice is limited due to the activation of an unwanted immune response (Kot et al, 2021). Similarly, IRES sequences are typically large in size (+600 bp) and are characterized by an expression variability of the downstream genes (Chai et al, 2018; Mizuguchi et al, 2000). Finally, the vectors carrying the P2A element have been described to only have a limited impact on survival (Woodley et al, 2019; Lahey et al, 2020).

In view of all the above, there is still a need for new treatments for Tay-Sachs and Sandhoff disease which are more effective, safer, and/or do not have the drawbacks of previous approaches. The present inventors have developed an improved gene therapy strategy based on a single vector encoding both the alpha and beta subunit of beta-hexosaminidase for treatment of GM2 gangliosidoses, including Tay-Sachs and Sandhoff disease. In contrast with other approaches, the alpha and beta subunit of beta-hexosaminidase are covalently linked. The long-term and effective expression of the alpha and beta subunit of beta-hexosaminidase provided by a single administration of the vectors of the present invention represents a significant advantage over other approaches. Particularly, as elaborated in the experimental part, the present inventors have found that the vectors of the present invention demonstrate the following unexpected advantages:
The gene constructs and vectors as described herein can obtain a robust and wide-spread increase in beta-hexosaminidase expression and activity in the brain, liver, and serum, and in HEK-293 cells (Examples 3, 4, 6, 7, 8, and 9).
In a widely used mouse model for Sandhoff disease, expression of the alpha and beta subunit of beta-hexosaminidase led to a normalization of GM2 storage in the cerebral cortex and cholesterol accumulation in the cerebral cortex and cerebellum (Examples 4 and 9).
In said mouse model, expression of the alpha and beta subunit of beta-hexosaminidase led to a normalization of lysosomal distension, lysosomal homeostasis, autophagy, myelinization and neuroinflammation in the CNS (Examples 4 and 9).
In said mouse model, expression of the alpha and beta subunit of beta-hexosaminidase led to a normalization of cholesterol in liver and correction of GAG storage in several peripheral tissues and a normalization of lysosomal homeostasis in the liver (Examples 4 and 9).
In said mouse model, expression of the alpha and beta subunit of beta-hexosaminidase led to a clear improvement of general locomotor and exploratory activity, as well as motor coordination, mobility and disease progression (Examples 4 and 9).
In said mouse model, expression of the alpha and beta subunit of beta-hexosaminidase led to a significant increase in survival (Example 4).

Accordingly, the aspects and embodiments of this disclosure solve at least some of the problems and needs as discussed herein.

In a first aspect this disclosure relates to a gene construct for expressing a covalently linked alpha beta (αβ) dimer of beta-hexosaminidase comprising:
a. a nucleotide sequence encoding an alpha subunit of a beta-hexosaminidase;
b. a nucleotide sequence encoding a peptide linker; and
c. a nucleotide sequence encoding a beta subunit of a beta-hexosaminidase.

In some embodiments, a gene construct according to this disclosure is such that the nucleotide sequence encoding an alpha subunit of a beta-hexosaminidase is positioned upstream of the nucleotide sequence encoding a beta subunit of a beta-hexosaminidase.

In some embodiments, a gene construct according to this disclosure is such that the peptide linker is a flexible peptide linker. In some embodiments, a gene construct according to this disclosure is such that the peptide linker is a non-cleavable peptide. In some embodiments, a gene construct according to this disclosure is such that the peptide linker is not a self-cleavable peptide. In some embodiments, a gene construct according to this disclosure is such that at least 30% of the amino acid residues of the peptide linker are glycine residues.

In some embodiments, a gene construct according to this disclosure is such that the nucleotide sequence encoding the peptide linker is selected from the group consisting of:
a. a nucleotide sequence encoding a polypeptide comprising an amino acid sequence that has at least 70% sequence identity with the amino acid sequence of SEQ ID NOs: 11, 12 or 13;
b. a nucleotide sequence comprising a sequence that has at least 70% sequence identity with the nucleotide sequence of SEQ ID NOs: 14, 15 or 16; and
c. a nucleotide sequence which differs from the sequence of a nucleotide sequence of (b) due to the degeneracy of the genetic code.

In some embodiments, a gene construct according to this disclosure is such that the gene construct further comprises a promoter, preferably wherein said promotor is a constitutive promoter, more preferably a CBA promoter or a derivative thereof, even more preferably a Cbh promoter. In some embodiments, the gene construct is flanked by adeno-associated viral ITRs, preferably AAV2 ITRs. In some embodiments, a gene construct according to this disclosure is such that the nucleotide sequence encoding an alpha subunit of a beta-hexosaminidase and/or the nucleotide sequence encoding a beta subunit of a beta-hexosaminidase are optimized, for example codon-optimized, preferably for expression in a human cell. In some embodiments, a gene construct according to this disclosure is such that the nucleotide sequence encoding an alpha subunit of a beta-hexosaminidase is selected from the group consisting of:
a. a nucleotide sequence encoding a polypeptide comprising an amino acid sequence that has at least 70% sequence identity with the amino acid sequence of SEQ ID NOs: 1 or 2;
b. a nucleotide sequence that has at least 70% sequence identity with the nucleotide sequence of SEQ ID NOs: 3, 4, 5, 64, 66, or 68; and
c. a nucleotide sequence which differs from the sequence of a nucleotide sequence of (b) due to the degeneracy of the genetic code.
   and/or wherein the nucleotide sequence encoding a beta subunit of a beta-hexosaminidase is selected from the group consisting of:
a. a nucleotide sequence encoding a polypeptide comprising an amino acid sequence that has at least 70% sequence identity with the amino acid sequence of SEQ ID NOs: 6 or 7;
b. a nucleotide sequence that has at least 70% sequence identity with the nucleotide sequence of SEQ ID NOs: 8, 9, 10, 65, 67, or 69; and
c. a nucleotide sequence which differs from the sequence of a nucleotide sequence of (b) due to the degeneracy of the genetic code.

Another aspect of the disclosure relates to an expression vector comprising a gene construct according to this disclosure. In some embodiments, an expression vector according to this disclosure is such that the expression vector is a viral vector, preferably an adeno-associated viral vector, more preferably an adeno-associated viral vector of serotype 1, 2, BR1, rh8, rh10, PHP.B, TT or 9, most preferably an adeno-associated viral vector of serotype 9.

Another aspect of the disclosure relates to a pharmaceutical composition comprising a gene construct and/or an expression vector according to this disclosure, optionally further comprising one or more pharmaceutically acceptable ingredients, for example selected from the group consisting of excipients, vehicles, carriers, and diluents.

Another aspect of the disclosure relates to a gene construct or an expression vector or a pharmaceutical composition according to this disclosure, for use as a medicament. In some embodiments, a gene construct for use according to this disclosure, an expression vector for use according to this disclosure, or a pharmaceutical composition for use according to this disclosure is for use in the treatment of GM2 gangliosidoses, preferably wherein the GM2 gangliosidosis is selected from the group consisting of Sandhoff disease and Tay-Sachs disease. In some embodiments, a gene construct for use according to this disclosure, an expression vector for use according to this disclosure, or a pharmaceutical composition for use according to this disclosure is such that the gene construct, expression vector or pharmaceutical composition is administered by intra-CSF administration.

### Description

Various features of the aspects and embodiments of this disclosure are further described below. It is noted that headings used throughout this specification are to assist navigation only and should not be interpreted as definitive, and that features described in different sections may be relevant for all aspects and embodiments described herein and may thus be combined as appropriate.

### Gene Construct

Described herein are gene constructs comprising:
a. a nucleotide sequence encoding an alpha subunit of a beta-hexosaminidase;
b. a nucleotide sequence encoding a peptide linker; and
c. a nucleotide sequence encoding a beta subunit of a beta-hexosaminidase.

In some embodiments, elements a (a nucleotide sequence encoding an alpha subunit of a beta-hexosaminidase), b (a nucleotide sequence encoding a peptide linker) and c (a nucleotide sequence encoding a beta subunit of a beta-hexosaminidase) are operably linked. A description of “operably linked” as used herein is provided in the section “general information”.

Such gene constructs are capable of expressing a covalently linked alpha beta (αβ) dimer of beta-hexosaminidase, i.e., a fusion protein comprising an alpha subunit of a beta-hexosaminidase, a peptide linker, and a beta subunit of a beta-hexosaminidase. Accordingly, in some embodiments, there is provided a gene construct for expressing a covalently linked alpha beta (αβ) dimer of beta-hexosaminidase comprising:
a. a nucleotide sequence encoding an alpha subunit of a beta-hexosaminidase;
b. a nucleotide sequence encoding a peptide linker; and
c. a nucleotide sequence encoding a beta subunit of a beta-hexosaminidase.

In other words, in some embodiments there is provided a gene construct for expressing a fusion protein comprising:
a. a nucleotide sequence encoding an alpha subunit of a beta-hexosaminidase;
b. a nucleotide sequence encoding a peptide linker; and
c. a nucleotide sequence encoding a beta subunit of a beta-hexosaminidase.

The fusion protein is then understood to comprise an alpha subunit of a beta-hexosaminidase, a peptide linker, and a beta subunit of a beta-hexosaminidase.

In humans, the HEXA gene (OMIM: 606869; NCBI Gene ID: 3073) encodes an alpha subunit of a beta-hexosaminidase, while the HEXB gene (OMIM: 606873; NCBI Gene ID: 3074) encodes a beta subunit of a beta-hexosaminidase. In mice, the Hexa gene (NCBI Gene ID: 15211) encodes an alpha subunit of a beta-hexosaminidase, while the Hexb gene (NCBI Gene ID: 15212) encodes a beta subunit of a beta-hexosaminidase. Homologous genes exist in other mammalian species and can be readily identified by the skilled person using sequence databases that are commonly available in the art. The skilled person also understands that genes encoding an alpha subunit of a beta-hexosaminidase and genes encoding a beta subunit of a beta-hexosaminidase, such as the HEXA, HEXB, Hexa and Hexb genes mentioned above, may give rise to different isoforms, such as different splice variants, at the mRNA and/or protein level. The number of different isoforms may vary depending on the organism. It is understood that nucleotide sequences encoding any isoforms may be suitable in the context of this disclosure.

In some embodiments, a gene construct as described herein is for expression in a vertebrate, preferably a mammal (e.g., murines such as rat or mice, humans), more preferably a human. In some embodiments, a gene construct as described herein is for expression in a brain, preferably a mammalian (e.g., murine such as rat or mice, human) brain, more preferably a human brain.

As used herein, “for expression” or “suitable for expression” may mean that the gene construct includes one or more regulatory sequences, selected on the basis of the host cells such as brain cells of the vertebrate or mammal or human to be used for expression, which is operatively linked to the nucleotide sequence to be expressed. Preferably, host cells to be used for expression are human or murine (such as rat or mouse) cells, preferably human cells.

In any embodiment described herein, the term “promoter” may be replaced by “transcription regulatory sequence” or “regulatory sequence”. Definitions of these terms are provided in the “general information” section. A “gene construct” as described herein has its customary and ordinary meaning as understood by one of skill in the art in view of this disclosure. A “gene construct” can also be called “expression cassette” or “expression construct” or the like and refers to a gene or a group of genes, including a gene that encodes a protein of interest, which is operably linked to a regulatory sequence that controls its expression. The part of this application entitled “general information” comprises more detail as to a “gene construct”. “Operably linked” as used herein is further described in the part of this application entitled “general information”.

In some embodiments, a gene construct as described herein is suitable for expression in the CNS preferably in the brain of a vertebrate, preferably of a mammal, more preferably of a human. In some embodiments, a gene construct as described herein is suitable for expression in a mammalian brain, more preferably in a human or murine (such as mouse) brain. In preferred embodiments, a gene construct as described herein is suitable for expression in a human brain. As used herein, “suitable for expression in a brain” may mean that the gene construct includes one or more regulatory sequences that are capable of directing expression of the nucleotide sequence to be expressed in said brain, such as in the cortex (which includes the frontal cortex, the parietal cortex, the temporal cortex and the occipital cortex), the thalamus, the hypothalamus, the subthalamus, the epithalamus, the hippocampus, the basal ganglia (which include the striatum, globus pallidus, ventral pallidum, substantia nigra and subthalamic nucleus), the amygdala, the brain stem (which includes the midbrain, pons and medulla oblongata), and/or cerebellum. Accordingly, expression of the gene construct in the brain may mean expression of the gene construct in at least one, at least two, at least three or at least four or all brain regions selected from the group consisting of the cortex, the thalamus, the hypothalamus, the subthalamus, the epithalamus, the hippocampus, the basal ganglia, the amygdala, the brain stem, and the cerebellum.

In some embodiments, a gene construct as described herein is suitable for expression in the liver. In some embodiments, a gene construct as described herein is suitable for expression in the CNS (preferably the brain) and suitable for expression in the liver. While the CNS (preferably the brain) is the main target for expression, expression in the liver may increase circulating levels of beta-hexosaminidase. Without wishing to be bound by theory, this may contribute to correction of somatic pathology observed in GM2 gangliosidoses, as shown in the Examples.

### Alpha Subunit of a Beta-Hexosaminidase

As described elsewhere herein, a gene construct according to this disclosure comprises a nucleotide sequence encoding an alpha subunit of a beta-hexosaminidase. Preferred features of such nucleotide sequences are described in this section.

A nucleotide sequence encoding an alpha subunit of a beta-hexosaminidase as described herein may be derived from any gene or coding sequence encoding an alpha subunit of a beta-hexosaminidase. In some embodiments, a nucleotide sequence encoding an alpha subunit of a beta-hexosaminidase as described herein is derived from, or comprises, the coding region (i.e., the CDS) of said gene.

In some embodiments, a nucleotide sequence encoding an alpha subunit of a beta-hexosaminidase as described herein is derived from a mammalian gene or coding sequence encoding an alpha subunit of a beta-hexosaminidase.

In humans, the HEXA gene (OMIM: 606869; NCBI Gene ID: 3073) encodes an alpha subunit of a beta-hexosaminidase. In mice, the Hexa gene (NCBI Gene ID: 15211) encodes an alpha subunit of a beta-hexosaminidase. Homologous genes exist in other mammalian species and can be readily identified by the skilled person using sequence databases that are commonly available in the art. The skilled person also understands that genes encoding an alpha subunit of a beta-hexosaminidase, such as the HEXA and Hexa genes mentioned above, may give rise to different isoforms, such as different splice variants, at the mRNA and/or protein level. The number of different isoforms may vary depending on the organism. It is understood that nucleotide sequences encoding any isoforms may be suitable in the context of this disclosure.

In some embodiments, a nucleotide sequence encoding an alpha subunit of a beta-hexosaminidase as described herein is derived from a human or murine (such as mouse or rat) gene or coding sequence encoding an alpha subunit of a beta-hexosaminidase, preferably from a human HEXA gene or a mouse Hexa gene as described above. In some embodiments, a nucleotide sequence encoding an alpha subunit of a beta-hexosaminidase as described herein is derived from, or comprises, the coding region (i.e., the CDS) of a human or murine (such as mouse or rat) gene or coding sequence encoding an alpha subunit of a beta-hexosaminidase, preferably from a human HEXA gene or a mouse Hexa gene as described elsewhere.

In some embodiments, a nucleotide sequence encoding an alpha subunit of a beta-hexosaminidase is optimized, for example codon-optimized, preferably for expression in a mammalian (e.g., murine such as rat or mouse, human) cell, more preferably for expression in a human cell.

In some embodiments, a nucleotide sequence encoding an alpha subunit of a beta-hexosaminidase as described herein is an optimized, preferably codon-optimized, sequence derived from a human or murine (such as mouse or rat) gene or coding sequence encoding an alpha subunit of a beta-hexosaminidase, preferably from a human HEXA gene or a mouse Hexa gene as described above. In some embodiments, a nucleotide sequence encoding an alpha subunit of a beta-hexosaminidase as described herein is an optimized, preferably codon-optimized, sequence derived from, or comprising, the coding region (i.e., the CDS) of a human or murine (such as mouse or rat) gene or coding sequence encoding an alpha subunit of a beta-hexosaminidase, preferably from a human HEXA gene or a mouse Hexa gene as described elsewhere.

Accordingly, in some embodiments, a preferred nucleotide sequence encoding an alpha subunit of a beta-hexosaminidase encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 1. SEQ ID NO: 1 represents the canonical amino acid sequence of the human alpha subunit of a beta-hexosaminidase.

In some embodiments, a preferred nucleotide sequence encoding an alpha subunit of a beta-hexosaminidase encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 2. SEQ ID NO: 2 represents an amino acid sequence of the murine alpha subunit of a beta-hexosaminidase.

In some embodiments, a nucleotide sequence encoding an alpha subunit of a beta-hexosaminidase present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with any sequence selected from the group consisting of SEQ ID NO: 3, 64, 66 and 68. SEQ ID NO: 3 represents a nucleotide sequence encoding the human alpha subunit of a beta-hexosaminidase. SEQ ID NOs: 64, 66, and 68 represent optimized sequences encoding the human alpha subunit of a beta-hexosaminidase.

In some embodiments, a nucleotide sequence encoding an alpha subunit of a beta-hexosaminidase present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with any one of SEQ ID NOs: 4-5. SEQ ID NO: 4 represents a nucleotide sequence encoding the murine alpha subunit of a beta-hexosaminidase. SEQ ID NO: 5 represents an optimized sequence encoding the murine alpha subunit of a beta-hexosaminidase.

A description of “identity” or “sequence identity” and “similarity” or “sequence similarity” has been provided under the section entitled “general information”.

In some embodiments, there is provided a gene construct as described herein, wherein the nucleotide sequence encoding an alpha subunit of a beta-hexosaminidase is selected from the group consisting of:
(a) a nucleotide sequence encoding a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or similarity with the amino acid sequence of any one of SEQ ID NOs: 1-2, preferably SEQ ID NO: 1;
(b) a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with the nucleotide sequence of any one of SEQ ID NOs: 3-5, 64, 66, or 68, preferably SEQ ID NO: 3, 64, 66 or 68, more preferably SEQ ID NO: 3, 64 or 68;
(c) a nucleotide sequence the sequence of which differs from the sequence of a nucleotide sequence of (b) due to the degeneracy of the genetic code.

In some embodiments, there is provided a gene construct as described herein, wherein the nucleotide sequence encoding an alpha subunit of beta-hexosaminidase is optimized, for example, codon-optimized, preferably for expression in a human cell. In some embodiments, a nucleotide sequence encoding an alpha subunit of a beta-hexosaminidase present in a gene construct according to the invention is an optimized sequence, for example a codon optimized sequence, preferably an optimized human or murine sequence.

In some embodiments, it has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 5. SEQ ID NO: 5 represents optimized nucleotide sequences encoding an alpha subunit of a beta-hexosaminidase with an amino acid sequence of SEQ ID NO: 2.

In some embodiments, it has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NOs: 64, 66, or 68. SEQ ID NOs: 64, 66, or 68 represent optimized nucleotide sequences encoding an alpha subunit of a beta-hexosaminidase with an amino acid sequence of SEQ ID NO: 1.

A description of “sequence optimization” and “codon optimization” has been provided under the section entitled “general information”.

In preferred embodiments, a nucleotide sequence encoding an alpha subunit of a beta-hexosaminidase may further comprise a sequence encoding a signal peptide. In other words, in the context of this disclosure, a preferred alpha subunit of a beta-hexosaminidase is one with a signal peptide, for example with its nascent signal peptide. The term “signal peptide” is understood herein as a peptide operably linked (fused) in frame to the amino terminus of a polypeptide having biological activity and directing the polypeptide towards one or more cellular compartments, preferably to the lysosome. Typically, the signal peptide is removed by signal peptidases once the polypeptide has reached its designated cellular compartment(s). In some embodiments, the signal peptide directs the alpha subunit of a beta-hexosaminidase (and thus the covalently linked alpha beta (αβ) dimer) to a specific cellular compartment of a cell, preferably the lysosomal compartment.

In mice, the nascent signal sequence is MAGCRLWVSLLLAAALACLATA (SEQ ID NO: 57). In humans, the nascent signal sequence is MTSSRLWFSLLLAAAFAGRATA (SEQ ID NO: 58). Accordingly, in some embodiments, a signal peptide as described herein comprises the sequence of SEQ ID NO: 57 or 58, or a sequence having up to 1, 2, 3, 4, 5, 6, 7, 8, 9 or 10 amino acids deleted, added, and/or substituted compared to SEQ ID NO: 57 or 58. In some embodiments, a signal peptide as described herein comprises the sequence SEQ ID NO: 57 or 58, or a sequence having 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 57 or 58.

### Beta Subunit of a Beta-Hexosaminidase

As described elsewhere herein, a gene construct according to this disclosure comprises a nucleotide sequence encoding a beta subunit of a beta-hexosaminidase. Preferred features of such nucleotide sequences are described in this section.

A nucleotide sequence encoding a beta subunit of a beta-hexosaminidase as described herein may be derived from any gene or coding sequence encoding a beta subunit of a beta-hexosaminidase. In some embodiments, a nucleotide sequence encoding a beta subunit of a beta-hexosaminidase as described herein is derived from, or comprises, the coding region (i.e., the CDS) of said gene.

In some embodiments, a nucleotide sequence encoding a beta subunit of a beta-hexosaminidase as described herein is derived from a mammalian gene or coding sequence encoding a beta subunit of a beta-hexosaminidase.

In humans, the HEXB gene (OMIM: 606873; NCBI Gene ID: 3074) encodes a beta subunit of a beta-hexosaminidase. In mice, the Hexb gene (NCBI Gene ID: 15212) encodes a beta subunit of a beta-hexosaminidase. Homologous genes exist in other mammalian species and can be readily identified by the skilled person using sequence databases that are commonly available in the art. The skilled person also understands that genes encoding a beta subunit of a beta-hexosaminidase, such as the HEXB and Hexb genes mentioned above, may give rise to different isoforms, such as different splice variants, at the mRNA and/or protein level. The number of different isoforms may vary depending on the organism. It is understood that nucleotide sequences encoding any isoforms may be suitable in the context of this disclosure.

In some embodiments, a nucleotide sequence encoding a beta subunit of a beta-hexosaminidase as described herein is derived from a human or murine (such as mouse or rat) gene or coding sequence encoding a beta subunit of a beta-hexosaminidase, preferably a human HEXB gene or a mouse Hexb gene as described elsewhere. In some embodiments, a nucleotide sequence encoding a beta subunit of a beta-hexosaminidase as described herein is derived from, or comprises, the coding region (i.e., the CDS) of a human or murine (such as mouse or rat) gene or coding sequence encoding a beta subunit of a beta-hexosaminidase, preferably from a human HEXB gene or a mouse Hexb gene as described elsewhere.

In some embodiments, a nucleotide sequence encoding a beta subunit of a beta-hexosaminidase is optimized, for example codon-optimized, preferably for expression in a mammalian (e.g., murine such as rat or mouse, human) cell, more preferably for expression in a human cell.

In some embodiments, a nucleotide sequence encoding a beta subunit of a beta-hexosaminidase as described herein is an optimized, preferably codon-optimized, sequence derived from a human or murine (such as mouse or rat) gene or coding sequence encoding a beta subunit of a beta-hexosaminidase, preferably from a human HEXB gene or a mouse Hexb gene as described above. In some embodiments, a nucleotide sequence encoding a beta subunit of a beta-hexosaminidase as described herein is an optimized, preferably codon-optimized, sequence derived from, or comprising, the coding region (i.e., the CDS) of a human or murine (such as mouse or rat) gene or coding sequence encoding a beta subunit of a beta-hexosaminidase, preferably from a human HEXB gene or a mouse Hexb gene as described elsewhere.

Accordingly, in some embodiments, a preferred nucleotide sequence encoding a beta subunit of a beta-hexosaminidase encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 6. SEQ ID NO: 6 represents the canonical amino acid sequence of the human beta subunit of a beta-hexosaminidase.

In some embodiments, a preferred nucleotide sequence encoding a beta subunit of a beta-hexosaminidase encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 7. SEQ ID NO: 7 represents an amino acid sequence of the murine beta subunit of a beta-hexosaminidase.

In some embodiments, a nucleotide sequence encoding a beta subunit of a beta-hexosaminidase present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with any sequence selected from the group consisting of SEQ ID NO: 8, 65, 67, and 69. SEQ ID NO: 8 represents a nucleotide sequence encoding the human beta subunit of a beta-hexosaminidase. SEQ ID NOs: 65, 67, and 69 represent an optimized sequence encoding the human beta subunit of beta-hexosaminidase.

In some embodiments, a nucleotide sequence encoding a beta subunit of a beta-hexosaminidase present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with any one of SEQ ID NOs: 9-10. SEQ ID NO: 9 represents a nucleotide sequence encoding the murine beta subunit of a beta-hexosaminidase. SEQ ID NO: 10 represents an optimized sequence encoding the murine beta subunit of a beta-hexosaminidase.

A description of “identity” or “sequence identity” and “similarity” or “sequence similarity” has been provided under the section entitled “general information”.

In some embodiments, there is provided a gene construct as described herein, wherein the nucleotide sequence encoding a beta subunit of a beta-hexosaminidase is selected from the group consisting of:
(a) a nucleotide sequence encoding a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or similarity with the amino acid sequence of any one of SEQ ID NOs: 6-7, preferably SEQ ID NO: 6;
(b) a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with the nucleotide sequence of any one of SEQ ID NOs: 8-10, 65, 67, or 69, preferably SEQ ID NO: 8, 65, 67 or 69, more preferably 8, 65 or 69;
(c) a nucleotide sequence the sequence of which differs from the sequence of a nucleotide sequence of (b) due to the degeneracy of the genetic code.

In some embodiments, there is provided a gene construct as described herein, wherein the nucleotide sequence encoding a beta subunit of beta-hexosaminidase is optimized, for example, codon-optimized, preferably for expression in a human cell.

In some embodiments, a nucleotide sequence encoding a beta subunit of a beta-hexosaminidase present in a gene construct according to the invention is an optimized sequence, for example a codon-optimized sequence, preferably an optimized human or murine sequence. In some embodiments, it has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 10. SEQ ID NO: 10 represents optimized nucleotide sequences encoding a beta subunit of a beta-hexosaminidase with an amino acid sequence of SEQ ID NO: 7.

In some embodiments, it has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NOs: 65, 67, or 69. SEQ ID NOs: 65, 67, or 69 represent optimized nucleotide sequences encoding a beta subunit of a beta-hexosaminidase with an amino acid sequence of SEQ ID NO: 6.

A description of “sequence optimization” and “codon optimization” has been provided under the section entitled “general information”.

In some embodiments, a nucleotide sequence encoding a beta subunit of a beta-hexosaminidase does not comprise a sequence encoding a signal peptide. In other words, in the context of this disclosure, a preferred beta subunit of a beta-hexosaminidase is one without a signal peptide, for example without its nascent signal peptide.

### Peptide Linker

As described elsewhere herein, a gene construct according to this disclosure comprises a nucleotide sequence encoding a peptide linker. Preferred features of such peptide linkers are described in this section.

As shown in the examples section, advantageous effects are achieved with peptide linkers of various lengths. Thus, the length of the peptide linker as described herein is not crucial and is not particularly limited. In some embodiments, the peptide linker has a length of 6 to 50 amino acids, preferably 8 to 44 amino acids, more preferably 10 to 38 amino acids, even more preferably 11 to 35 amino acids.

In some embodiments, the peptide linker has a minimum length of 6, 7, 8, 9, 10 or 11 amino acids and/or a maximum length of 50, 47, 44, 41, 38 or 35 amino acids.

In some embodiments, the peptide linker has a length of 10-14, preferably 12 amino acids. In some embodiments, the peptide linker has a length of 18-22, preferably 20 amino acids. In some embodiments, the peptide linker has a length of 31-35, preferably 33 amino acids.

In some embodiments, a peptide linker as described herein is a flexible peptide linker. The person skilled in the art understands that flexible linkers are generally composed of small, non-polar (e.g., glycine) or polar (e.g., serine and threonine) amino acids, allowing them to provide flexibility and mobility of the connecting functional domains (as reviewed in Chen et al., Adv Drug Deliv Rev 2013; 65(10): 1357-1369). The flexibility thus allows for achieving the proper conformation of each subunit as required for obtaining functional fusion proteins. Flexible peptide linkers, such as the GS-rich linkers, are used in the synthesis of fusion proteins or peptide conjugates that are not intended to be cleaved by cellular machinery in vivo. These stable linkers covalently join functional domains together, allowing them to function as a single molecule throughout in vivo cellular processes. Therefore, in this disclosure, flexible peptide linkers are understood to be, and may be referred to as, stable flexible peptide linkers.

Cleavable linkers, or “in vivo” cleavable linkers, such as the 2A sequences, on the other hand, are used to release separate free functional domains in vivo. 2A peptides are widely used in molecular biology and genetic engineering to express multiple proteins from a single transcript. Furthermore, the 2A peptides contain functional sequences that induce efficient ribosome skipping, allowing the coordinated production of distinct proteins from a single mRNA transcript. Although a small portion of the two units could be covalently linked, the lack of flexibility of cleavable linkers hinders the proper folding of the entire protein.

In some embodiments, a peptide linker as described herein, preferably a flexible peptide linker as described herein, is such that at least 5%, 10%, 15%, 20%, 25% or 30% of the amino acid residues of the peptide linker are glycine residues. In a preferred embodiment, a peptide linker as described herein, preferably a flexible peptide linker as described herein, is such that at least 30% of the amino acid residues of the peptide linker are glycine residues.

In a further embodiment, at least one, two, three or four amino acid residues of the peptide linker, preferably of the flexible peptide linker, are glycine residues. A preferred number of glycine residues of the peptide linker, preferably of the flexible peptide linker, is at least four.

In some embodiments, a peptide linker as described herein, preferably a flexible peptide linker as described herein, is such that at least 5%, 10%, 15% or 20% of the amino acid residues of the peptide linker are serine residues. In a preferred embodiment, a peptide linker as described herein, preferably a flexible peptide linker as described herein, is such that at least 20% of the amino acid residues of the peptide linker are serine residues. In a further embodiment, at least one, two or three amino acid residues of the peptide linker, preferably of the flexible peptide linker, are serine residues. A preferred number of serine residues of the peptide linker, preferably of the flexible peptide linker, is at least three.

In some embodiments, a peptide linker as described herein, preferably a flexible peptide linker as described herein, is such that at least 5%, 10%, 15%, 20%, 25% or 30% of the amino acid residues of the peptide linker are glycine residues and wherein at least 5%, 10%, 15% or 20% of the amino acid residues of the peptide linker are serine residues. In a preferred embodiment, a peptide linker as described herein, preferably a flexible peptide linker as described herein, is such that at least 30% of the amino acid residues of the peptide linker are glycine residues and at least 20% of the amino acid residues of the peptide linker are serine residues.

In a further embodiment, at least one, two, three or four amino acid residues of the peptide linker, preferably of the peptide linker, are glycine residues and at least one, two or three amino acid residues of the peptide linker, preferably of the peptide linker, are serine residues.

In some embodiments, a peptide linker as described herein, preferably a flexible peptide linker as described herein, is such that at least 10%, 20%, 30%, 40% or 50% of the amino acid residues of the peptide linker are glycine or serine residues. In a preferred embodiment, a peptide linker as described herein, preferably a flexible peptide linker as described herein, is such that at least 50% of the amino acid residues of the peptide linker are glycine or serine residues.

In a further embodiment, at least one, two, three, four, five, six, seven, eight, nine or ten amino acid residues of the peptide linker, preferably of the flexible peptide linker, are glycine or serine residues.

Specific types of flexible peptide linkers suitable for gene constructs of this disclosure include:
(GGGGS) n (SEQ ID NO: 52), wherein “n” is the number of repeats which is preferably 2-7, more preferably 3-6, even more preferably 4 ((GGGGS) 4; SEQ ID NO: 11);
Gly6 (SEQ ID NO: 53);
Gly8 (SEQ ID NO: 54);
KESGSVSSEQLAQFRSLD (SEQ ID NO: 55);
SGGSSGGSSGSETPGTSESATPESSGGSSGGSS (SEQ ID NO: 12);
EGKSSGSGSESKST (SEQ ID NO: 56); and
GSAGSAAGSGEF (SEQ ID NO: 13),
as well as linkers derived thereof, for example linkers having up to 1, 2, 3, 4, 5, 6, 7, 8, 9 or 10 amino acids deleted, added, and/or substituted.

For example, flexible peptide linkers suitable for gene constructs of this disclosure include:
(GGGGS) n (SEQ ID NO: 52), wherein “n” is the number of repeats which is preferably 2-7, more preferably 3-6, even more preferably 4 ((GGGGS) 4; SEQ ID NO: 11);
SGGSSGGSSGSETPGTSESATPESSGGSSGGSS (SEQ ID NO: 12); and
GSAGSAAGSGEF (SEQ ID NO: 13),
as well as linkers derived thereof, for example linkers having up to 1, 2, 3, 4, 5, 6, 7, 8, 9 or 10 amino acids deleted, added, and/or substituted.

Preferred specific types of flexible peptide linkers suitable for gene constructs of this disclosure include:
(GGGGS) 4 (SEQ ID NO: 11);
SGGSSGGSSGSETPGTSESATPESSGGSSGGSS (SEQ ID NO: 12); and
GSAGSAAGSGEF (SEQ ID NO: 13),
as well as linkers derived thereof, for example linkers having up to 1, 2, 3, 4, 5, 6, 7, 8, 9 or 10 amino acids deleted, added, and/or substituted.

In some embodiments, a peptide linker as described herein, preferably a flexible peptide linker, is free of repetitive sequences. Without wishing to be bound by theory, this kind of linkers may be capable of avoiding undesired recombination.

In some embodiments, a peptide linker as described herein is a non-cleavable peptide linker, preferably an in vivo non-cleavable peptide linker. A non-cleavable peptide linker, as understood by the person skilled in the art, refers to a peptide linker which does not contain a protease recognition site or a protease sensitive sequence, which is not sensitive to reductive cleavage, and which is not prone to ribosome skipping. An in vivo non-cleavable peptide linker as used herein refers to a peptide linker which is non-cleavable under in vivo conditions, preferably in the context of a cell such as a brain cell (including any specific brain cell type described herein).

Accordingly, an in vivo non-cleavable peptide linker may be a peptide linker which is not cleavable by the endogenous machinery of a cell, e.g., by endogenous enzyme cleavage or ribosome skipping.

In some embodiments, a peptide linker as described herein is not a cleavable peptide linker, preferably not an in vivo cleavable peptide linker. A cleavable peptide linker, as understood by the person skilled in the art, refers to a peptide linker which contains a protease recognition site or a protease sensitive sequence, which is sensitive to reductive cleavage, or which is prone to ribosome skipping. An in vivo cleavable peptide linker as used herein refers to a peptide linker which is cleavable under in vivo conditions, preferably in the context of a cell such as a brain cell (including any specific brain cell type described herein). Accordingly, an in vivo cleavable peptide linker may be a peptide linker which is cleavable by the endogenous machinery of a cell, e.g., by endogenous enzyme cleavage or ribosome skipping.

In some embodiments, there is provided a gene construct as described herein, wherein the peptide linker is not a self-cleaving peptide. In some embodiments, a peptide linker as described herein is not a P2A self-cleaving peptide, e.g., a P2A self-cleaving peptide as described in Woodley et al. Mol Ther 2019; 12:47-57. A description of “self-cleaving peptide” has been provided under the section entitled “general information”.

In some embodiments, a preferred nucleotide sequence encoding a peptide linker encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with any one of SEQ ID NOs: 11-13. SEQ ID NO: 11 represents an amino acid sequence of a 20 aa long glycine-rich (GGGGS)₄ flexible peptide linker. SEQ ID NO: 12 (SGGSSGGSSGSETPGTSESATPESSGGSSGGSS) represents an amino acid sequence of a 33 aa long flexible peptide linker (Anzalone et al, 2019). SEQ ID NO: 13 (GSAGSAAGSGEF) represents an amino acid sequence of a 12 aa long flexible peptide linker (Waldo et al, 1999).

In some embodiments, a nucleotide sequence encoding a peptide linker present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with any one of SEQ ID NOs: 14-16.

In some embodiments, there is provided a gene construct as described herein, wherein the nucleotide sequence encoding a peptide linker is selected from the group consisting of:
(a) a nucleotide sequence encoding a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or similarity with the amino acid sequence of any one of SEQ ID NOs: 11-13;
(b) a nucleotide sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with the nucleotide sequence of any one of SEQ ID NOs: 14-16;
(c) a nucleotide sequence the sequence of which differs from the sequence of a nucleotide sequence of (b) due to the degeneracy of the genetic code.

### Covalently Linked Alpha Beta (αβ) Dimer of Beta-Hexosaminidase

As described elsewhere herein, a gene construct as described herein comprises a nucleotide sequence encoding an alpha subunit of a beta-hexosaminidase, a nucleotide sequence encoding a peptide linker, and a nucleotide sequence encoding a beta subunit of a beta-hexosaminidase.

Such a gene construct is capable of expressing a covalently linked alpha beta (αβ) dimer of beta-hexosaminidase, i.e., a fusion protein comprising an alpha subunit of a beta-hexosaminidase, a peptide linker, and a beta subunit of a beta-hexosaminidase. Accordingly, in some embodiments there is provided a gene construct for expressing a fusion protein comprising:
a. a nucleotide sequence encoding an alpha subunit of a beta-hexosaminidase;
b. a nucleotide sequence encoding a peptide linker; and
c. a nucleotide sequence encoding a beta subunit of a beta-hexosaminidase.

The fusion protein is then understood to comprise an alpha subunit of a beta-hexosaminidase, a peptide linker, and a beta subunit of a beta-hexosaminidase, all of which are described elsewhere herein.

Any of the fusion proteins that can be encoded by any of the gene constructs described herein, are also an aspect of this disclosure.

In some embodiments, a preferred nucleotide sequence encoding a covalently linked alpha beta (αβ) dimer of beta-hexosaminidase encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with any one of SEQ ID NOs: 17-19. SEQ ID NO: 17 represents an amino acid sequence of a murine covalently linked (L1 linker) alpha beta (αβ) dimer of beta-hexosaminidase. SEQ ID NO: 18 represents an amino acid sequence of a murine covalently linked (L2 linker) alpha beta (αβ) dimer of beta-hexosaminidase. SEQ ID NO: 19 represents an amino acid sequence of a murine covalently linked (L3 linker) alpha beta (αβ) dimer of beta-hexosaminidase.

In some embodiments, a nucleotide sequence encoding a covalently linked alpha beta (αβ) dimer of beta-hexosaminidase present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with any one of SEQ ID NOs: 20-22. SEQ ID NO: 20 represents a nucleotide sequence encoding a murine covalently linked (L1 linker) alpha beta (αβ) dimer of beta-hexosaminidase. SEQ ID NO: 21 represents a nucleotide sequence encoding a murine covalently linked (L2 linker) alpha beta (αβ) dimer of beta-hexosaminidase. SEQ ID NO: 22 represents a nucleotide sequence encoding a murine covalently linked (L3 linker) alpha beta (αβ) dimer of beta-hexosaminidase.

In some embodiments, a preferred nucleotide sequence encoding a covalently linked alpha beta (αβ) dimer of beta-hexosaminidase encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 70. SEQ ID NO: 70 represents an amino acid sequence of a human covalently linked (L1 linker) alpha beta (αβ) dimer of beta-hexosaminidase. In some embodiments, a nucleotide sequence encoding a covalently linked alpha beta (αβ) dimer of beta-hexosaminidase present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with any one of SEQ ID NOs: 71-74. SEQ ID NO: 71 represents a nucleotide sequence encoding a human covalently linked (L1 linker) alpha beta (αβ) dimer of beta-hexosaminidase. SEQ ID NOs: 72-74 represent optimized nucleotide sequences encoding a human covalently linked (L1 linker) alpha beta (αβ) dimer of beta-hexosaminidase.

In some embodiments, a gene construct according to this disclosure is such that expression of the gene construct, optionally expression of the gene construct in a cell (preferably a brain cell), results in at least 50%, 60%, 70%, 80%, 90%, 95%, or 99% of covalently linked alpha beta (αβ) dimer of beta-hexosaminidase, i.e., at least 50%, 60%, 70%, 80%, 90%, 95%, or 99% of fusion protein. In some embodiments, a gene construct according to this disclosure is such that expression of the gene construct, optionally expression of the gene construct in a cell (preferably a brain cell), does not result in a detectable level of covalently linked alpha beta (αβ) dimer of beta-hexosaminidase, i.e., of fusion protein. In this context, detection of the covalently linked alpha beta (αβ) dimer, i.e., of the fusion protein, may be performed by any suitable method known to the person skilled in the art, e.g., methods to measure expression as described in the section “general information”, preferably by a Western blot assay, such as a Western blot assay as described in the examples section.

In some embodiments, there is provided a gene construct as described herein, wherein the nucleotide sequence encoding an alpha subunit of beta-hexosaminidase and/or the nucleotide sequence encoding a beta subunit of a beta-hexosaminidase are optimized, for example, codon-optimized, preferably for expression in a human cell. In some embodiments, there is provided a gene construct as described herein, wherein the nucleotide sequence encoding an alpha subunit of a beta-hexosaminidase is positioned upstream of the nucleotide sequence encoding a beta subunit of a beta-hexosaminidase. As used herein, “upstream” refers to a location within the gene construct which is toward the 5′ end of the polynucleotide from a specific reference point. In some embodiments, there is provided a gene construct as described herein, wherein the nucleotide sequence encoding an alpha subunit of a beta-hexosaminidase is positioned upstream of the nucleotide sequence encoding a peptide linker, and wherein the nucleotide sequence encoding the peptide linker is positioned upstream of the nucleotide sequence encoding a beta subunit of a beta-hexosaminidase. In some embodiments, a nucleotide sequence encoding a peptide linker is positioned between the nucleotide sequence encoding an alpha subunit of a beta-hexosaminidase and the nucleotide sequence encoding a beta subunit of a beta-hexosaminidase.

An alpha subunit of a beta-hexosaminidase and a beta subunit of a beta-hexosaminidase encoded by the nucleotide sequences described herein exert at least a detectable level of an activity of a beta-hexosaminidase as known to a person of skill in the art. In other words, in some embodiments, a covalently linked alpha beta (αβ) dimer of beta-hexosaminidase as described herein maintains beta-hexosaminidase function. Beta-hexosaminidase catalyzes the hydrolysis of terminal N-acetyl-D-hexosamine residues in N-acetyl-B-D-hexosaminides. Means and methods to measure this activity are commonly known in the art, for example as described in the Examples section. In the context of this disclosure, an activity of a beta-hexosaminidase can also be to normalize GM2 ganglioside accumulation of GM2 gangliosidosis patients as described in more detail later herein. An activity of a beta-hexosaminidase can also be to normalize lysosomal distension, lysosomal homeostasis, autophagy, myelinization and neuroinflammation in the CNS of GM2 gangliosidosis patients. An activity of a beta-hexosaminidase can also be to normalize cholesterol in liver and GAG storage in several peripheral tissues and to normalize lysosomal homeostasis in the liver of GM2 gangliosidosis patients. An activity of a beta-hexosaminidase can also be to improve general locomotor and exploratory activity, as well as motor coordination, mobility, and disease progression in GM2 gangliosidosis patients. Another activity of a beta-hexosaminidase can also be to improve survival of GM2 gangliosidosis patients. These activities could be assessed by methods known to a person of skill in the art, for example by using enzymatic assays or behavioral test (such as the open field test, the righting reflex test, the mesh test, the hindlimb clasping test or the rotarod test).

In some embodiments, the nucleotide sequence encoding a covalently linked alpha beta (αβ) dimer of beta-hexosaminidase as described herein is operably linked to a promoter. Accordingly, in some embodiments, a gene construct as described herein further comprises a promoter. In preferred embodiments, there is provided a gene construct as described herein, wherein the promoter is a constitutive promoter. A description of “promoter” has been provided under the section entitled “general information”.

A promoter as used herein encompasses derivatives of promoters and should exert at least an activity of a promoter as known to a person of skill in the art (especially when the promoter sequence is described as having a minimal identity percentage with a given SEQ ID NO). Preferably, a promoter described as having a minimal identity percentage with a given SEQ ID NO should control transcription of the nucleotide sequence to which it is operably linked as assessed in an assay known to a person of skill in the art. For example, such assay could involve measuring expression of the transgene. Expression may be assessed as described under the section entitled “general information” or as shown in the Examples.

In some embodiments, a constitutive promoter as described herein is selected from the group consisting of a CAG promoter, a CMV promoter, a Cbh promoter, a mini-CMV promoter, a chicken beta-actin promoter (CBA), a rous-sarcoma-virus (RSV) promoter, an elongation factor 1 alpha (EF1alpha) promoter, an early growth response factor-1 (Egr-1) promoter, an Eukaryotic Initiation Factor 4A (elF4A) promoter, a ferritin heavy chain-encoding gene (FerH) promoter, a ferritin heavy light-encoding gene (FerL) promoter, a glyceraldehyde-3-phosphate dehydrogenase (GAPDH) promoter, a GRP78 promoter, a GRP94 promoter, a heat-shock protein 70 (hsp70) promoter, an ubiquitin B promoter, a SV40 promoter, a Beta-Kinesin promoter, a ROSA26 promoter, a PGK-1 promoter, and derivatives thereof.

Derivatives of promoters as described herein comprise promoters that have been mutated as to differentiate the directed expression of the transgenes operably linked to said promoters as compared to the non-mutated promoters, which can be increased or decreased, preferably increased. Methods of mutating nucleotide sequences are known to the skilled person and can comprise any of introduction of single nucleotide polymorphisms, nucleotide insertions and nucleotide deletions. CBA promoters and their derivatives are particularly useful for expression of gene constructs in the CNS.

The skilled person understands that derivatives of promoters can also encompass promoters that have been shortened (by nucleotide deletions) or elongated (by nucleotide insertions) compared to their wild-type sequences, with shortened promoters being preferred.

In a preferred embodiment, the constitutive promoter is a chicken beta-actin (CBA) promoter or a derivative thereof. Accordingly, in some embodiments, a gene construct as described herein, further comprises a promoter, wherein said promotor is a constitutive promoter, preferably a CBA promoter or a derivative thereof, more preferably a Cbh promoter.

In some embodiments, a CBA promoter (for mammalian expression) comprises, consists essentially of, or consists of the nucleotide sequence of SEQ ID NO: 23, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In a more preferred embodiment, the CBA promoter or a derivative thereof is a novel hybrid form of the CBA promoter (Cbh). Accordingly, in more preferred embodiments, the constitutive promoter is a Cbh promoter. In some embodiments, a Cbh promoter or a derivative thereof is suitable for promoting the expression of genes in mammals.

In some embodiments, a (mammalian) Cbh promoter comprises, consists essentially of, or consists of a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 24.

Additional sequences may be present in a gene construct as described herein. Exemplary additional sequences suitable for gene constructs described herein include inverted terminal repeats (ITRs) and an SV40 polyadenylation (polyA) signal (SEQ ID NOs: 25-27). Within the context of the invention, “ITRs” is intended to encompass one 5′ITR and one 3′ITR, each being derived from the genome of an AAV. Preferred ITRs are from AAV2 and are represented by SEQ ID NO: 25 (5′ ITR) and SEQ ID NO: 26 (3′ ITR). Each of these additional sequences may be present in a gene construct according to the invention. Accordingly, in some embodiments, there is provided a gene construct as described herein, wherein the gene construct is flanked by adeno-associated viral ITRs. In preferred embodiments, said adeno-associated viral ITRs are AAV2 ITRs. In some embodiments, a gene construct as described herein is flanked by adeno-associated viral ITRs, preferably ITRs are AAV2 ITRs. In preferred embodiments, the AAV2 ITRs are represented by SEQ ID NO: 25 (5′ ITR) and SEQ ID NO: 26 (3′ ITR).

In some embodiments, there is provided a gene as described herein, which further comprises a polyA sequence. In preferred embodiments, said polyA sequence is a SV40 polyA sequence. In even more preferred embodiments, the SV40 polyA sequence is represented by SEQ ID NO: 27.

Optionally, additional nucleotide sequences may be operably linked to the nucleotide sequence(s) encoding a covalently linked alpha beta (αβ) dimer of beta-hexosaminidase, such as nucleotide sequences encoding signal sequences, nuclear localization signals, expression enhancers, and the like.

In some embodiments, the additional sequences are operably linked to the nucleotide sequences described elsewhere. In some embodiments, any of the nucleotide sequences described herein may be operably linked with each other within the gene construct of the disclosure.

In some embodiments, a gene construct of the disclosure comprises a Cbh promoter. Optionally, the gene construct further includes 5′ and 3′ flanks of inverted terminal repeats (ITRs) derived from the genome of an AAV, preferably from AAV2. Optionally, the gene construct further includes a polyA sequence, preferably SV40 polyA. In some embodiments, such gene construct has the nucleotide sequence of SEQ ID NOs: 75-84, preferably SEQ ID NOs: 75-77, or 81-84, more preferably SEQ ID NOs: 81-84, even more preferably SEQ ID NOs: 82 or 84, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith. In some embodiments, such gene construct has the nucleotide sequence of SEQ ID NOs: 81-84, preferably SEQ ID NOs: 82 or 84, except that the L1 linker is replaced with another peptide linker as described herein, for example the L2 or L3 linker as described herein.

For any sequence described herein, in some embodiments, the level of sequence identity or similarity as used herein is preferably 70%. Another preferred level of sequence identity or similarity is 80%. Another preferred level of sequence identity or similarity is 90%. Another preferred level of sequence identity or similarity is 95%. Another preferred level of sequence identity or similarity is 99%.

### Expression Vector

Gene constructs described herein can be placed in expression vectors. Thus, in another aspect there is provided an expression vector comprising a gene construct as described in any of the preceding embodiments.

A description of “expression vector”, or in brief “vector”, has been provided under the section entitled “general information”. The skilled person understands that the term “expression vector” includes non-viral and viral vectors. Suitable expression vectors may be selected from any genetic element which can facilitate transfer of genes or nucleic acids between cells, such as, but not limited to, a plasmid, phage, transposon, cosmid, chromosome, artificial chromosome, virus, virion, etc. A suitable expression vector may also be a chemical vector, such as a lipid complex or naked DNA. “Naked DNA” or “naked nucleic acid” refers to a nucleic acid molecule that is not contained within a viral particle, bacterial cell, or other encapsulating means that facilitates delivery of nucleic acid into the cytoplasm of the target cell. Optionally, a naked nucleic acid can be associated with standard means used in the art for facilitating its delivery of the nucleic acid to the target cell, for example to facilitate the transport of the nucleic acid through the alimentary canal, to protect the nucleic acid from stomach acid and/or nucleases, and/or serve to penetrate intestinal mucus.

In a preferred embodiment, the expression vector is a viral expression vector. A description of “viral expression vector” or “viral vector” in short has been provided under the section entitled “general information”.

A viral vector may be a viral vector selected from the group consisting of adenoviral vectors, adeno-associated viral vectors, retroviral vectors, and lentiviral vectors. An adenoviral vector is also known as an adenovirus derived vector, an adeno-associated viral vector is also known as an adeno-associated virus derived vector, a retroviral vector is also known as a retrovirus derived vector and a lentiviral vector is also known as a lentivirus derived vector. A preferred viral vector is an adeno-associated viral vector. In some embodiments, the expression vector is selected from the group consisting of adenoviral vectors, adeno-associated viral vectors, retroviral vectors, and lentiviral vectors. In preferred embodiments, the expression vector is an adeno-associated viral vector. A description of “adeno-associated viral vector” has been provided under the section entitled “general information”.

In some embodiments, the viral vector is an adeno-associated vector (AAV) selected from the group consisting of AAV of serotype 1 (AAV1), AAV of serotype 2 (AAV2), AAV of serotype 3 (AAV3), AAV of serotype 4 (AAV4), AAV of serotype 5 (AAV5), AAV of serotype 6 (AAV6), AAV of serotype 7 (AAV7), AAV of serotype 8 (AAV8), AAV of serotype 9 (AAV9), AAV of serotype rh10 (AAVrh10), AAV of serotype rh8 (AAVrh8), AAV of serotype Cb4 (AAVCb4), AAV of serotype rh74 (AAVrh74), AAV of serotype DJ (AAVDJ), AAV of serotype 2.5 (AAV2.5), AAV of serotype BR1 (AAV-BR1), AAV of serotype PHP.B (AAV-PHP.B), AAV of serotype PHP.eB (AAV-PHP.eB), AAV of serotype TT (AAV-TT) and AAV of serotype Anc80 (AAVAnc80), preferably the viral vector is an AAV of serotype AAV1, AAV2, AAV2.5, AAV-BR1, AAV-PHP.B, AAV-PHP.eB, AAV-TT, AAVrh8, AAVrh10 or AAV9, more preferably the viral vector is an AAV of serotype AAV9. Serotypes AAV1, AAV2, AAV-BR1, AAV-PHP.B, AAV-PHP.eB, AAV-TT, AAVrh8, AAVrh10 and AAV9 are advantageous in the context of achieving brain expression of hexosaminidase.

In a preferred embodiment, the vector is an AAV of serotype 9, PHP.B, TT, or rh10. In another preferred embodiment, the vector is AAV1, AAV2, AAV-BR1, AAVrh8, or AAV9. In a more preferred embodiment, the vector is an AAV of serotype 9. This serotype is demonstrated in the examples to be especially advantageous for use as an expression vector according to the invention.

It is understood that the AAV expression vectors described herein may also be denoted as recombinant AAV or rAAV vectors.

In some embodiments, the expression vector is an AAV9 and comprises a gene construct comprising a nucleotide sequence encoding a covalently linked alpha beta (αβ) dimer of beta-hexosaminidase, operably linked to a Cbh promoter. Optionally, the gene construct further includes 5′ and 3′ flanks of inverted terminal repeats (ITRs) derived from the genome of an AAV, preferably from AAV2. Optionally, the gene construct further includes a polyA sequence, preferably SV40 polyA. In some embodiments, such expression vector comprises a gene construct having the nucleotide sequence of SEQ ID NOs: 75-84, preferably SEQ ID NOs: 75-77 or 81-84, more preferably SEQ ID NOs: 81-84, even more preferably SEQ ID NOs: 82 or 84, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

The production of recombinant AAV (rAAV) for vectorizing transgenes have been described previously. See Ayuso E, et al., Curr. Gene Ther. 2010; 10:423-436, Okada T, et al., Hum. Gene Ther. 2009; 20:1013-1021, Zhang H, et al., Hum. Gene Ther. 2009; 20:922-929, and Virag T, et al., Hum. Gene Ther. 2009; 20:807-817; all of which are incorporated herein by reference. These protocols can be used or adapted to generate the AAV of this disclosure. Thus, in another aspect there is provided a method for producing an adeno-associated viral vector as described herein.

In short, the methods generally involve (a) the introduction of the AAV genome comprising the gene construct to be expressed into a cell, (b) the presence or introduction of an AAV helper construct in the cell, wherein the helper construct comprises the viral functions missing from the AAV genome and, optionally, (c) the introduction of a helper virus into the host cell. All components for AAV vector replication and packaging need to be present, to achieve replication and packaging of the AAV genome into AAV vectors. These typically include AAV cap proteins, AAV rep proteins and, optionally, viral proteins upon which AAV is dependent for replication. Rep and cap regions are well known in the art, see e.g., Chiorini et al. (1999, J. of Virology, Vol 73 (2): 1309-1319, incorporated herein by reference) or U.S. Pat. No. 5,139,941 (incorporated herein by reference). The AAV cap and rep proteins may derive from the same AAV serotype, or they can derive from a combination of different serotypes, preferably they derive from the serotypes described elsewhere herein. The viral proteins upon which AAV is dependent for replication may derive from any virus, such as an herpes simplex viruses (such as HSV types 1 and 2), a vaccinia virus, an adeno-associated virus or an adenovirus, preferably from an adenovirus.

In some embodiments, the producer cell line is transfected transiently with the polynucleotide of the invention (comprising the expression cassette flanked by ITRs) and with construct(s) that encode(s) rep and cap proteins and provide(s) helper functions. In some embodiments, the cell line supplies stably the helper functions and is transfected transiently with the polynucleotide of the invention (comprising the expression cassette flanked by ITRs) and with construct(s) that encode(s) rep and cap proteins. In some embodiments, the cell line supplies stably the rep and cap proteins and the helper functions and is transiently transfected with the polynucleotide of the invention. In another embodiment, the cell line supplies stably the rep and cap proteins and is transfected transiently with the polynucleotide of the invention and a polynucleotide encoding the helper functions. In some embodiments, the cell line supplies stably the polynucleotide of the invention, the rep and cap proteins and the helper functions. Methods of making and using these and other AAV production systems have been described in the art. See Muzyczka N, et al., U.S. Pat. No. 5,139,941, Zhou X, et al., U.S. Pat. No. 5,741,683, Samulski R, et al., U.S. Pat. No. 6,057,152, Samulski R, et al., U.S. Pat. No. 6,204,059, Samulski R, et al., U.S. Pat. No. 6,268,213, Rabinowitz J, et al., U.S. Pat. No. 6,491,907, Zolotukhin S, et al., U.S. Pat. No. 6,660,514, Shenk T, et al., U.S. Pat. No. 6,951,753, Snyder R, et al., U.S. Pat. No. 7,094,604, Rabinowitz J, et al., U.S. Pat. No. 7,172,893, Monahan P, et al., U.S. Pat. No. 7,201,898, Samulski R, et al., U.S. Pat. No. 7,229,823, and Ferrari F, et al., U.S. Pat. No. 7,439,065, all of which are incorporated herein by reference.

The recombinant AAV (rAAV) genome present in a rAAV vector comprises at least the nucleotide sequences of the inverted terminal repeat regions (ITRs) of one of the AAV serotypes (preferably the ones of serotype AAV2 as disclosed herein), or nucleotide sequences substantially identical thereto or nucleotide sequences having at least 60%, 70%, 80%, 90%, 95% or 99% identity thereto, and a nucleotide sequence encoding a covalently linked alpha beta (αβ) dimer of beta-hexosaminidase (under control of a suitable regulatory element) inserted between the two ITRs. A vector genome generally requires the use of flanking 5′ and 3′ ITR sequences to allow for efficient packaging of the vector genome into the rAAV capsid.

The complete genome of several AAV serotypes and corresponding ITRs has been sequenced (Chiorini et al. 1999, J. of Virology Vol. 73, No. 2, p1309-1319, incorporated herein by reference). They can be either cloned or made by chemical synthesis as known in the art, using for example an oligonucleotide synthesizer as supplied e.g., by Applied Biosystems Inc. (Fosters, CA, USA) or by standard molecular biology techniques. The ITRs can be cloned from the AAV viral genome or excised from a vector comprising the AAV ITRs. The ITR nucleotide sequences can be either ligated at either end to the nucleotide sequence comprising one or more genes using standard molecular biology techniques, or the AAV sequence between the ITRs can be replaced with the desired nucleotide sequence.

Preferably, the rAAV genome as present in a rAAV vector does not comprise any nucleotide sequences encoding viral proteins, such as the rep (replication) or cap (capsid) genes of AAV. This rAAV genome may further comprise a marker or reporter gene, such as a gene for example encoding an antibiotic resistance gene, a fluorescent protein (e.g., gfp) or a gene encoding a chemically, enzymatically, or otherwise detectable and/or selectable product (e.g., lacZ, aph, etc.) known in the art.

The rAAV genome as present in said rAAV vector further comprises a promoter sequence operably linked to the nucleotide sequence encoding a covalently linked alpha beta (αβ) dimer of beta-hexosaminidase.

A suitable 3′ untranslated sequence may also be operably linked to the nucleotide sequence encoding a covalently linked alpha beta (αβ) dimer of beta-hexosaminidase. Suitable 3′ untranslated regions may be those naturally associated with the nucleotide sequence or may be derived from different genes, such as for example the SV40 polyadenylation signal (SEQ ID NO: 27).

The introduction into a producer cell can be carried out using standard virological techniques, such as transformation, transduction and transfection. Most vectors do not replicate in the producer cells infected with the vector. Examples of workable combinations of cell lines and expression vectors are described in Sambrook and Green, Molecular Cloning. A Laboratory Manual, 4th Edition (2012), Cold Spring Harbor Laboratory Press (incorporated herein by reference), and in Metzger et al (1988) Nature 334:31-36 (incorporated herein by reference). For example, suitable expression vectors can be expressed in, yeast, e.g., S. cerevisiae, e.g., insect cells, e.g., Sf9 cells, mammalian cells, e.g., CHO cells and bacterial cells, e.g., E. coli. A cell may thus be a prokaryotic or eukaryotic producer cell. A cell may be a cell that is suitable for culture in liquid or on solid media. Finally, the producer cells are cultured under standard conditions known in the art to produce the assembled AAV vectors which are then purified using standard techniques such as polyethylene glycol precipitation or CsCl gradients (Xiao et al. 1996, J. Virol. 70:8098-8108, incorporated herein by reference). Residual helper virus activity can be inactivated using known methods, such as for example heat inactivation.

The gene constructs and expression vectors as described herein may be introduced into a host cell using standard molecular techniques, as discussed in standard handbooks such as Current Protocols in Molecular Biology (Ausubel et al.), 3^{rd} edition (2003), John Wiley & Sons, Inc (US) (incorporated herein by reference) and Sambrook and Green (2012, supra). Accordingly, this disclosure further provides a host cell transduced or transfected with any of the gene constructs or expression vectors described herein. In some embodiments, a host cell transduced or transfected with any of the gene constructs or expression vectors described herein is a brain cell, preferably a brain cell of a mammal. In some embodiments, a host cell transduced or transfected with any of the gene constructs or expression vectors described herein is a murine brain cell (such as a brain cell of a rat or mouse) or a human brain cell, preferably of a mouse or a human, more preferably of a human.

In some embodiments, a host cell as described herein is an isolated host cell.

In the case of viral vectors, transduction is preferably used. The transduced host cell may or may not comprise the packaging components of the viral vectors. “Host cell” or “target cell” refers to the cell into which the DNA delivery takes place, such as the brain cells of a mammalian subject as described elsewhere herein. AAV vectors in particular are able to transduce both dividing and non-dividing cells.

### Composition

In a further aspect there is provided a composition comprising a gene construct as described herein and/or an expression vector as described herein, optionally further comprising one or more pharmaceutically acceptable ingredients. Such composition may be called a gene therapy composition. Preferably, the composition is a pharmaceutical composition.

As used herein, “pharmaceutically acceptable ingredients” include pharmaceutically acceptable carriers, fillers, preservatives, solubilizers, vehicles, diluents and/or excipients. Accordingly, the one or more pharmaceutically acceptable ingredients may be selected from the group consisting of pharmaceutically acceptable carriers, fillers, preservatives, solubilizers, vehicles, diluents, and excipients, preferably selected from the group consisting of excipients, vehicles, carriers, and diluents. Such pharmaceutically acceptable carriers, fillers, preservatives, solubilizers, vehicles, diluents and/or excipients may for instance be found in Remington: The Science and Practice of Pharmacy, 23rd edition. Elsevier (2020), incorporated herein by reference. A further compound may be present in a composition of the invention. Said compound may help in delivery of the composition. Suitable compounds in this context are: compounds capable of forming complexes, nanoparticles, micelles and/or liposomes. It is understood that these compounds are capable of delivering gene constructs and expression vectors as described herein, complexed or trapped in a vesicle or liposome, through a cell membrane. Many of these compounds are known in the art. Suitable compounds comprise polyethylenimine (PEI), or similar cationic polymers, including polypropyleneimine or polyethylenimine copolymers (PECs) and derivatives; synthetic amphiphiles (SAINT-18); lipofectin™, DOTAP. A person of skill in the art will know which type of formulation is the most appropriate for a composition as described herein.

The compositions as described herein, can be formulated in viral genome (vg) dosage units to contain an amount of viral genomes that is in the range of about 10\hat{ }9-10\hat{ }16 vg, preferably 10\hat{ }10-10\hat{ }16 vg, more preferably 10\hat{ }11-10\hat{ }15 vg, even more preferably 10\hat{ }12-10\hat{ }14 vg.

### Method and Use

Also provided herein are gene constructs, expression vectors and compositions as described herein for use in therapy. In some embodiments, gene constructs, expression vectors and compositions as described herein are for use as a medicament.

In preferred embodiments, gene constructs, expression vectors, and compositions as described herein are provided for use in the treatment of GM2 gangliosidoses. Complications of GM2 gangliosidoses may also be encompassed. GM2 gangliosidoses are a group of three neurodegenerative lysosomal storage diseases characterized by a beta-hexosaminidase deficiency. This beta-hexosaminidase enzyme is composed of two subunits, alpha and beta, in which dimer formation is required for catalytic activity. Mutations in the alpha subunit lead to Tay-Sachs disease, while mutations in the beta subunit lead to Sandhoff disease. Accordingly, in preferred embodiments, gene constructs, expression vectors, and compositions as described herein are provided for use in the treatment of Sandhoff disease or Tay-Sachs disease.

In a further aspect there is provided a method of treatment of GM2 gangliosidoses, preferably Sandhoff disease or Tay-Sachs disease, comprising administering a gene construct, an expression vector and/or a composition as described herein. In some embodiments, administering a gene construct, an expression vector or a composition means administering to a subject such as a subject in need thereof. In a preferred embodiment, a therapeutically effective amount of a gene construct, an expression vector or a composition is administered. In a further aspect there is provided a use of a gene construct, an expression vector or a composition as described herein, for the manufacture of a medicament for the treatment of GM2 gangliosidoses, preferably Sandhoff disease or Tay-Sachs disease.

In a further aspect there is provided a use of a gene construct, an expression vector or a composition as described herein, for the treatment of GM2 gangliosidoses, preferably Sandhoff disease or Tay-Sachs disease.

Within the context of gene constructs for use, expression vectors for use, compositions for use, methods and uses according to the disclosure, an effective amount or therapeutically (and/or prophylactically) effective amount may be administered.

As used herein, an “effective amount” is an amount sufficient to exert beneficial or desired results. Accordingly, a “therapeutically effective amount” is an amount that, when administered to a subject in need thereof, is sufficient to exert some therapeutic effect as described herein, such as, but not limited to, increased beta-hexosaminidase activity, normalization of GM2 in the cerebral cortex as well as cholesterol accumulation in the cortex and cerebellum, normalization of lysosomal distension, lysosomal homeostasis, autophagy, myelinization and neuroinflammation in the CNS, normalization of cholesterol and GAG storage in several peripheral tissues and normalization of lysosomal homeostasis in the liver, improved general locomotor and exploratory activity, as well as motor coordination, mobility and disease progression, and improved survival compared to an untreated subject. An amount that is “therapeutically effective” will vary from subject to subject, depending on the age, the disease progression and overall general condition of the individual. An appropriate “therapeutically effective” amount in any individual case may be determined by the skilled person using routine experimentation, such as the methods described later herein, and/or the methods of the experimental part herein. In some embodiments, the therapeutically effective amount is a vg dosage unit of 10\hat{ }9-10\hat{ }16 vg, preferably 10\hat{ }10-10\hat{ }16 vg, more preferably 10\hat{ }11-10\hat{ }15 vg, even more preferably 10\hat{ }12-10\hat{ }14 vg. It is understood that this effective amount is expressed as the total dose per subject. The human brain volume is around 1200 cm\hat{ }3. Thus, these doses correspond with about 0.83×10\hat{ }6-0.83×10\hat{ }13 vg, preferably 0.83×10\hat{ }7-0.83×10\hat{ }13 vg, more preferably 0.83×10\hat{ }8-0.83×10\hat{ }12 vg, even more preferably 0.83×10\hat{ }9-0.83×10\hat{ }11 vg when expressed per ml of brain.

Within the context of gene constructs for use, expression vectors for use, compositions for use, methods and uses according to the disclosure, the gene constructs, expression vectors and composition may be administered to a subject, such as a subject in need thereof. In some embodiments, the subject (in need) can be a healthy, asymptomatic or partially symptomatic subject. In preferred embodiments, the subject (in need) may also suffer from or be at risk for developing any of the symptoms, diseases, and conditions described herein. In some embodiments, the subject (in need) may be a subject inflicted with any of the symptoms, diseases, and conditions described herein.

Within the context of gene constructs for use, expression vectors for use, compositions for use, methods and uses according to the disclosure, the therapy and/or treatment and/or medicament may involve expression of a covalently linked alpha beta (αβ) dimer of beta-hexosaminidase in the CNS, preferably the brain, and/or transduction of the CNS, preferably the brain. In some embodiments, expression of the gene construct in the brain may mean expression of the gene construct in the hypothalamus and/or the thalamus and/or the subthalamus and/or the epithalamus and/or the cortex and/or the hippocampus and/or the basal ganglia and/or the amygdala and/or the cerebellum and/or the brain stem. Accordingly, expression of the gene construct in the brain may mean expression of the gene construct in at least one or at least two or at least three or at least four or all brain regions selected from the group consisting of the hypothalamus and/or the thalamus and/or the subthalamus and/or the epithalamus, and/or the cortex and/or the hippocampus and/or the basal ganglia and/or the amygdala and/or the cerebellum and/or the brain stem. In some embodiments, expression in the CNS and/or the brain and/or the hypothalamus and/or the thalamus and/or the subthalamus and/or the epithalamus and/or the cortex and/or the hippocampus and/or the basal ganglia and/or the amygdala and/or the cerebellum and/or the brain stem may mean specific expression in the CNS and/or the brain and/or the hypothalamus and/or the thalamus and/or the subthalamus and/or the epithalamus and/or the cortex and/or the hippocampus and/or the basal ganglia and/or the amygdala and/or the cerebellum and/or the brain stem.

As explained elsewhere herein, in some embodiments of gene constructs for use, expression vectors for use, compositions for use, methods and uses according to the disclosure, the therapy and/or treatment and/or medicament may involve expression of a covalently linked alpha beta (αβ) dimer of beta-hexosaminidase in the liver.

Within the context of gene constructs for use, expression vectors for use, compositions for use, methods and uses according to the invention, “involving the expression of a gene construct” may be replaced by “causing the expression of a gene construct” or “inducing the expression of a gene construct” or “involving transduction” or the like.

In a preferred embodiment, a treatment or a therapy or a use or the administration of a medicament as described herein does not have to be repeated. In some embodiments, a treatment or a therapy or a use or the administration of a medicament as described herein may be repeated each year or each 2, 3, 4, 5, 6, 7, 8, 9 or 10, including intervals between any two of the listed values, years.

The subject treated may be a vertebrate, preferably a mammal, such as a rodent (preferably mice, rats), or a human. In preferred embodiments, the subject treated is a human.

Within the context of gene constructs for use, expression vectors for use, compositions for use, methods and uses according to the invention, a gene construct and/or an expression vector and/or a composition and/or a medicament as described herein preferably exhibits at least one, at least two, at least three, or all of the following effects:
increase of beta-hexosaminidase activity;
restoration of lysosomal degradation of GM2 gangliosides;
alleviating a symptom of GM2 gangliosidoses, preferably Sandhoff disease or Tay-Sachs disease (as described herein); and
improving a parameter associated with GM2 gangliosidoses, preferably Sandhoff disease or Tay-Sachs disease (as described herein).

In some embodiments, a gene construct and/or an expression vector and/or a composition and/or a medicament as described herein preferably exhibits at least one, at least two, at least three, or all of the following effects:
normalization of GM2 and cholesterol accumulation;
normalization of lysosomal distension, lysosomal homeostasis, autophagy, myelinization and neuroinflammation in the CNS; and/or
normalization of cholesterol and GAG storage in several peripheral tissues and a normalization of lysosomal homeostasis in the liver; and/or
improvement of general locomotor and exploratory activity, as well as motor coordination, mobilty and disease progression; and/or
increase in survival.

Alleviating a symptom of GM2 gangliosidoses may mean that a symptom of GM2 gangliosidoses (e.g., developmental regression, neurological deterioration, motor deficits and vision deterioration) is improved or decreased or that the progression of a typical symptom has been slowed down in an individual, in a cell, tissue or organ of said individual as assessed by a physician. A decrease or improvement of a typical symptom may mean a slowdown in progression of symptom development or a complete disappearance of symptoms.

Symptoms, and thus also a decrease in symptoms, can be assessed using a variety of methods, to a large extent the same methods as used in diagnosis of GM2 gangliosidoses, including clinical examination and routine laboratory tests. Laboratory tests may include both macroscopic and microscopic methods, molecular methods, radiographic methods such as X-rays, Magnetic Resonance Imaging, biochemical methods, immunohistochemical methods and others. Beta-hexosaminidase levels and activity could be assessed using techniques known to a person of skill in the art, for example as done in the experimental part. This diagnostic could be further confirmed by a DNA-based test to identify the underlying mutation causing the GM2 gangliosidosis. In this context, “decrease” (respectively “improvement”) means at least a detectable decrease (respectively a detectable improvement) using an assay known to a person of skill in the art, such as assays as carried out in the experimental part. The decrease may be a decrease of at least 5%, at least 10%, at least 20%, at least 30%, at least 40%, at least 50%, at least 60%, at least 70%, at least 80%, at least 90% or at least 100%. The decrease may be seen after at least one week, one month, six months, one year or more of treatment using a gene construct and/or an expression vector and/or a composition of the invention. Preferably, the decrease is observed after a single administration. In some embodiments, the decrease is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Improving a parameter may mean that the value of a typical parameter associated with GM2 gangliosidoses (e.g., beta-hexosaminidase expression and/or activity) is improved in an individual or in a cell, tissue, or organ of said individual, as assessed by a physician. In this context, improvement of a parameter may be interpreted as to mean that said parameter assumes a value closer to the value displayed by a healthy individual. The improvement of a parameter may be seen after at least one week, one month, six months, one year or more of treatment using a gene construct and/or an expression vector and/or a composition of the invention. Preferably, the improvement is observed after a single administration. In some embodiments, the improvement is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

A gene construct and/or an expression vector and/or a composition as described herein is preferably able to alleviate a symptom or a parameter or a characteristic of GM2 gangliosidoses, preferably Sandhoff disease or Tay-Sachs disease, in a patient or a cell, tissue or organ of said patient if after at least one week, one month, six months, one year or more of treatment using a gene construct and/or an expression vector and/or a composition of the invention, said symptom or parameter or characteristic has decreased (e.g. is no longer detectable or has slowed down), as described herein.

A gene construct and/or an expression vector and/or a composition as described herein may be suitable for administration to a cell, tissue and/or an organ in vivo of individuals affected by or at risk of developing GM2 gangliosidoses, preferably Sandhoff disease or Tay-Sachs disease, and may be administered in vivo, ex vivo or in vitro. Said gene construct and/or expression vector and/or composition may be directly or indirectly administered to a cell, tissue and/or an organ in vivo of an individual affected by or at risk of developing GM2 gangliosidoses, preferably Sandhoff disease or Tay-Sachs disease, and may be administered directly or indirectly in vivo, ex vivo or in vitro.

Within the context of gene constructs for use, expression vectors for use, compositions for use, methods and uses according to the invention, a gene construct and/or an expression vector and/or a composition may be administered by different administration modes. An administration mode may be intravenous, intramuscular, intraperitoneal, intranasal, subcutaneous, intraarticular, intra-adipose tissue, oral, intrahepatic, intrasplanchnic, intraductal, intra-ear, intracranial, intraparenchymal, intrathecal, intracerebroventricular, intracerebral, hippocampal, striatal administration, ophthalmic administration, administration via the cerebrospinal fluid (CSF) and/or administration via the cisterna magna. A more preferred administration mode is intracranial, intraparenchymal, intrathecal, intracerebroventricular, intracerebral, hippocampal, striatal administration, administration via the cisterna magna and/or administration via the CSF. An even more preferred administration mode is intra-CSF administration, intracerebroventricular, intrathecal administration and/or administration via the cisterna magna. Accordingly, in some embodiments, a gene construct as described herein is for use in therapy, wherein the gene construct is administered by intra-CSF.

“Intra-CSF administration”, “intranasal administration”, “intraparenchymal administration” “intra-cisterna magna administration”, “intrathecal administration”, “intravenous administration”, “intramuscular administration”, “intra-adipose tissue administration”, “intraperitoneal administration”, “subcutaneous administration”, “intraarticular administration”, “intrahepatic administration”, “intrasplanchnic administration”, “intracranial administration”, “intracerebral administration”, “hippocampal administration”, “intraductal administration”, “striatal administration”, “oral administration”, “ophthalmic administration”, “intra-ear” administration, and “intracerebroventricular administration”, as used herein, are described in the part of this application entitled “general information”.

A gene construct and/or an expression vector and/or a composition of the invention may be directly or indirectly administered using suitable means known in the art. Improvements in means for providing an individual or a cell, tissue, or organ of said individual with a gene construct and/or an expression vector and/or a composition of the invention are anticipated, considering the progress that has already thus far been achieved. Such future improvements may of course be incorporated to achieve the mentioned effect of the invention. A gene construct and/or an expression vector and/or a composition can be delivered as is to an individual, a cell, tissue or organ of said individual. Depending on the disease or condition, a cell, tissue or organ of said individual may be as earlier described herein. When administering a gene construct and/or an expression vector and/or a composition of the invention, it is preferred that such gene construct and/or an expression vector and/or a composition is dissolved in a solution that is compatible with the delivery method.

As encompassed herein, a therapeutically effective dose of a gene construct and/or an expression vector and/or a composition as mentioned above is preferably administered in a single and unique dose, hence avoiding repeated periodical administration.

“Treating” or “treatment” as used herein may include delaying (e.g., delaying progression or delaying deterioration), preventing, ameliorating, or curing. Accordingly, throughout this disclosure, “treating” and the like may be replaced with “treating, delaying, ameliorating or curing” and the like.

Within the context of compositions for use, methods and uses provided herein, “treating” as used herein may be understood to treat a disease, condition or symptom as compared to said disease, condition, or symptom prior to administering the composition. In some embodiments, “treating” as used herein may be understood to treat a disease, condition or symptom as compared to said disease, condition or symptom in a subject inflicted with the same disease, condition or symptom receiving a placebo. Similarly, the occurrence of any of the effects described elsewhere herein may be compared in the same way.

### General Information

Unless stated otherwise, all technical and scientific terms used herein have the same meaning as customarily and ordinarily understood by a person of ordinary skill in the art to which this invention belongs and read in view of this disclosure.

### Beta-Hexosaminidase

Beta-hexosaminidase, also named beta-N-acetylhexosaminidase, (EC 3.2.1.52) is a lysosomal enzyme composed of dimers of alpha- and/or beta-subunits. The alpha and beta subunits are synthesized in the rough endoplasmic reticulum and transported through the Golgi apparatus to the lysosome. Thus, in order to target each monomer to the lysosomal compartment, each subunit contains an amino-terminal signal peptide that is subsequently cleaved by signal peptidase. Beta-hexosaminidase catalyzes the hydrolysis of terminal N-acetyl-D-hexosamine residues in N-acetyl-β-D-hexosaminides. Means and methods to measure this activity are commonly known in the art, for example as described in the Examples section. As used herein and unless explicitly stated otherwise, “beta-N-acetylhexosaminidase”, and “beta-hexosaminidase” and the like refer to the mammalian enzyme beta-hexosaminidase. As stated elsewhere herein, three isoforms of the beta-hexosaminidase exist. The hexosaminidase A (HexA) is composed of an alpha and a beta subunit, the hexosaminidase B (HexB) is composed of two beta subunits, and the hexosaminidase S (HexS) is composed of two alpha subunits. In humans, the alpha and beta subunits are encoded by the HEXA and HEXB genes, respectively, and in mice the alpha and beta subunits are encoded by the Hexa and Hexb genes, respectively, as described in more detail elsewhere herein. Given that all three isoforms have enzymatic activity, the total beta-hexosoaminidase activity in a cell or tissue is determined by the sum of the activities of all three isoforms. Accordingly, as used herein and unless explicitly stated otherwise, “total beta-hexosaminidase activity” and “beta-HEXO” and the like refer to the activity of HexA, HexB, and HexS summed together. Unless explicitly stated otherwise, the terms “beta-hexosaminidase A”, “HexA”, and “beta-hexosaminidase isoform A” and the like as used herein encompasses full-length molecules, variants, isoforms, and fragments that retain enzymatic activity against HexA substrates, such as GM2 ganglioside. The term also encompasses natural and engineered molecules identical or substantially identical to these sequences. The term “HexA expression constructs” or “HexA expression vectors” refers to constructs encoding both the alpha and beta subunits of beta-hexosaminidase. As mentioned elsewhere herein, most enzymatic assays or other laboratory test used for quantifying total HexA activity also measure residual HexS activity. Accordingly, as used herein and unless explicitly stated otherwise, the terms “beta-hexosaminidase A activity” and “HexA activity” refers to the activity of HexA and HexS summed together.

### Beta-Hexosaminidase Deficiency

As used herein, the term “beta-hexosaminidase deficiency” or “hexosaminidase deficiency” or the like refers to reduced expression or function of hexosaminidase compared to normal levels for sex and age matched subjects. Deficiencies may be the result of genetic mutations or other molecular events that impair transcription, translation, post-translational modification, sub-cellular localization, dimerization, or enzymatic function of the hexosaminidase alpha and beta subunits. The severity of hexosaminidase deficiency may vary across subjects and may or may not result in clinical symptoms associated with lysosomal storage disorders.

### Lysosomal Storage Disease

The term “lysosomal storage disease” as used herein refers to a group of diseases that are caused by a lack of enzymes that normally serve as catalyst for the breakdown of substances in the cells of the body. These enzymes are found in sac-like structures in cells called lysosomes. Lysosomes act as the “recycling center” of the cell, breaking down molecules into simple products for the cell to use to build new material. The lack of certain enzymes causes an accumulation within the cell of the substance that the enzyme would normally help eliminate. Abnormal storage causes inefficient functioning and damage of the body's cells, which can lead to serious health problems.

### Self-Cleaving Peptide

The term “self-cleaving peptide” as used herein refers to a peptide sequence that is associated with a cleavage activity that occurs between two amino acid residues within the peptide sequence itself. For example, in P2A peptides, cleavage occurs between the proline (P) and glycine (G) in the C-terminal of the peptide resulting in the peptide located upstream of the 2A peptide to have extra amino acids on its C-terminal end while the peptide located downstream the 2A peptide will have an extra P on its N-terminal end. This cleavage occurs through a ‘ribosomal skip mechanism’ during translation wherein normal peptide bond formation between the P and G residue is impaired, without affecting the translation of the rest of the peptide. Such ribosomal skip mechanisms are well known in the art and are known to be used by several viruses for the expression of several proteins encoded by a single messenger RNA.

### CNS and Brain

As used herein, “central nervous system” or “CNS” refers to the part of the nervous system that comprises the brain and the spinal cord, to which sensory impulses are transmitted and from which motor impulses pass out, and which coordinates the activity of the entire nervous system.

As used herein, “brain” refers to the central organ of the nervous system and consists of the cerebrum, the brain stem, and the cerebellum. It controls most of the activities of the body, processing, integrating, and coordinating the information it receives from the sense organs, and making decisions as to the instructions sent to the rest of the body.

In particular, as used herein, ‘hypothalamus” refers to a region of the forebrain below the thalamus which coordinates both the autonomic nervous system and the activity of the pituitary, controlling body temperature, thirst, hunger, and other homeostatic systems, and involved in sleep and emotional activity. “Hippocampus”, as used herein, belongs to the limbic system, and plays important roles in the consolidation of information from short-term memory to long-term memory, and in spatial memory that enables navigation. The hippocampus is located under the cerebral cortex (allocortical) and in primates in the medial temporal lobe. The “cortex” or “cerebral cortex”, as used herein, is the outer layer of neural tissue of the cerebrum of the brain, in humans and other mammals. It plays a key role in memory, attention, perception, awareness, thought, language, and consciousness. It includes the frontal cortex, the parietal cortex, the temporal cortex, and the occipital cortex. “Cerebellum”, as used herein, refers to a major feature in the hindbrain of all vertebrates. In humans, it plays an important role in motor control. It may also be involved in some cognitive functions such as attention and language as well as in regulating fear and pleasure responses. “Thalamus” as used herein, refers to a walnut-sized structure located in the forebrain of all vertebrates. It functions as a relay station of all incoming motor and sensory information. “Subthalamus” as used herein, is a structure located between the thalamus and the midbrain. It contains the subthalamic nucleus and functions in the regulation of movements controlled by skeletal muscles. “Epithalamus” as used herein, refers to a small structure which is located behind the third ventricle and dorsal and caudal to the thalamus. It acts as a connection between the limbic system and other parts of the brain. The basal ganglia as used herein, refer to a collection of subcortical nuclei, such as the striatum, globus pallidus, ventral pallidum, substantia nigra and subthalamic nucleus, in the brains of all vertebrates. These ganglia are primarily responsible for motor control, as well as other roles such as motor learning, executive functions and behaviors, and emotions. The “amygdala” as used herein, is a complex structure of cells nested in the middle of the brain, adjacent to the hippocampus. It plays a key role in the fight-or-flight response, emotion, and memory. The “brain stem” as used herein, refers to a stalk-like portion of the brain which connects the brain to the spinal cord. It includes the midbrain, pons, and medulla oblongata, and mainly controls subconscious body functions, such as breathing and maintaining the heart rate.

### Sequence Identity

In the context of the invention, a nucleic acid molecule such as a nucleic acid molecule encoding a covalently linked alpha beta (αβ) dimer of beta-hexosaminidase, is represented by a nucleic acid or nucleotide sequence which encodes a protein fragment or a polypeptide or a peptide or a derived peptide. In the context of the invention, a covalently linked alpha beta (αβ) dimer of beta-hexosaminidase protein fragment or a polypeptide or a peptide or a derived peptide are represented by an amino acid sequence.

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60%, 70%, 80%, 90%, 95% or 99% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 60%, 70%, 80%, 90%, 95% or 99% amino acid identity or similarity with an amino acid sequence encoded by a nucleotide sequence SEQ ID NO: X.

Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide represented by an amino acid sequence comprising a sequence that has at least 60%, 70%, 80%, 90%, 95% or 99% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%.

Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

Each non-coding nucleotide sequence (i.e., of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: A. In a preferred embodiment, such non-coding nucleotide sequence such as a promoter exhibits or exerts at least an activity of such a non-coding nucleotide sequence such as an activity of a promoter as known to a person of skill in the art.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g., Needleman-Wunsch) which aligns the sequences optimally over the entire length, while sequences of substantially different lengths are preferably aligned using a local alignment algorithm (e.g., Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference).

Alternatively, percentage similarity or identity may be determined by searching against public databases, using algorithms such as FASTA, BLAST, etc. Thus, the nucleic acid and protein sequences of some embodiments of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged | Ser (S), Thr (T), Asn (N), and |
| Residues | Gln (Q) |
| Aliphatic Uncharged | Gly (G), Ala (A), Val (V), Leu (L), |
| Residues | and Ile (I) |
| Non-polar Uncharged | Cys (C), Met (M), and Pro (P) |
| Residues | |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, |
| | R, T, V, W, and Y |
| Negatively charged residues | D and E |
| Polar residues | C, D, E, H, K, N, Q, R, |
| | S, and T |
| Positively charged residues | H, K, and R |
| Small residues | A, C, D, G, N, P, S, T, |
| | and V |
| Very small residues | A, G, and S |
| Residues involved in turn | A, C, D, E, G, H, K, N, |
| formation | Q, R, S, P and T |
| Flexible residues | Q, T, K, S, G, P, D, E, |
| | and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; IIe to Leu or Val; Leu to IIe or Val; Lys to Arg, Gln or Glu; Met to Leu or IIe; Phe to Met, Leu or Tyr; Ser to Thr; Thr to Ser; Trp to Tyr; Tyr to Trp or Phe; and, Val to IIe or Leu.

### Gene or Coding Sequence

The term “gene” means a DNA fragment comprising a region (transcribed region), which is transcribed into an RNA molecule (e.g., an mRNA) in a cell, operably linked to suitable regulatory regions (e.g., a promoter). A gene will usually comprise several operably linked fragments, such as a promoter, a 5′ leader sequence, a coding region and a 3′-nontranslated sequence (3′-end) e.g., comprising a polyadenylation- and/or transcription termination site. The coding region of a gene, also known as the coding sequence (CDS), is the portion of a gene that codes for protein. A chimeric or recombinant gene (such as an HEXA gene) is a gene not normally found in nature, such as a gene in which for example the promoter is not associated in nature with part or all of the transcribed DNA region. “Expression of a gene” refers to the process wherein a DNA region which is operably linked to appropriate regulatory regions, particularly a promoter, is transcribed into an RNA, which is biologically active, i.e., which is capable of being translated into a biologically active protein or peptide.

A “transgene” is herein described as a gene or a coding sequence or a nucleic acid molecule (i.e., a molecule encoding a covalently linked alpha beta (αβ) dimer of beta-hexosaminidase) that has been newly introduced into a cell, i.e., a gene that may be present but may normally not be expressed or expressed at an insufficient level in a cell. In this context, “insufficient” means that although said beta-hexosaminidase dimer is expressed in a cell, a condition and/or disease as described herein could still be developed. In this case, the invention allows the over-expression of a beta-hexosaminidase. The transgene may comprise sequences that are native to the cell, sequences that naturally do not occur in the cell and it may comprise combinations of both. A transgene may contain sequences coding for a beta-hexosaminidase and/or additional proteins as earlier identified herein that may be operably linked to appropriate regulatory sequences for expression of the sequences coding for a beta-hexosaminidase in the cell. Preferably, the transgene is not integrated into the host cell's genome.

### Promoter

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions.

A “ubiquitous promoter” is active in substantially all tissues, organs, and cells of an organism.

Assessment of the ubiquitous or tissue-specific nature of a promoter can be performed by standard molecular toolbox techniques, such as, for example, described in Sambrook and Green (supra). As a non-limiting example, any expression vector comprising any of the gene construct as described herein, wherein the covalently linked alpha beta (αβ) dimer of beta-hexosaminidase nucleotide sequence has been replaced by a nucleotide sequence encoding for GFP, can be produced. Cells transduced as described herein can then be assessed for fluorescence intensity according to standard protocols.

Promoters that are capable of initiating transcription in brain cells, whilst still allowing for any leaky expression in other (maximum five, six, seven or eight) organs and parts of the body, are advantageous.

As used herein, a “regulator” or “transcriptional regulator” is a protein that controls the rate of transcription of genetic information from DNA to messenger RNA, by binding to a specific DNA sequence.

Expression may be assessed as described elsewhere under the section entitled “general information”.

### Operably Linked

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame. Linking can be accomplished by ligation at convenient restriction sites or at adapters or linkers inserted in lieu thereof, or by gene synthesis. In some embodiments, all of the nucleotide sequences described in this document may be operably linked with each other within the gene construct of this disclosure.

### Proteins and Amino Acids

The terms “protein” or “polypeptide” or “amino acid sequence” are used interchangeably and refer to molecules consisting of a chain of amino acids, without reference to a specific mode of action, size, 3-dimensional structure, or origin. In amino acid sequences as described herein, amino acids or “residues” are denoted by three-letter symbols. These three-letter symbols as well as the corresponding one-letter symbols are well known to a person of skill in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (IIe) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid.

### Gene Constructs

Gene constructs as described herein could be prepared using any cloning and/or recombinant DNA techniques, as known to a person of skill in the art, in which a nucleotide sequences encoding said covalently linked alpha beta (αβ) dimer of beta-hexosaminidase are expressed in a suitable cell, e.g. cultured cells or cells of a multicellular organism, such as described in Ausubel et al., “Current Protocols in Molecular Biology”, (2003, supra) and in Sambrook and Green (2012, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34:315 (describing cassette mutagenesis).

### Expression Vectors

The phrase “expression vector” or “vector” or “delivery vector” generally refers to a tool in molecular biology used to obtain gene expression in a cell, for example by introducing a nucleotide sequence that is capable of effecting expression of a gene or a coding sequence in a host compatible with such sequences. An expression vector carries a genome that is able to stabilize and remain episomal in a cell. Within the context of the invention, a cell may mean to encompass a cell used to make the construct or a cell wherein the construct will be administered. Alternatively, a vector is capable of integrating into a cell's genome, for example through homologous recombination or otherwise.

These expression vectors typically include at least suitable promoter sequences and optionally, transcription termination signals. An additional factor necessary or helpful in effecting expression can also be used as described herein. A nucleic acid or DNA or nucleotide sequences encoding a covalently linked alpha beta (αβ) dimer of beta-hexosaminidase is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, (e.g., Sf9), yeast, fungi, or other eukaryotic cell lines.

A DNA construct prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been described herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art (see, e.g., Sambrook and Green, 2012, supra). A transcriptional regulatory sequence typically includes a heterologous enhancer or promoter that is recognized by the host. The selection of an appropriate promoter depends upon the host, but promoters such as the trp, lac and phage promoters, tRNA promoters and glycolytic enzyme promoters are known and available (see, e.g., Sambrook and Green, 2012, supra). An expression vector includes the replication system and transcriptional and translational regulatory sequences together with the insertion site for the polypeptide encoding segment. In most cases, the replication system is only functional in the cell that is used to make the vector (bacterial cell as E. Coli). Most plasmids and vectors do not replicate in the cells infected with the vector. Examples of workable combinations of cell lines and expression vectors are described in Sambrook and Green (2012, supra) and in Metzger et al. (1988) Nature 334:31-36. For example, suitable expression vectors can be expressed in, yeast, e.g., S. Cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media.

Alternatively, a host cell is a cell that is part of a multicellular organism such as a transgenic plant or animal.

### Viral Vector

A viral vector or a viral expression vector or a viral gene therapy vector is a vector that comprises a gene construct as described herein.

A viral vector or viral expression vector or a viral gene therapy vector is a vector that is suitable for gene therapy. Vectors that are suitable for gene therapy are described in Anderson 1998, Nature 392:25-30; Walther and Stein, 2000, Drugs 60:249-71; Kay et al., 2001, Nat. Med. 7:33-40; Russell, 2000, J. Gen. Virol. 81:2573-604; Amado and Chen, 1999, Science 285:674-6; Federico, 1999, Curr. Opin. Biotechnol. 10:448-53; Vigna and Naldini, 2000, J. Gene Med. 2:308-16; Marin et al., 1997, Mol. Med. Today 3:396-403; Peng and Russell, 1999, Curr. Opin. Biotechnol. 10:454-7; Sommerfelt, 1999, J. Gen. Virol. 80:3049-64; Reiser, 2000, Gene Ther. 7:910-3; and references cited therein; all of which are incorporated herein by reference.

A particularly suitable gene therapy vector includes an adenoviral and adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications, (Russell, 2000, J. Gen. Virol. 81:2573-2604; Goncalves, 2005, Virol J. 2(1):43; incorporated herein by reference) as indicated above. AAV vectors are even more preferred since they are known to result in very stable long-term expression of transgene expression (up to 9 years in dog (Niemeyer et al, Blood. 2009 Jan. 22; 113(4):797-806) and ~10 years in human (Buchlis, G. et al., Blood. 2012 Mar. 29; 119(13):3038-41)). Preferred adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11):1049-55, Nathwani et al, N Engl J Med. 2011 Dec. 22; 365(25):2357-65, Apparailly et al, Hum Gene Ther. 2005 April; 16(4):426-34; all of which are incorporated herein by reference.

Another suitable gene therapy vector includes a retroviral vector. A preferred retroviral vector for application in the present invention is a lentiviral based expression construct. Lentiviral vectors have the ability to infect and to stably integrate into the genome of dividing and non-dividing cells (Amado and Chen, 1999 Science 285:674-6, incorporated herein by reference). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10:448-53) and Vigna et al. (2000, J Gene Med 2000; 2:308-16); all of which are incorporated herein by reference.

Other suitable gene therapy vectors include an adenovirus vector, a herpes virus vector, a polyoma virus vector or a vaccinia virus vector.

### Adeno-Associated Virus Vector (AAV Vector)

The terms “adeno associated virus”, “adeno-associated virus vector”, “AAV vector”, “AAV virus”, “AAV virion”, “AAV viral particle” and “AAV particle”, used as synonyms herein, refer to a viral particle composed of at least one capsid protein of AAV (preferably composed of all capsid protein of a particular AAV serotype) and an encapsulated polynucleotide of the AAV genome. If the particle comprises a heterologous polynucleotide (i.e., a polynucleotide different from a wild-type AAV genome, such as a transgene to be delivered to a mammalian cell) flanked by AAV inverted terminal repeats, then they are typically known as an “AAV vector particle” or “AAV viral vector” or “AAV vector”. AAV refers to a virus that belongs to the genus Dependovirus family Parvoviridae. The AAV genome is approximately 4.7 Kb in length, and it consists of single strand deoxyribonucleic acid (ssDNA) that can be positive or negative detected. The invention also encompasses the use of double stranded AAV also called dsAAV or scAAV. The genome includes inverted terminal repeats (ITR) at both ends of the DNA strand, and two open reading frames (ORFs): rep and cap. The frame rep is made of four overlapping genes that encode proteins Rep necessary for AAV lifecycle. The frame cap contains nucleotide sequences overlapping with capsid proteins: VP1, VP2 and VP3, which interact to form a capsid of icosahedral symmetry (see Carter and Samulski, 2000, and Gao et al, 2004, incorporated herein by reference).

A preferred viral vector or a preferred gene therapy vector is an AAV vector. An AAV vector as used herein preferably comprises a recombinant AAV vector (rAAV vector). A “rAAV vector” as used herein refers to a recombinant vector comprising part of an AAV genome encapsidated in a protein shell of capsid protein derived from an AAV serotype as explained herein. Part of an AAV genome may contain the inverted terminal repeats (ITR) derived from an adeno-associated virus serotype, such as AAV1, AAV2, AAV3, AAV4, AAV5 and others.

Preferred ITRs are those of AAV2 which are represented by sequences comprising, consisting essentially of, or consisting of SEQ ID NO: 25 (5′ ITR) and SEQ ID NO: 26 (3′ ITR). The invention also preferably encompasses the use of a sequence having at least 80% (or at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with SEQ ID NO: 25 as 5′ ITR and a sequence having at least 80% (or at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with SEQ ID NO: 26 as 3′ ITR.

Protein shell comprised of capsid protein may be derived from any AAV serotype. A protein shell may also be named a capsid protein shell. rAAV vector may have one or preferably all wild type AAV genes deleted but may still comprise functional ITR nucleic acid sequences. Functional ITR sequences are necessary for the replication, rescue, and packaging of AAV virions. The ITR sequences may be wild type sequences or may have at least 80%, at least 85%, at least 90%, at least 95%, at least 97%, at least 98%, at least 99% or 100% sequence identity with wild type sequences or may be altered for example by insertion, mutation, deletion, or substitution of nucleotides, as long as they remain functional. In this context, functionality refers to the ability to direct packaging of the genome into the capsid shell and then allow for expression in the host cell to be infected or target cell. In the context of the present invention a capsid protein shell may be of a different serotype than the rAAV vector genome ITR.

A nucleic acid molecule represented by a nucleic acid sequence of choice is preferably inserted between the rAAV genome or ITR sequences as identified above, for example an expression construct comprising an expression regulatory element operably linked to a coding sequence and a 3′ termination sequence. Said nucleic acid molecule may also be called a transgene.

“AAV helper functions” generally refers to the corresponding AAV functions required for rAAV replication and packaging supplied to the rAAV vector in trans. AAV helper functions complement the AAV functions which are missing in the rAAV vector, but they lack AAV ITRs (which are provided by the rAAV vector genome). AAV helper functions include the two major ORFs of AAV, namely the rep coding region and the cap coding region or functional substantially identical sequences thereof. Rep and Cap regions are well known in the art, see e.g., Chiorini et al. (1999, J. of Virology, Vol 73 (2): 1309-1319) or U.S. Pat. No. 5,139,941, incorporated herein by reference. The AAV helper functions can be supplied on an AAV helper construct. Introduction of the helper construct into the host cell can occur e.g., by transformation, transfection, or transduction prior to or concurrently with the introduction of the rAAV genome present in the rAAV vector as identified herein. The AAV helper constructs of the invention may thus be chosen such that they produce the desired combination of serotypes for the rAAV vector's capsid protein shell on the one hand and for the rAAV genome present in said rAAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via plasmids, as described in U.S. Pat. No. 6,531,456 incorporated herein by reference.

“Transduction” refers to the delivery of a covalently linked alpha beta (αβ) dimer of beta-hexosaminidase into a recipient host cell by a viral vector. For example, transduction of a target cell by a rAAV vector of the invention leads to transfer of the rAAV genome contained in that vector into the transduced cell. “Host cell” or “target cell” refers to the cell into which the DNA delivery takes place, such as the muscle cells of a subject. AAV vectors are able to transduce both dividing and non-dividing cells.

### Expression

Expression may be assessed by any method known to a person of skill in the art. For example, expression may be assessed by measuring the levels of transgene expression in the transduced tissue on the level of the mRNA or the protein by standard assays known to a person of skill in the art, such as qPCR, RNA sequencing, Northern blot analysis, Western blot analysis, mass spectrometry analysis of protein-derived peptides or ELISA.

Expression may be assessed at any time after administration of the gene construct, expression vector or composition as described herein. In some embodiments herein, expression may be assessed after 1 week, 2 weeks, 3 weeks, 4, weeks, 5 weeks, 6 weeks, 7 weeks, 8 weeks, 9, weeks, 10 weeks, 11 weeks, 12 weeks, 14 weeks, 16 weeks, 18 weeks, 20 weeks, 22 weeks, 24 weeks, 28 weeks, 32 weeks, 36 weeks, 40 weeks, or more.

In the context of the invention, CNS- and/or brain-specific expression refers to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of a covalently linked alpha beta (αβ) dimer of beta-hexosaminidase, in the CNS and/or brain as compared to other organs or tissues. Other organs or tissues may be the liver, adipose tissue, skeletal muscle, pancreas, heart, kidney, colon, hematopoietic tissue, lung, ovary, spleen, stomach, testis, and others.

### Sequence Optimization

“Sequence optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. An example of sequence optimization is codon optimization. Codon optimization includes, but is not limited to, processes including selecting codons for the coding sequence to suit the codon preference of the expression host organism. For example, to suit the codon preference of mammalians, preferably of murine, canine, or human expression hosts. Optimization, such as codon optimization, can also eliminate elements that potentially impact negatively RNA stability and/or translation (e. g. termination sequences, TATA boxes, splice sites, ribosomal entry sites, repetitive and/or GC rich sequences and RNA secondary structures or instability motifs). In some embodiments, optimized sequences show at least 3%, 5%, 10%, 15%, 20%, 25%, 30%, 40%, 50%, 60%, 70%, 80%, 90%, 100% or more increase in gene expression, transcription, RNA stability and/or translation compared to the original, not codon-optimized sequence.

### Administration

As used herein, “intra-CSF administration” means direct administration into the CSF, located in the subarachnoid space between the arachnoid and pia mater layers of the meninges surrounding the brain. Intra-CSF administration can be performed via intra-cisterna magna, intracerebroventricular or intrathecal administration. As used herein, “intra-cisterna magna administration” means administration into the cisterna magna, an opening of the subarachnoid space located between the cerebellum and the dorsal surface of the medulla oblongata. As used herein, “intracerebroventricular administration” means administration into the either of both lateral ventricles of the brain. As used herein, “intrathecal administration” involves the direct administration into the CSF within the intrathecal space of the spinal column. As used herein, “intraparenchymal administration” means local administration directly into any region of the brain parenchyma. As used herein, “intranasal administration” means administration by way of the nasal structures. As used herein, “intravenous administration” refers to direct administration into a vein, typically by injection. As used herein, “intramuscular administration” means direct administration a muscle. As used herein, “intra-adipose tissue administration” involves direct administration into adipose tissue. As used herein, “intraperitoneal administration” means administration into the peritoneum (or body cavity). As used herein, “subcutaneous administration” involves administration in the adipose tissue below the skin. As used herein, “intraarticular administration” refers to direct administration into a joint. As used herein, “intrahepatic administration” involves the direct administration to the liver, predominantly via a hepatic vein or artery. As used herein, “intrasplanchnic administration” means administration to the splanchninc administration. As used herein, “intracranial administration” refers to administration into the skull. Intracranial administration, therefore, also encompasses the administration to any brain region which is accessible after penetration of the skull. For example, intracranial administration also includes intracerebral administration. As used herein “striatal administration” involves direct administration into the striatum or corpus striatum. As used herein, “ophthalmic administration” refers to direct administration to the eyes. As used herein, “intracerebral administration”, means direct administration into the cerebrum. As used herein, “hippocampal administration” involves administration into the hippocampus. As used herein “oral administration” refers to a route of administration where a substance is taken through the mouth. As used herein “intra-ear administration” involves direct administration into the ear. As used herein, “intraductal administration” refers to administration within the duct of a gland.

In a preferred embodiment, gene constructs, expression vectors and compositions according to the invention are administered as a single dose.

### Effective Amount

As used herein, the term “effective amount” or “pharmaceutically effective amount” or “therapeutically effective amount” or “prophylactically effective amount” of a composition, is a quantity sufficient to achieve or maintain a desired therapeutic and/or prophylactic effect, e.g., an amount which results in the prevention of, or a decrease in, the symptoms associated with a disease that is being treated, e.g., a GM2 gangliosidosis. The amount of a composition of the invention administered to the subject will depend on the type and severity of the disease and on the characteristics of the individual, such as general health, age, sex, body weight and tolerance to drugs. It will also depend on the degree, severity, and type of disease. The skilled artisan will be able to determine appropriate dosages depending on these and other factors. In the context of treating a lysosomal storage disorder, in some embodiments, an effective amount is the amount sufficient to cause a decrease in the severity of symptoms associated with the disorder. In the context of prophylactic administrations, in some embodiments, an effective amount is the amount sufficient to delay the onset of or decrease the likelihood of onset of GM2 gangliosidoses.

In this document and in its claims, the verb “to comprise” and its conjugations is used in its non-limiting sense to mean that items following the word are included or contained, but items not specifically mentioned are not excluded. Thus, the terms ‘comprising’, ‘comprises’, ‘comprised of’ and the like as used herein are synonymous with ‘including’, ‘includes’ or ‘containing’, ‘contains’, and are inclusive or open-ended and do not exclude additional, non-recited members, elements, or method steps.

In addition, the verb “to consist of” may be replaced by “to consist essentially of” meaning that a composition as described herein may comprise additional component(s) than the ones specifically identified, said additional component(s) not altering the unique characteristic of the invention. In addition, the verb “to consist of” may be replaced by “to consist essentially of” meaning that a method as described herein may comprise additional step(s) than the ones specifically identified, said additional step(s) not altering the unique characteristic of the invention.

Throughout this disclosure, the term “comprising” may be replaced with the term “consisting essentially of” or with the term “consisting of”.

As used herein, the singular forms ‘a’, ‘an’, and ‘the’ include both singular and plural referents unless the context clearly dictates otherwise; for example, “a gene construct” is understood to represent one or more gene constructs. As such, the terms “a” (or “an”), “one or more,” and “at least one” can be used interchangeably herein.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments described herein are capable of operation in other sequences than described or illustrated herein. The word “about” or “approximately” when used in association with a numerical value (e.g. about 10) preferably means that the value may be the given value (of 10) more or less 10%, preferably 5%, more preferably 1% of the value.

As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated. Titles, subtitles, headings, and the likes are used herein solely for ease of reading and are not intended to limit or restrict the disclosure in any way.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1. Design of the AAV constructs. The AAV constructs used in the present patent application were Null, L1 AB mouse (SEQ ID NO: 75), L2 AB mouse (SEQ ID NO: 76), L3 AB mouse (SEQ ID NO: 77), L1 BA mouse (SEQ ID NO: 78), L2 BA mouse (SEQ ID NO: 79), L3 BA mouse (SEQ ID NO: 80), Hexa+Hexb (SEQ ID NOs: 85-86), P2A AB mouse (SEQ ID NO: 87), L1 AB human noh (SEQ ID NO: 81), L1 AB human GA (SEQ ID NO: 82), L1 AB human IDT (SEQ ID NO:83), L1 AB human NV (SEQ ID NO:84), and P2A BA mouse (SEQ ID NO: 88) under the control of the ubiquitous promoter Cbh (a short version of the widely used CAG promoter composed of the cytomegalovirus (CMV) early enhancer, chicken beta-actin and a hybrid intron) (SEQ ID NO: 24). All expression cassettes also included the polyA sequence from SV40 (SEQ ID NO: 27). (A) Null construct is a noncoding plasmid carrying the Cbh promoter and the SV40 polyA sequence, but no transgene. (B) L1 AB, L2 AB and L3 AB are three different mouse constructs encoding first the optimized murine Hexa and then the optimized murine Hexb coding-sequence fused with a short linker: L1, L2 or L3. The human L1 AB noh, L1 AB GA, L1 AB IDT, and L1 1B NV constructs have the same design as their corresponding mouse constructs, except that they comprise the non-optimized or optimized human HEXA and HEXB coding sequences. (C) L1 BA, L2 BA and L3 BA are three different mouse constructs encoding first the optimized murine Hexb and then the optimized murine Hexa coding-sequence fused with a short linker: L1, L2 or L3. (D) Hexa+Hexb constructs are two mouse constructs, one of them consists of the optimized murine Hexa coding-sequence while the other consists of the optimized murine Hexb coding-sequence. (E) P2A AB construct is a mouse construct composed of the optimized murine Hexa and Hexb coding-sequence fused with the self-cleaving linker P2A.

FIG. 2. HEK293 cell transfection of plasmids encoding both Hexa and Hexb fused with three different short linkers. HEK293 cells were transfected with 0.8 μg of plasmid encoding the following mouse constructs: L1 AB mouse, L2 AB mouse, L3 AB mouse, L1 BA mouse, L2 BA mouse, and L3 BA mouse (SEQ ID NOs: 75-80). Histograms depict total beta-hexosaminidase activity (i.e., activity of HexA, HexB and HexS summed together; marked on the graphs as beta-HEXO) in cells (A) and medium (B) and HexA activity (i.e., activity of HexA and HexS summed together) in cells (C) and medium (D) measured 48 h after transfection. Total beta-hexosaminidase and HexA activities of non-transfected (NT) cells were set to 100%. Results are expressed as mean±SEM. n=3 wells/group. ***P<0.001 vs. NT.

FIG. 3. Increased HexA activity in the CNS after intra-CSF gene transfer of AAV9 vectors encoding both Hexa and Hexb genes in Sandhoff mice. Determination of HexA activity in different brain sections (I-V) of 4-month-old male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes fused with a short linker (L1 AB mouse, L2 AB mouse, and L3 AB mouse) into the CSF at 1 month of age. WT HexA activity was set to 100%. Results are expressed as mean±SEM. n=5-6 animals/group. *P<0.05, **P<0.01, and ***P<0.001 vs. Null.

FIG. 4. GM2 accumulation in the CNS following an intra-CSF delivery of AAV9 vectors encoding both Hexa and Hexb genes in Sandhoff mice. Representative images of an immunohistochemistry against GM2 in brain sections from 4-month-old male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes fused with a short linker (L1 AB mouse and L3 AB mouse) into the CSF at 1 month of age. n=5 animals/group. Scale bars, 25 μm.

FIG. 5. Correction of secondary lipid storage in the CNS following an intra-CSF delivery of AAV9 vectors encoding both Hexa and Hexb genes in Sandhoff mice. Representative images of filipin staining to detect unesterified cholesterol in cerebral cortex and cerebellum from 4-month-old male wild-type (healthy) mice, untreated Sandhoff mice and Sandhoff mice that received a total dose of 1×10\hat{ }11 vg/mouse of AAV9 vectors encoding both Hexa and Hexb genes fused with a short linker (L1 AB mouse, L2 AB mouse, and L3 AB mouse) into the CSF at 1 month of age. n=3 animals/group. Scale bars, 25 μm.

FIG. 6. Normalization of CNS lysosomal compartment size after an intra-CSF gene transfer of AAV9 vectors encoding both Hexa and Hexb genes in Sandhoff mice. Evaluation of the size of lysosomal compartment by LIMP2 immunostaining in different brain regions of 4-month-old male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes fused with a short linker (L1 AB mouse and L3 AB mouse) into the CSF at 1 month of age. Histograms depict the corresponding LIMP2+ signal quantification for each brain region in each cohort. Results are expressed as mean±SEM. n=5 animals/group. **P<0.01 and ***P<0.001 vs. Null.

FIG. 7. Restoration of lysosomal homeostasis in the CNS following an intra-CSF delivery of AAV9 vectors encoding both Hexa and Hexb genes in Sandhoff mice. Percentage of WT activity of several lysosomal enzymes analyzed in brain extracts from 4-month-old male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes fused with a short linker (L1 AB mouse, L2 AB mouse, and L3 AB mouse) into the CSF at 1 month of age. Restoration of altered activities of alphaN-acetylgalactosamine-6 sulfatase (GALNS), beta-glucuronidase (GUSB), heparan-alpha-glucosaminide N-acetyltransferase (HGSNAT), alpha-N-acetylglucosaminidase (NAGLU) Id N-sulphoglucosamine sulphohydrolase (SGSH) in all treated Sandhoff mice. Results are expressed as mean±SEM. n=4-6 animals/group. **P<0.01 and ***P<0.001 vs. Null.

FIG. 8. Normalization of autophagic flux in the CNS after an intra-CSF delivery of AAV9 vectors encoding both Hexa and Hexb genes in Sandhoff mice. Analysis of autophagic flux in the CNS of 4-month-old male wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes fused with a short linker (L1 AB mouse and L3 AB mouse) into the CSF at 1 month of age. Histograms depict the signal quantification of LC3BII/LC3 BI ratio of two independent Western blot membranes. Results are expressed as mean±SEM. n=4-5 animals/group. **P<0.01 and ***P<0.001 vs. Null.

FIG. 9. Recovery of myelinization process in the CNS after an intra-CSF delivery of AAV9 vectors encoding both Hexa and Hexb genes in Sandhoff mice. Assessment of myelinization by qPCR quantification of the expression of Proteolipid protein 1 (Plp1) and UDP glycosyltransferase 8 (Ugt8) in the brain and spinal cord (upper and lower regions) of 4-month-old male and female wild-type (healthy) mice, untreated Sandhoff mice and Sandhoff mice that received a total dose of 1×10\hat{ }11 vg/mouse of either control vectors (Null) or AAV9 vectors encoding both HexA and HexB genes fused with a short linker (L1 AB mouse and L3 AB mouse) into the CSF at 1 month of age. Results are expressed as mean±SEM. n=4-7 animals/group. *P<0.05, **P<0.01, and ***P<0.001 vs. Null.

FIG. 10. Correction of astrogliosis after an intra-CSF delivery of AAV9 vectors encoding both Hexa and Hexb genes in Sandhoff mice. Immunostaining with an antibody specific for the astrocyte marker GFAP in different brain regions of 4-month-old male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes fused with a short linker (L1 AB mouse and L3 AB mouse) into the CSF at 1 month of age. Histograms depict the corresponding GFAP+ signal quantification for each brain region in each cohort. n=5 animals/group. Results are expressed as mean±SEM. *P<0.05, **P<0.01, and ***P<0.001 vs. Null.

FIG. 11. Correction of microglial infiltration after an intra-CSF delivery of AAV9 vectors encoding both Hexa and Hexb genes in Sandhoff mice. Quantification by qPCR of CD68 expression, a marker of microglia, in section I and V, the most rostral and caudal regions, respectively, of the brain in 4-month-old male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes fused with a short linker (L1 AB mouse and L3 AB mouse) into the CSF at 1 month of age. n=5 animals/group. Results are expressed as mean±SEM. ***P<0.001 vs. Null.

FIG. 12. Increased hepatic and circulating HexA activity after intra-CSF administration of AAV9 vectors encoding both Hexa and Hexb genes in Sandhoff mice. Measurement of HexA activity in liver and serum of 4-month-old male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes (L1 AB mouse, L2 AB mouse, and L3 AB mouse) into the CSF at 1 month of age. WT HexA activity were set to 100%. Results are expressed as mean±SEM. n=4-6 animals/group. *P<0.05, **P<0.01, and ***P<0.001 vs. Null.

FIG. 13. Intra-CSF delivery of AAV9 vectors encoding both Hexa and Hexb genes in Sandhoff mice corrects secondary storage pathology in peripheral tissues. (A, B) Determination of total cholesterol content in the liver (A) and quantification of glycosaminoglycans (GAGs) in liver, spleen, lung, and kidney (B) of 4-month-old male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes fused with a short linker (L1 AB mouse, L2 AB mouse, and L3 AB mouse) into the CSF at 1 month of age. Results are expressed as mean±SEM. n=4-10 animals/group. *P<0.05, **P<0.01, and ***P<0.001 vs. Null.

FIG. 14. Intra-CSF delivery of AAV9 vectors encoding both Hexa and Hexb genes in Sandhoff mice restores hepatic lysosomal homeostasis. Activity, as % of WT, of N-sulphoglucosamine sulphohydrolase (SGSH) and heparan-alpha-glucosaminide N-acetyltransferase (HGSNAT) in liver extracts obtained from 4-month-old male and female wild-type (healthy) mice, untreated Sandhoff mice and Sandhoff mice that received a total dose of 1×10\hat{ }11 vg/mouse of either control vectors (Null) or AAV9 vectors encoding both HexA and HexB genes fused with a short linker (L1 AB mouse, L2 AB mouse, and L3 AB mouse) into the CSF at 1 month of age. Results are expressed as mean±SEM. n=4-6 animals/group. *P<0.05, **P<0.01, and ***P<0.001 vs. Null.

FIG. 15. Normalization of behavioral deficits after intra-CSF administration of AAV9 vectors encoding both Hexa and Hexb genes in Sandhoff mice. The Open-Field test was performed in 4-month-old naïve-tested male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes (L1 AB mouse, L2 AB mouse, and L3 AB mouse) into the CSF at 1 month of age. Data corresponds to the locomotor activity recorded during the first 3 minutes. Histograms depict the results of the parameters analyzed: Total distance travelled, Distance in border, Resting time, and Slow time. Results are expressed as mean±SEM. n=8-25 animals/group. *P<0.05, **P<0.01, and ***P<0.001 vs. Null.

FIG. 16. Correction of motor coordination, mobility, and disease progression deficits after intra-CSF administration of AAV9 vectors encoding both Hexa and Hexb genes in Sandhoff mice. Motor coordination, mobility, and disease progression was assessed with the following tests: righting reflex, mesh, hindlimb clasping and rotarod tests. All tests were performed in 4-month-old male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes fused with a short linker (L1 AB mouse, L2 AB mouse, and L3 AB mouse) into the CSF at 1 month of age. Data from righting reflex test corresponds to the latency to flip over themselves when mice are laid on their back; mesh test, to the latency to fall from the wire; hindlimb clasping test, to the score assigned after assessing the hindlimb position when mice are suspended by the tail; and rotarod test, to the maximum latency to fall from the accelerating rotarod. Results are expressed as mean±SEM. n=5-29 animals/group. Righting reflex, hindlimb clasping and mesh tests: *P<0.05, **P<0.01, and ***P<0.001 vs. Null. Rotarod test: *P<0.05 and ***P<0.001 vs. WT.

FIG. 17. Prolonged survival following intra-CSF delivery of AAV9 vectors encoding both Hexa and Hexb genes in Sandhoff mice. Kaplan-Meier survival curves in male and female wild-type (healthy) mice, untreated Sandhoff mice and Sandhoff mice that received a total dose of 1×10\hat{ }11 vg/mouse of either control vectors (Null) or AAV9 vectors encoding both Hexa and Hexb genes (L1 AB mouse and L3 AB mouse). n=19-33 males/group; n=18-33 females/group. Males: P<0.0001 for WT, L1 AB mouse, and L3 AB mouse vs. Null; P=0.8776 for Sandhoff vs. Null. Females: P<0.0001 for WT, L1 AB mouse, and L3 AB mouse vs. Null; P=0.7626 for Sandhoff vs. Null.

FIG. 18. Increased HexA activity in the CNS after intra-CSF gene transfer of P2A AB mouse AAV9 vectors encoding both Hexa and Hexb genes in Sandhoff mice. Determination of HexA activity in different brain sections (I-V) of 4-month-old female wild-type (healthy) mice, untreated Sandhoff mice and Sandhoff mice that received a total dose of 1×10 11 vg/mouse of either control vectors (Null) or AAV9 vectors encoding both Hexa and Hexb genes fused with the self-cleaving linker (P2A AB mouse) into the CSF at 1 month of age. WT HexA activity was set to 100%. Results are expressed as mean±SEM. n=5 animals/group. *P<0.05, **P<0.01, and ***P<0.001 vs. Null.

FIG. 19. Increased HexA activity in the CNS after intra-CSF gene transfer of two AAV9 vectors encoding the Hexa or Hexb gene in Sandhoff mice. Determination of HexA activity in different brain sections (I-V) of 4-month-old male and female wild-type (healthy) mice, untreated Sandhoff mice and Sandhoff mice that received a total dose of either 1×10\hat{ }11 vg/mouse of control vectors (Null) or 2×10\hat{ }11 vg/mouse (1×10\hat{ }11 vg/mouse for Hexa and 1×10\hat{ }11 vg/mouse for Hexb) of AAV9 vectors (Hexa+Hexb). WT HexA activity was set to 100%. Results are expressed as mean±SEM. n=5 animals/group. *P<0.05, **P<0.01, and ***P<0.001 vs. Null.

FIG. 20. Survival study after an intra-CSF administration of AAV9 vectors encoding Hexa and Hexb genes in Sandhoff mice. Kaplan-Meier survival curves in male and female wild-type (healthy) mice, untreated Sandhoff mice and Sandhoff mice that received a total dose of 1×10¹¹ vg/mouse of control vectors (Null), P2A AB mouse AAV9 vectors encoding both Hexa and Hexb genes fused with a self-cleaving linker, or two AAV9 vectors encoding Hexa or Hexb genes (Hexa+Hexb). Hexa+Hexb cohort were Sandhoff mice treated at a total dose of 2×10\hat{ }11 vg/mouse (1×10\hat{ }11 vg/mouse for Hexa and 1×10\hat{ }11 vg/mouse for Hexb). n=14-33 males/group; n=16-33 females/group. Males: P<0.0001 for WT, P2A AB mouse, and Hexa+Hexb vs. Null; P=0.8776 for Sandhoff vs. Null. Females: P<0.0001 for WT, P2A AB mouse, and Hexa+Hexb vs. Null; P=0.7626 for Sandhoff vs. Null.

FIG. 21. Increased HexA activity in the CNS after intra-CSF gene transfer of AAV9 vectors encoding both Hexa and Hexb genes in Tay-Sachs mice. Measurement of HexA activity in different brain sections (I-V) of 4-month-old male and female wild-type (healthy) mice, untreated Tay-Sachs mice and Tay-Sachs mice that received a total dose of 1×10\hat{ }11 vg/mouse of either control vectors (Null) or AAV9 vectors encoding both Hexa and Hexb genes fused with a short linker (L1 AB mouse and L3 AB mouse) into the CSF at 1 month of age. WT HexA activity was set to 100%. Results are expressed as mean±SEM. n=4-5 animals/group. *P<0.05, **P<0.01, and ***P<0.001 vs. Null.

FIG. 22. Increased hepatic and circulating HexA activity after intra-CSF administration of AAV9 vectors encoding both Hexa and Hexb genes in Tay-Sachs mice. Measurement of HexA activity in liver and serum of 4-month-old male and female wild-type (healthy) mice, untreated Tay-Sachs mice and Tay-Sachs mice that received a total dose of 1×10\hat{ }11 vg/mouse of either control vectors (Null) or AAV9 vectors encoding both HexA and HexB genes fused with a short linker (L1 AB mouse and L3 AB mouse) into the CSF at 1 month of age. WT HexA activity was set to 100%. Results are expressed as mean±SEM. n=4-5 animals/group. *P<0.05, **P<0.01, and ***P<0.001 vs. Null.

FIG. 23. HEK293 cell transfection of plasmids encoding both HEXA and HEXB fused with a peptide linker. HEK293 cells were transfected with 0.8 μg of plasmid encoding the following human constructs: L1 AB human noh, L1 AB human GA, L1 AB human IDT and L1 AB human NV (SEQ ID NOs: 81-84). Histograms depict HexA activity (i.e., activity of HexA and HexS summed together) in cells (A) and medium (B) measured 48 h after transfection. HexA activity of non-transfected (NT) cells was set to 100%. Results are expressed as mean±SEM. n=4 wells/group. *P<0.05 and ***P<0.001 vs. L1 AB human noh.

FIG. 24. HEK293 cell transfection of plasmids encoding both Hexa and Hexb fused with three different short linkers. HEK293 cells were transfected with 4 μg of plasmid encoding the following mouse constructs: L1 AB mouse (SEQ ID NO: 75), L2 AB mouse (SEQ ID NO: 76), L3 AB mouse (SEQ ID NO: 77), Hexa+Hexb (SEQ ID NOs: 85-86), P2A AB mouse (SEQ ID NO: 87) and P2A BA mouse (SEQ ID NO: 88). Representative images of Western blots illustrating the content of HEXA (A) and HEXB (B) in medium of HEK-293 cells analyzed 48 h after transfection. n=2-3 wells/group.

FIG. 25. Long-term increased HexA activity in the CNS after intra-CSF gene transfer of AAV9 vectors encoding both Hexa and Hexb genes in Sandhoff mice. Determination of HexA activity in different brain sections (I-V) of 8-month-old male and female wild-type (healthy) mice and Sandhoff mice that received a total dose of 1×10\hat{ }11 vg/mouse of AAV9 vectors encoding both Hexa and Hexb genes fused with a short linker (L1 AB mouse and L3 AB mouse) into the CSF at 1 month of age. Sandhoff mice received a total dose of 1×10\hat{ }11 vg/mouse of control vectors (Null) into the CSF at 1 month of age and were euthanized at 4 months of age. WT HexA activity was set to 100%. Results are expressed as mean±SEM. n=4-5 animals/group. *P<0.05, **P<0.01, and ***P<0.001 vs. Null.

FIG. 26. Long-term correction of secondary lipid storage in the CNS following an intra-CSF delivery of AAV9 vectors encoding both Hexa and Hexb genes in Sandhoff mice. Representative images of filipin staining to detect unesterified cholesterol in cerebral cortex and cerebellum from 8-month-old male wild-type (healthy) mice and Sandhoff mice that received a total dose of 1×10\hat{ }11 vg/mouse of AAV9 vectors encoding both Hexa and Hexb genes fused with a short linker (L1 AB mouse and L3 AB mouse) into the CSF at 1 month of age. Sandhoff mice received a total dose of 1×10\hat{ }11 vg/mouse of control vectors (Null) into the CSF at 1 month of age and were euthanized at 4 months of age. n=3 animals/group. Scale bars, 25 μm.

FIG. 27. Long-term normalization of CNS lysosomal compartment size after an intra-CSF gene transfer of AAV9 vectors encoding both Hexa and Hexb genes in Sandhoff mice. Evaluation of the size of lysosomal compartment by LIMP2 immunostaining in different brain regions of 8-month-old male and female wild-type (healthy) mice and Sandhoff mice that received a total dose of 1×10\hat{ }11 vg/mouse of AAV9 vectors encoding both Hexa and Hexb genes fused with a short linker (L1 AB mouse and L3 AB mouse) into the CSF at 1 month of age. Sandhoff mice received a total dose of 1×10\hat{ }11 vg/mouse of control vectors (Null) into the CSF at 1 month of age and were euthanized at 4 months of age. Histograms depict the corresponding LIMP2+ signal quantification for each brain region in each cohort. Results are expressed as mean±SEM. n=4-5 animals/group. **P<0.01 and ***P<0.001 vs. Null.

FIG. 28. Long-term restoration of lysosomal homeostasis in the CNS following an intra-CSF delivery of AAV9 vectors encoding both Hexa and Hexb genes in Sandhoff mice. Percentage of WT activity of several lysosomal enzymes analyzed in brain extracts from 8-month-old male and female wild-type (healthy) mice and Sandhoff mice that received a total dose of 1×10\hat{ }11 vg/mouse of AAV9 vectors encoding both Hexa and Hexb genes fused with a short linker (L1 AB mouse and L3 AB mouse) into the CSF at 1 month of age. Sandhoff mice received a total dose of 1×10\hat{ }11 vg/mouse of control vectors (Null) into the CSF at 1 month of age and were euthanized at 4 months of age. Restoration of altered activities of alpha-N-acetylgalactosamine-6 sulfatase (GALNS), beta-glucuronidase (GUSB), heparan-alpha-glucosaminide N-acetyltransferase (HGSNAT), alpha-N-acetylglucosaminidase (NAGLU) and N-sulphoglucosamine sulphohydrolase (SGSH) in all treated Sandhoff mice. Results are expressed as mean±SEM. n=5 animals/group. *P<0.05, **P<0.01 and ***P<0.001 vs. Null.

FIG. 29. Long-term correction of astrogliosis after an intra-CSF delivery of AAV9 vectors encoding both Hexa and Hexb genes in Sandhoff mice. Immunostaining with an antibody specific for the astrocyte marker GFAP in different brain regions of 8-month-old male and female wild-type (healthy) mice and Sandhoff mice that received a total dose of 1×10\hat{ }11 vg/mouse of AAV9 vectors encoding both Hexa and Hexb genes fused with a short linker (L1 AB mouse and L3 AB mouse) into the CSF at 1 month of age. Sandhoff mice received a total dose of 1×10\hat{ }11 vg/mouse of control vectors (Null) into the CSF at 1 month of age and were euthanized at 4 months of age. Histograms depict the corresponding GFAP+signal quantification for each brain region in each cohort. n=5 animals/group. Results are expressed as mean±SEM. *P<0.05, **P<0.01, and ***P<0.001 vs. Null.

FIG. 30. Long-term correction of microglial infiltration after an intra-CSF delivery of AAV9 vectors encoding both Hexa and Hexb genes in Sandhoff mice. Quantification by qPCR of CD68 expression, a marker of microglia, in section I and V, the most rostral and caudal regions, respectively, of the brain in 8-month-old male and female wild-type (healthy) mice and Sandhoff mice that received a total dose of 1×10\hat{ }11 vg/mouse of AAV9 vectors encoding both Hexa and Hexb genes fused with a short linker (L1 AB mouse and L3 AB mouse) into the CSF at 1 month of age. Sandhoff mice received a total dose of 1×10\hat{ }11 vg/mouse of control vectors (Null) into the CSF at 1 month of age and were euthanized at 4 months of age. n=5 animals/group. Results are expressed as mean±SEM. ***P<0.001 vs. Null.

FIG. 31. Long-term increased hepatic and circulating HexA activity after intra-CSF administration of AAV9 vectors encoding both Hexa and Hexb genes in Sandhoff mice. Measurement of HexA activity in liver and serum of 8-month-old male and female wild-type (healthy) mice and Sandhoff mice that received a total dose of 1×10\hat{ }11 vg/mouse of AAV9 vectors encoding both Hexa and Hexb genes (L1 AB mouse and L3 AB mouse) into the CSF at 1 month of age. Sandhoff mice received a total dose of 1×10\hat{ }11 vg/mouse of control vectors (Null) into the CSF at 1 month of age and were euthanized at 4 months of age. WT HexA activity were set to 100%. Results are expressed as mean±SEM. n=5 animals/group. *P<0.05, **P<0.01, and ***P<0.001 vs. Null.

FIG. 32. Long-term intra-CSF delivery of AAV9 vectors encoding both Hexa and Hexb genes in Sandhoff mice restores hepatic lysosomal homeostasis. Activity, as % of WT, of N-sulphoglucosamine sulphohydrolase (SGSH) in liver extracts obtained from 8-month-old male and female wild-type (healthy) mice and Sandhoff mice that received a total dose of 1×10\hat{ }11 vg/mouse of AAV9 vectors encoding both HexA and HexB genes fused with a short linker (L1 AB mouse and L3 AB mouse) into the CSF at 1 month of age. Sandhoff mice received a total dose of 1×10\hat{ }11 vg/mouse of control vectors (Null) into the CSF at 1 month of age and were euthanized at 4 months of age. Results are expressed as mean±SEM. n=5 animals/group. ***P<0.001 vs. Null.

FIG. 33. Long-term normalization of behavioral deficits after intra-CSF administration of AAV9 vectors encoding both Hexa and Hexb genes in Sandhoff mice. The Open-Field test was performed in 8-month-old naïve-tested male and female wild-type (healthy) mice and Sandhoff mice that received a total dose of 1×10\hat{ }11 vg/mouse of AAV9 vectors encoding both Hexa and Hexb genes (L1 AB mouse and L3 AB mouse) into the CSF at 1 month of age. Sandhoff mice received a total dose of 1×10\hat{ }11 vg/mouse of control vectors (Null) into the CSF at 1 month of age. Untreated and Null-injected Sandhoff mice were euthanized at 4 months of age. Data corresponds to the locomotor activity recorded during the first 3 minutes. Histograms depict the results of the parameters analyzed: Total distance travelled, Distance in border, Resting time, and Slow time. Results are expressed as mean±SEM. n=10-16 animals/group. **P<0.01 and ***P<0.001 vs. Null.

FIG. 34. Long-term correction of mobility and disease progression deficits after intra-CSF administration of AAV9 vectors encoding both Hexa and Hexb genes in Sandhoff mice. Mobility and disease progression was assessed with the righting reflex test in 8-month-old male and female wild-type (healthy) mice and Sandhoff mice that received a total dose of 1×10\hat{ }11 vg/mouse of AAV9 vectors encoding both Hexa and Hexb genes fused with a short linker (L1 AB mouse and L3 AB mouse) into the CSF at 1 month of age. Sandhoff mice received a total dose of 1×10\hat{ }11 vg/mouse of control vectors (Null) into the CSF at 1 month of age. Untreated and Null-injected Sandhoff mice were euthanized at 4 months of age. Data corresponds to the latency to flip over themselves when mice are laid on their back. Results are expressed as mean±SEM. n=11-16 animals/group. **P<0.01 and ***P<0.001 vs. Null.

### EXAMPLES

As described in the Examples section, the following constructs were tested:
L1 AB mouse gene construct (SEQ ID NO:75)
L2 AB mouse gene construct (SEQ ID NO:76)
L3 AB mouse gene construct (SEQ ID NO:77)
L1 BA mouse gene construct (SEQ ID NO:78)
L2 BA mouse gene construct (SEQ ID NO:79)
L3 BA mouse gene construct (SEQ ID NO:80)
Hexa single gene construct (SEQ ID NO:85)
Hexb single gene construct (SEQ ID NO:86)
P2A AB mouse gene construct (SEQ ID NO:87)
P2A BA mouse gene construct (SEQ ID NO:88)
L1 AB human noh gene construct (SEQ ID NO: 81)
L1 AB human GA gene construct (SEQ ID NO:82)
L1 AB human IDT gene construct (SEQ ID NO:83)
L1 AB human NV gene construct (SEQ ID NO:84)

### General Procedures to the Examples

### Cell Culture and Transfection

Human embryonic kidney 293 (HEK293) cells were cultured at 37° C. under humidified 5% CO₂ in Dulbecco's Modified Eagle Medium (DMEM high glucose, Thermofisher Scientific) containing 10% Gibco fetal bovine serum (FBS) (ThermoFischer Scientific). The day before transfection, HEK293 cells were seeded in 6- or 24-well plates at a cell density of 1.2×10\hat{ }6 or 2.25-2.5×10\hat{ }5 cells/well in 2 or 1 ml/well of total volume, respectively. Prior to transfection, cells were assessed for correct cell confluence (90%) at a bright field microscope. Cells were transfected using Lipofectamine® 2000 (ThermoFisher Scientific), following the protocol provided by the manufacturer. Briefly, 4 or 0.8 μg/well of plasmid DNA and 10 or 2 μl/well of Lipofectamine® 2000 were each diluted in 250 or 50 μl OptiMEM prepared for 6- or 24-well plates, respectively. Mixed reagents were incubated for 5 minutes. Next, the Lipofectamine mix was added to the DNA mix, then gently mixed, and incubated for 20 minutes prior to drop-wise addition to wells (500 or 100 μl/well to 6- or 24 well plates, respectively). Each plasmid (pAAV-Cbh-omHexa-L1-omHexb (SEQ ID NO:28), pAAV-Cbh-omHexa-L2-omHexb (SEQ ID NO:29), pAAV-Cbh-omHexa-L3-omHexB (SEQ ID NO: 30), pAAV-Cbh-omHexb-L1-omHexA (SEQ ID NO: 31), pAAV-Cbh-omHexb-L2-omHexA (SEQ ID NO:32), pAAV-Cbh-omHexb-L3-omHexa (SEQ ID NO:33), pAAV-Cbh-omHexa-P2A-omHexb (SEQ ID NO:36), pAAV-Cbh-omHexb-P2A-omHexa (SEQ ID NO:59), pAAV-Cbh-omHexa (SEQ ID NO: 34), pAAV-Cbh-omHexb (SEQ ID NO:35), pAAV-Cbh-hsHEXA-L1-hsHEXB (SEQ ID NO:60), pAAV-Cbh-ohsHEXA-L1-ohsHEXB (GA) (SEQ ID NO:61), pAAV-Cbh-ohsHEXA-L1-ohsHEXB (IDT) (SEQ ID NO:62), and pAAV-Cbh-ohsHEXA-L1-ohsHEXB (NV) (SEQ ID NO:63)) was transfected in triplicate/quadruplicate for measurement of total beta-hexosaminidase activity (beta-HEXO; sum of HexA, HexB and HexS activities) and HexA activity (i.e., activity of HexA and HexS summed together). HEK293 cells transfected with the pCAG-GFP-WPRE plasmid were used as positive controls, while non-transfected HEK293 cells (NT) were used as negative controls.

### Animal Model

A mutant C57B129SF2/J HexB-deficient mouse (Sandhoff) in which a neomycin resistance cassette was inserted into and disrupted exon 13 of the Hexb gene was purchased from The Jackson Laboratory (Sango et al, 1995). This targeted mutation resulted in no detectable functional protein. Sandhoff and healthy control mice were inbred from heterozygous founders.

A mutant C57B129SF2/J HexA-deficient mouse (Tay-Sachs) in which a neomycin resistance cassette was inserted into and disrupted exon 8 of the Hexa gene was purchased from The Jackson Laboratory (Sango et al, 1995). This targeted mutation resulted in neither detectable RNA transcript nor functional protein. Tay-Sachs and healthy control mice were inbred from heterozygous founders.

In both Sandhoff and Tay-Sachs mice, genotype was determined on genomic DNA from tail-clipped samples with a PCR analysis that amplifies a sequence encompassing the targeted mutation. For Sandhoff mice, the sequences of the respective sense and antisense primers were: Forward Common primer: 5′-ATT TTA AAA TTC AGG CCT CGA-3′ (SEQ ID NO: 45); Reverse WT primer: 5′-CAT TCT GCA GCG GTG CAC GGC)-3′ (SEQ ID NO: 46); and Reverse Mutant primer: 5′-CAT AGC GTT GGC TAC CCG TGA-3′ (SEQ ID NO: 47). Bands of ~100 bp and ~200 bp were obtained for WT and knockout alleles, respectively. For Tay-Sachs mice, the sequences of the respective sense and antisense primers were: Forward WT primer: 5′-GGT GTA TGT GTA ACA CTC GTG G-3′ (SEQ ID NO: 48); Reverse WT primer: 5′-CAG TTC TAG GCT CAG AAT GAG G-3′ (SEQ ID NO: 49); Forward Mutant primer: 5′-CTT GGG TGG AGA GGC TAT TC-3′ (SEQ ID NO: 50); and Reverse Mutant primer: 5′-AGG TGA GAT GAC AGG AGA TC-3′ (SEQ ID NO: 51). Bands of 430 bp and 280 bp were obtained for WT and knockout alleles, respectively. Mice were fed ad libitum with a standard diet (2018S Teklad Global Diets®, Harlan Labs) and maintained under a light-dark cycle of 12 h and stable temperature (22° C.±2). All experimental procedures were approved by the Ethics Committee for Animal and Human Experimentation of the Universitat Autonoma de Barcelona.

Recombinant AAV vectors Single-stranded AAV vectors of serotype 9 were produced by triple transfection of HEK293 cells according to standard methods (Ayuso et al, 2010). Cells were cultured to 80% confluence in roller bottles (RB) (850 cm², flat; Corning™, Sigma-Aldrich Co., Saint Louis, MO, US) in DMEM supplemented with 10% FBS and then co-transfected by calcium phosphate method with: 1) a plasmid carrying the expression cassette flanked by the AAV2 ITRs (SEQ ID NOs: 25-26); 2) a helper plasmid carrying the AAV2 rep gene and the AAV of serotype 9 cap gene; and 3) a plasmid carrying the adenovirus helper functions. Transgenes used were: a) the murine optimized Hexa and Hexb coding-sequence fused with a short linker (L1-SEQ ID NOs: 11 and 14, L2-SEQ ID NOs: 12 and 15 or L3-SEQ ID NOs: 13 and 16); b) the murine optimized Hexa and Hexb coding-sequence fused with a self-cleaving linker (P2A); c) the murine optimized Hexa coding-sequence (SEQ ID NO: 5); d) the murine optimized Hexb coding-sequence (SEQ ID NO: 10); e) the human HEXA coding-sequence (SEQ ID NO: 3); f) the human coding HEXB coding-sequence (SEQ ID NO:8); g) the human optimized HEXA coding-sequence (SEQ ID NOs: 64, 66, 68); h) the human optimized HEXB coding sequence (SEQ ID NOs: 65, 67, 69). Gene expression of each cassette was driven by the ubiquitous promoter Cbh (a short version of the widely used CAG promoter composed of the cytomegalovirus (CMV) early enhancer, chicken beta-actin promoter and a hybrid intron) (SEQ ID NO: 24). The expression cassette also included the polyA sequence from SV40 (SEQ ID NO: 27). A noncoding plasmid carrying the Cbh promoter and the SV40 sequence, but no transgene, was used to produce null vectors. AAV vectors were purified with an optimized method based on a polyethylene glycol precipitation step and two consecutive cesium chloride (CsCl) gradients. This second-generation CsCl-based protocol reduced empty AAV capsids and DNA and protein impurities dramatically (Ayuso et al, 2010). Purified AAV vectors were dialyzed against PBS, filtered, and stored at −80° C. Titers of viral genomes were determined by quantitative PCR following the protocol described for the AAV2 reference standard material using linearized plasmid DNA as standard curve (Lock et al, 2010). The vectors were constructed according to molecular biology techniques well known in the art.

### In Vivo Intra-CSF Administration of AAV Vectors

Mice were anesthetized with an intraperitoneal injection of ketamine (100 mg/kg) and xylazine (10 mg/kg), and the skin of the posterior part of the head, from behind the ears to approximately between the scapulae, was shaved. Mice were held in prone position, with the head at a slightly downward inclination. A 2-mm rostro-caudal incision was made to introduce a Hamilton syringe at an angle of 45-55° into the cisterna magna, between the occiput and the C1-vertebra and 5 μl of vector dilution was administered. Given that the CNS is the main target compartment for vector delivery, mice were dosed with the same number of vector genomes/mouse irrespective of body weight (1×10\hat{ }11 vg/mouse). The mouse brain volume is about 0.45 cm\hat{ }3, so this dose corresponds to about 2.22×10\hat{ }11 vg per ml of brain. In the case of the single Hexa or Hexb gene vectors, a mix of AAV-Cbh-Hexa (1×10\hat{ }11 vg) and AAV-Cbh-Hexb (1×10\hat{ }11 vg) resulting in a total dose of 2×10\hat{ }11 vg was administered in mice to achieve an equal number of alpha subunits (encoded by Hexa) and beta subunits (encoded by Hexb) compared to the single AAV vectors (1×10\hat{ }11 vg), in which each vector included both Hexa and Hexb genes.

### Sample Collection

Three or seven months after vector administration, mice were anesthetized by intraperitoneal injection of ketamine (100 mg/kg) and xylazine (10 mg/kg). Blood was extracted by cardiac puncture and, afterwards, animals were transcardially perfused with 10 ml of PBS. This procedure cleared blood from tissues thus removing all traces of circulating beta-hexosaminidase. Then, the entire brain and multiple somatic tissues were collected. The encephalon was longitudinally divided into two hemispheres (left and right), and each half was then coronally sectioned in five regions (I to V), section I being the most frontal and section V the most caudal. All samples were either snap frozen in liquid nitrogen and stored at −80° C. or immersed in formalin or 4% PFA for subsequent histological analyses.

### Activity of Lysosomal Enzymes

Brain and liver samples were sonicated in 250-300 and 500 μl of water, respectively. Homogenized tissues were clarified by centrifugation and supernatants were recovered to assay enzyme activities using 4-methylumbelliferone-derived fluorogenic substrates based on standard protocols. Total beta-hexosaminidase activity was determined using the 4-methylumbelliferyl-2-acetamido-2-deoxy-beta-D-glucopyranoside (4-MUG) assay, whereas the HexA enzyme activity was determined using the 4-methylumbelliferyl-6-sulfo-2-acetamido-2-deoxy-beta-D-glucopyranoside (4-MUGS) assay. Total beta-hexosaminidase (total beta-HEXO) was assayed in 0.2 μg of protein or 1 μl of medium at pH 4.5 for 1 h at 37° C. with 5 mM 4-MUG (Sigma). HexA was assayed in 0.2 μg of protein, 1 μl of medium or 0.4 μl of serum at pH 4.4 for 1 h at 37° C. with 5 mM 4-MUGS (Sigma). Heparan acetylCoA: alpha-Glucosaminide N-Acetyltransferase (heparan-alpha-glucosaminide N-acetyltransferase; HGSNAT) activity was assayed in 30 μg of protein extracts incubated with 3 mM 4-methylumbelliferyl-beta-D-glucosamine (Moscerdam substrates) supplemented with 12 mM Acetyl coenzyme A (Sigma) for 17 h at 37° C. N-sulfoglucosamine sulfohydrolase (SGSH) was assayed in a two-step protocol. Briefly, the first step consisted of the incubation of 30 μg of tissue protein extract with 10 mM of 4-MU-alphaGIcNS for 17 hours at 47° C. The second incubation was carried out in the presence of 10 U/ml of alpha-glucosidase (Sigma-Aldrich) in 0.2% BSA for 24 hours at 37° C. Alpha-N-acetylgalactosamine-sulfate (NAGLU) activity was assayed in 30 μg of tissue protein extract incubated with 2.5 nmol/l 4-methylumbelliferyl-alpha-d-N-acetyl-glucosaminide (MU-alphaGlcNAc, Moscerdam Substrates) for 3 h at 37° C. N-acetylgalactosamine-6-sulfatase (GALNS) activity was assayed in 10 μg of total protein with a first incubation step with 10 mM 4-methylumbelliferyl beta-D-galactopyranoside-6-sulfate (Toronto Research Chemicals) for 17 h at 37° C. followed by a second incubation with beta-galactosidase (Sigma) for 2 h at 37° C. For beta-glucuronidase (GUSB), activity was assayed in 15 μg of protein incubated for 1 h with 2 mM 4-methylumbelliferyl-beta-D-glucuronide (Sigma) at pH 4.8 and 37° C. After stopping each enzymatic reaction by increasing the pH, the released fluorescence was measured with a Synergy HTX fluorimeter (BioTek Instruments). Each activity was normalized against serum volume (expressed as nmol/h/mL) or total protein content (expressed as nmol/h/mg protein) quantified by Bradford assay (Bio-Rad), and then referred as % of WT activity, which was set to 100%.

### Histological Analyses

For immunohistochemical detection of LIMP2 and GFAP, tissues were fixed for 12-24 h in formalin, embedded in paraffin and sectioned. Tissue sections were incubated overnight at 4° C. with rabbit anti-LIMP2 antibody (NB400; Novus Biologicals) and rabbit anti-GFAP antibody (Ab6673; Abcam) and subsequently incubated with biotinylated goat anti-rabbit antibody (31820; Vector Laboratories), used as secondary antibody. LIMP2 and GFAP signals were amplified by incubating sections with ABC-Peroxidase staining kit (Thermo Scientific), visualized using 3,3-diaminobenzidine (Sigma-Aldrich) as a chromogen, and counterstained with hematoxylin. Brightfield images were obtained with an optical microscope (Eclipse 90i; Nikon). LIMP2 and GFAP signals were quantified with the NIS-Elements Advanced Research 2.20 software in 3-4 images of each brain region (original magnification, 20×) per animal, using the same signal threshold settings for all images. Then, the percentage of positive area was calculated, i.e., the area, in pixels, with a positive signal over the total tissue area in the image.

For filipin staining and immunohistochemical detection of GM2, tissues were fixed overnight in 4% PFA. Afterwards, tissues were dehydrated with 30% sucrose in PBS at 4° C. for 48 h for cryoprotection, subsequently embedded in Optimal Cutting Temperature (OCT) compound and kept at −80° C. until processing. Brains were then cryosectioned at −15° C. using a cryostat microtome to 14 μm thickness. For filipin staining, tissue sections were rehydrated in PBS, and incubated with filipin complex diluted in PBS to a working concentration of 0.05 mg/mL for 2 h at room temperature in dark. For GM2 immunohistochemistry, tissue sections were rehydrated in PBS, incubated overnight at 4° C. with mouse anti-GM2 antibody (AMS A2576; Amsbio) and subsequently incubated with biotinylated horse anti-mouse antibody (BA2000; Vector Laboratories), used as secondary antibody. GM2 signal was visualized as previously described herein for immunohistochemical detection of LIMP2 and GFAP. Fluorescence (filipin) and bright-field (GM2) images were obtained with an optical microscope (Eclipse 90i; Nikon).

### Western Blot Analysis

For total protein extracts from brain, tissues were homogenized in 250-300 μl of milliQ water and centrifuged at 12,000 g for 10 minutes at 4° C. Proteins were separated by 17% wt/vol sodium dodecyl sulfate (SDS)-polyacrylamide gel electrophoresis (PAGE), transferred to polyvinylidene difluoride (PVDF) membrane, and incubated overnight at 4° C. with the following antibody anti-rabbit anti-LC3B (NB100-2220, Novus Biologicals). Proteins from medium of HEK-293 cells were separated by 10% wt/vol sodium dodecyl sulfate (SDS)-polyacrylamide gel electrophoresis (PAGE), transferred to polyvinylidene difluoride (PVDF) membrane, and incubated overnight at 4° C. with the following antibodies anti-rabbit anti-HEXA (PAA195Mu01, Cloud-Clone Corp) and anti-rabbit anti-HEXB (PAA637Mu01, Cloud-Clone Corp).

Signal detection was performed using the swine anti-rabbit immunoglobulins horseradish peroxidase-labelled secondary antibody (P0217, Dako) and western blotting detection reagent (ECL Plus, Amersham). Signal intensity was quantified using Image J software.

### RNA Analysis

Total RNA was purified from brain and liver homogenized in Tripure Isolation Reagent (Roche) with RNeasy Mini kit (Qiagen). To eliminate the residual DNA, total RNA was treated with DNAsel (Qiagen). RNA was quantified in a NanoDrop ND-1000 spectrophotometer (NanoDrop). cDNA was synthesized with a Transcriptor First Strand cDNA Synthesis kit (Roche). Real-time quantitative PCR was performed in a QuantStudio™ 5 (Thermofisher) using Lightcycler 480 SyBr Green I Master Mix (Roche). Primers used to detect the expression of the following Mus musculus genes: CD68: Forward, 5′-TGG CGG TGG AAT ACA ATG TG-3′ (SEQ ID NO: 37) and Reverse, 5′-TGC TTG CAT TTC CAC AGC AG-3′ (SEQ ID NO: 38); Plp1: Forward, 5′-TTC CAG AGG CCA ACA TCA AG-3′ (SEQ ID NO: 39) and Reverse, 5′-AGG AGC CAT ACA ACA GTC AG-3′(SEQ ID NO: 40); and Ugt8: Forward, 5′-AGT TTC CAA GAC CAA CGC TGC-3′ (SEQ ID NO: 41) and Reverse, 5′-TGT TCC TGA GCA CCA CTT ACC-3′ (SEQ ID NO: 42). Data was normalized to the expression of the Mus musculus Tbp gene: Forward 5′-TGA CTC CTG GAA TTC CCA TC-3′ (SEQ ID NO: 43), Reverse 5′-TGC TGC TGT CTT TGT TGC TC-3′ (SEQ ID NO: 44).

### Cholesterol Determination

For lipid extraction, ~100 mg of tissue was homogenized in 7.5 ml chloroform-methanol (2:1 vol/vol) and then 1.5 ml 0.05% H₂SO₄ was added to the mixture. After an overnight at 4° C. to separate the organic from the aqueous phase, 1 ml of the organic phase was recovered and mixed with 1 ml of X-100 Triton 1% in chloroform. After evaporation at 90° C., 1 ml of chloroform was added and then evaporated, this process was performed twice. In the end, extracted lipids were resuspended in 500 μl of milliQ water. Total cholesterol determination was quantified in lipid extracts spectrophotometrically using an enzymatic assay (ABX Pentra Cholesterol CP, Horiba) in a Pentra 400 Analyzer (Horiba).

### Glycosaminoglycans Quantification

Tissue samples were weighed and then digested with proteinase K. The resultant extracts were clarified by centrifugation and filtration. GAG content was determined in tissue extracts with the Blyscan sulfated glycosaminoglycan kit (Biocolor), using chondroitin 4-sulfate as standard. GAG content was normalized to wet tissue weight.

### Open Field Test

The open field test was performed between 9:00 am and 2:00 μm, to minimize influence of circadian cycles. Briefly, animals were placed in the lower right corner of a brightly lit chamber (41×41×30 cm) divided in three squared concentric regions (center, 14×14 cm; periphery, 27×27 cm; and border, 41×41 cm). Exploratory behavior and general activity were recorded during the first three minutes using a video-tracking system (SmartJunior v3, Panlab).

### Righting Reflex Test

Righting reflex was used to assess mobility. A mouse was placed on its back on a tabletop (supine position) and the time elapsed for the animal to right itself through 180° (all 4 paws were right on the floor) was measured, with a maximum of 10s allowed. The test was performed 3 times on each mouse and the mean of all three trials was used for analysis.

### Mesh Test

To evaluate coordination and muscle strength, mice were tested in the mesh test. The mouse was placed right in the center of a wire mesh that then was rotated 180° to an inverted position (over the course of about 2 s) with the head of the mouse declining first. The mouse was held 40 cm above a soft padded surface, and the latency to hang upside down from the wire mesh was recorded to a maximum time of 60 s. Mice were given three trials, spaced out by 5 min, and the mean of all three trials was recorded for analysis. If animals did not explore during the trial, they were not included in the data analysis.

### Hindlimb Clasping Test

Hindlimb clasping test was performed to evaluate disease progression. Mouse was grasped by the tail near its base and suspended by the tail in an area clear of any objects. Then, hindlimb position was observed for 10 s to write down hindlimb clasping score, which was as follows: Score=0, both hindlimbs are consistently splayed outward, away from the abdomen, when mouse is suspended by the tail; Score=1; one hindlimb is retracted toward the abdomen for more than 50% of the time suspended; Score=2, both hindlimbs are partially retracted toward the abdomen for more than 50% of the time suspended; and Score=3, hindlimbs are entirely retracted and touching the abdomen for more than 50% of the time suspended.

### Rotarod Test

Mice were tested on an accelerating rotarod (Rotarod LE8200; Panlab), spinning at 4 RPM. Lane width, 50 mm; rod diameter, 30 mm. Before the trial, mice were gently placed on the rod with the speed set at 4 rpm and trained to remain on the rod. After training, mice were given three consecutive trials of 90 seconds in which the rod accelerated from 4-40 rpm in a 5-minute interval. Between trials, animals rest for 90 seconds. The next day, mice took 3 more trials on the rod. Latencies to fall from the rod were recorded and the maximum latency of all trials was used for analysis.

### Statistical Analysis

All values are expressed as mean±SEM. Statistical comparisons were made using one-way ANOVA. Multiple comparisons between control and treatment groups were made using Dunnett's post-test. Statistical significance was considered if P<0.05. The Kaplan-Meier method was used for survival analysis, and the log-rank test for comparisons.

### Example 1. Construction of Plasmids

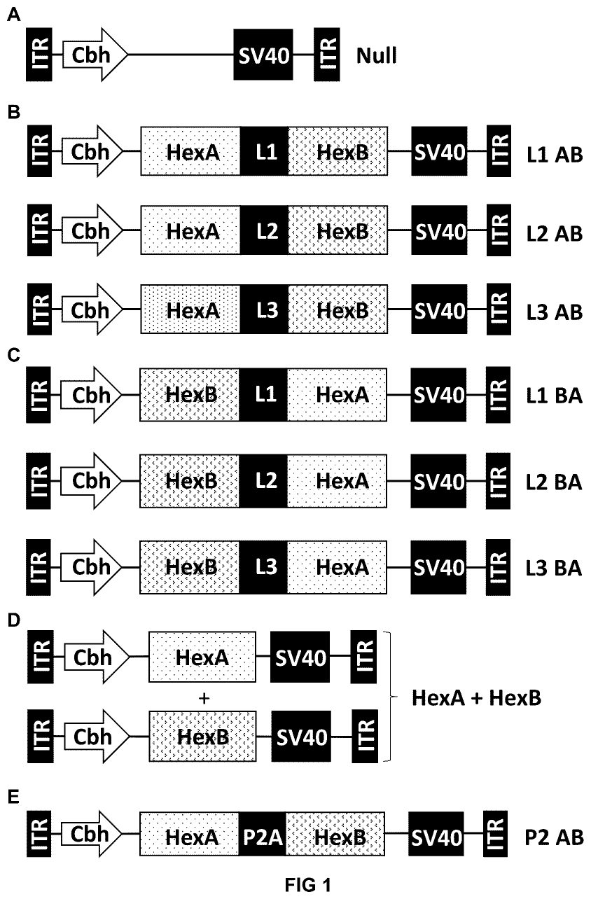

The ubiquitous promoter Cbh (SEQ ID NO: 24) (a short version of the widely used CAG promoter composed of the cytomegalovirus (CMV) early enhancer, chicken beta-actin and a hybrid intron) together with the polyA sequence from SV40 (SEQ ID NO: 27) were chemically synthetized (GeneArt; Invitrogen). Afterwards, the Cbh+SV40 was excised by Notl digestion and then cloned inside the Notl restriction site of the AAV backbone plasmid pAAV-MCS (multicloning site) (AmpR). The pAAV-MCS plasmid was previously generated and contained the ITRs from the AAV2 genome (SEQ ID NOs: 25-26), as well as the multicloning site for cloning the coding sequences (CDS) of interest. After this cloning, the resulting plasmid was named pAAV-Cbh-SV40 (FIG. 1A).

For the L1 AB, L2 AB and L3 AB mouse constructs (SEQ ID NOs: 75-77), a 3′ fragment of the optimized murine Hexa CDS (omHexa) and a 5′ fragment of the optimized murine Hexb CDS (omHexb) were fused with one of the short linkers (L1, L2 or L3-SEQ ID NOs: 11-16) and used as starting material and chemically synthetized for this purpose (GeneArt; Invitrogen). These CDS were cloned inside the pAAV-Cbh-omHexa-P2A-omHexb-SV40 plasmid flanked by Afel and BbvCl restriction sites at 5′ and 3′ ends, respectively. The 3′ fragment of the optimized murine Hexa CDS and the 5′ end fragment of the optimized murine Hexb CDS fused with one of the short linkers (L1, L2 or L3) was excised by Afel/BbvCl digestion and then cloned between the Afel and BbvCI restrictions sites of the AAV backbone plasmid pAAV-Cbh-omHexa-P2A-omHexb-SV40 (AmpR). The resulting plasmid was named pAAV-Cbh-omHexa-L1-omHexb-SV40 (SEQ ID NO: 28), pAAV-Cbh-omHexa-L2-omHexb-SV40 (SEQ ID NO: 29) and pAAV-Cbh-omHexa-L3-omHexb-SV40 (SEQ ID NO: 30) (FIG. 1B).

For the L1 BA, L2 BA and L3 BA mouse constructs (SEQ ID NOs: 78-80), a 3′ fragment of the optimized murine Hexb CDS (omHexb) and a 5′ fragment of the optimized murine Hexa CDS (omHexa) was fused with one of the short linkers (L1, L2 or L3-SEQ ID NOs: 11-16) and used as starting material and chemically synthetized for this purpose (GeneArt; Invitrogen). These CDS were cloned inside the pAAV-Cbh-omHexb-P2A-omHexa-SV40 plasmid (previously generated) flanked by Alel and Avril restriction sites at 5′ and 3′ ends, respectively. The 3′ fragment of the optimized murine Hexb CDS and the 5′ fragment of the optimized murine Hexa CDS fused with one of the short linkers (L1, L2 or L3) was excised by Alel/Avril digestion and then cloned between the Alel and Avrll restriction sites at 5′ and 3′ ends, respectively. The optimized murine HexB CDS and then the optimized murine Hexa CDS fused with one of the short linkers (L1, L2 or L3) was excised by Alel/Avril digestion and then cloned between the Alel and Avrll restrictions sites of the AAV backbone plasmid pAAV-Cbh-omHexb-P2A-omHexa-SV40 (AmpR). The resulting plasmids was named pAAV-Cbh-omHexb-L1-omHexa-SV40 (SEQ ID NO: 31), pAAV-Cbh-omHexb-L2-omHexa-SV40 (SEQ ID NO: 32) and pAAV-Cbh-omHexb-L3-omHexa-SV40 (SEQ ID NO: 33) (FIG. 1C).

For constructs carrying only one Hexa or Hexb gene, the optimized murine either Hexa or Hexb CDS were used as starting material and chemically synthetized for this purpose (GeneArt; Invitrogen). Either of these CDS was cloned inside the pAAV-Cbh-SV40 plasmid flanked by NheI and BamHI restriction sites at 5′ and 3′ ends, respectively. Optimized murine either Hexa or Hexb was excised by NheI/BamHI digestion and then cloned between the NheI and BamHI restriction sites of the AAV backbone plasmid pAAV-Cbh-SV40 (AmpR). The resulting plasmids were named pAAV-Cbh-omHexa-SV40 (SEQ ID NO: 34) or pAAV-Cbh-omHexb-SV40 (SEQ ID NO: 35) (FIG. 1D).

For the P2A AB mouse construct (SEQ ID NO: 87), the optimized murine Hexa and Hexb CDS fused with the self-cleaving linker P2A was used as starting material and chemically synthetized for this purpose (GeneArt; Invitrogen). This CDS was cloned inside the pAAV-Cbh-SV40 plasmid flanked by NheI and BamHI restriction sites at 5′ and 3′ ends, respectively. The optimized murine Hexa and Hexb CDS fused with the self-cleaving linker P2A (Hexa-P2A-Hexb) was excised by NheI/BamHI digestion and then cloned between the NheI and BamHI restrictions sites of the AAV backbone plasmid pAAV-Cbh-SV40 (AmpR). The resulting plasmid was named pAAV-Cbh-omHexa-P2A-omHexb-SV40 (SEQ ID NO; 36) (FIG. 1E).

For the L1 AB noh, L1 AB GA, L1 AB IDT, and L1 AB NV human constructs (SEQ ID NOs: 81-84), the non-optimized and GA-, IDT- and NV-optimized human HEXA and HEXB CDS fused with a short linker (L1-SEQ ID NOs: 11-14) was used as starting material and chemically synthetized for this purpose (GeneArt; Invitrogen). This CDS was cloned inside the pAAV-Cbh-SV40 plasmid flanked by NheI and BamHI restriction sites at 5′ and 3′ ends, respectively. The non-optimized and GA-, IDT- and NV-optimized human HEXA and HEXB CDS fused with a short linker (HEXA-L1-HEXB) was excised by NheI/BamHI digestion and then cloned between the NheI and BamHI restrictions sites of the AAV backbone plasmid pAAV-Cbh-SV40 (AmpR). The resulting plasmid was named pAAV-Cbh-noh HEXA-L1-nohHEXB-SV40 (SEQ ID NO; 60), pAAV-Cbh-ohHEXA-L1-ohHEXB-SV40 GA (SEQ ID NO; 61), pAAV-Cbh-ohHEXA-L1-ohHEXB-SV40 IDT (SEQ ID NO; 62) or pAAV-Cbh-ohHEXA-L1-ohHEXB-SV40 NV (SEQ ID NO; 63) (FIG. 1B).

For the P2A BA mouse construct (SEQ ID NO: 88), the optimized murine Hexb and Hexa CDS fused with the self-cleaving linker P2A was used as starting material and chemically synthetized for this purpose (GeneArt; Invitrogen). This CDS was cloned inside the pAAV-Cbh-SV40 plasmid flanked by NheI and BamHI restriction sites at 5′ and 3′ ends, respectively. The optimized murine Hexb and Hexa CDS fused with the self-cleaving linker P2A (Hexb-P2A-Hexa) was excised by NheI/BamHI digestion and then cloned between the NheI and BamHI restrictions sites of the AAV backbone plasmid pAAV-Cbh-SV40 (AmpR). The resulting plasmid was named pAAV-Cbh-omHexb-P2A-omHexa-SV40 (SEQ ID NO; 59).

### Example 2. Production of AAV9 Vectors

AAV9-Cbh-SV40, AAV9-Cbh-omHexa-SV40, AAV9-Cbh-omHexb-SV40, AAV9-Cbh-omHexa-P2A-omHexb-SV40, AAV9-Cbh-omHexa-L1-omHexb-SV40, AAV9-Cbh-omHexa-L2-omHexb-SV40, AAV9-Cbh-omHexa-L3-omHexb-SV40, AAV9-Cbh-omHexb-L1-omHexa-SV40, AAV9-Cbh-omHexb-L2-omHexa-SV40 and AAV9-Cbh-omHexb-L3-omHexa-SV40 were generated by helper virus-free transfection of HEK293 cells using three plasmids with modifications. Cells were cultured to 80% confluence in roller bottles (RB) (Corning) in DMEM supplemented with 10% FBS and then co-transfected with: 1) a plasmid carrying the expression cassette flanked by AAV2 ITRs; 2) a plasmid carrying the AAV2 rep and the AAV9 cap genes (pREP2CAP9); and 3) a plasmid carrying the adenovirus helper functions. Vectors were purified by two consecutives cesium chloride gradients using either a standard protocol or an optimized protocol as previously described (Ayuso et al, 2010). Vectors were dialyzed against PBS, filtered, titred by qPCR and stored at −80° C. until use.

### Example 3. Increased Beta-Hexosaminidase in HEK293 Cells after Plasmid Transfection

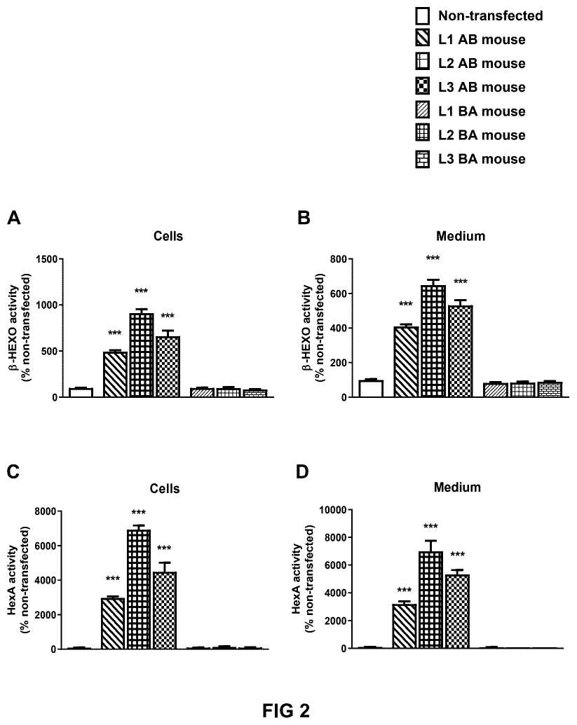

To evaluate our constructs and their capacity to produce active beta-hexosaminidase, 0.8 μg of plasmid DNA encoding the following constructs (L1 AB mouse, L2 AB mouse, L3 AB mouse, L1 BA mouse, L2 BA mouse and L3 BA mouse) (SEQ ID NOs: 75-80) (FIGS. 1B and 1C) was transfected in HEK293 cells. Forty-eight hours after transfection, a statistically significant increase in total beta-HEXO activity (FIG. 2A) was documented in cell extracts of L1 AB, L2 AB, and L3 AB mouse constructs, reaching values of 495%, 911% and 662% of non-transfected, respectively (FIG. 2A). A similar pattern of total beta-HEXO activity was also detected in medium of L1 AB, L2 AB, and L3 AB mouse constructs, with average activities of 409%, 648% and 532% of non-transfected, respectively (FIG. 2B). For HexA activity, the isoenzyme required to catabolize GM2 gangliosides, values of 2974%, 6933% and 4487% were quantified in cell extracts after L1 AB, L2 AB, and L3 AB mouse constructs transfection (FIG. 2C). Likewise, a statistically significant raise in medium of L1 AB, L2 AB, and L3 AB mouse constructs was observed, with average activities of 3199%, 7000% and 5333% of non-transfected, respectively (FIG. 2D). Yet, the L1 BA, L2 BA and L3 BA mouse constructs showed no increase at all neither in beta-HEXO nor in HexA activities in cell extracts and medium compared to non-transfected.

### Example 4. Therapeutic Efficacy after Intra-CSF Delivery of AAV9 Vectors Encoding Both Hexa and Hexb Genes (L1 AB Mouse, L2 AB Mouse and L3 AB Mouse) in Sandhoff Mice

To assess whether genetic engineering of the CNS with vectors encoding both optimized murine Hexa and Hexb genes (FIG. 1B) may exert therapeutic efficacy, a total dose of 1×10\hat{ }11 vector genomes (vg)/mouse of AAV9 vectors comprising both Hexa and Hexb fused with a short linker L1, L2 or L3 under the control of the ubiquitous promoter Cbh was administered into the cisterna magna of 1-month-old Sandhoff mice. Age-matched Sandhoff animals injected into the cisterna manga with a total dose of 1×10\hat{ }11 vg/mouse of AAV9-Cbh-Null vectors were used as control. Healthy wild-type and Sandhoff mice served as additional control groups.

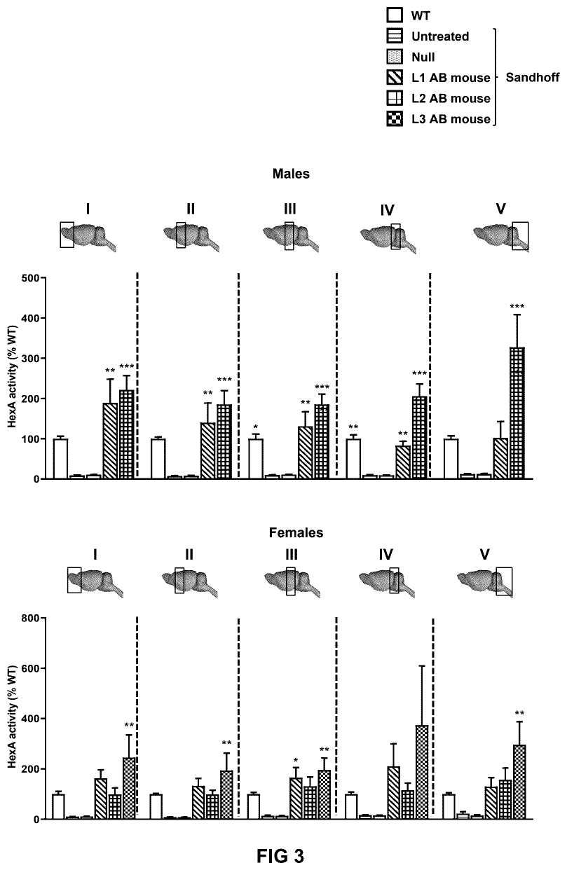

The intra-CSF administration of AAV9 vectors comprising both Hexa and Hexb fused with a short linker L1, L2 or L3 led to very high levels of HexA activity, the isoenzyme required to catabolize GM2 gangliosides (FIG. 3). In all 4-month-old Sandhoff-treated male mice, HexA activity reached at least the levels of healthy WT animals in all CNS areas analyzed (FIG. 3). In females, similar results were observed in L1 AB mouse-, L2 AB mouse- and L3-AB mouse-treated Sandhoff mice, In which levels achieved were quite similar to those documented in healthy WT animals in most encephalon regions (FIG. 3). Of note, the levels of HexA activity in all AAV-treated Sandhoff mice showed an even distribution throughout the CNS, from the most distal to the closest point of the injection (FIG. 3). As expected, untreated and Null-injected Sandhoff mice showed almost undetectable HexA activity in brain extracts at 4 months of age.

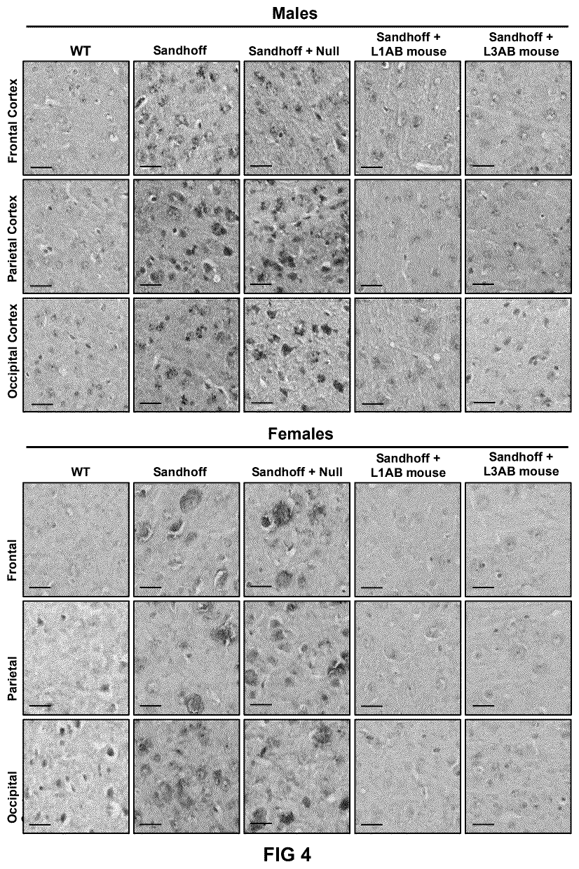

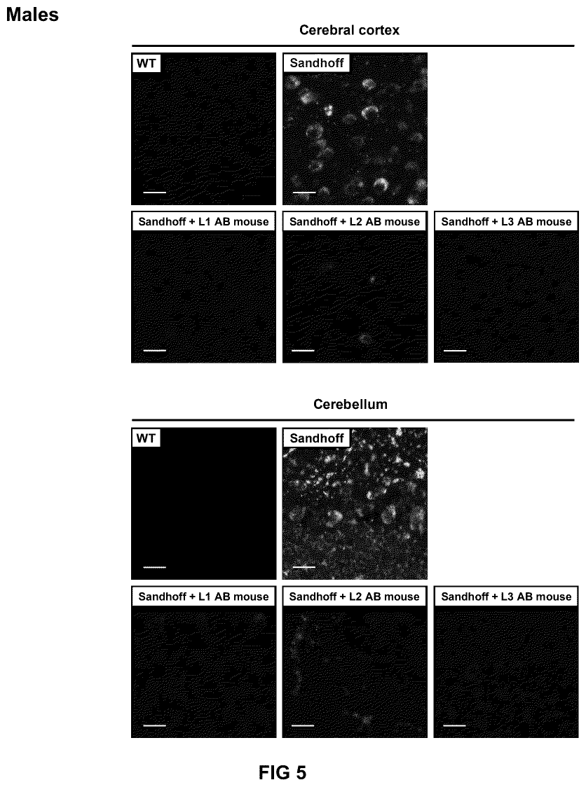

To evaluate the therapeutic efficacy of the treatment, an immunohistochemistry with an antibody against GM2, a marker of the primary storage in GM2 gangliosidosis, was performed in encephalon sections. As soon as three months post vector administration of our AAV9 vectors L1 AB mouse and L3 AB mouse, the GM2 immunostaining on brain sections revealed a complete absence of positive signal in 4-month-old treated males in comparison to untreated and Null-injected Sandhoff male mice, in which a strong positive signal was detected in all CNS areas assessed (FIG. 4). Similarly, complete correction of primary GM2 storage was also achieved in females when L1 AB mouse and L3 AB mouse AAV9 vectors were administered into the CSF of Sandhoff mice (FIG. 4). The increased transgene expression in the CNS of Sandhoff-treated animals also resulted in normalization of cholesterol accumulation in the cerebral cortex and cerebellum of untreated Sandhoff mice, as shown in the photomicrographs of filipin staining (FIG. 5). These results indicated efficient correction of primary GM2 and secondary cholesterol storage pathology in the CNS after L1 AB mouse, L2 AB mouse and L3 AB mouse treatment (FIGS. 4 and 5).

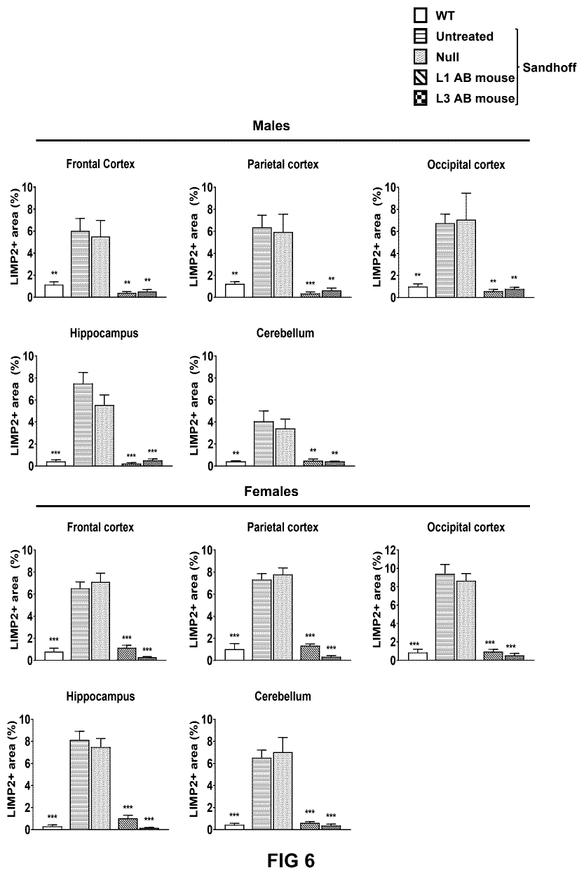

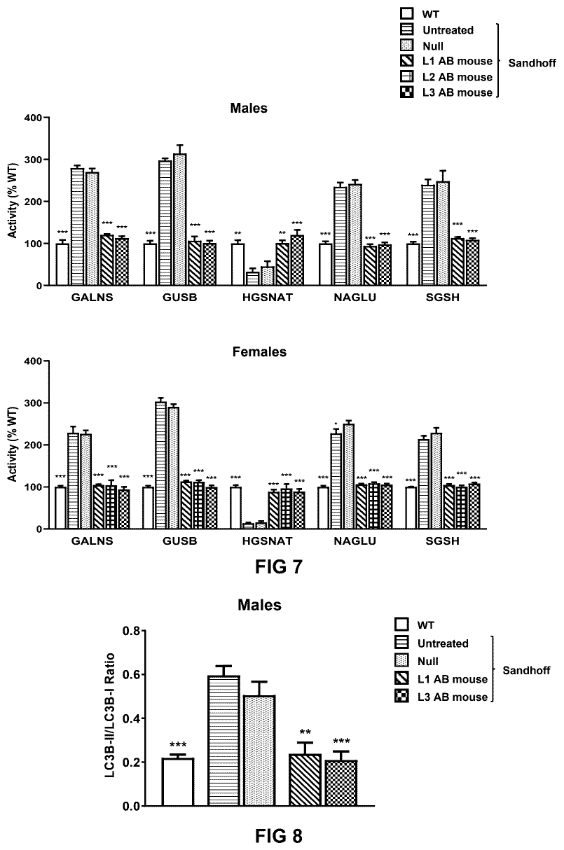

Afterwards, CNS lysosomal distension due to GM2 and cholesterol burden was assessed on brain sections by means of an immunohistochemistry with an antibody against LIMP2, a protein integral membrane to the lysosomal membrane commonly used as a marker of these organelles (Hemsley 2009 1197; Ruzo 2012 Hum Gene Ther). In agreement with correction of primary and secondary storage pathology after treatment, quantification of LIMP2+ signal intensity showed full correction in all treated Sandhoff males and females in all CNS regions analyzed (FIG. 6). These data indicated a reduction in the number and/or size of lysosomes compared to untreated and Null-injected Sandhoff mice. In LSD, the perturbation of lysosomal homeostasis can end up disturbing the normal activity of other lysosomal enzymes different from the one directly affected by the inherited gene mutation (Ribera, 2015; Motas, 2016; Roca, 2017; Marcó, 2015, Sardiello, 2008). In brain extracts of 4-month-old untreated and Null-injected Sandhoff mice, the activities of alpha-N-acetylglucosaminidase (NAGLU), N-acetylgalactosamine-6-sulfatase (GALNS), beta-glucuronidase (GUSB) and N-sulphoglucosamine sulphohydrolase (SGSH) were highly increased in the brain, up to ~2 and 3 folds, in comparison to those values measured in WT littermates (FIG. 7). On the contrary, heparan-alpha-glucosaminide N-acetyltransferase (HGSNAT) was remarkably decreased in untreated and Null-injected Sandhoff mice (FIG. 7). In both genders, there was an altered lysosomal homeostasis in untreated and Null-injected Sandhoff mice. Consistent with the reduction in primary and secondary lipid storage pathology 3 months after intra-CSF delivery of the AAV9 vectors, the activity of NAGLU, GALNS, GUSB, SGSH, and HGSNAT in the brain of L1 AB mouse- and L3 AB mouse-treated Sandhoff males and L1 AB mouse-, L2 AB mouse- and L3 AB mouse-treated Sandhoff females returned to healthy WT levels (FIG. 7). Thus, these results evidenced the reinstatement of lysosomal homeostasis in the CNS of all treated Sandhoff mice.

The endo-lysosomal compartment is an organelle also involved in autophagy; therefore, the perturbation of lysosomal distension and dysfunction could lead to an altered autophagic efflux. To determine the effect of the treatment in the autophagic flux of Sandhoff mouse model, the detection of LC3B-II/LC3B-I, a ratio of the cytosolic form (LC3B-I) versus the covalently linked to a lipid in the phagosome membrane (LC3B-I), was assessed by Western-blot. Regardless of the AAV treatment (L1 AB mouse and L3 AB mouse AAV9 vectors), a normalization of the quantification of LC3B-II/LC3B-I ratio was achieved when compared to the altered autophagic flux detected in untreated and Null-injected Sandhoff male mice (FIG. 8).

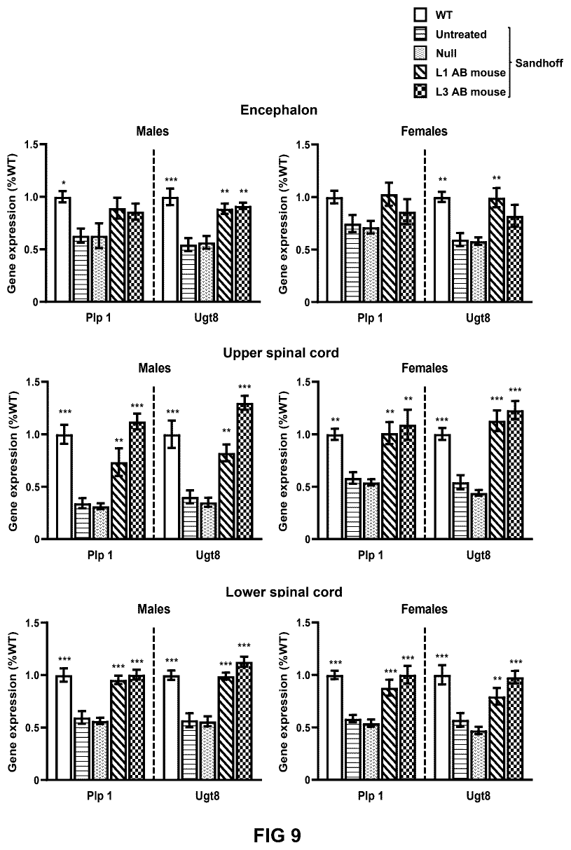

In LSD, deficits in myelinization are a common feature in neurodegenerative disorders where the primary storage pathology are lipids, such as GM2 gangliosidosis (Cachón-González, 2014). To evaluate the alteration of myelinization, expression of Plp1 and Ugt8, genes crucial as a component of myelin or involved in the myelinization process, was determined by qPCR. In brain and spinal cord, untreated and Null-injected Sandhoff male mice showed a drastic reduction of myelinization, as shown by a drop in Plp1 and Ugt8 gene expression of at least 25% and 40%, respectively (FIG. 9). In accordance with this reduction in myelinization, similar results were also observed in brain and spinal cord of female untreated and Null-injected Sandhoff mice (FIG. 9). Otherwise, intra-CSF delivery of L1 AB mouse and L3 AB mouse AAV9 vectors led to a great correction of altered myelinization in the brain of Sandhoff male mice, while in most AAV-treated Sandhoff females was detected a complete normalization (FIG. 9). Similar to what was previously reported in the brain, the upper and lower spinal cord sections of all AAV-treated Sandhoff male and female animals showed no signs of altered myelinization, reaching levels of Plp1 and Ugt8 expression similar to those values measured in WT littermates (FIG. 9).

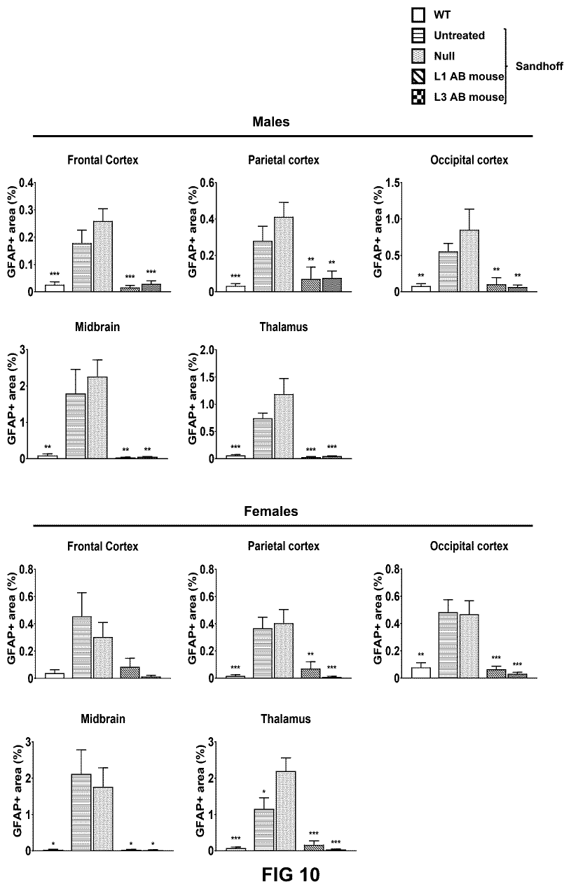

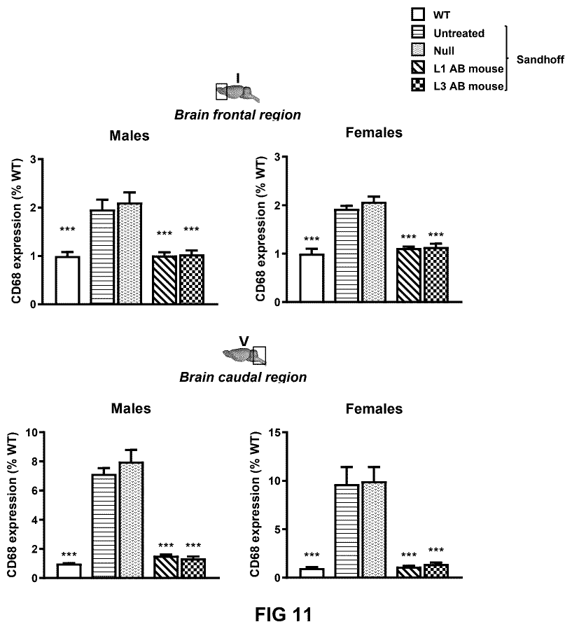

Secondary to the lysosomal pathology in neurons and glial cells, neuroinflammation is a common hallmark in Tay-Sachs and Sandhoff patients (Mierowitz 2002). We then stained encephalon sections of 4-month-old male and female mice with an antibody against glial fibrillary acidic protein (GFAP), whose expression is upregulated in activated astrocytes (Eng, 1994). In both genders, untreated and Null-injected Sandhoff mice showed a strong GFAP+ signal in all CNS areas analyzed, thus confirming the presence of astrogliosis in this animal model (FIG. 10). According to the correction of the lysosomal pathology, all signs of neuroinflammation disappeared from the CNS of treated Sandhoff male and female mice (FIG. 10). Aside from astrogliosis, untreated and Null-injected Sandhoff males and females had a striking increase in microglial infiltration in the CNS, detected by the quantification of CD68 gene expression by qPCR (FIG. 11). In both genders, all AAV-treated Sandhoff mice showed a complete absence of microglial infiltration in all CNS areas analyzed (FIG. 11). Altogether these results demonstrated the complete correction of neuroinflammation after an intra-CSF delivery of single AAV9 vectors comprising both Hexa and Hexb genes fused with a short linker L1, L2 or L3 in Sandhoff mice of both genders.

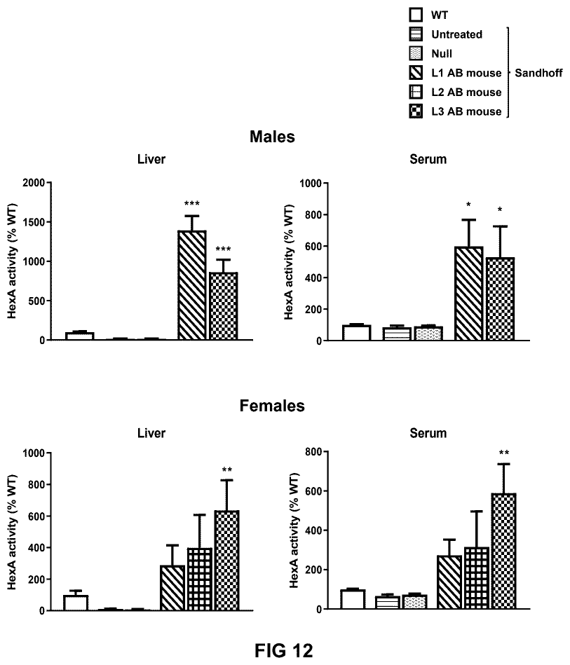

All in all, three months after a single intra-CSF administration of single AAV9 vectors comprising both Hexa and Hexb genes fused with a short linker (L1 AB mouse, L2 AB mouse and L3 AB mouse) resulted in an increase in HexA activity which in turn led to correction of the primary and secondary storage pathology, lysosomal distension and homeostasis, autophagy, myelinization, and the neuroinflammation characteristic of the disease. When AAV9 vectors are administered into the CSF, some of the vector injected leaks to the circulation and efficiently transduces the liver (Haurigot et al, 2013; Ribera et al, 2015; Roca et al, 2017; Motas et al, 2016). Therefore, a rise in HexA activity was detected in the liver of 4-month-old Sandhoff male mice treated with AAV9 vectors simultaneously coding for Hexa and Hexb fused with a short linker L1 or L3 (L1 AB mouse and L3 AB mouse) (FIG. 12). In females, similar results were observed in all treated Sandhoff mice (L1 AB mouse, L2 AB mouse and L3 AB mouse) (FIG. 12). Hepatic overexpression of HexA and HexB genes resulted in increased secretion of HexA activity into the bloodstream with values several folds higher than those documented in WT littermates (FIG. 12). As expected, hepatic and circulating HexA activity were almost undetectable in 4-month-old untreated and Null-injected Sandhoff mice. In general, the levels of HexA activity were considerably lower in females than in males, reproducing observation made in other studies in liver (Haurigot et al, 2013; Ribera et al, 2015; Roca et al, 2017; Motas et al, 2016). This gender difference is due to certain AAV serotypes are more efficient at transducing the liver of male rodents (Ruzo et al, 2012a, 2012b; Davidoff et al, 2003). Other studies, however, have demonstrated equivalent levels of liver transduction in adult male and female macaques after intravascular delivery of self-complementary AAV5, suggesting the absence of sex-dependent differences in AAV-mediated transduction in primates (Binny et al, 2012). Altogether these results indicated that the administration of both alpha and beta subunits (Hexa and Hexb genes) in a single vector led to a good production of circulating HexA isoenzyme, crucial in the stepwise degradation of GM2 gangliosides.

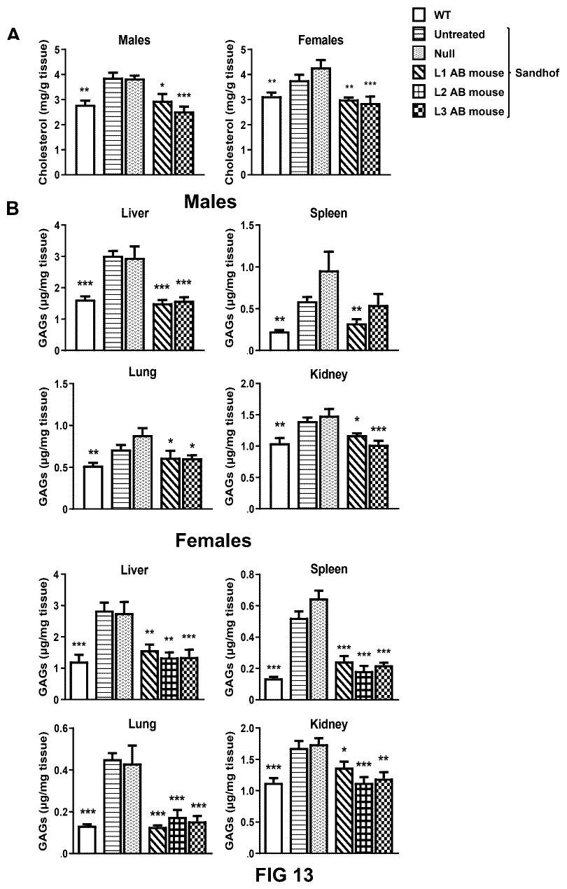

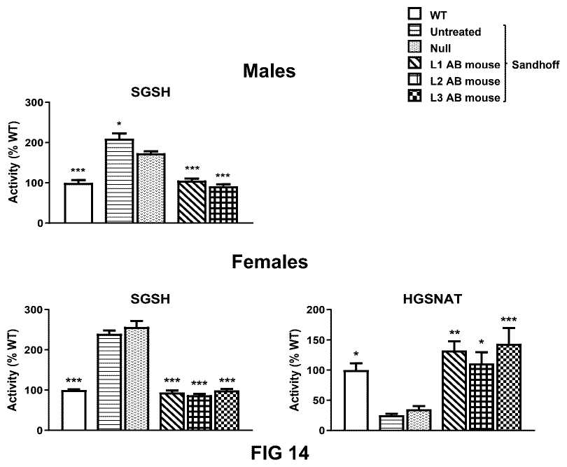

In periphery, the therapeutic efficacy of our gene therapy approach was evaluated through quantification of the secondary storage (GAGs and cholesterol) and measurement of lysosomal activities. Untreated and Null-injected Sandhoff mice of both genders showed a clear increase in cholesterol content in liver, thus indicating an evident secondary lipid storage pathology in this animal model (FIG. 13A). Three months after vector administration of L1 AB mouse and L3 AB mouse, normalization of cholesterol accumulation in liver was observed in treated Sandhoff males and females (FIG. 13A). Furthermore, the hepatic and circulating increase in transgene expression also resulted in a similar degree of correction in GAG content in all treated Sandhoff male and female mice (FIG. 13B). A great reduction of GAG content was observed in most peripheral tissues, including liver, kidney, lung, and spleen (FIG. 13B). Eventually, primary and secondary storage pathology in the lysosomes led to a severe lysosomal dysfunction detected by perturbation of other lysosomal enzymes aside from the one affected by mutations in HexB gene in Sandhoff disease. After a rise in SGSH activity in the liver, suggestive of altered lysosomal homeostasis in untreated and Null-injected Sandhoff male mice, this activity returned to healthy WT values in all treated Sandhoff male mice (FIG. 14). In females, normalization of SGSH and HGSNAT activities was observed in all treated cohorts (FIG. 14). Therefore, an efficient correction of peripheral secondary storage pathology in Sandhoff mouse model treated with L1 AB mouse, L2 AB mouse or L3 AB mouse also led to a reinstatement of lysosomal homeostasis.

Sandhoff disease is a neurodegenerative disorder that mainly affects the CNS together with a regress in developmental milestones (Regier, 2016). The juvenile and late-onset forms presented gait disturbance, incoordination and imbalance; while infantile forms showed a developmental arrest, exaggerated startle response, and hypotonia (Regier, 2016; Bley 2011 Pediatrics). To further characterize the behavioral, locomotor and coordination alterations in Sandhoff mouse model, a battery of non-invasively behavioral tests (Open Field, Rotarod, Mesh, Righting reflex and Hindlimb clasping tests) was performed.

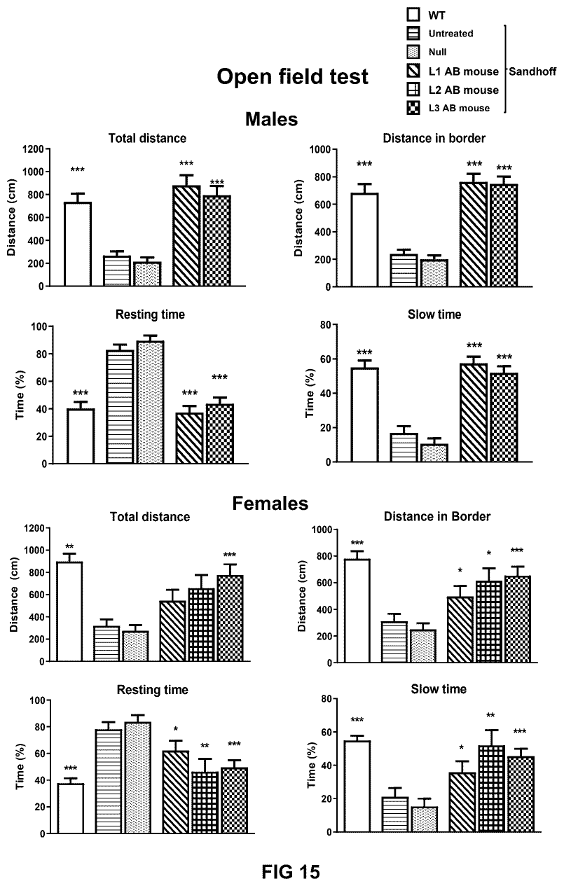

The impact of the intra-CSF administration of AAV9 vectors encoding simultaneously both Hexa and Hexb genes on behavior was assessed in the open field test, which evaluates the general locomotor and exploratory activity of mice in unknown surroundings. Untreated and Null-injected Sandhoff mice displayed reduced locomotor and exploratory activity compared to healthy WT counterparts in terms of total distance travelled, distance travelled in border, resting time and slow time (FIG. 15), which was suggestive of hypoactive and less exploratory behavior. By contrast, Sandhoff mice that received an intra-CSF administration of AAV9 vectors encoding simultaneously both Hexa and Hexb genes fused with a short linker L1, L2 or L3 at 1 month of age behaved similar to healthy WT littermates (FIG. 15). These results indicated that the behavioral disturbances induced by the genetic defect were completely corrected in L1 AB mouse-, L2 AB mouse- and L3 AB mouse-treated male and female mice.

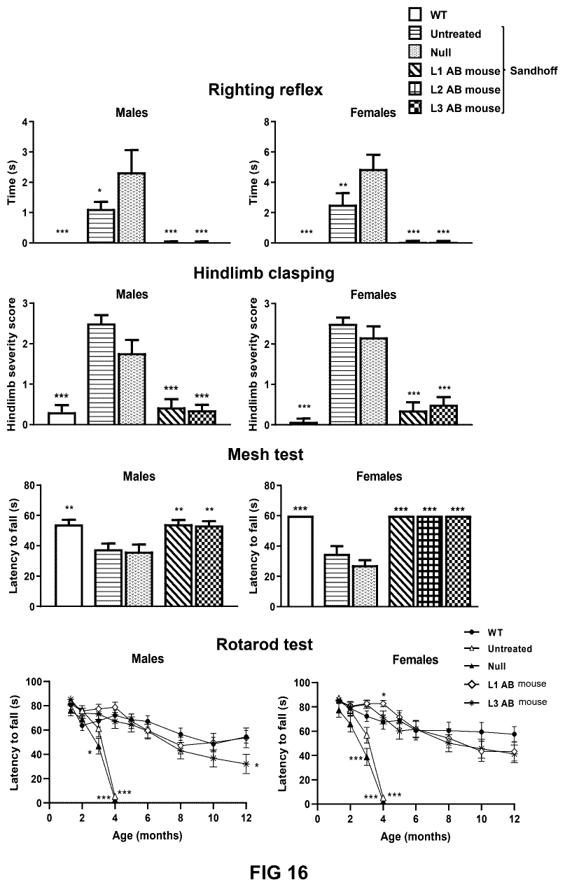

As an indicative of mobility and disease progression, the righting reflex and hindlimb clasping tests were evaluated after AAV treatment. Untreated and Null-injected Sandhoff male mice had a delay in righting reflex, whereas all AAV-treated males behaved as WT animals, reaching the lowest score possible, 0 s, a result indicative of mobility recovery (FIG. 16). Likewise, a significant delay in the righting reflex was observed in female untreated and Null-injected Sandhoff mice, while WT and all AAV-treated female cohorts had no evident delay in this reflex (FIG. 16). Another test to further evaluate disease progression, which was commonly used in different neurodegenerative animal models, is the hindlimb clasping test (Gueyenet, 2010, Lalonde 2011). The hindlimb clasping score of WT animals is around 0 in both genders, whereas untreated and Null-injected Sandhoff male and female mice had an average score of 2-3 (FIG. 16). On the contrary, a statistically significant difference was achieved when Sandhoff male and female mice received a gene therapy treatment with AAV9 vectors (L1 AB mouse or L3 AB mouse) (FIG. 16).

On the other hand, the impact of AAV9 gene transfer with our constructs (L1 AB mouse and L3 AB mouse) on motor coordination and muscle strength was evaluated through mesh test, which also allowed us to assess, at the same time, muscle strength. While 4-month-old untreated and Null-injected Sandhoff male mice averaged 37.8 s and 36.2 s on the inverted wire of the mesh test, respectively; healthy WT littermates scored 60s (FIG. 16). All treated Sandhoff male mice outperformed they untreated and Null-injected Sandhoff counterparts, with a mean score of 54 s, which was a similar latency to fall than that of healthy WT mice (FIG. 16). In females, an identical performance on mesh test was recorded (FIG. 16), thus indicating that both genders could recover their motor coordination and muscle strength to a similar degree than that of healthy WT animals. To further confirmed the recovery of motor coordination, the rotarod test was performed in all cohorts at different ages. During the first two months, there was not an obvious difference among all experimental groups in both genders (FIG. 16). As disease progressed, untreated and Null-injected Sandhoff male and female mice showed a decline in rotarod performance up to 4 months of age, which was close to the human endpoint and animals could not stand on the accelerating rotarod for more than few seconds (FIG. 16). At the same time, all cohorts treated with L1 AB mouse and L3 AB mouse AAV9 vectors had a similar performance as healthy WT littermates, with no obvious differences between males and females (FIG. 16). Thus, altered motor coordination in Sandhoff mouse model was reestablished after AAV treatment.

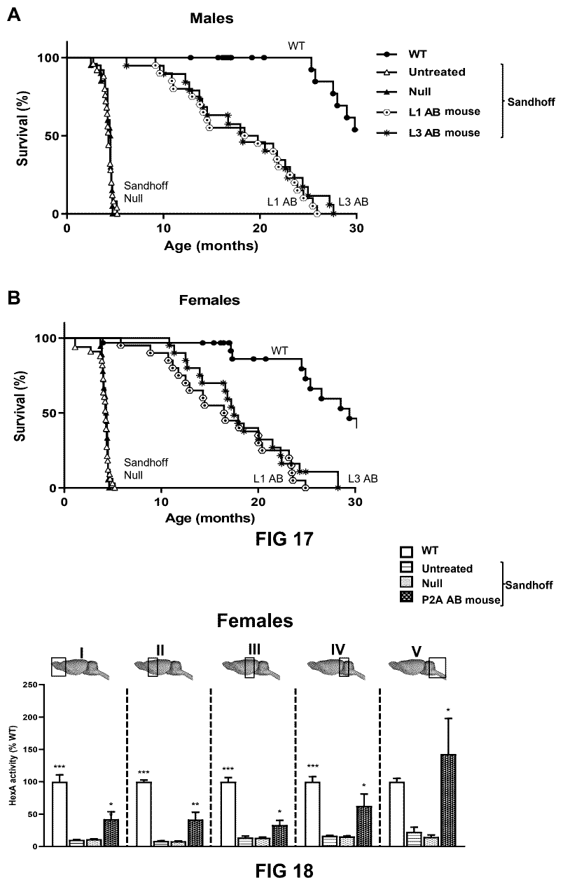

Finally, the effect of Hexa and Hexb deficiency and AAV treatment on lifespan was assessed. In both genders, untreated and Null-injected Sandhoff mice had a lifespan around 4.3-4.5 months of age (FIG. 17). On the contrary, all treatment groups significantly extended the lifespan of Sandhoff mice (FIG. 17). In males, L1 AB mouse cohort showed a median survival of 19.1 months of age, whereas L3 AB mouse group was 18.2 months of age (FIG. 17). In females, L1 AB mouse- and L3 AB mouse-treated cohorts reached a median survival rate of 16.5 and 17.5 months of age, respectively (FIG. 17). The normalization of behavioral responses and the greater survival of treated Sandhoff mice using a single AAV9 vector encoding simultaneously both Hexa and Hexb genes with a short linker L1, L2 or L3 further demonstrate the therapeutic efficacy of our gene therapy approach.

Overall, these results indicated that treatment with a single AAV9 vector encoding simultaneously both Hexa and Hexb genes fused with a short liker L1, L2 or L3 led to the efficient production of HexA isoenzyme (alpha beta (αβ) dimer), the enzyme required for the degradation of GM2 gangliosides. This increase led to the correction of CNS and peripheral pathology of the GM2 gangliosidosis, as well as functional correction of behavioral deficits and markedly extended the lifespan.

### Example 5. Therapeutic Efficacy after Intra-CSF Delivery of Two AAV9 Vectors Encoding Hexa or Hexb (Hexa+Hexb) or AAV9 Vectors Encoding Both Hexa and Hexb Genes Fused with the Self-Cleaving Linker P2A (P2A AB) in Sandhoff Mice

Next, we evaluated the HexA activity and its impact on lifespan after an intra-CSF administration of two AAV9 vectors encoding optimized murine Hexa or Hexb under the control of the ubiquitous promoter Cbh (Hexa+Hexb) (FIG. 1D) or a single AAV9 vector encoding both optimized murine Hexa and Hexb genes fused with the self-cleaving linker P2A under the control of the ubiquitous promoter Cbh (P2A AB mouse) (FIG. 1E). A total dose of either 2×10\hat{ }11 vg/mouse for the single Hexa and Hexb AAV9 vectors (1×10\hat{ }11 vg/mouse for each vector) or 1×10\hat{ }11 vg/mouse for P2A AB mouse AAV9 vector was administered into the cisterna magna of 1-month-old Sandhoff mice. These animals were then analyzed 3 months after treatment. Age-matched Sandhoff animals injected into the cisterna manga with a total dose of 1×10\hat{ }11 vg/mouse of AAV9-Cbh-Null vectors were used as control. Healthy wild-type and untreated Sandhoff mice served as additional control groups. In this study, the designed constructs used the Cbh ubiquitous promoter, the optimized murine Hexa and Hexb genes and the polyA sequence that were exactly the same as our previous studies with a covalently linked beta-hexosaminidase with a short flexible linker (L1, L2 or L3). Furthermore, Sandhoff mice were treated in the same conditions (at 1 month of age with the same dose (1×10\hat{ }11 vg/animal)) and followed up to 3 months post-treatment.

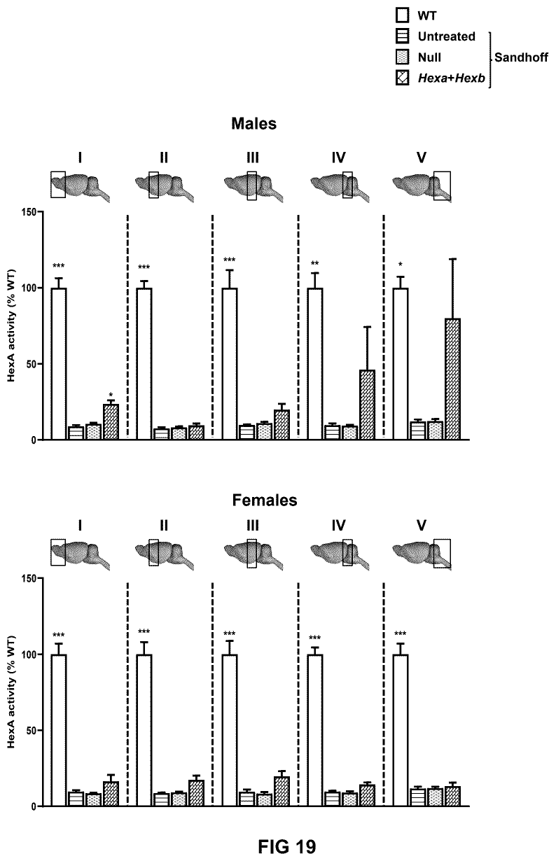

The intra-CSF administration of AAV9 vectors encoding both Hexa and Hexb fused with the self-cleaving linker, P2A AB mouse, led to a statistically significant rise in HexA activity in the encephalon of Sandhoff female mice (FIG. 18). In the majority of the CNS areas analyzed, the levels of HexA activity reached 33% to 63%, but for the closest area to the point of injection, where 143% was achieved (FIG. 18). Nevertheless, P2A AB mouse-treated Sandhoff mice showed considerably lower levels of HexA activity in nearly all CNS regions analyzed when compared to L1 AB mouse-, L2 AB mouse- and L3-AB mouse-treated Sandhoff mice (FIGS. 3 and 18). On the other hand, the intracisternal administration of two AAV9 vectors encoding Hexa or Hexb genes (Hexa+Hexb) led to a moderate increase in CNS HexA activity in treated males, detecting the highest values on the most caudal areas of the encephalon (FIG. 19). By contrast, Sandhoff female mice that received an intra-CSF administration of these two AAV9 vectors (Hexa+Hexb) only led to a slight and even increase in HexA activity in all CNS areas analyzed, with values from 13% to 20% of WT levels (FIG. 19). In general, these HexA activity values achieved after the two-vector treatment were much lower than those documented in L1 AB mouse, L2 AB mouse and L3 AB mouse-treated Sandhoff mice (FIGS. 3 and 19).

Overall, altogether these results indicated that the administration of both alpha and beta subunits (Hexa and Hexb genes) in a single construct led to a better production of HexA isoenzyme, crucial in the stepwise degradation of GM2 gangliosides. Furthermore, our AAV-based gene therapy approach based on a covalently linked beta-hexosaminidase with a short linker (L1 AB mouse, L2 AB mouse, and L3 AB mouse) demonstrated a greater capacity to efficiently produce larger amounts of active protein in comparison to the therapeutic approach based on the use of the self-cleaving linker, P2A.

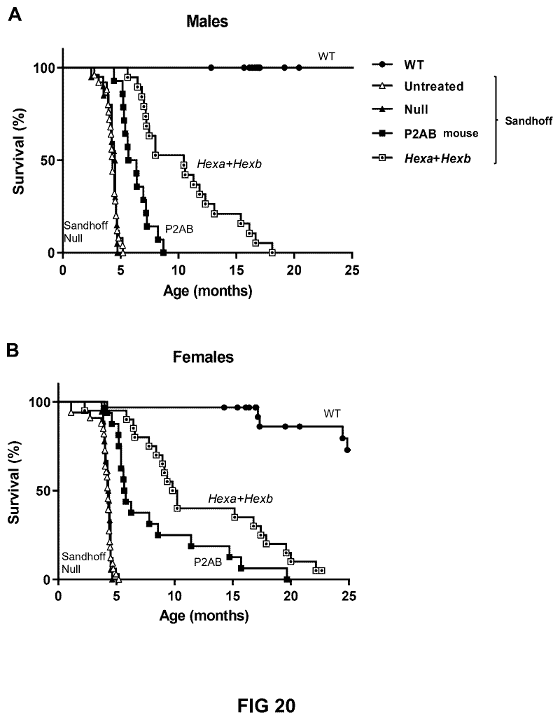

To gain insight into the therapeutic efficacy of the two AAV9 vectors encoding Hexa or Hexb genes (Hexa+Hexb) and the single AAV9 vector P2A AB mouse, the effect of the treatment was then evaluated on lifespan. As we previously observed, both genders of untreated and Null-injected Sandhoff mice had a lifespan around 4.3-4.5 months of age (FIG. 17). On the contrary, all treatment groups significantly extended the lifespan of Sandhoff mice, albeit not in the same degree (FIGS. 17 and 20). By 9 months of age, P2A AB mouse-treated Sandhoff male mice had already died, with a median survival of 6 months of age; while nearly all (95%) L1 AB mouse- and L3 AB mouse-treated animals were still alive with a survival rate of 19.1 and 18.2 months of age, respectively (FIGS. 17 and 20). Similar to what was observed in males, P2A AB mouse-treated Sandhoff females showed a median survival of 5.7 months of age, compared to 16.5 and 17.5 months of age in L1 AB mouse- and L3 AB mouse-treated Sandhoff mice (FIGS. 17 and 20). Unexpectedly, considering the very low levels of HexA activity in the CNS of Hexa+Hexb cohorts, they extended their survival rate beyond P2A AB mouse-treated Sandhoff mice (FIG. 20). Nonetheless, the median survival rate did not surpass the L1 AB mouse-nor L3 AB mouse-treated Sandhoff animals (FIGS. 17 and 20). By 10 months of age, ~50 of Hexa+Hexb-treated males and females were still alive, whereas more than 90% of L1 AB mouse- and L3 AB mouse-treated Sandhoff mice of both genders were alive (FIGS. 17 and 20). Therefore, the greater survival of treated Sandhoff mice using a single AAV9 vector encoding simultaneously both Hexa and Hexb genes with a short linker L1, L2 or L3 to produce a covalently-linked protein strongly demonstrated the superior therapeutic efficacy compared to the AAV9 vector encoding both Hexa and Hexb genes using the self-cleaving linker P2A or two AAV9 vectors encoding Hexa or Hexb genes (Hexa+Hexb).

### Example 6. Increased Beta-Hexosaminidase after Intra-CSF Delivery of AAV9 Vectors Encoding Hexa and Hexb Genes (L1 AB Mouse and L3 AB Mouse Construct) in Tay-Sachs Mice

To assess whether genetic engineering of the CNS with a single vector encoding both optimized murine Hexa and Hexa genes (FIGS. 1B and 1C) may exert therapeutic efficacy, a total dose of 1×10\hat{ }11 vg/mouse of each AAV9 vectors comprising both Hexa and Hexb fused with a short linker (L1 or L3) under the control of the ubiquitous promoter Cbh was administered into the cisterna magna of 2-month-old Tay-Sachs mice. Age-matched Tay-Sachs animals injected into the cisterna manga with a total dose of 1×10\hat{ }11 vg/mouse of AAV9-Cbh-Null vectors were used as control. Healthy wild-type and Tay-Sachs mice served as additional control groups. This animal model does not develop the clinical phenotype of Tay-Sachs disease due to a partial catabolism of accumulated GM2 via GA2 (asialo-GM2) through the combined action of sialidase and HexB isoform of beta-hexosaminidase (Phaneuf et al, 1996). Therefore, the activity of HexA isoform (alpha beta (αβ) dimer), the enzyme required for the degradation of GM2 gangliosides, was determined in the CNS, liver, and serum of all experimental groups.

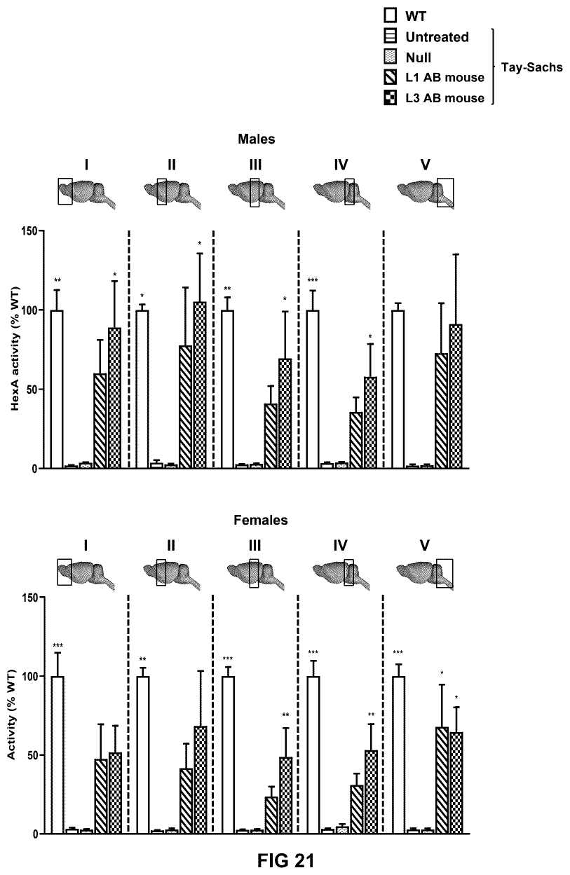

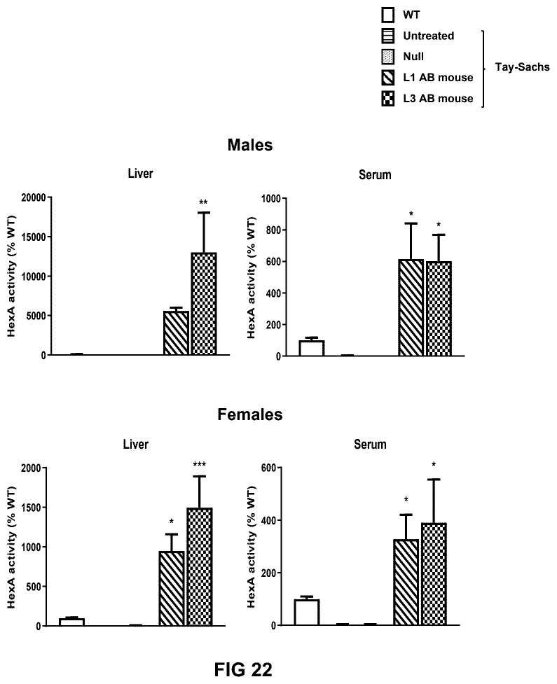

In both genders, the intra-CSF administration of AAV9 vectors encoding both Hexa and Hexb fused with a short linker L1 or L3 led to very high levels of HexA activity in all encephalon areas analyzed (FIG. 21). In males, L1 AB mouse- and L3-AB mouse-treated Tay-Sachs mice reached 58% to 105% and 36% to 78% of WT values, respectively (FIG. 21), whereas L1 AB mouse- and L3 AB mouse-treated Tay-Sachs females achieved 49% to 68% and 24% to 68% of WT values, respectively (FIG. 21). As expected, untreated and Null-injected Sandhoff mice showed almost undetectable HexA activity in brain extracts at 4 months of age (FIG. 21). As previously observed in Sandhoff-treated animals, AAV9 vectors administered into the CSF also leaked to the circulation and then efficiently transduced the liver, detected by a clear increase in hepatic and circulating HexA activity in Tay-Sachs-treated mice (FIG. 22). Nonetheless, the levels of HexA activity were much lower in females than in males, reproducing observation previously made in other studies (Haurigot et al, 2013; Ribera et al, 2015; Roca et al, 2017; Motas et al, 2016; Ruzo et al, 2012a, 2012b). This gender difference is due to the fact that certain AAV serotypes are more efficient at transducing the liver of male rodents (Ruzo et al, 2012a, 2012b; Davidoff et al, 2003). Other studies, however, have demonstrated equivalent levels of liver transduction in adult male and female macaques after intravascular delivery of self-complementary AAV5, suggesting the absence of sex-dependent differences in AAV-mediated transduction in primates (Binny et al, 2012).

Altogether these results indicated that the intra-CSF administration of both alpha and beta subunits (Hexa and Hexb genes) in a single construct in AAV9 vectors (L1 AB mouse and L3 AB mouse) led to an efficient production of HexA isoenzyme, crucial in the stepwise degradation of GM2 gangliosides.

### Example 7. Increased Beta-Hexosaminidase in HEK293 Cells after Transfection with Plasmids Encoding Both HEXA and HEXB Genes Fused with a Short Linker

First, we cloned three different optimized versions (denoted as “GA”, “IDT” and “NV”) of the human HEXA and HEXB genes fused with the peptide Linker 1 (L1 AB human GA, L1 AB human IDT and L1 AB human NV) (SEQ ID NOs: 82-84) (FIG. 1B) to evaluate our constructs and their capacity to produce active beta-hexosaminidase in comparison to the non-optimized version of human HEXA and HEXB genes also fused with the peptide Linker 1 (L1 AB human noh) (SEQ ID NO: 81) (FIG. 1B). The “GA”, “IDT” and “NV” optimized sequences of the human HEXA gene are represented by SEQ ID NO: 64, 66 and 68. The “GA”, “IDT” and “NV” optimized sequences of the human HEXB gene are represented by SEQ ID NO: 65, 67, and 69.

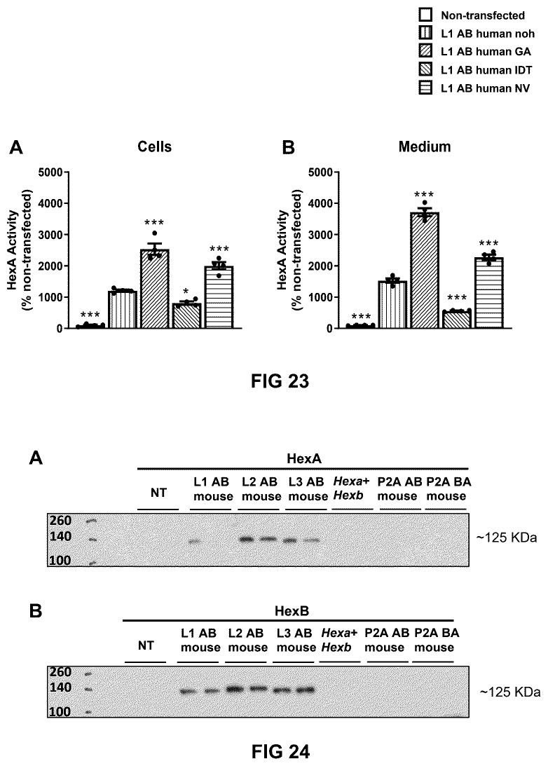

Subsequently, 0.8 μg of plasmid DNA encoding the following constructs (L1 AB human noh, L1 AB human GA, L1 AB human IDT and L1 AB human NV) (SEQ ID NOs: 60-63) (FIG. 1B) was transfected in HEK293 cells. Forty-eight hours after transfection, two (L1 AB human GA and L1 AB human NV) (SEQ ID NOs: 82 and 84) out of three optimized versions of the human HEXA and HEXB genes fused with the peptide Linker 1 reached a statistically significant increase of HexA activity in cell extracts, reaching values of 2534% and 2003% of non-transfected, respectively, when compared to non-optimized human version (FIG. 23A). Likewise, a statistically significant raise in medium of L1 AB human GA and L1 AB human NV constructs was also observed, with average activities of 3717% and 2278% of non-transfected, respectively (FIG. 23B). One optimized version (L1 AB human IDT) (SEQ ID NO: 83) of the human HEXA and HEXB genes fused with the peptide Linker 1 achieved values of 810% and 557% of non-transfected, respectively.

### Example 8. Detection of Covalently Linked Alpha Beta (αβ) Dimers in HEK293 Cells after Plasmid Transfection

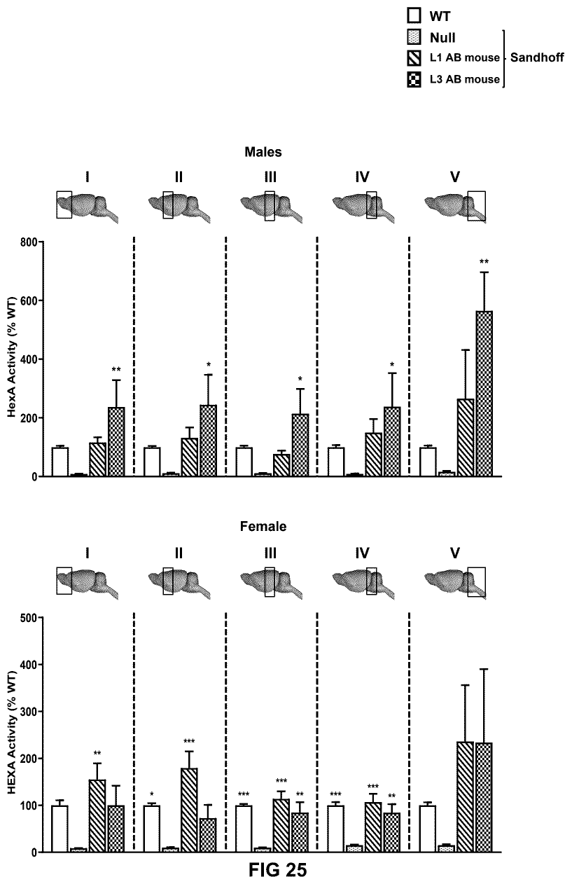

To assess the capacity to produce a covalently linked alpha beta (αβ) dimers, 4 μg of DNA were transfected in HEK293 cells to obtain medium, analyzed by Western blot. Forty-eight hours after transfection, a protein of ~125 KDa (approximately the molecular weight of covalently linked alpha beta (αβ) dimers) was detected with HEXA and HEXB antibodies in medium of our linker-based constructs (L1 AB mouse, L2 AB mouse and L3 AB mouse) (SEQ ID NOs: 75-77), which could be identified as our fused protein (HEXA+linker+HEXB) (FIG. 24). These results demonstrated that our L1 AB mouse, L2 AB mouse and L3 AB mouse constructs give rise to a covalently linked alpha (αβ) dimers (HEXA+linker+HEXB), regardless of our peptide linker used. In contrast, when HEK293 were co-transfected with Hexa and Hexb genes (SEQ ID NOs: 85-86) or transfected with P2A-based mouse constructs (SEQ ID NOs: 87-88), we could not detect any band of high molecular weight which could be indicative of a fused protein (HEXA+HEXB), thus showing that no covalently linked (αβ) dimers were produced (FIG. 24). Taken together, our data demonstrated that the Hexa and Hexb genes fused with any of the peptide linkers as disclosed herein leads to a covalently linked alpha beta (αβ) dimers.

### Example 9. Long-Term Therapeutic Efficacy after Intra-CSF Delivery of AAV9 Vectors Encoding Both Hexa and Hexb Genes (L1 AB Mouse and L3 AB Mouse) in Sandhoff Mice

The therapeutic effects described in Example 4 were also observed in Sandhoff mice 7 months after intra-CSF delivery of AAV9 vectors encoding both Hexa and Hexb genes fused with a short linker L1 or L3 (L1 AB mouse and L3 AB mouse) under the control of the ubiquitous promoter Cbh a total dose of 1×10\hat{ }11 vg/mouse. Age-matched Sandhoff animals injected into the cisterna manga with a total dose of 1×10\hat{ }11 vg/mouse of AAV9-Cbh-Null vectors were used as control. Healthy wild-type and Sandhoff mice served as additional control groups. After seven months, the intra-CSF administration of AAV9 vectors comprising both Hexa and Hexb fused with a short linker L1 or L3 led to, at least, similar levels of HexA activity as those documented in healthy WT animals in most CNS areas analyzed (FIG. 25). In females, similar results were observed in L1 AB mouse- and L3-AB mouse-treated Sandhoff mice, in which HexA activity reached at least similar levels of healthy WT animals in most encephalon regions (FIG. 25). Of note, the levels of HexA activity in all AAV-treated Sandhoff mice showed an even distribution throughout the CNS, from the most distal to the closest point of the injection (FIG. 25). As expected, 4-month-old Null-injected Sandhoff mice showed almost undetectable HexA activity in brain extracts at 4 months of age, once a human endpoint is reached and animals were euthanized (FIG. 25).

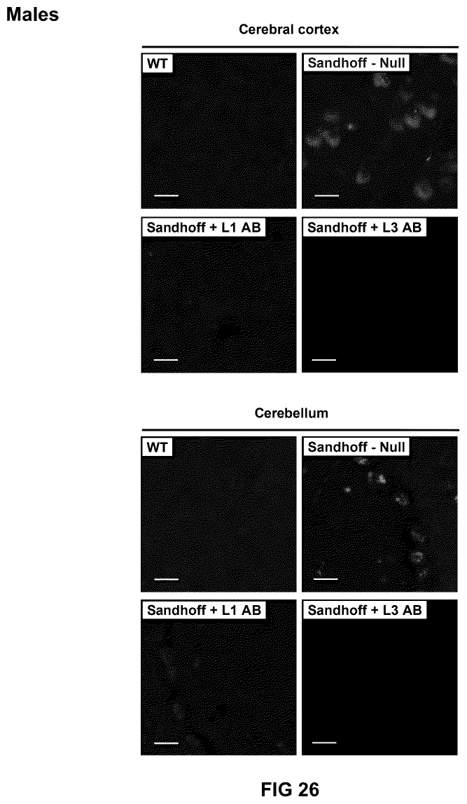

To further evaluate the therapeutic efficacy of the treatment at long-term, a filipin staining was performed in encephalon sections of Sandhoff-treated mice. The increased transgene expression in the CNS of Sandhoff-treated animals also resulted in normalization of cholesterol accumulation in the cerebral cortex and cerebellum observed in Null-injected Sandhoff mice, as shown in the photomicrographs of filipin staining (FIG. 26). These results indicated long-term correction of secondary cholesterol storage pathology in the CNS after L1 AB mouse and L3 AB mouse treatment (FIG. 26).

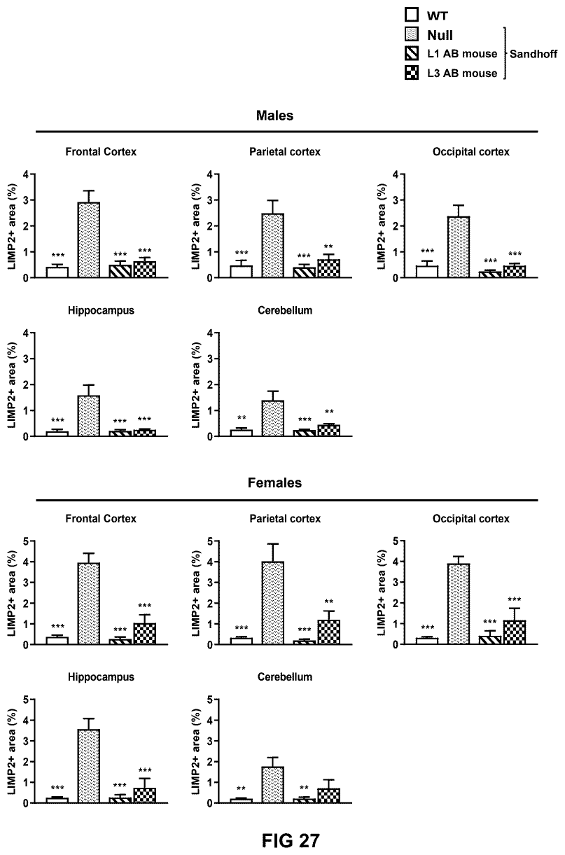

Due to primary GM2 and secondary cholesterol burden, Null-injected Sandhoff mice showed a severe CNS lysosomal distension detected in brain sections by means of an immunohistochemistry with an antibody against LIMP2, a protein integral membrane to the lysosomal membrane commonly used as a marker of these organelles (Hemsley 2009; Ruzo 2012b). In agreement with long-term correction of storage pathology after treatment, quantification of LIMP2+ signal intensity showed complete correction, similar to what was previously observed three months after gene transfer, in all treated Sandhoff males and females in most CNS regions analyzed (FIG. 27). These data indicated long-term reduction in the number and/or size of lysosomes compared to Null-injected Sandhoff mice.

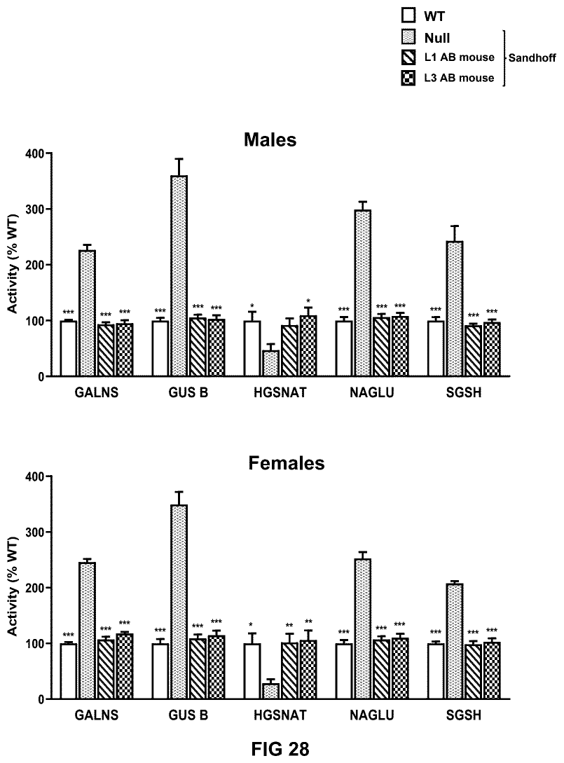

In LSD, such as Sandhoff disease, the perturbation of lysosomal homeostasis ended up disturbing the normal activity of other lysosomal enzymes different from the one directly affected by the inherited gene mutation, such as GALNS, GUSB, HGSNAT, NAGLU and SGSH (FIG. 28). In both genders, seven months after intra-CSF delivery of the AAV9 vectors, the activity of NAGLU, GALNS, GUSB, SGSH, and HGSNAT in the brain of L1 AB mouse- and L3 AB mouse-treated Sandhoff mice returned to healthy WT levels (FIG. 28). Thus, these results evidenced the long-term correction of altered lysosomal homeostasis in the CNS of all treated Sandhoff mice.

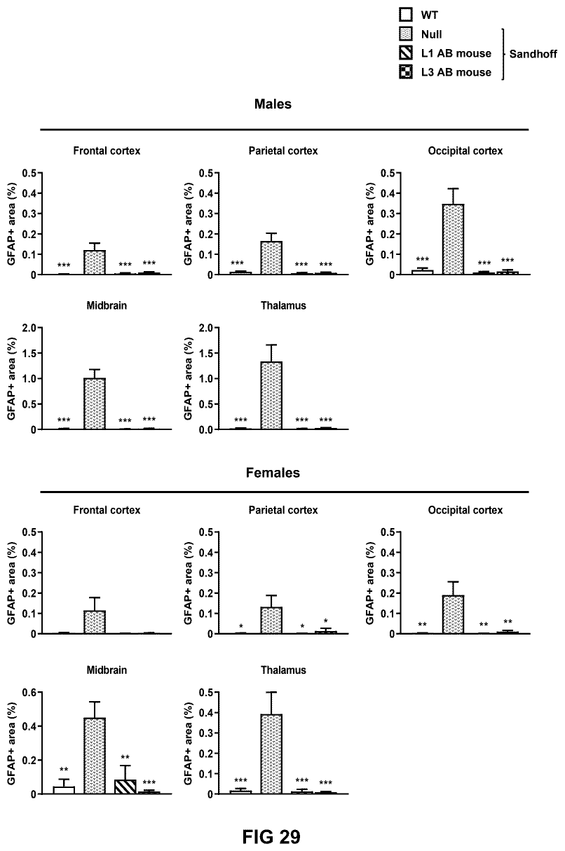

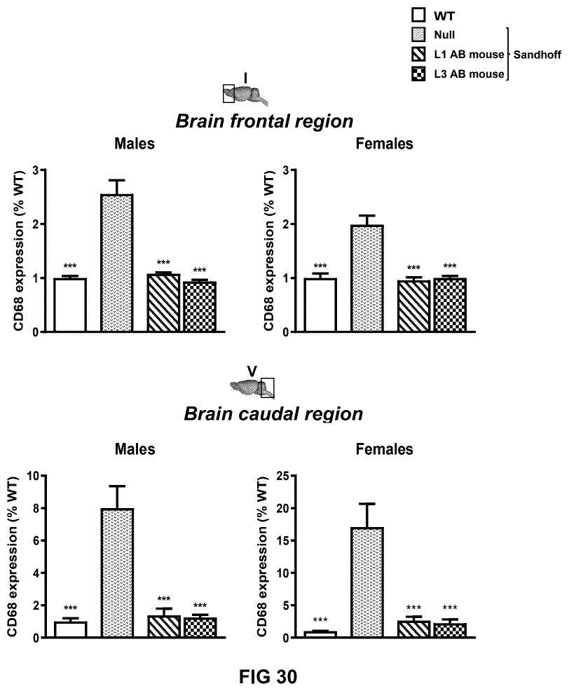

Secondary to the lysosomal pathology in neurons and glial cells, 4-month-old Null-injected Sandhoff mice also presented severe neuroinflammation, detected after an immunostaining with GFAP in encephalon sections. According to long-term correction of lysosomal pathology, all signs of astrogliosis were completely absent from the CNS of treated Sandhoff male and female mice seven months after treatment (FIG. 29). Aside from astrogliosis, disappearance of the striking increase in microglial infiltration detected in the CNS of Null-injected Sandhoff males and females, after quantification of CD68 gene expression by qPCR, was maintained up to seven months after gene transfer in all AAV-treated Sandhoff mice in both genders (FIG. 30). Altogether these results demonstrated the long-term correction of neuroinflammation after a single intra-CSF delivery of AAV9 vectors comprising both Hexa and Hexb genes fused with a short linker in Sandhoff mice of both genders.

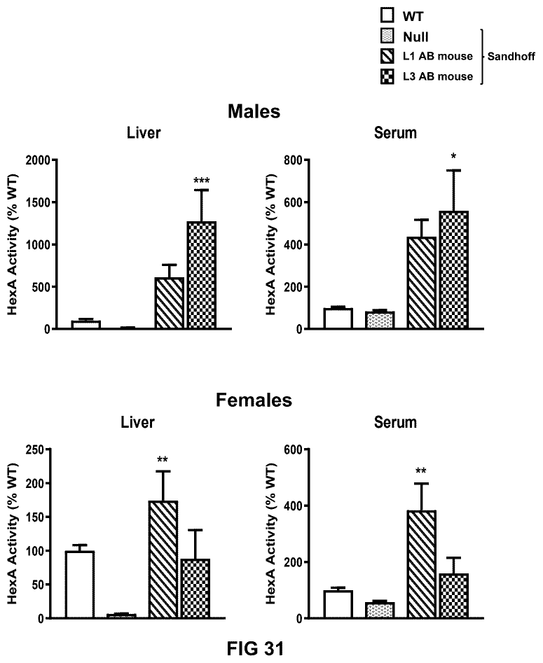

When AAV9 vectors were administered into the CSF, some of the vector injected leaked to the circulation and efficiently transduced the liver. Therefore, seven months after treatment, a rise in HexA activity was also detected in the liver of 8-month-old Sandhoff male mice treated with AAV9 vectors simultaneously coding for Hexa and Hexb fused with a short linker L1 or L3 (L1 AB mouse and L3 AB mouse) (FIG. 31). In females, similar results were observed at long term in all treated Sandhoff mice (L1 AB mouse and L3 AB mouse) (FIG. 31). Hepatic overexpression of HexA and HexB genes also resulted in increased secretion of HexA activity, at long term, into the bloodstream with values several folds higher than those documented in WT littermates (FIG. 31). In general, the levels of HexA activity were considerably lower in females than in males, reproducing observation made in other studies in liver (Haurigot et al, 2013; Ribera et al, 2015; Roca et al, 2017; Motas et al, 2016). This gender difference is due to certain AAV serotypes are more efficient at transducing the liver of male rodents (Ruzo et al, 2012a, 2012b; Davidoff et al, 2003). Other studies, however, have demonstrated equivalent levels of liver transduction in adult male and female macaques after intravascular delivery of self-complementary AAV5, suggesting the absence of sex-dependent differences in AAV-mediated transduction in primates (Binny et al, 2012). Altogether these results indicated that the administration of both alpha beta subunits (Hexa and Hexb genes) in a single construct in AAV9 vectors led to a good production of circulating HexA isoenzyme, crucial in the stepwise degradation of GM2 gangliosides.

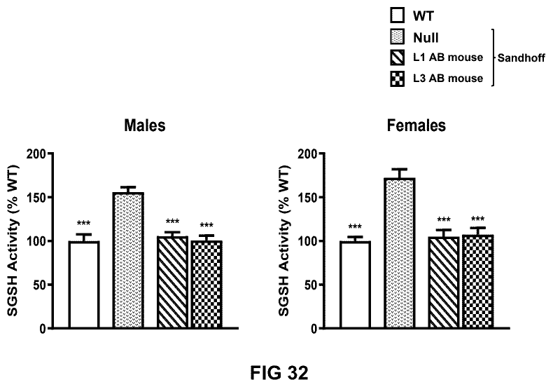

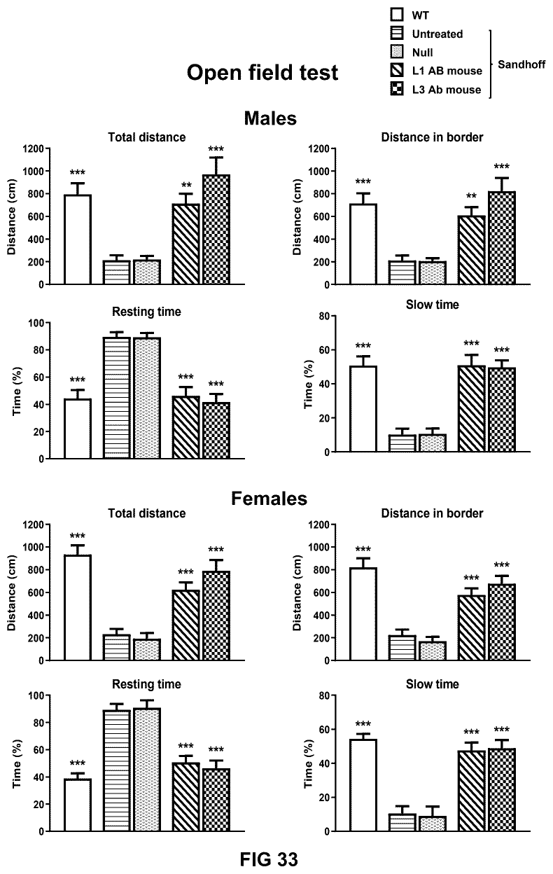

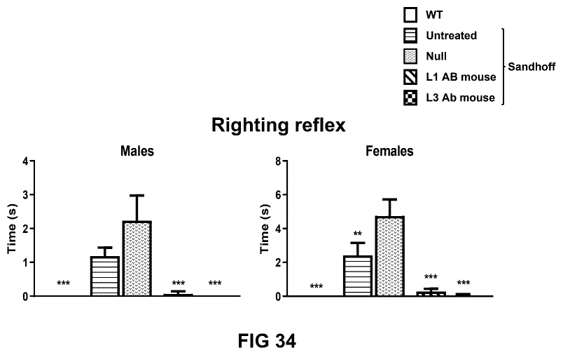

In periphery, the therapeutic efficacy of our gene therapy approach was evaluated through assessment of lysosomal dysfunction detected by alteration of another lysosomal enzyme (SGSH) aside from the one affected by mutations in HexB gene in Sandhoff disease. The rise in SGSH activity in the liver, suggestive of altered lysosomal homeostasis in 4-month-old Null-injected Sandhoff male mice, was returned to healthy WT values seven months after treatment (FIG. 32). In females, long-term normalization of SGSH activity was also observed in all treated cohorts (FIG. 32). Therefore, seven months after an intra-CSF delivery of AAV9 vectors encoding both Hexa and Hexb genes in Sandhoff mice led to long-term reinstatement of lysosomal homeostasis. Since Sandhoff disease is a neurodegenerative disorder that mainly affects the CNS together with a regress in developmental milestones (Regier, 2016), some non-invasively behavioral tests (Open Field and Righting reflex tests) were performed to further assess the long-term therapeutic effects of our gene therapy approach on behavioral alterations in Sandhoff mouse model. In the open field test, untreated and Null-injected Sandhoff mice displayed reduced locomotor and exploratory activity compared to healthy WT counterparts in terms of total distance travelled, distance travelled in border, resting time and slow time (FIG. 33), which was suggestive of hypoactive and less exploratory behavior. By contrast, Sandhoff mice that received a single intra-CSF administration of AAV9 vectors encoding simultaneously both Hexa and Hexb genes fused with a short linker L1 or L3 at 1 month of age behaved as healthy WT littermates seven months after treatment (FIG. 33). Furthermore, the righting reflex test (indicative of mobility and disease progression), was also performed seven months after AAV treatment. Untreated and Null-injected Sandhoff male mice had a delay in righting reflex, whereas all AAV-treated males behaved as WT animals at long-term, reaching the lowest score possible, 0 s, a result indicative of mobility recovery (FIG. 34). Likewise, a significant delay in the righting reflex was observed in female untreated and Null-injected Sandhoff mice, while WT and all AAV-treated female cohorts had no evident delay in this reflex seven months after gene transfer (FIG. 34). These results indicated that the behavioral disturbances induced by the genetic defect were completely corrected in L1 AB mouse- and L3 AB mouse-treated male and female mice.

All in all, seven months after a single intra-CSF administration of single AAV9 vectors comprising both Hexa and Hexb genes fused with a short linker (L1 AB mouse and L3 AB mouse) resulted in an increase in HexA activity which in turn led to correction of CNS and peripheral pathology of the GM2 gangliosidosis, as well as functional correction of behavioral deficits. Therefore, altogether these results show that the gene constructs described herein lead to long-term clinical improvements.

### REFERENCES

Arfi A, Bourgoin C, Basso L, Emiliani C, Tancini B, Chigorno V, Li Y T, Orlacchio A, Poenaru L, Sonnino S, et al (2005) Bicistronic lentiviral vector corrects β-hexosaminidase deficiency in transduced and cross-corrected human Sandhoff fibroblasts. Neurobiol Dis 20:583-593
Ayuso E, Mingozzi F & Bosch F (2010) Production, purification and characterization of adeno-associated vectors. Curr Gene Ther 10:423-436
Batista L, Miller F, Clave C, Arfi A, Douillard-Guilloux G, Couraud P-O & Caillaud C (2010) Induced secretion of β-hexosaminidase by human brain endothelial cells: A novel approach in Sandhoff disease? Neurobiol Dis 37:656-660
Binny C, McIntosh J, della Peruta M, Kymalainen H, Tuddenham E G, Buckley S M, Waddington S N, McVey J H, Spence Y, Morton C L, et al (2012) AAV-mediated gene transfer in the perinatal period results in expression of FVII at levels that protect against fatal spontaneous hemorrhage. Blood 119:957-966
Cachón-González M B, Wang S Z, Ziegler R, Cheng SH & Cox™ (2014) Reversibility of neuropathology in Tay-Sachs-related diseases. Hum Mol Genet 23:730-748
Cachon-Gonzalez M B, Zaccariotto E & Cox™ (2018) Genetics and Therapies for GM2 Gangliosidosis. Curr Gene Ther 18 Chai Y R, Ge M M, Wei T T, Jia Y L, Guo X & Wang T Y (2018) Human rhinovirus internal ribosome entry site element enhances transgene expression in transfected CHO-S cells. Sci Rep 8
Davidoff A M, Ng C Y, Zhou J, Spence Y & Nathwani A C (2003) Sex significantly influences transduction of murine liver by recombinant adeno-associated viral vectors through an androgen-dependent pathway. Blood 102:480-488
Eng L F & Ghirnikar R S (1994) GFAP and astrogliosis. Brain Pathol 4:229-37
Flotte T R, Cataltepe O, Puri A, Batista A R, Moser R, McKenna-Yasek D, Douthwright C, Gernoux G, Blackwood M, Mueller C, et al (2022) AAV gene therapy for Tay-Sachs disease. Nat Med 28:251-259
Guyenet S J, Furrer S A, Damian V M, Baughan T D, Ia Spada AR & Garden GA (2010) A Simple Composite Phenotype Scoring System for Evaluating Mouse Models of Cerebellar Ataxia. J Vis Exp
Hall P, Minnich S, Teigen C & Raymond K (2014) Diagnosing Lysosomal Storage Disorders: The GM2 Gangliosidoses. Curr Protoc Hum Genet 83
Haurigot V, Marcó S, Ribera A, Garcia M, Ruzo A, Villacampa P, Ayuso E, Añ or S, Andaluz A, Pineda M, et al (2013) Whole body correction of mucopolysaccharidosis IIIA by intracerebrospinal fluid gene therapy. Journal of clinical Investigation 123:3254-3271
Hemsley K M, Luck A J, Crawley A C, Hassiotis S, Beard H, King B, Rozek T, Rozaklis T, Fuller M & Hopwood J J (2009) Examination of intravenous and intra-CSF protein delivery for treatment of neurological disease. Eur J Neurosci 29:1197-1214
Jain A, Kohli A & Sachan D (2010) Infantile Sandhoff's disease with peripheral neuropathy. Pediatr Neurol 42:459-461
Karumuthil-Melethil S, Nagabhushan Kalburgi S, Thompson P, Tropak M, Kaytor M D, Keimel J G, Mark B L, Mahuran D, Walia J S & Gray S J (2016a) Novel Vector Design and Hexosaminidase Variant Enabling Self-Complementary Adeno-Associated Virus for the Treatment of Tay-Sachs Disease. Hum Gene Ther 27:509-21
Karumuthil-Melethil S, Nagabhushan Kalburgi S, Thompson P, Tropak M, Kaytor M D, Keimel J G, Mark B L, Mahuran D, Walia J S & Gray S J (2016b) Novel Vector Design and Hexosaminidase Variant Enabling Self-Complementary Adeno-Associated Virus for the Treatment of Tay-Sachs Disease. Hum Gene Ther 27:509-521
Kot S, Karumuthil-Melethil S, Woodley E, Zaric V, Thompson P, Chen Z, Lykken E, Keimel J G, Kaemmerer W F, Gray S J, et al (2021) Investigating Immune Responses to the scAAV9-HEXM Gene Therapy Treatment in Tay-Sachs Disease and Sandhoff Disease Mouse Models. Int J Mol Sci 22
Lahey H G, Webber C J, Golebiowski D, Izzo C M, Horn E, Taghian T, Rodriguez P, Batista A R, Ellis L E, Hwang M, et al (2020) Pronounced Therapeutic Benefit of a Single Bidirectional AAV Vector Administered Systemically in Sandhoff Mice. Molecular Therapy 28:2150-2160
Lalonde R & Strazielle C (2011) Brain regions and genes affecting limb-clasping responses. Brain Res Rev 67:252-259
Lawson C & Martin D (2016) Animal models of GM2 gangliosidosis: utility and limitations. Appl Clin Genet Volume 9:111-120
Leal A F, Benincore-Flórez E, Solano-Galarza D, Jaramillo R G G, Echeverri-Peñ a O Y, Suarez D A, Alméciga-Diaz C J & Espejo-Mojica A J (2020) Gm2 gangliosidoses: Clinical features, pathophysiological aspects, and current therapies. Int J Mol Sci 21:1-27 doi: 10.3390/ijms21176213 [PREPRINT]
Lock M, McGorray S, Auricchio A, Ayuso E, Beecham E J, Blouin-Tavel V, Bosch F, Bose M, Byrne B J, Caton T, et al (2010) Characterization of a recombinant adeno-associated virus type 2 Reference Standard Material. Hum Gene Ther 21:1273-1285
Lopez PHH & Báez BB (2018) Gangliosides in Axon Stability and Regeneration. Prog Mol Biol Transl Sci 156:383-412
Lowden J A, Skomorowski M A, Henderson F & Kaback M (1973) Automated Assay of Hexosaminidases in Serum. Clin Chem 19:1345-1349
Marcó S, Pujol A, Roca C, Motas S, Ribera A, Garcia M, Molas M, Villacampa P, Melia CS, Sanchez V, et al (2016) Progressive neurologic and somatic disease in a novel mouse model of human mucopolysaccharidosis type IIIC. Dis Model Mech 9:999-1013
Masingue M, Dufour L, Lenglet T, Saleille L, Goizet C, Ayrignac X, Ory-Magne F, Barth M, Lamari F, Mandia D, et al (2020) Natural History of Adult Patients with GM2 Gangliosidosis. Ann Neurol 87:609-617
Motas S, Haurigot V, Garcia M, Marcó S, Ribera A, Roca C, Sánchez X, Sánchez V, Molas M, Bertolin J, et al (2016)CNS-directed gene therapy for the treatment of neurologic and somatic mucopolysaccharidosis type II (Hunter syndrome). JCI Insight 1: e86696
Myerowitz R, Lawson D, Mizukami H, Mi Y, Tifft C J & Proia R L (2002) Molecular pathophysiology in Tay-Sachs and Sandhoff diseases as revealed by gene expression profiling. Hum Mol Genet 11:1343-1350
Okada S & O'Brien J S (1969) Tay-Sachs disease: generalized absence of a beta-D-N-acetylhexosaminidase component. Science 165:698-700
Ornaghi F, Sala D, Tedeschi F, Maffia M C, Bazzucchi M, Morena F, Valsecchi M, Aureli M, Martino S & Gritti A (2020) Novel bicistronic lentiviral vectors correct B-Hexosaminidase deficiency in neural and hematopoietic stem cells and progeny: implications for in vivo and ex vivo gene therapy of GM2 gangliosidosis. Neurobiol Dis 134
Osmon K J L, Woodley E, Thompson P, Ong K, Karumuthil-Melethil S, Keimel J G, Mark B L, Mahuran D, Gray S J & Walia J S (2016) Systemic Gene Transfer of a Hexosaminidase Variant Using an scAAV9.47 Vector Corrects GM2 Gangliosidosis in Sandhoff Mice. Hum Gene Ther 27:497-508
Ou L, Przybilla M J, Tabăran A F, Overn P, O'Sullivan M G, Jiang X, Sidhu R, Kell P J, Ory D S & Whitley C B (2020) A novel gene editing system to treat both Tay-Sachs and Sandhoff diseases. Gene Ther
Phaneuf D, Wakamatsu N, Huang J Q, Borowski A, Peterson A C, Fortunato S R, Ritter G, Igdoura S A, Morales C R, Benoit G, et al (1996) Dramatically different phenotypes in mouse models of human Tay-Sachs and Sandhoff diseases. Hum Mol Genet 5:1-14
Rahman M M, Chang H S, Mizukami K, Hossain M A, Yabuki A, Tamura S, Kitagawa M, Mitani S, Higo T, Uddin M M, et al (2012) A frameshift mutation in the canine HEXB gene in toy poodles with GM2 gangliosidosis variant 0 (Sandhoff disease). Vet J 194:412-416
Regier D S, Proia R L, D′Azzo A & Tifft C J (2016) The GM1 and GM2 Gangliosidoses: Natural History and Progress toward Therapy. Pediatr Endocrinol Rev 13:663
Regina Todeschini A & Hakomori S itiroh (2008) Functional role of glycosphingolipids and gangliosides in control of cell adhesion, motility, and growth, through glycosynaptic microdomains. Biochim Biophys Acta 1780:421-433
Ribera A, Haurigot V, Garcia M, Marcó S, Motas S, Villacampa P, Maggioni L, León X, Molas M, Sánchez V, et al (2015) Biochemical, histological and functional correction of mucopolysaccharidosis Type IIIB by intra-cerebrospinal fluid gene therapy. Hum Mol Genet 24:2078-2095
Roca C, Motas S, Marcó S, Ribera A, Sánchez V, Sánchez X, Bertolin J, León X, Pérez J, Garcia M, et al (2017) Disease correction by AAV-mediated gene therapy in a new mouse model of mucopolysaccharidosis type IIID. Hum Mol Genet 26:1535-1551
Rubovitch V, Zilberstein Y, Chapman J, Schreiber S & Pick CG (2017) Restoring GM1 ganglioside expression ameliorates axonal outgrowth inhibition and cognitive impairments induced by blast traumatic brain injury. Sci Rep 7
Ruzo A, Garcia M, Ribera A, Villacampa P, Haurigot V, Marco S, Ayuso E, Anguela X M, Roca C, Agudo J, et al (2012a) Liver production of sulfamidase reverses peripheral and ameliorates CNS pathology in mucopolysaccharidosis IIIA mice. Mol Ther 20:254-266
Ruzo A, Marcó S, Garcia M, Villacampa P, Ribera A, Ayuso E, Maggioni L, Mingozzi F, Haurigot V & Bosch F (2012b) Correction of pathological accumulation of glycosaminoglycans in central nervous system and peripheral tissues of MPSIIIA mice through systemic AAV9 gene transfer. Hum Gene Ther 23:1237-1246
Sanders D N, Zeng R, Wenger D A, Johnson G S, Johnson G C, Decker J E, Katz M L, Platt S R & O'Brien D P (2013) GM2 gangliosidosis associated with a HEXA missense mutation in Japanese Chin dogs: a potential model for Tay Sachs disease. Mol Genet Metab 108:70-75
Sandhoff K & Harzer K (2013) Gangliosides and gangliosidoses: principles of molecular and metabolic pathogenesis. J Neurosci 33:10195-10208
Sango K, Yamanaka S, Hoffmann A, Okuda Y, Grinberg A, Westphal H, McDonald M P, Crawley J N, Sandhoff K, Suzuki K, et al (1995) Mouse models of Tay-Sachs and Sandhoff diseases differ in neurologic phenotype and ganglioside metabolism. Nat Genet 11:170-6
Sardiello M, Palmieri M, di Ronza A, Medina D L, Valenza M, Gennarino V A, di Malta C, Donaudy F, Embrione V, Polishchuk R S, et al (2009) A gene network regulating lysosomal biogenesis and function. Science 325:473-7
Schnaar R L (2010) Brain gangliosides in axon-myelin stability and axon regeneration. FEBS Lett 584:1741-1747
Schnaar R L (2019) The Biology of Gangliosides. Adv Carbohydr Chem Biochem 76:113-148
Seyrantepe V, Demir S A, Timur Z K, von Gerichten J, Marsching C, Erdemli E, Oztas E, Takahashi K, Yamaguchi K, Ates N, et al (2018) Murine Sialidase Neu3 facilitates GM2 degradation and bypass in mouse model of Tay-Sachs disease. Exp Neurol 299:26-41
Shaimardanova A A, Chulpanova D S, Solovyeva V v., Aimaletdinov A M & Rizvanov A A (2022) Functionality of a bicistronic construction containing HEXA and HEXB genes encoding B-hexosaminidase A for cell-mediated therapy of GM2 gangliosidoses. Neural Regen Res 17:122-129
Solovyeva V v., Shaimardanova A A, Chulpanova D S, Kitaeva K v., Chakrabarti L & Rizvanov A A (2018) New approaches to Tay-Sachs disease therapy. Front Physiol 9
Sonnino S, Chiricozzi E, Grassi S, Mauri L, Prioni S & Prinetti A (2018) Gangliosides in Membrane Organization. Prog Mol Biol Trans/Sci 156:83-120
Sun A, Chang I J, Lam C & Berry G T (2021) Lysosomal Storage Disorders. Emery and Rimoin's Principles and Practice of Medical Genetics and Genomics: 563-682
Torres P A, Zeng B J, Porter B F, Alroy J, Horak F, Horak J & Kolodny E H (2010) Tay-Sachs disease in Jacob sheep. Mol Genet Metab 101:357-363
Tropak M B, Yonekawa S, Karumuthil-Melethil S, Thompson P, Wakarchuk W, Gray S J, Walia J S, Mark B L & Mahuran D (2016) Construction of a hybrid B-hexosaminidase subunit capable of forming stable homodimers that hydrolyze GM2 ganglioside in vivo. Mol Ther Methods Clin Dev 3:15057
Venugopalan P & Joshi SN (2002) Cardiac involvement in infantile Sandhoff disease. J Paediatr Child Health 38:98-100
Woodley E, Osmon K J L, Thompson P, Richmond C, Chen Z, Gray S J & Walia J S (2019) Efficacy of a Bicistronic Vector for Correction of Sandhoff Disease in a Mouse Model. Mol Ther Methods Clin Dev 12:47-57
Yu R K, Tsai Y T, Ariga T & Yanagisawa M (2011) Structures, biosynthesis, and functions of gangliosides—an overview. J Oleo Sci 60:537-544
Zeller C B & Marchase R B (1992) Gangliosides as modulators of cell function. Am J Physiol 262
Zeng B J, Torres P A, Viner T C, Wang Z H, Raghavan S S, Alroy J, Pastores G M & Kolodny E H (2008) Spontaneous appearance of Tay-Sachs disease in an animal model. Mol Genet Metab 95:59-65
Zhang J, Chen H, Kornreich R & Yu C (2019) Prenatal diagnosis of tay-sachs disease. Methods in Molecular Biology 1885:233-250

Sequences

| SEQ | |
|---|---|
| ID NO: | Description of the sequence |
| 1 | Amino acid sequence of Homo sapiens alpha subunit of beta-hexosaminidase |
| 2 | Amino acid sequence of Mus musculus alpha subunit of beta-hexosaminidase |
| 3 | Nucleotide sequence of Homo sapiens alpha subunit of beta-hexosaminidase |
| 4 | Nucleotide sequence of Mus musculus alpha subunit of beta-hexosaminidase |
| 5 | Optimized nucleotide sequence of Mus musculus alpha subunit of beta-hexosaminidase |
| 6 | Amino acid sequence of Homo sapiens beta subunit of beta-hexosaminidase |
| 7 | Amino acid sequence of Mus musculus beta subunit of beta-hexosaminidase |
| 8 | Nucleotide sequence of Homo sapiens beta subunit of beta-hexosaminidase |
| 9 | Nucleotide sequence of Mus musculus beta subunit of beta-hexosaminidase |
| 10 | Optimized nucleotide sequence of Mus musculus beta subunit of beta-hexosaminidase |
| 11 | Amino acid sequence of peptide linker L1 |
| 12 | Amino acid sequence of peptide linker L2 |
| 13 | Amino acid sequence of peptide linker L3 |
| 14 | Nucleotide sequence of peptide linker L1 |
| 15 | Nucleotide sequence of peptide linker L2 |
| 16 | Nucleotide sequence of peptide linker L3 |
| 17 | Amino acid sequence of a Mus musculus covalently linked alpha beta (αß) dimer of beta- |
| | hexosaminidase L1 |
| 18 | Amino acid sequence of a Mus musculus covalently linked alpha beta (αß) dimer of beta- |
| | hexosaminidase L2 |
| 19 | Amino acid sequence of a Mus musculus covalently linked alpha beta (αß) dimer of beta- |
| | hexosaminidase L3 |
| 20 | Nucleotide sequence of a Mus musculus covalently linked alpha beta (αß) dimer of beta- |
| | hexosaminidase L1 |
| 21 | Nucleotide sequence of a Mus musculus covalently linked alpha beta (αß) dimer of beta- |
| | hexosaminidase L2 |
| 22 | Nucleotide sequence of a Mus musculus covalently linked alpha beta (αß) dimer of beta- |
| | hexosaminidase L3 |
| 23 | Nucleotide sequence of mammalian CBA promoter |
| 24 | Nucleotide sequence of mammalian Cbh promoter |
| 25 | AAV2 5′ ITR |
| 26 | AAV2 3′ ITR |
| 27 | SV40 polyadenylation signal |
| 28 | pAAV-Cbh-omHexa-L1-omHexb-SV40 |
| 29 | pAAV-Cbh-omHexa-L2-omHexb-SV40 |
| 30 | pAAV-Cbh-omHexa-L3-omHexb-SV40 |
| 31 | pAAV-Cbh-omHexb-L1-omHexa-SV40 |
| 32 | pAAV-Cbh-omHexb-L2-omHexa-SV40 |
| 33 | pAAV-Cbh-omHexb-L3-omHexa-SV40 |
| 34 | pAAV-Cbh-omHexa-SV40 |
| 35 | pAAV-Cbh-omHexb-SV40 |
| 36 | pAAV-Cbh-omHexa-P2A-omHexb-SV40 |
| 37, 38 | CD68 forward and reverse primers |
| 39, 40 | Plp1 forward and reverse primers |
| 41, 42 | Ugt8 forward and reverse primers |
| 43, 44 | Tbp forward and reverse primers |
| 45 | Forward WT/mutant common primer Sandhoff mice |
| 46 | Reverse WT primer Sandhoff mice |
| 47 | Reverse mutant primer Sandhoff mice |
| 48, 49 | Forward and reverse WT primers Tay-Sachs mice |
| 50,51 | Forward and reverse mutant primers Tay-Sachs mice |
| 52 | Amino acid sequence of a (GGGGS)n peptide linker |
| 53 | Amino acid sequence of a Gly6 peptide linker |
| 54 | Amino acid sequence of a Gly8 peptide linker |
| 55 | Amino acid sequence of a KESGSVSSEQLAQFRSLD peptide linker |
| 56 | Amino acid sequence of a EGKSSGSGSESKST peptide linker |
| 57 | Signal peptide (mouse) |
| 58 | Signal peptide (human) |
| 59 | pAAV-Cbh-omHexb-P2A-omHexa-SV40 |
| 60 | pAAV-Cbh-nohHEXA-L1-nohHEXB-SV40 |
| 61 | pAAV-Cbh-ohHEXA-L1-ohHEXB-SV40 GA |
| 62 | pAAV-Cbh-oh HEXA-L1-ohHEXB-SV40 IDT |
| 63 | pAAV-Cbh-oh HEXA-L1-ohHEXB-SV40 NV |
| 64 | GA optimized nucleotide sequence of Homo sapiens alpha subunit of beta hexosaminidase |
| 65 | GA optimized nucleotide sequence of Homo sapiens beta subunit of beta hexosaminidase |
| 66 | IDT optimized nucleotide sequence of Homo sapiens alpha subunit of beta hexosaminidase |
| 67 | IDT optimized nucleotide sequence of Homo sapiens beta subunit of beta hexosaminidase |
| 68 | NV optimized nucleotide sequence of Homo sapiens alpha subunit of beta hexosaminidase |
| 69 | NV optimized nucleotide sequence of Homo sapiens beta subunit of beta hexosaminidase |
| 70 | Amino acid sequence of a covalently linked human alpha beta dimer of beta hexosaminidase |
| | L1 |
| 71 | Nucleotide sequence of a covalently linked human alpha beta dimer of beta hexosaminidase |
| | L1 |
| 72 | Nucleotide sequence of a covalently linked GA optimized human alpha beta dimer of beta |
| | hexosaminidase L1 |
| 73 | Nucleotide sequence of a covalently linked IDT optimized human alpha beta dimer of beta |
| | hexosaminidase L1 |
| 74 | Nucleotide sequence of a covalently linked NV optimized human alpha beta dimer of beta |
| | hexosaminidase L1 |
| 75 | L1 AB mouse gene construct |
| 76 | L2 AB mouse gene construct |
| 77 | L3 AB mouse gene construct |
| 78 | L1 BA mouse gene construct |
| 79 | L2 BA mouse gene construct |
| 80 | L3 BA mouse gene construct |
| 81 | L1 AB human noh gene construct |
| 82 | L1 AB human GA gene construct |
| 83 | L1 AB human IDT gene construct |
| 84 | L1 AB human NV gene construct |
| 85 | Hexa single gene construct |
| 86 | Hexb single gene construct |
| 87 | P2A AB mouse gene construct |
| 88 | P2A BA mouse gene construct |

Optimized nucleotide sequence of Mus musculus alpha subunit of beta-hexosaminidase
SEQ ID NO: 5
ATGGCCGGCTGTAGACTGTGGGTTTCACTGCTGCTGGCTGCCGCTCTGGCTTGTCTGGCTACAGCTCTTTGGCCCTGGCCTCAG
TACATCCAGACCTACCACAGACGGTACACACTGTACCCCAACAACTTCCAGTTCCGCTACCACGTGTCCTCTGCTGCTCAGGCTG
GATGTGTGGTGCTGGACGAGGCCTTCAGAAGATACAGAAACCTGCTGTTCGGCAGCGGCAGCTGGCCTAGACCTAGCTTCTCTA
ACAAGCAGCAGACCCTGGGCAAGAACATCCTGGTGGTGTCTGTGGTCACCGCCGAGTGCAACGAGTTCCCCAACCTGGAAAGCG
TGGAAAACTACACCCTGACCATCAACGACGACCAGTGCCTGCTGGCCTCTGAGACAGTTTGGGGAGCCCTGAGAGGCCTGGAAA
CCTTCTCTCAGCTCGTGTGGAAGTCTGCCGAGGGCACCTTCTTCATCAACAAGACCAAGATCAAGGACTTCCCTAGGTTCCCTCA
CAGAGGCGTGCTGCTGGACACCAGCAGACACTACCTGCCACTGTCCTCCATCCTGGACACCCTGGATGTGATGGCCTACAACAA
GTTCAACGTGTTCCACTGGCACCTGGTGGACGACAGCAGCTTCCCTTACGAGAGCTTCACATTCCCCGAGCTGACCAGAAAGGG
CAGCTTCAACCCCGTGACACACATCTACACAGCCCAGGACGTGAAAGAAGTGATCGAGTACGCCAGACTGAGAGGCATCAGAGT
GCTGGCCGAGTTCGACACACCTGGCCACACACTTTCTTGGGGACCTGGTGCTCCTGGCCTGCTGACACCTTGTTACTCTGGCTCT
CACCTGAGCGGCACATTCGGCCCTGTGAACCCTAGCCTGAACAGCACCTACGACTTTATGAGCACCCTGTTCCTCGAGATCAGCA
GCGTGTTCCCCGACTTCTACCTGCACCTCGGCGGAGATGAGGTGGACTTCACCTGTTGGAAGTCTAACCCCAACATCCAGGCTTT
CATGAAGAAGAAGGGCTTCACCGACTTCAAGCAGCTGGAAAGCTTCTACATTCAGACCCTGCTGGATATCGTGTCCGACTACGAC
AAGGGCTACGTCGTGTGGCAAGAGGTGTTCGACAACAAAGTGAAAGTGCGGCCCGACACCATCATCCAAGTGTGGCGGGAAGAG
ATGCCCGTCGAGTACATGCTGGAAATGCAGGACATCACCAGAGCCGGCTTCAGGGCTCTGCTTAGCGCTCCCTGGTATCTGAAC
AGAGTGAAGTACGGCCCCGACTGGAAGGACATGTACAAGGTGGAACCCCTGGCCTTCCACGGCACCCCTGAACAGAAGGCTCTG
GTTATCGGAGGCGAGGCCTGTATGTGGGGAGAGTACGTGGACAGCACCAACCTGGTGCCAAGACTGTGGCCTAGAGCTGGCGC
TGTGGCTGAGAGACTGTGGTCCAGCAACCTGACCACCAACATCGACTTCGCCTTCAAGAGACTGAGCCACTTCAGATGCGAACTC
GTGCGGAGAGGAATCCAGGCTCAGCCTATCTCTGTGGGCTACTGCGAGCAAGAGTTCGAGCAGACA
Optimized nucleotide sequence of Mus musculus beta subunit of beta-hexosaminidase
SEQ ID NO: 10
CAGCCTGCTCTGTGGCCATTTCCTAGAAGCGTGCAGATGTTCCCCAGACTGCTGTACATCAGCGCCGAGGACTTCAGCATCGATC
ACAGCCCCAACAGCACAGCTGGCCCATCTTGTAGCCTGCTGCAAGAGGCCTTTAGACGGTACTACAACTACGTGTTCGGCTTCTA
CAAGAGGCACCACGGACCTGCCAGATTCAGAGCCGAACCTCAGCTGCAGAAGCTGCTCGTCAGCATCACCCTGGAATCCGAGTG
CGAGAGCTTTCCCAGCCTGTCCAGCGACGAGACATACTCCCTGCTGGTGCAAGAGCCTGTGGCTGTGCTGAAGGCCAACTCTGT
GTGGGGTGCTCTGCGCGGACTGGAAACATTTTCCCAGCTGGTGTACCAGGACAGCTTCGGCACCTTCACAATCAACGAGTCCTCT
ATCGCCGACTCTCCTAGATTCCCACACAGGGGCATCCTGATCGATACCTCCAGGCACTTCCTGCCTGTGAAAACCATCCTCAAGA
CCCTGGACGCCATGGCTTTCAACAAGTTTAATGTGCTGCATTGGCACATCGTGGATGACCAAAGCTTCCCCTACCAGTCCACCAC
CTTTCCAGAGCTGTCCAACAAGGGCTCCTACAGCCTGTCTCACGTGTACACCCCTAACGACGTGOGGATGGTCCTGGAATACGCT
AGGCTGCGGGGAATCAGAGTGATCCCTGAGTTCGATACTCCCGGACACACACAGAGCTGGGGCAAGGGACAGAAGAACCTGCT
GACCCCATGCTACAACCAGAAAACAAAGACCCAGGTGTTCGGCCCAGTGGACCCAACCGTGAACACAACCTACGCTTTCTTCAAC
ACATTCTTCAAAGAAATCAGCTCCGTGTTTCCCGACCAGTTCATCCATCTCGGCGGCGACGAAGTCGAGTTCCAGTGTTGGGCCA
GCAATCCCAATATCCAAGGATTCATGAAGCGGAAAGGCTTCGGCTCCGACTTCAGAAGGCTGGAATCTTTCTACATCAAGAAGAT
CCTCGAAATCATCAGCAGCCTGAAAAAGAACAGCATCGTCTGGCAAGAAGTCTTTGACGACAAGGTCGAGCTGCAGCCAGGCACT
GTGGTGGAAGTGTGGAAAAGCGAGCACTACAGCTACGAGCTGAAGCAAGTGACAGGCAGCGGCTTCCCTGCTATCCTCTCTGCC
CCTTGGTATCTGGACCTGATCAGCTACGGCCAGGATTGGAAGAACTACTACAAGGTTGAGCCCCTCAACTTCGAGGGCAGCGAG
AAGCAGAAACAGCTGGTCATTGGCGGCGAGGCTTGTCTCTGGGGCGAGTTTGTGGATGCCACCAATCTGACCCCTAGGCTGTGG
CCAAGGGCTTCTGCTGTGGGAGAGAGACTTTGGAGCCCCAAGACCGTGACCGACCTGGAAAACGCCTATAAGAGACTGGCCGTG
CACAGATGCAGAATGGTGTCCCGAGGAATCGCCGCTCAGCCTCTGTACACCGGCTACTGCAACTACGAGAACAAGATCTGA
Amino acid sequence of peptide linker L1:
SEQ ID NO: 11
GGGGSGGGGSGGGGSGGGGS
Amino acid sequence of peptide linker L2:
SEQ ID NO: 12
SGGSSGGSSGSETPGTSESATPESSGGSSGGSS
Amino acid sequence of peptide linker L3:
SEQ ID NO: 13
GSAGSAAGSGEF
Nucleotide sequence of peptide linker L1
SEQ ID NO: 14
GGTGGTGGTGGATCTGGAGGCGGAGGATCAGGGGGGGGAGGTTCTGGAGGTGGAGGTAGT
Nucleotide sequence of peptide linker L2
SEQ ID NO: 15
TCTGGAGGATCTAGCGGAGGATCCTCTGGCAGCGAGACACCAGGAACAAGCGAGTCAGCAACACCAGAGAGCAGTGGCGGCAG
CAGCGGCGGCAGCAGC
Nucleotide sequence of peptide linker L3:
SEQ ID NO: 16
GGCAGCGCCGGCAGCGCCGCCGGCAGCGGCGAGTTC
SEQ ID NO: 28-pAAV-Cbh-omHexa-L1-omHexb-SV40
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGGGGGG
CGAGGGGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGGGGGCGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGGGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGGCCGGCTGTAGACTGTGGGTTTCACTGCTGCTGGC
TGCCGCTCTGGCTTGTCTGGCTACAGCTCTTTGGCCCTGGCCTCAGTACATCCAGACCTACCACAGACGGTACACACTGTACCCC
AACAACTTCCAGTTCCGCTACCACGTGTCCTCTGCTGCTCAGGCTGGATGTGTGGTGCTGGACGAGGCCTTCAGAAGATACAGAA
ACCTGCTGTTCGGCAGCGGCAGCTGGCCTAGACCTAGCTTCTCTAACAAGCAGCAGACCCTGGGCAAGAACATCCTGGTGGTGT
CTGTGGTCACCGCCGAGTGCAACGAGTTCCCCAACCTGGAAAGCGTGGAAAACTACACCCTGACCATCAACGACGACCAGTGCC
TGCTGGCCTCTGAGACAGTTTGGGGAGCCCTGAGAGGCCTGGAAACCTTCTCTCAGCTCGTGTGGAAGTCTGCCGAGGGCACCT
TCTTCATCAACAAGACCAAGATCAAGGACTTCCCTAGGTTCCCTCACAGAGGCGTGCTGCTGGACACCAGCAGACACTACCTGCC
ACTGTCCTCCATCCTGGACACCCTGGATGTGATGGCCTACAACAAGTTCAACGTGTTCCACTGGCACCTGGTGGACGACAGCAGC
TTCCCTTACGAGAGCTTCACATTCCCCGAGCTGACCAGAAAGGGCAGCTTCAACCCCGTGACACACATCTACACAGCCCAGGACG
TGAAAGAAGTGATCGAGTACGCCAGACTGAGAGGCATCAGAGTGCTGGCCGAGTTCGACACACCTGGCCACACACTTTCTTGGG
GACCTGGTGCTCCTGGCCTGCTGACACCTTGTTACTCTGGCTCTCACCTGAGCGGCACATTCGGCCCTGTGAACCCTAGCCTGAA
CAGCACCTACGACTTTATGAGCACCCTGTTCCTCGAGATCAGCAGCGTGTTCCCCGACTTCTACCTGCACCTCGGCGGAGATGAG
GTGGACTTCACCTGTTGGAAGTCTAACCCCAACATCCAGGCTTTCATGAAGAAGAAGGGCTTCACCGACTTCAAGCAGCTGGAAA
GCTTCTACATTCAGACCCTGCTGGATATCGTGTCCGACTACGACAAGGGCTACGTCGTGTGGCAAGAGGTGTTCGACAACAAAGT
GAAAGTGCGGCCCGACACCATCATCCAAGTGTGGCGGGAAGAGATGCCCGTCGAGTACATGCTGGAAATGCAGGACATCACCAG
AGCCGGCTTCAGGGCTCTGCTTAGCGCTCCCTGGTATCTGAACAGAGTGAAGTACGGCCCCGACTGGAAGGACATGTACAAGGT
GGAACCCCTGGCCTTCCACGGCACCCCTGAACAGAAGGCTCTGGTTATCGGAGGCGAGGCCTGTATGTGGGGAGAGTACGTGG
ACAGCACCAACCTGGTGCCAAGACTGTGGCCTAGAGCTGGCGCTGTGGCTGAGAGACTGTGGTCCAGCAACCTGACCACCAACA
TCGACTTCGCCTTCAAGAGACTGAGCCACTTCAGATGCGAACTCGTGCGGAGAGGAATCCAGGCTCAGCCTATCTCTGTGGGCTA
CTGCGAGCAAGAGTTCGAGCAGACAGGTGGTGGTGGATCTGGAGGCGGAGGATCAGGGGGGGGAGGTTCTGGAGGTGGAGGT
AGTCAGCCTGCTCTGTGGCCATTTCCTAGAAGCGTGCAGATGTTCCCCAGACTGCTGTACATCAGCGCCGAGGACTTCAGCATCG
ATCACAGCCCCAACAGCACAGCTGGCCCATCTTGTAGCCTGCTGCAAGAGGCCTTTAGACGGTACTACAACTACGTGTTCGGCTT
CTACAAGAGGCACCACGGACCTGCCAGATTCAGAGCCGAACCTCAGCTGCAGAAGCTGCTCGTCAGCATCACCCTGGAATCCGA
GTGCGAGAGCTTTCCCAGCCTGTCCAGCGACGAGACATACTCCCTGCTGGTGCAAGAGCCTGTGGCTGTGCTGAAGGCCAACTC
TGTGTGGGGTGCTCTGCGCGGACTGGAAACATTTTCCCAGCTGGTGTACCAGGACAGCTTCGGCACCTTCACAATCAACGAGTC
CTCTATCGCCGACTCTCCTAGATTCCCACACAGGGGCATCCTGATCGATACCTCCAGGCACTTCCTGCCTGTGAAAACCATCCTC
AAGACCCTGGACGCCATGGCTTTCAACAAGTTTAATGTGCTGCATTGGCACATCGTGGATGACCAAAGCTTCCCCTACCAGTCCA
CCACCTTTCCAGAGCTGTCCAACAAGGGCTCCTACAGCCTGTCTCACGTGTACACCCCTAACGACGTGCGGATGGTCCTGGAATA
CGCTAGGCTGCGGGGAATCAGAGTGATCCCTGAGTTCGATACTCCCGGACACACACAGAGCTGGGGCAAGGGACAGAAGAACC
TGCTGACCCCATGCTACAACCAGAAAACAAAGACCCAGGTGTTCGGCCCAGTGGACCCAACCGTGAACACAACCTACGCTTTCTT
CAACACATTCTTCAAAGAAATCAGCTCCGTGTTTCCCGACCAGTTCATCCATCTCGGCGGCGACGAAGTCGAGTTCCAGTGTTGG
GCCAGCAATCCCAATATCCAAGGATTCATGAAGCGGAAAGGCTTCGGCTCCGACTTCAGAAGGCTGGAATCTTTCTACATCAAGA
AGATCCTCGAAATCATCAGCAGCCTGAAAAAGAACAGCATCGTCTGGCAAGAAGTCTTTGACGACAAGGTCGAGCTGCAGCCAGG
CACTGTGGTGGAAGTGTGGAAAAGCGAGCACTACAGCTACGAGCTGAAGCAAGTGACAGGCAGCGGCTTCCCTGCTATCCTCTC
TGCCCCTTGGTATCTGGACCTGATCAGCTACGGCCAGGATTGGAAGAACTACTACAAGGTTGAGCCCCTCAACTTCGAGGGCAG
CGAGAAGCAGAAACAGCTGGTCATTGGCGGCGAGGCTTGTCTCTGGGGCGAGTTTGTGGATGCCACCAATCTGACCCCTAGGCT
GTGGCCAAGGGCTTCTGCTGTGGGAGAGAGACTTTGGAGCCCCAAGACCGTGACCGACCTGGAAAACGCCTATAAGAGACTGGC
CGTGCACAGATGCAGAATGGTGTCCCGAGGAATCGCCGCTCAGCCTCTGTACACCGGCTACTGCAACTACGAGAACAAGATCTG
AGGATCCAACTTGTTTATTGCAGCTTATAATGGTTACAAATAAAGCAATAGCATCACAAATTTCACAAATAAAGCATTTTTTTCACTG
CATTCTAGTTGTGGTTTGTCCAAACTCATCAATGTATOTTATGCGGCCGCAGGAACCCCTAGTGATGGAGTTGGCCACTCCCTCTC
TGCGCGCTCGCTCGCTCACTGAGGCCGGGCGACCAAAGGTCGCCCGACGCCCGGGCTTTGCCCGGGGGGCCTCAGTGAGCGA
GCGAGCGCGCAGCTGCCTGCAGGGGCGCCTGATGCGGTATTTTCTCCTTACGCATCTGTGCGGTATTTCACACCGCATACGTCA
AAGCAACCATAGTACGCGCCCTGTAGCGGCGCATTAAGCGCGGCGGGTGTGGTGGTTACGCGCAGCGTGACCGCTACACTTGC
CAGCGCCCTAGCGCCCGCTCCTTTCGCTTTCTTCCCTTCCTTTCTCGCCACGTTCGCCGGCTTTCCCCGTCAAGCTCTAAATCGG
GGGCTCCCTTTAGGGTTCCGATTTAGTGCTTTACGGCACCTCGACCCCAAAAAACTTGATTTGGGTGATGGTTCACGTAGTGGGC
CATCGCCCTGATAGACGGTTTTTCGCCCTTTGACGTTGGAGTCCACGTTCTTTAATAGTGGACTCTTGTTCCAAACTGGAACAACA
CTCAACCCTATCTCGGGCTATTCTTTTGATTTATAAGGGATTTTGCCGATTTCGGCCTATTGGTTAAAAAATGAGCTGATTTAACAA
AAATTTAACGCGAATTTTAACAAAATATTAACGTTTACAATTTTATGGTGCACTCTCAGTACAATCTGCTCTGATGCCGCATAGTTAA
GCCAGCCCCGACACCCGCCAACACCCGCTGACGCGCCCTGACGGGCTTGTCTGCTCCCGGCATCCGCTTACAGACAAGCTGTG
ACCGTCTCCGGGAGCTGCATGTGTCAGAGGTTTTCACCGTCATCACCGAAACGCGCGAGACGAAAGGGCCTCGTGATACGCCTA
TTTTTATAGGTTAATGTCATGATAATAATGGTTTCTTAGACGTCAGGTGGCACTTTTCGGGGAAATGTGCGCGGAACCCCTATTTGT
TTATTTTTCTAAATACATTCAAATATGTATCCGCTCATGAGACAATAACCCTGATAAATGCTTCAATAATATTGAAAAAGGAAGAGTA
TGAGTATTCAACATTTCCGTGTCGCCCTTATTCCCTTTTTTGCGGCATTTTGCCTTCCTGTTTTTGCTCACCCAGAAACGCTGGTGA
AAGTAAAAGATGCTGAAGATCAGTTGGGTGCACGAGTGGGTTACATCGAACTGGATCTCAACAGCGGTAAGATCCTTGAGAGTTT
TCGCCCCGAAGAACGTTTTCCAATGATGAGCACTTTTAAAGTTCTGCTATGTGGCGCGGTATTATCCCGTATTGACGCCGGGCAA
GAGCAACTCGGTCGCCGCATACACTATTCTCAGAATGACTTGGTTGAGTACTCACCAGTCACAGAAAAGCATCTTACGGATGGCA
TGACAGTAAGAGAATTATGCAGTGCTGCCATAACCATGAGTGATAACACTGCGGCCAACTTACTTCTGACAACGATCGGAGGACC
GAAGGAGCTAACCGCTTTTTTGCACAACATGGGGGATCATGTAACTCGCCTTGATCGTTGGGAACCGGAGCTGAATGAAGCCATA
CCAAACGACGAGCGTGACACCACGATGCCTGTAGCAATGGCAACAACGTTGCGCAAACTATTAACTGGCGAACTACTTACTCTAG
CTTCCCGGCAACAATTAATAGACTGGATGGAGGCGGATAAAGTTGCAGGACCACTTCTGCGCTCGGCCCTTCCGGCTGGCTGGT
TTATTGCTGATAAATCTGGAGCCGGTGAGCGTGGGTCTCGCGGTATCATTGCAGCACTGGGGCCAGATGGTAAGCCCTCCCGTA
TCGTAGTTATCTACACGACGGGGAGTCAGGCAACTATGGATGAACGAAATAGACAGATCGCTGAGATAGGTGCCTCACTGATTAA
GCATTGGTAACTGTCAGACCAAGTTTACTCATATATACTTTAGATTGATTTAAAACTTCATTTTTAATTTAAAAGGATCTAGGTGAAG
ATCCTTTTTGATAATCTCATGACCAAAATCCCTTAACGTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACCACCGCTACCAGCGGTGGTTTGTTTGCCGGA
TCAAGAGCTACCAACTCTTTTTCCGAAGGTAACTGGCTTCAGCAGAGCGCAGATACCAAATACTGTCCTTCTAGTGTAGCCGTAGT
TAGGCCACCACTTCAAGAACTCTGTAGCACCGCCTACATACCTCGCTCTGCTAATCCTGTTACCAGTGGCTGCTGCCAGTGGCGA
TAAGTCGTGTCTTACCGGGTTGGACTCAAGACGATAGTTACCGGATAAGGCGCAGCGGTCGGGCTGAACGGGGGGTTCGTGCAC
ACAGCCCAGCTTGGAGCGAACGACCTACACCGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCTTTTGCTCACATGT
pAAV-Cbh-omHexa-L2-omHexb-SV40
SEQ ID NO: 29
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGGGGG
CGAGGGGGGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGGGGGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGGGGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGGCCGGCTGTAGACTGTGGGTTTCACTGCTGCTGGC
TGCCGCTCTGGCTTGTCTGGCTACAGCTCTTTGGCCCTGGCCTCAGTACATCCAGACCTACCACAGACGGTACACACTGTACCCC
AACAACTTCCAGTTCCGCTACCACGTGTCCTCTGCTGCTCAGGCTGGATGTGTGGTGCTGGACGAGGCCTTCAGAAGATACAGAA
ACCTGCTGTTCGGCAGCGGCAGCTGGCCTAGACCTAGCTTCTCTAACAAGCAGCAGACCCTGGGCAAGAACATCCTGGTGGTGT
CTGTGGTCACCGCCGAGTGCAACGAGTTCCCCAACCTGGAAAGCGTGGAAAACTACACCCTGACCATCAACGACGACCAGTGCC
TGCTGGCCTCTGAGACAGTTTGGGGAGCCCTGAGAGGCCTGGAAACCTTCTCTCAGCTCGTGTGGAAGTCTGCCGAGGGCACCT
TCTTCATCAACAAGACCAAGATCAAGGACTTCCCTAGGTTCCCTCACAGAGGCGTGCTGCTGGACACCAGCAGACACTACCTGCC
ACTGTCCTCCATCCTGGACACCCTGGATGTGATGGCCTACAACAAGTTCAACGTGTTCCACTGGCACCTGGTGGACGACAGCAGC
TTCCCTTACGAGAGCTTCACATTCCCCGAGCTGACCAGAAAGGGCAGCTTCAACCCCGTGACACACATCTACACAGCCCAGGACG
TGAAAGAAGTGATCGAGTACGCCAGACTGAGAGGCATCAGAGTGCTGGCCGAGTTCGACACACCTGGCCACACACTTTCTTGGG
GACCTGGTGCTCCTGGCCTGCTGACACCTTGTTACTCTGGCTCTCACCTGAGCGGCACATTCGGCCCTGTGAACCCTAGCCTGAA
CAGCACCTACGACTTTATGAGCACCCTGTTCCTCGAGATCAGCAGCGTGTTCCCCGACTTCTACCTGCACCTCGGCGGAGATGAG
GTGGACTTCACCTGTTGGAAGTCTAACCCCAACATCCAGGCTTTCATGAAGAAGAAGGGCTTCACCGACTTCAAGCAGCTGGAAA
GCTTCTACATTCAGACCCTGCTGGATATCGTGTCCGACTACGACAAGGGCTACGTCGTGTGGCAAGAGGTGTTCGACAACAAAGT
GAAAGTGCGGCCCGACACCATCATCCAAGTGTGGCGGGAAGAGATGCCCGTCGAGTACATGCTGGAAATGCAGGACATCACCAG
AGCCGGCTTCAGGGCTCTGCTTAGCGCTCCCTGGTATCTGAACAGAGTGAAGTACGGCCCCGACTGGAAGGACATGTACAAGGT
GGAACCCCTGGCCTTCCACGGCACCCCTGAACAGAAGGCTCTGGTTATCGGAGGCGAGGCCTGTATGTGGGGAGAGTACGTGG
ACAGCACCAACCTGGTGCCAAGACTGTGGCCTAGAGCTGGCGCTGTGGCTGAGAGACTGTGGTCCAGCAACCTGACCACCAACA
TCGACTTCGCCTTCAAGAGACTGAGCCACTTCAGATGCGAACTCGTGCGGAGAGGAATCCAGGCTCAGCCTATCTCTGTGGGCTA
CTGCGAGCAAGAGTTCGAGCAGACATCTGGAGGATCTAGCGGAGGATCCTCTGGCAGCGAGACACCAGGAACAAGCGAGTCAG
CAACACCAGAGAGCAGTGGCGGCAGCAGCGGCGGCAGCAGCCAGCCTGCTCTGTGGCCATTTCCTAGAAGCGTGCAGATGTTC
CCCAGACTGCTGTACATCAGCGCCGAGGACTTCAGCATCGATCACAGCCCCAACAGCACAGCTGGCCCATCTTGTAGCCTGCTG
CAAGAGGCCTTTAGACGGTACTACAACTACGTGTTCGGCTTCTACAAGAGGCACCACGGACCTGCCAGATTCAGAGCCGAACCTC
AGCTGCAGAAGCTGCTCGTCAGCATCACCCTGGAATCCGAGTGCGAGAGCTTTCCCAGCCTGTCCAGCGACGAGACATACTCCC
TGCTGGTGCAAGAGCCTGTGGCTGTGCTGAAGGCCAACTCTGTGTGGGGTGCTCTGCGCGGACTGGAAACATTTTCCCAGCTGG
TGTACCAGGACAGCTTCGGCACCTTCACAATCAACGAGTCCTCTATCGCCGACTCTCCTAGATTCCCACACAGGGGCATCCTGAT
CGATACCTCCAGGCACTTCCTGCCTGTGAAAACCATCCTCAAGACCCTGGACGCCATGGCTTTCAACAAGTTTAATGTGCTGCATT
GGCACATCGTGGATGACCAAAGCTTCCCCTACCAGTCCACCACCTTTCCAGAGCTGTCCAACAAGGGCTCCTACAGCCTGTCTCA
CGTGTACACCCCTAACGACGTGOGGATGGTCCTGGAATACGCTAGGCTGCGGGGAATCAGAGTGATCCCTGAGTTCGATACTCC
CGGACACACACAGAGCTGGGGCAAGGGACAGAAGAACCTGCTGACCCCATGCTACAACCAGAAAACAAAGACCCAGGTGTTCGG
CCCAGTGGACCCAACCGTGAACACAACCTACGCTTTCTTCAACACATTCTTCAAAGAAATCAGCTCCGTGTTTCCCGACCAGTTCA
TCCATCTCGGCGGCGACGAAGTCGAGTTCCAGTGTTGGGCCAGCAATCCCAATATCCAAGGATTCATGAAGCGGAAAGGCTTCG
GCTCCGACTTCAGAAGGCTGGAATCTTTCTACATCAAGAAGATCCTCGAAATCATCAGCAGCCTGAAAAAGAACAGCATCGTCTG
GCAAGAAGTCTTTGACGACAAGGTCGAGCTGCAGCCAGGCACTGTGGTGGAAGTGTGGAAAAGCGAGCACTACAGCTACGAGCT
GAAGCAAGTGACAGGCAGCGGCTTCCCTGCTATCCTCTCTGCCCCTTGGTATCTGGACCTGATCAGCTACGGCCAGGATTGGAA
GAACTACTACAAGGTTGAGCCCCTCAACTTCGAGGGCAGCGAGAAGCAGAAACAGCTGGTCATTGGCGGCGAGGCTTGTCTCTG
GGGCGAGTTTGTGGATGCCACCAATCTGACCCCTAGGCTGTGGCCAAGGGCTTCTGCTGTGGGAGAGAGACTTTGGAGCCCCAA
GACCGTGACCGACCTGGAAAACGCCTATAAGAGACTGGCCGTGCACAGATGCAGAATGGTGTCCCGAGGAATCGCCGCTCAGCC
TCTGTACACCGGCTACTGCAACTACGAGAACAAGATCTGAGGATCCAACTTGTTTATTGCAGCTTATAATGGTTACAAATAAAGCAA
TAGCATCACAAATTTCACAAATAAAGCATTTTTTTCACTGCATTCTAGTTGTGGTTTGTCCAAACTCATCAATGTATOTTATGCGGCC
GCAGGAACCCCTAGTGATGGAGTTGGCCACTCCCTCTCTGCGCGCTCGCTCGCTCACTGAGGCCGGGCGACCAAAGGTCGCCC
GACGCCCGGGCTTTGCCCGGGGGGCCTCAGTGAGCGAGCGAGCGCGCAGCTGCCTGCAGGGGCGCCTGATGCGGTATTTTCT
CCTTACGCATCTGTGCGGTATTTCACACCGCATACGTCAAAGCAACCATAGTACGCGCCCTGTAGCGGCGCATTAAGCGCGGCG
GGTGTGGTGGTTACGCGCAGCGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAGTGCTTTACGGCACCTCGACCC
CAAAAAACTTGATTTGGGTGATGGTTCACGTAGTGGGCCATCGCCCTGATAGACGGTTTTTCGCCCTTTGACGTTGGAGTCCACG
TTCTTTAATAGTGGACTCTTGTTCCAAACTGGAACAACACTCAACCCTATCTCGGGCTATTCTTTTGATTTATAAGGGATTTTGCCG
ATTTCGGCCTATTGGTTAAAAAATGAGCTGATTTAACAAAAATTTAACGCGAATTTTAACAAAATATTAACGTTTACAATTTTATGGT
GCACTCTCAGTACAATCTGCTCTGATGCCGCATAGTTAAGCCAGCCCCGACACCCGCCAACACCCGCTGACGCGCCCTGACGGG
CTTGTCTGCTCCCGGCATCCGCTTACAGACAAGCTGTGACCGTCTCCGGGAGCTGCATGTGTCAGAGGTTTTCACCGTCATCACC
GAAACGCGCGAGACGAAAGGGCCTCGTGATACGCCTATTTTTATAGGTTAATGTCATGATAATAATGGTTTCTTAGACGTCAGGTG
GCACTTTTCGGGGAAATGTGCGCGGAACCCCTATTTGTTTATTTTTCTAAATACATTCAAATATGTATCCGCTCATGAGACAATAAC
CCTGATAAATGCTTCAATAATATTGAAAAAGGAAGAGTATGAGTATTCAACATTTCCGTGTCGCCCTTATTCCCTTTTTTGCGGCAT
TTTGCCTTCCTGTTTTTGCTCACCCAGAAACGCTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTTTTCCAATGATGAGCACTTTTAAAGTTCTGCT
ATGTGGCGCGGTATTATCCCGTATTGACGCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATGCAGTGCTGCCATAACCATGAGTGATAACAC
TGCGGCCAACTTACTTCTGACAACGATCGGAGGACCGAAGGAGCTAACCGCTTTTTTGCACAACATGGGGGATCATGTAACTCGC
CTTGATCGTTGGGAACCGGAGCTGAATGAAGCCATACCAAACGACGAGCGTGACACCACGATGCCTGTAGCAATGGCAACAACG
TTGCGCAAACTATTAACTGGCGAACTACTTACTCTAGCTTCCCGGCAACAATTAATAGACTGGATGGAGGCGGATAAAGTTGCAGG
ACCACTTCTGCGCTCGGCCCTTCCGGCTGGCTGGTTTATTGCTGATAAATCTGGAGCCGGTGAGCGTGGGTCTCGCGGTATCATT
GCAGCACTGGGGCCAGATGGTAAGCCCTCCCGTATCGTAGTTATCTACACGACGGGGAGTCAGGCAACTATGGATGAACGAAAT
AGACAGATCGCTGAGATAGGTGCCTCACTGATTAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCATGACCAAAATCCCTTAACGTGAGTTTTCGTTCC
ACTGAGCGTCAGACCCCGTAGAAAAGATCAAAGGATCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAACTCTTTTTCCGAAGGTAACTGGCTTCAGCAGAGCGC
AGATACCAAATACTGTCCTTCTAGTGTAGCCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTGTCGGGTTTCGCCACCTCTGACTTGAGCGTCGATTT
TTGTGATGCTCGTCAGGGGGGCGGAGCCTATGGAAAAACGCCAGCAACGCGGCCTTTTTACGGTTCCTGGCCTTTTGCTGGCCT
TTTGCTCACATGT
pAAV-Cbh-omHexa-L3-omHexb-SV40
SEQ ID NO: 30
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGGGGGG
CGAGGGGCGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGGGGGGGGGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGGGGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGGCCGGCTGTAGACTGTGGGTTTCACTGCTGCTGGC
TGCCGCTCTGGCTTGTCTGGCTACAGCTCTTTGGCCCTGGCCTCAGTACATCCAGACCTACCACAGACGGTACACACTGTACCCC
AACAACTTCCAGTTCCGCTACCACGTGTCCTCTGCTGCTCAGGCTGGATGTGTGGTGCTGGACGAGGCCTTCAGAAGATACAGAA
ACCTGCTGTTCGGCAGCGGCAGCTGGCCTAGACCTAGCTTCTCTAACAAGCAGCAGACCCTGGGCAAGAACATCCTGGTGGTGT
CTGTGGTCACCGCCGAGTGCAACGAGTTCCCCAACCTGGAAAGCGTGGAAAACTACACCCTGACCATCAACGACGACCAGTGCC
TGCTGGCCTCTGAGACAGTTTGGGGAGCCCTGAGAGGCCTGGAAACCTTCTCTCAGCTCGTGTGGAAGTCTGCCGAGGGCACCT
TCTTCATCAACAAGACCAAGATCAAGGACTTCCCTAGGTTCCCTCACAGAGGCGTGCTGCTGGACACCAGCAGACACTACCTGCC
ACTGTCCTCCATCCTGGACACCCTGGATGTGATGGCCTACAACAAGTTCAACGTGTTCCACTGGCACCTGGTGGACGACAGCAGC
TTCCCTTACGAGAGCTTCACATTCCCCGAGCTGACCAGAAAGGGCAGCTTCAACCCCGTGACACACATCTACACAGCCCAGGACG
TGAAAGAAGTGATCGAGTACGCCAGACTGAGAGGCATCAGAGTGCTGGCCGAGTTCGACACACCTGGCCACACACTTTCTTGGG
GACCTGGTGCTCCTGGCCTGCTGACACCTTGTTACTCTGGCTCTCACCTGAGCGGCACATTCGGCCCTGTGAACCCTAGCCTGAA
CAGCACCTACGACTTTATGAGCACCCTGTTCCTCGAGATCAGCAGCGTGTTCCCCGACTTCTACCTGCACCTCGGCGGAGATGAG
GTGGACTTCACCTGTTGGAAGTCTAACCCCAACATCCAGGCTTTCATGAAGAAGAAGGGCTTCACCGACTTCAAGCAGCTGGAAA
GCTTCTACATTCAGACCCTGCTGGATATCGTGTCCGACTACGACAAGGGCTACGTCGTGTGGCAAGAGGTGTTCGACAACAAAGT
GAAAGTGCGGCCCGACACCATCATCCAAGTGTGGCGGGAAGAGATGCCCGTCGAGTACATGCTGGAAATGCAGGACATCACCAG
AGCCGGCTTCAGGGCTCTGCTTAGCGCTCCCTGGTATCTGAACAGAGTGAAGTACGGCCCCGACTGGAAGGACATGTACAAGGT
GGAACCCCTGGCCTTCCACGGCACCCCTGAACAGAAGGCTCTGGTTATCGGAGGCGAGGCCTGTATGTGGGGAGAGTACGTGG
ACAGCACCAACCTGGTGCCAAGACTGTGGCCTAGAGCTGGCGCTGTGGCTGAGAGACTGTGGTCCAGCAACCTGACCACCAACA
TCGACTTCGCCTTCAAGAGACTGAGCCACTTCAGATGCGAACTCGTGCGGAGAGGAATCCAGGCTCAGCCTATCTCTGTGGGCTA
CTGCGAGCAAGAGTTCGAGCAGACAGGCAGCGCCGGCAGCGCCGCCGGCAGCGGCGAGTTCCAGCCTGCTCTGTGGCCATTTC
CTAGAAGCGTGCAGATGTTCCCCAGACTGCTGTACATCAGCGCCGAGGACTTCAGCATCGATCACAGCCCCAACAGCACAGCTG
GCCCATCTTGTAGCCTGCTGCAAGAGGCCTTTAGACGGTACTACAACTACGTGTTCGGCTTCTACAAGAGGCACCACGGACCTGC
CAGATTCAGAGCCGAACCTCAGCTGCAGAAGCTGCTCGTCAGCATCACCCTGGAATCCGAGTGCGAGAGCTTTCCCAGCCTGTC
CAGCGACGAGACATACTCCCTGCTGGTGCAAGAGCCTGTGGCTGTGCTGAAGGCCAACTCTGTGTGGGGTGCTCTGCGCGGACT
GGAAACATTTTCCCAGCTGGTGTACCAGGACAGCTTCGGCACCTTCACAATCAACGAGTCCTCTATCGCCGACTCTCCTAGATTC
CCACACAGGGGCATCCTGATCGATACCTCCAGGCACTTCCTGCCTGTGAAAACCATCCTCAAGACCCTGGACGCCATGGCTTTCA
ACAAGTTTAATGTGCTGCATTGGCACATCGTGGATGACCAAAGCTTCCCCTACCAGTCCACCACCTTTCCAGAGCTGTCCAACAAG
GGCTCCTACAGCCTGTCTCACGTGTACACCCCTAACGACGTGCGGATGGTCCTGGAATACGCTAGGCTGCGGGGAATCAGAGTG
ATCCCTGAGTTCGATACTCCCGGACACACACAGAGCTGGGGCAAGGGACAGAAGAACCTGCTGACCCCATGCTACAACCAGAAA
ACAAAGACCCAGGTGTTCGGCCCAGTGGACCCAACCGTGAACACAACCTACGCTTTCTTCAACACATTCTTCAAAGAAATCAGCTC
CGTGTTTCCCGACCAGTTCATCCATCTCGGCGGCGACGAAGTCGAGTTCCAGTGTTGGGCCAGCAATCCCAATATCCAAGGATTC
ATGAAGCGGAAAGGCTTCGGCTCCGACTTCAGAAGGCTGGAATCTTTCTACATCAAGAAGATCCTCGAAATCATCAGCAGCCTGA
AAAAGAACAGCATCGTCTGGCAAGAAGTCTTTGACGACAAGGTCGAGCTGCAGCCAGGCACTGTGGTGGAAGTGTGGAAAAGCG
AGCACTACAGCTACGAGCTGAAGCAAGTGACAGGCAGCGGCTTCCCTGCTATCCTCTCTGCCCCTTGGTATCTGGACCTGATCAG
CTACGGCCAGGATTGGAAGAACTACTACAAGGTTGAGCCCCTCAACTTCGAGGGCAGCGAGAAGCAGAAACAGCTGGTCATTGG
CGGCGAGGCTTGTCTCTGGGGCGAGTTTGTGGATGCCACCAATCTGACCCCTAGGCTGTGGCCAAGGGCTTCTGCTGTGGGAGA
GAGACTTTGGAGCCCCAAGACCGTGACCGACCTGGAAAACGCCTATAAGAGACTGGCCGTGCACAGATGCAGAATGGTGTCCCG
AGGAATCGCCGCTCAGCCTCTGTACACCGGCTACTGCAACTACGAGAACAAGATCTGAGGATCCAACTTGTTTATTGCAGCTTATA
ATGGTTACAAATAAAGCAATAGCATCACAAATTTCACAAATAAAGCATTTTTTTCACTGCATTCTAGTTGTGGTTTGTCCAAACTCAT
CAATGTATOTTATGCGGCCGCAGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGGGGCCTCAGTGAGCGAGCGAGCGCGCAGCTGCCTGCAGGGGC
GCCTGATGCGGTATTTTCTCCTTACGCATCTGTGCGGTATTTCACACCGCATACGTCAAAGCAACCATAGTACGCGCCCTGTAGC
GGCGCATTAAGCGCGGCGGGTGTGGTGGTTACGCGCAGCGTGACCGCTACACTTGCCAGCGCCCTAGCGCCCGCTCCTTTCGC
TTTCTTCCCTTCCTTTCTCGCCACGTTCGCCGGCTTTCCCCGTCAAGCTCTAAATCGGGGGCTCCCTTTAGGGTTCCGATTTAGTG
CTTTACGGCACCTCGACCCCAAAAAACTTGATTTGGGTGATGGTTCACGTAGTGGGCCATCGCCCTGATAGACGGTTTTTCGCCC
TTTGACGTTGGAGTCCACGTTCTTTAATAGTGGACTCTTGTTCCAAACTGGAACAACACTCAACCCTATCTCGGGCTATTCTTTTGA
TTTATAAGGGATTTTGCCGATTTCGGCCTATTGGTTAAAAAATGAGCTGATTTAACAAAAATTTAACGCGAATTTTAACAAAATATTA
ACGTTTACAATTTTATGGTGCACTCTCAGTACAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGTGACCGTCTCCGGGAGCTGCATGTGTCAGA
GGTTTTCACCGTCATCACCGAAACGCGCGAGACGAAAGGGCCTCGTGATACGCCTATTTTTATAGGTTAATGTCATGATAATAATG
GTTTCTTAGACGTCAGGTGGCACTTTTCGGGGAAATGTGCGCGGAACCCCTATTTGTTTATTTTTCTAAATACATTCAAATATGTAT
CCGCTCATGAGACAATAACCCTGATAAATGCTTCAATAATATTGAAAAAGGAAGAGTATGAGTATTCAACATTTCCGTGTCGCCCTT
ATTCCCTTTTTTGCGGCATTTTGCCTTCCTGTTTTTGCTCACCCAGAAACGCTGGTGAAAGTAAAAGATGCTGAAGATCAGTTGGG
TGCACGAGTGGGTTACATCGAACTGGATCTCAACAGCGGTAAGATCCTTGAGAGTTTTCGCCCCGAAGAACGTTTTCCAATGATG
AGCACTTTTAAAGTTCTGCTATGTGGCGCGGTATTATCCCGTATTGACGCCGGGCAAGAGCAACTCGGTCGCCGCATACACTATT
CTCAGAATGACTTGGTTGAGTACTCACCAGTCACAGAAAAGCATCTTACGGATGGCATGACAGTAAGAGAATTATGCAGTGCTGC
CATAACCATGAGTGATAACACTGCGGCCAACTTACTTCTGACAACGATCGGAGGACCGAAGGAGCTAACCGCTTTTTTGCACAAC
ATGGGGGATCATGTAACTCGCCTTGATCGTTGGGAACCGGAGCTGAATGAAGCCATACCAAACGACGAGCGTGACACCACGATG
CCTGTAGCAATGGCAACAACGTTGCGCAAACTATTAACTGGCGAACTACTTACTCTAGCTTCCCGGCAACAATTAATAGACTGGAT
GGAGGCGGATAAAGTTGCAGGACCACTTCTGCGCTCGGCCCTTCCGGCTGGCTGGTTTATTGCTGATAAATCTGGAGCCGGTGA
GCGTGGGTCTCGCGGTATCATTGCAGCACTGGGGCCAGATGGTAAGCCCTCCCGTATCGTAGTTATCTACACGACGGGGAGTCA
GGCAACTATGGATGAACGAAATAGACAGATCGCTGAGATAGGTGCCTCACTGATTAAGCATTGGTAACTGTCAGACCAAGTTTACT
CATATATACTTTAGATTGATTTAAAACTTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTCTTGAGATCCTTTTTTTCTGCGCGT
AATCTGCTGCTTGCAAACAAAAAAACCACCGCTACCAGCGGTGGTTTGTTTGCCGGATCAAGAGCTACCAACTCTTTTTCCGAAGG
TAACTGGCTTCAGCAGAGCGCAGATACCAAATACTGTCCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGATAAGTCGTGTCTTACCGGGTTGGACTCAA
GACGATAGTTACCGGATAAGGCGCAGCGGTCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCTGGTATCTTTATAGTCCTGTCGGGTTTCGCCA
CCTCTGACTTGAGCGTCGATTTTTGTGATGCTCGTCAGGGGGGCGGAGCCTATGGAAAAACGCCAGCAACGCGGCCTTTTTACG
GTTCCTGGCCTTTTGCTGGCCTTTTGCTCACATGT
GA optimized nucleotide sequence of Homo sapiens alpha subunit of beta hexosaminidase
SEQ ID NO: 64
TGACATCTAGCAGACTGTGGTTCAGCCTGCTGCTGGCCGCTGCTTTTGCTGGCAGAGCTACAGCTCTTTGGCCCTGGCCTCAGAA
CTTCCAGACCAGCGACCAGAGATACGTGCTGTACCCCAACAACTTCCAGTTCCAGTACGACGTGTCCAGCGCCGCTCAGCCTGG
ATGTTCTGTGCTGGATGAGGCCTTCCAGCGGTACAGGGATCTGCTGTTTGGCAGCGGCTCTTGGCCCAGACCTTACCTGACAGG
CAAGCGGCACACCCTGGAAAAGAACGTGCTGGTGGTGTCCGTGGTCACCCCTGGCTGTAATCAGCTGCCCACACTGGAAAGCGT
GGAAAACTACACCCTGACCATCAACGACGACCAGTGTCTGCTGCTGAGCGAGACAGTTTGGGGAGCCCTGAGAGGCCTGGAAAC
CTTTTCTCAGCTCGTGTGGAAGTCCGCCGAGGGCACCTTCTTCATCAACAAGACCGAGATCGAGGACTTCCCCAGATTTCCCCAC
AGAGGACTGCTGCTCGACACCAGCAGACATTACCTGCCTCTGAGCAGCATCCTGGATACCCTGGACGTGATGGCCTACAACAAG
CTGAACGTGTTCCACTGGCACCTGGTGGACGACCCTAGCTTCCCTTACGAGAGCTTCACATTCCCCGAGCTGATGCGGAAGGGC
AGCTACAACCCTGTGACACACATCTACACAGCCCAGGACGTGAAAGAAGTGATCGAGTACGCCCGGCTGCGGGGCATTAGAGTG
CTGGCCGAATTTGACACCCCTGGACACACCCTGTCTTGGGGCCCTGGAATTCCTGGACTGCTGACCCCTTGTTACAGCGGCAGC
GAGCCTTCTGGCACATTCGGCCCTGTGAACCCCAGCCTGAACAACACCTACGAGTTTATGAGCACATTCTTTCTGGAAGTCTCCA
GCGTGTTCCCCGACTTCTATCTGCATCTCGGAGGCGACGAGGTGGACTTCACCTGTTGGAAGTCTAACCCCGAGATCCAGGATTT
CATGCGGAAGAAAGGCTTCGGCGAGGATTTCAAGCAGCTGGAAAGCTTCTACATCCAGACACTGCTGGACATCGTGTCTAGCTAC
GGCAAGGGCTACGTCGTGTGGCAAGAGGTGTTCGACAACAAAGTGAAGATCCAGCCTGACACCATCATCCAAGTGTGGCGCGAG
GACATCCCCGTGAACTACATGAAGGAACTCGAGCTGGTCACCAAGGCCGGCTTTAGAGCACTGCTGTCTGCCCCTTGGTATCTGA
ACCGGATCAGCTACGGCCCCGACTGGAAGGATTTCTACATCGTGGAACCCCTGGCCTTCGAGGGCACACCTGAACAGAAGGCCC
TGGTTATTGGAGGCGAGGCCTGTATGTGGGGCGAGTACGTGGACAACACCAACCTGGTGCCTAGACTGTGGCCTAGAGCTGGC
GCTGTGGCCGAAAGACTCTGGTCCAACAAACTGACCTCCGACCTGACCTTCGCCTACGAGAGACTGAGCCACTTCAGATGCGAG
CTGCTGAGAAGAGGCGTGCAGGCCCAGCCTCTGAATGTGGGCTTCTGCGAGCAAGAGTTCGAGCAGACA
GA optimized nucleotide sequence of Homo sapiens beta subunit of beta hexosaminidase
SEQ ID NO: 65
GCTAGAGCCCCTAGCGTGTCAGCCAAACCTGGACCTGCTTTGTGGCCTCTGCCTCTGCTGGTCAAGATGACCCCTAACCTGCTG
CATCTGGCCCCTGAGAACTTTTACATCTCTCACAGCCCCAACAGCACCGCCGGACCTAGCTGTACACTGCTCGAGGAAGCCTTCA
GAAGATACCACGGCTACATCTTCGGCTTCTACAAGTGGCACCACGAGCCTGCCGAGTTCCAGGCCAAAACACAGGTGCAGCAGC
TCCTCGTGTCTATCACCCTGCAGAGCGAGTGCGACGCCTTTCCTAACATCAGCAGCGACGAGAGCTATACCCTGCTTGTGAAAGA
ACCCGTGGCCGTGCTGAAGGCCAACAGAGTGTGGGGAGCACTGCGGGGACTCGAGACATTTTCCCAGCTGGTGTACCAGGACT
CCTACGGCACCTTCACCATCAATGAGAGCACAATCATCGACAGCCCCAGATTCAGCCACCGGGGCATCCTGATCGATACCAGCA
GGCACTATCTGCCCGTGAAGATCATCCTGAAAACACTGGACGCCATGGCCTTTAACAAGTTCAATGTGCTGCACTGGCATATCGT
GGACGATCAGAGCTTCCCCTACCAGTCCATCACCTTTCCAGAGCTGAGCAACAAGGGCTCCTACAGCCTGAGCCACGTGTACAC
CCCTAACGACGTGOGGATGGTCATCGAGTATGCCAGACTGAGAGGCATCAGGGTGCTGCCCGAGTTCGATACACCAGGCCATAC
ACTGAGCTGGGGCAAGGGCCAGAAAGACCTGCTGACACCCTGCTACTCCCGGCAGAACAAGCTGGACAGCTTTGGCCCCATCAA
TCCCACACTGAATACCACCTACAGCTTTCTGACCACCTTTTTCAAAGAAATCAGCGAGGTGTTCCCGGACCAGTTCATCCATCTCG
GCGGAGATGAAGTGGAATTCAAGTGCTGGGAGAGCAACCCCAAGATTCAGGACTTTATGAGACAGAAAGGATTCGGGACCGACT
TCAAGAAACTTGAGAGCTTCTATATTCAGAAGGTCCTGGACATCATTGCCACCATTAACAAGGGCAGCATTGTCTGGCAAGAAGTC
TTTGACGACAAGGCCAAGCTGGCCCCAGGCACAATCGTGGAAGTGTGGAAGGACAGCGCTTACCCCGAGGAACTGAGCAGAGT
GACAGCCAGCGGCTTCCCTGTGATTCTGAGCGCTCCCTGGTATCTGGATCTGATCTCTTACGGCCAGGACTGGCGGAAGTACTA
CAAGGTGGAACCTCTGGATTTCGGCGGCACCCAGAAGCAGAAGCAGCTGTTTATTGGCGGAGAAGCCTGCCTGTGGGGAGAATA
TGTGGACGCCACCAATCTGACCCCTCGGTTGTGGCCCAGAGCCTCTGCAGTGGGAGAGAGACTTTGGAGCAGCAAGGACGTGC
GCGACATGGACGACGCCTATGACAGACTGACCCGGCACAGATGCCGGATGGTGGAAAGAGGAATTGCCGCACAGCCTCTGTAC
GCCGGCTACTGCAACCACGAGAATATGTAG
IDT optimized nucleotide sequence of Homo sapiens alpha subunit of beta hexosaminidase
SEQ ID NO: 66
ATGACAAGCAGCAGACTGTGGTTCTCCCTGCTTTTGGCGGCGGCTTTTGCCGGACGGGCCACAGCCCTGTGGCCGTGGCCGCA
AAATTTTCAGACGAGCGACCAACGGTATGTTCTTTATCCAAATAACTTTCAATTTCAGTATGATGTGTCCTCCGCTGCACAGCCTGG
CTGCAGCGTCCTGGACGAGGCATTTCAGCGCTATAGAGATCTCCTCTTTGGGTCTGGAAGCTGGCCCAGACCATACTTGACTGGC
AAGAGGCACACCCTCGAGAAGAATGTTTTGGTGGTTTCTGTAGTAACCCCTGGGTGCAATCAACTTCCGACCCTTGAGTCAGTAG
AGAATTATACGTTGACAATAAATGACGATCAATGCCTTCTTCTTAGTGAGACGGTTTGGGGAGCGCTGCGAGGGCTTGAGACATTT
AGCCAGCTTGTATGGAAGAGTGCGGAGGGTACCTTCTTCATAAATAAAACGGAGATCGAAGATTTTCCGCGATTCCCTCACAGGG
GTCTGCTTCTGGATACCAGCAGGCATTATCTGCCTCTTAGCAGCATACTCGACACACTGGACGTGATGGCGTACAACAAACTCAA
CGTGTTCCACTGGCATCTGGTGGACGACCCAAGCTTTCCATACGAGAGTTTTACGTTTCCAGAGCTCATGAGAAAGGGTAGTTATA
ATCCCGTGACCCATATATATACCGCGCAGGACGTGAAAGAAGTCATTGAGTACGCGCGACTCCGAGGAATTAGAGTACTGGCAGA
GTTCGACACTCCTGGGCACACGCTCAGTTGGGGGCCTGGAATACCTGGATTGTTGACTCCCTGCTATAGTGGCAGTGAGCCCTC
AGGGACCTTTGGTCCGGTTAATCCCAGTCTTAACAACACATACGAGTTCATGTCTACGTTTTTTCTGGAGGTGTCTTCAGTATTCCC
TGACTTCTACCTGCACCTTGGGGGAGATGAGGTTGACTTCACTTGTTGGAAGTCTAACCCTGAAATACAGGACTTCATGCGGAAAA
AAGGATTCGGGGAGGATTTCAAACAACTGGAGTCCTTTTATATTCAGACCCTTTTGGACATTGTTTCCAGCTATGGCAAAGGGTAT
GTGGTGTGGCAGGAAGTTTTCGACAACAAAGTCAAGATCCAACCGGATACCATTATACAGGTCTGGAGGGAGGACATCCCCGTAA
ACTACATGAAGGAACTTGAACTGGTCACCAAAGCTGGCTTTAGAGCGCTGCTCTCTGCGCCTTGGTATTTGAATAGGATTAGTTAT
GGGCCCGATTGGAAGGACTTTTATATCGTCGAGCCGCTTGCCTTTGAGGGAACCCCTGAGCAAAAAGCACTTGTGATTGGGGGA
GAGGCTTGTATGTGGGGAGAATATGTCGATAATACGAACCTGGTACCCCGGCTGTGGCCTCGCGCTGGGGCCGTGGCGGAGCG
CCTCTGGTCCAATAAGTTGACAAGTGATTTGACCTTCGCTTACGAACGGTTGTCTCATTTCAGATGTGAGCTGCTGAGACGCGGC
GTACAAGCACAACCCCTGAACGTTGGTTTTTGCGAACAAGAATTCGAACAGACC
IDT optimized nucleotide sequence of Homo sapiens beta subunit of beta hexosaminidase
SEQ ID NO: 67
CCAGGGCGCCTTCAGTAAGCGCCAAACCCGGCCCAGCCCTGTGGCCTTTGCCACTCCTGGTAAAAATGACTCCTAATCTGCTGC
ATCTGGCGCCTGAAAATTTTTATATAAGCCACTCTCCTAATAGTACTGCAGGTCCCTCTTGCACTTTGCTCGAAGAAGCTTTTCGAA
GGTATCACGGTTATATTTTTGGATTTTATAAGTGGCACCATGAACCTGCGGAATTCCAAGCGAAAACTCAAGTCCAACAGTTGCTC
GTAAGTATTACACTTCAGAGCGAGTGTGATGCTTTCCCTAATATCAGTTCTGACGAAAGTTACACGCTCCTGGTAAAGGAGCCTGT
CGCTGTACTGAAGGCGAATCGCGTTTGGGGCGCCTTGCGAGGCCTGGAAACTTTCAGTCAGTTGGTGTATCAGGATTCCTATGG
GACATTCACGATCAATGAATCCACTATTATAGACAGCCCTAGGTTTAGTCATAGGGGCATACTGATTGACACTTCTAGACATTACCT
GCCAGTCAAAATCATCCTGAAAACACTTGATGCCATGGCATTCAATAAGTTTAATGTTCTCCATTGGCACATCGTGGACGATCAAA
GCTTTCCATACCAGTCTATAACATTTCCGGAGCTTAGTAACAAGGGGAGCTATTCACTCTCACACGTTTACACCCCGAACGACGTG
CGCATGGTTATTGAATACGCCAGACTTCGAGGCATTAGAGTTCTTCCAGAATTTGATACACCAGGCCACACGCTCTCCTGGGGCA
AAGGCCAGAAGGACCTGCTTACCCCATGTTACTCCCGACAAAACAAACTCGATTCTTTTGGGCCAATAAACCCAACGTTGAACACT
ACGTACTCCTTCCTGACTACGTTTTTTAAGGAAATAAGTGAGGTATTCCCTGACCAGTTTATTCACTTGGGTGGTGATGAAGTCGAA
TTTAAGTGTTGGGAGTCAAACCCCAAAATACAAGACTTCATGCGCCAGAAGGGGTTTGGGACCGACTTTAAAAAACTCGAGTCTTT
CTACATTCAAAAGGTTCTCGATATTATCGCAACAATTAACAAAGGGAGCATTGTGTGGCAAGAGGTATTTGACGATAAAGCCAAAC
TGGCGCCCGGAACGATAGTCGAGGTCTGGAAAGACAGCGCCTATCCCGAGGAGCTTAGTCGAGTCACGGCTTCAGGTTTTCCAG
TTATCCTTAGTGCCCCGTGGTACTTGGATTTGATTTCCTACGGTCAAGACTGGCGCAAGTATTATAAGGTTGAACCTCTTGATTTTG
GTGGGACCCAAAAACAAAAGCAGTTGTTCATCGGTGGGGAAGCTTGCTTGTGGGGTGAGTACGTAGACGCGACTAATCTTACGC
CTCGATTGTGGCCTCGGGCATCTGCAGTTGGGGAGCGCTTGTGGAGCTCAAAGGACGTGCGAGACATGGATGACGCTTATGATA
GACTGACCCGACATAGGTGTCGAATGGTGGAGAGAGGTATAGCGGCCCAACCTCTTTATGCCGGATACTGCAACCATGAAAACAT
GTAA
NV optimized nucleotide sequence of Homo sapiens alpha subunit of beta hexosaminidase
SEQ ID NO: 68
ATGACCTCATCCCGCTTGTGGTTCTCTCTGTTGTTGGCGGCCGCCTTTGCAGGAAGGGCTACTGCCTTGTGGCCCTGGCCACAAA
ATTTCCAGACCAGTGATCAGCGATATGTACTCTATCCAAATAACTTCCAGTTTCAATACGACGTATCCAGCGCCGCTCAGCCAGGC
TGTAGCGTGCTGGATGAAGCGTTTCAACGCTATCGAGATCTGTTGTTCGGGTCAGGTTCTTGGCCCAGGCCGTATCTCACTGGGA
AAAGGCACACCCTGGAAAAGAATGTGCTGGTTGTCAGTGTGGTTACACCAGGATGCAATCAGCTCCCAACACTTGAGAGCGTGGA
AAACTATACTCTCACAATCAACGACGATCAGTGTCTCCTGCTCTCCGAAACAGTCTGGGGCGCTCTGCGGGGGTTGGAAACGTTC
TCACAGCTGGTATGGAAGTCCGCCGAGGGAACTTTCTTCATCAACAAAACGGAGATCGAAGACTTTCCCAGGTTCCCACACAGAG
GTCTGCTTCTCGACACTAGCCGGCATTACCTTCCCCTGAGCTCTATTCTTGACACCCTCGACGTAATGGCCTATAACAAGCTGAAC
GTGTTTCATTGGCATCTCGTGGACGATCCAAGCTTCCCATACGAGTCCTTCACATTCCCAGAGCTGATGCGCAAAGGGTCATACA
ATCCAGTGACACATATTTACACGGCCCAGGATGTGAAAGAGGTTATTGAGTACGCCAGACTCAGGGGCATTAGGGTGCTGGCCG
AGTTTGATACACCAGGGCATACCCTCTCATGGGGCCCTGGTATCCCTGGGCTGCTGACTCCCTGCTATTCAGGAAGTGAGCCGA
GTGGGACTTTCGGGCCAGTGAACCCCTCTCTGAACAACACATACGAGTTCATGTCCACATTTTTCCTCGAGGTGAGTTCAGTGTTC
CCCGATTTTTATCTTCACCTTGGTGGAGACGAGGTTGACTTCACCTGTTGGAAGAGCAACCCCGAGATTCAGGACTTCATGCGCA
AAAAGGGGTTTGGAGAGGATTTTAAGCAGCTTGAGAGCTTTTATATACAGACACTGCTCGACATCGTGTCTTCATATGGAAAAGGC
TATGTGGTTTGGCAAGAGGTCTTTGACAACAAAGTCAAGATCCAGCCTGACACGATTATCCAAGTATGGAGAGAGGACATACCCG
TCAACTACATGAAGGAGCTGGAGCTGGTGACCAAGGCTGGGTTTAGGGCCCTGCTCTCAGCTCCCTGGTACCTGAACCGAATAT
CCTATGGGCCGGATTGGAAAGACTTCTACATAGTGGAACCACTGGCCTTCGAAGGGACTCCTGAGCAGAAGGCATTGGTGATCG
GCGGTGAGGCTTGTATGTGGGGCGAGTACGTAGACAATACGAACCTCGTGCCAAGGCTTTGGCCAAGAGCTGGTGCAGTTGCAG
AGCGACTGTGGAGCAATAAGCTGACAAGTGACCTCACATTTGCCTACGAGCGGCTGTCCCACTTTCGCTGTGAACTCCTGAGACG
GGGAGTTCAAGCCCAGCCTCTGAATGTTGGCTTTTGCGAGCAGGAGTTCGAACAGACA
NV optimized nucleotide sequence of Homo sapiens beta subunit of beta hexosaminidase
SEQ ID NO: 69
GCTAGAGCACCATCAGTTAGTGCCAAACCAGGGCCTGCATTGTGGCCACTGCCCCTCCTGGTCAAAATGACACCTAATCTGCTCC
ATCTTGCCCCGGAGAATTTCTATATCAGCCACTCCCCCAATTCAACCGCCGGGCCCTCATGCACCCTCCTCGAAGAGGCCTTTCG
GAGATACCATGGGTATATATTCGGCTTCTACAAATGGCATCACGAACCTGCTGAGTTCCAGGCCAAGACCCAAGTCCAACAGTTG
CTGGTGTCAATCACACTGCAGAGCGAATGCGATGCTTTTCCCAACATTTCTTCCGACGAGTCTTACACGCTCCTTGTGAAGGAGC
CAGTAGCTGTCTTGAAGGCCAATCGCGTGTGGGGTGCCTTGAGGGGCTTGGAAACATTTTCCCAGCTGGTCTACCAGGATTCCTA
TGGGACCTTTACCATCAACGAAAGTACGATAATTGACAGTCCTCGGTTCTCACACAGGGGCATCTTGATCGATACAAGCAGGCATT
ACTTGCCAGTCAAGATCATCCTTAAGACACTGGATGCCATGGCTTTCAATAAGTTCAACGTGCTTCATTGGCACATTGTGGATGAT
CAGAGCTTCCCATACCAGAGCATTACCTTCCCAGAGCTTTCCAATAAGGGTAGTTATTCTCTCAGCCACGTGTACACACCCAATGA
TGTGCGCATGGTCATCGAGTATGCCCGGCTGAGAGGGATTAGGGTCCTGCCCGAATTTGACACGCCAGGCCACACACTCTCCTG
GGGCAAGGGCCAGAAGGATCTTTTGACTCCATGTTATTCAAGACAAAATAAGTTGGACAGCTTTGGACCCATCAACCCCACCCTTA
ACACAACTTACTCATTCCTGACCACATTCTTTAAGGAGATCAGTGAGGTCTTCCCCGACCAGTTTATTCATCTCGGTGGCGATGAG
GTCGAGTTTAAATGCTGGGAAAGCAACCCCAAGATCCAGGACTTCATGCGGCAAAAGGGATTCGGAACTGATTTCAAAAAGCTCG
AGTCCTTCTATATTCAGAAGGTCTTGGACATCATCGCTACCATAAACAAGGGGAGCATCGTTTGGCAGGAGGTGTTCGACGATAAA
GCCAAGCTGGCTCCAGGGACTATTGTCGAGGTGTGGAAAGATAGTGCCTACCCCGAGGAACTTTCACGGGTGACCGCCTCTGGT
TTTCCTGTGATCCTCTCAGCACCGTGGTATCTTGATCTCATCTCTTACGGACAGGACTGGCGGAAGTACTACAAAGTTGAACCACT
GGATTTTGGGGGAACGCAGAAACAAAAGCAGCTGTTTATCGGAGGCGAAGCATGTCTGTGGGGAGAGTATGTAGATGCAACCAA
CCTTACCCCACGGCTGTGGCCACGGGCTTCTGCTGTAGGTGAGAGGCTCTGGAGCAGCAAAGACGTTCGCGACATGGACGACG
CATATGACCGGCTTACTAGACACCGCTGCAGAATGGTTGAGAGGGGAATTGCCGCCCAGCCCCTTTATGCCGGATATTGCAATCA
TGAAAATATGTAA
L1 AB mouse gene construct
SEQ ID NO: 75
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGGGGGG
CGAGGGGGGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGGGGGCGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGCGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGGCCGGCTGTAGACTGTGGGTTTCACTGCTGCTGGC
TGCCGCTCTGGCTTGTCTGGCTACAGCTCTTTGGCCCTGGCCTCAGTACATCCAGACCTACCACAGACGGTACACACTGTACCCC
AACAACTTCCAGTTCCGCTACCACGTGTCCTCTGCTGCTCAGGCTGGATGTGTGGTGCTGGACGAGGCCTTCAGAAGATACAGAA
ACCTGCTGTTCGGCAGCGGCAGCTGGCCTAGACCTAGCTTCTCTAACAAGCAGCAGACCCTGGGCAAGAACATCCTGGTGGTGT
CTGTGGTCACCGCCGAGTGCAACGAGTTCCCCAACCTGGAAAGCGTGGAAAACTACACCCTGACCATCAACGACGACCAGTGCC
TGCTGGCCTCTGAGACAGTTTGGGGAGCCCTGAGAGGCCTGGAAACCTTCTCTCAGCTCGTGTGGAAGTCTGCCGAGGGCACCT
TCTTCATCAACAAGACCAAGATCAAGGACTTCCCTAGGTTCCCTCACAGAGGCGTGCTGCTGGACACCAGCAGACACTACCTGCC
ACTGTCCTCCATCCTGGACACCCTGGATGTGATGGCCTACAACAAGTTCAACGTGTTCCACTGGCACCTGGTGGACGACAGCAGC
TTCCCTTACGAGAGCTTCACATTCCCCGAGCTGACCAGAAAGGGCAGCTTCAACCCCGTGACACACATCTACACAGCCCAGGACG
TGAAAGAAGTGATCGAGTACGCCAGACTGAGAGGCATCAGAGTGCTGGCCGAGTTCGACACACCTGGCCACACACTTTCTTGGG
GACCTGGTGCTCCTGGCCTGCTGACACCTTGTTACTCTGGCTCTCACCTGAGCGGCACATTCGGCCCTGTGAACCCTAGCCTGAA
CAGCACCTACGACTTTATGAGCACCCTGTTCCTCGAGATCAGCAGCGTGTTCCCCGACTTCTACCTGCACCTCGGCGGAGATGAG
GTGGACTTCACCTGTTGGAAGTCTAACCCCAACATCCAGGCTTTCATGAAGAAGAAGGGCTTCACCGACTTCAAGCAGCTGGAAA
GCTTCTACATTCAGACCCTGCTGGATATCGTGTCCGACTACGACAAGGGCTACGTCGTGTGGCAAGAGGTGTTCGACAACAAAGT
GAAAGTGCGGCCCGACACCATCATCCAAGTGTGGCGGGAAGAGATGCCCGTCGAGTACATGCTGGAAATGCAGGACATCACCAG
AGCCGGCTTCAGGGCTCTGCTTAGCGCTCCCTGGTATCTGAACAGAGTGAAGTACGGCCCCGACTGGAAGGACATGTACAAGGT
GGAACCCCTGGCCTTCCACGGCACCCCTGAACAGAAGGCTCTGGTTATCGGAGGCGAGGCCTGTATGTGGGGAGAGTACGTGG
ACAGCACCAACCTGGTGCCAAGACTGTGGCCTAGAGCTGGCGCTGTGGCTGAGAGACTGTGGTCCAGCAACCTGACCACCAACA
TCGACTTCGCCTTCAAGAGACTGAGCCACTTCAGATGCGAACTCGTGCGGAGAGGAATCCAGGCTCAGCCTATCTCTGTGGGCTA
CTGCGAGCAAGAGTTCGAGCAGACAGGTGGTGGTGGATCTGGAGGCGGAGGATCAGGGGGGGGAGGTTCTGGAGGTGGAGGT
AGTCAGCCTGCTCTGTGGCCATTTCCTAGAAGCGTGCAGATGTTCCCCAGACTGCTGTACATCAGCGCCGAGGACTTCAGCATCG
ATCACAGCCCCAACAGCACAGCTGGCCCATCTTGTAGCCTGCTGCAAGAGGCCTTTAGACGGTACTACAACTACGTGTTCGGCTT
CTACAAGAGGCACCACGGACCTGCCAGATTCAGAGCCGAACCTCAGCTGCAGAAGCTGCTCGTCAGCATCACCCTGGAATCCGA
GTGCGAGAGCTTTCCCAGCCTGTCCAGCGACGAGACATACTCCCTGCTGGTGCAAGAGCCTGTGGCTGTGCTGAAGGCCAACTC
TGTGTGGGGTGCTCTGCGCGGACTGGAAACATTTTCCCAGCTGGTGTACCAGGACAGCTTCGGCACCTTCACAATCAACGAGTC
CTCTATCGCCGACTCTCCTAGATTCCCACACAGGGGCATCCTGATCGATACCTCCAGGCACTTCCTGCCTGTGAAAACCATCCTC
AAGACCCTGGACGCCATGGCTTTCAACAAGTTTAATGTGCTGCATTGGCACATCGTGGATGACCAAAGCTTCCCCTACCAGTCCA
CCACCTTTCCAGAGCTGTCCAACAAGGGCTCCTACAGCCTGTCTCACGTGTACACCCCTAACGACGTGCGGATGGTCCTGGAATA
CGCTAGGCTGCGGGGAATCAGAGTGATCCCTGAGTTCGATACTCCCGGACACACACAGAGCTGGGGCAAGGGACAGAAGAACC
TGCTGACCCCATGCTACAACCAGAAAACAAAGACCCAGGTGTTCGGCCCAGTGGACCCAACCGTGAACACAACCTACGCTTTCTT
CAACACATTCTTCAAAGAAATCAGCTCCGTGTTTCCCGACCAGTTCATCCATCTCGGCGGCGACGAAGTCGAGTTCCAGTGTTGG
GCCAGCAATCCCAATATCCAAGGATTCATGAAGCGGAAAGGCTTCGGCTCCGACTTCAGAAGGCTGGAATCTTTCTACATCAAGA
AGATCCTCGAAATCATCAGCAGCCTGAAAAAGAACAGCATCGTCTGGCAAGAAGTCTTTGACGACAAGGTCGAGCTGCAGCCAGG
CACTGTGGTGGAAGTGTGGAAAAGCGAGCACTACAGCTACGAGCTGAAGCAAGTGACAGGCAGCGGCTTCCCTGCTATCCTCTC
TGCCCCTTGGTATCTGGACCTGATCAGCTACGGCCAGGATTGGAAGAACTACTACAAGGTTGAGCCCCTCAACTTCGAGGGCAG
CGAGAAGCAGAAACAGCTGGTCATTGGCGGCGAGGCTTGTCTCTGGGGCGAGTTTGTGGATGCCACCAATCTGACCCCTAGGCT
GTGGCCAAGGGCTTCTGCTGTGGGAGAGAGACTTTGGAGCCCCAAGACCGTGACCGACCTGGAAAACGCCTATAAGAGACTGGC
CGTGCACAGATGCAGAATGGTGTCCCGAGGAATCGCCGCTCAGCCTCTGTACACCGGCTACTGCAACTACGAGAACAAGATCTG
AGGATCCAACTTGTTTATTGCAGCTTATAATGGTTACAAATAAAGCAATAGCATCACAAATTTCACAAATAAAGCATTTTTTTCACTG
CATTCTAGTTGTGGTTTGTCCAAACTCATCAATGTATOTTATGCGGCCGCAGGAACCCCTAGTGATGGAGTTGGCCACTCCCTCTC
TGCGCGCTCGCTCGCTCACTGAGGCCGGGCGACCAAAGGTCGCCCGACGCCCGGGCTTTGCCCGGGGGGCCTCAGTGAGCGA
GCGAGCGCGCAGCTGCCTGCAGG
L2 AB mouse gene construct
SEQ ID NO: 76
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGGGGGG
CGAGGGGGGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGGGGGCGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGGGGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGGCCGGCTGTAGACTGTGGGTTTCACTGCTGCTGGC
TGCCGCTCTGGCTTGTCTGGCTACAGCTCTTTGGCCCTGGCCTCAGTACATCCAGACCTACCACAGACGGTACACACTGTACCCC
AACAACTTCCAGTTCCGCTACCACGTGTCCTCTGCTGCTCAGGCTGGATGTGTGGTGCTGGACGAGGCCTTCAGAAGATACAGAA
ACCTGCTGTTCGGCAGCGGCAGCTGGCCTAGACCTAGCTTCTCTAACAAGCAGCAGACCCTGGGCAAGAACATCCTGGTGGTGT
CTGTGGTCACCGCCGAGTGCAACGAGTTCCCCAACCTGGAAAGCGTGGAAAACTACACCCTGACCATCAACGACGACCAGTGCC
TGCTGGCCTCTGAGACAGTTTGGGGAGCCCTGAGAGGCCTGGAAACCTTCTCTCAGCTCGTGTGGAAGTCTGCCGAGGGCACCT
TCTTCATCAACAAGACCAAGATCAAGGACTTCCCTAGGTTCCCTCACAGAGGCGTGCTGCTGGACACCAGCAGACACTACCTGCC
ACTGTCCTCCATCCTGGACACCCTGGATGTGATGGCCTACAACAAGTTCAACGTGTTCCACTGGCACCTGGTGGACGACAGCAGC
TTCCCTTACGAGAGCTTCACATTCCCCGAGCTGACCAGAAAGGGCAGCTTCAACCCCGTGACACACATCTACACAGCCCAGGACG
TGAAAGAAGTGATCGAGTACGCCAGACTGAGAGGCATCAGAGTGCTGGCCGAGTTCGACACACCTGGCCACACACTTTCTTGGG
GACCTGGTGCTCCTGGCCTGCTGACACCTTGTTACTCTGGCTCTCACCTGAGCGGCACATTCGGCCCTGTGAACCCTAGCCTGAA
CAGCACCTACGACTTTATGAGCACCCTGTTCCTCGAGATCAGCAGCGTGTTCCCCGACTTCTACCTGCACCTCGGCGGAGATGAG
GTGGACTTCACCTGTTGGAAGTCTAACCCCAACATCCAGGCTTTCATGAAGAAGAAGGGCTTCACCGACTTCAAGCAGCTGGAAA
GCTTCTACATTCAGACCCTGCTGGATATCGTGTCCGACTACGACAAGGGCTACGTCGTGTGGCAAGAGGTGTTCGACAACAAAGT
GAAAGTGCGGCCCGACACCATCATCCAAGTGTGGCGGGAAGAGATGCCCGTCGAGTACATGCTGGAAATGCAGGACATCACCAG
AGCCGGCTTCAGGGCTCTGCTTAGCGCTCCCTGGTATCTGAACAGAGTGAAGTACGGCCCCGACTGGAAGGACATGTACAAGGT
GGAACCCCTGGCCTTCCACGGCACCCCTGAACAGAAGGCTCTGGTTATCGGAGGCGAGGCCTGTATGTGGGGAGAGTACGTGG
ACAGCACCAACCTGGTGCCAAGACTGTGGCCTAGAGCTGGCGCTGTGGCTGAGAGACTGTGGTCCAGCAACCTGACCACCAACA
TCGACTTCGCCTTCAAGAGACTGAGCCACTTCAGATGCGAACTCGTGCGGAGAGGAATCCAGGCTCAGCCTATCTCTGTGGGCTA
CTGCGAGCAAGAGTTCGAGCAGACATCTGGAGGATCTAGCGGAGGATCCTCTGGCAGCGAGACACCAGGAACAAGCGAGTCAG
CAACACCAGAGAGCAGTGGCGGCAGCAGCGGCGGCAGCAGCCAGCCTGCTCTGTGGCCATTTCCTAGAAGCGTGCAGATGTTC
CCCAGACTGCTGTACATCAGCGCCGAGGACTTCAGCATCGATCACAGCCCCAACAGCACAGCTGGCCCATCTTGTAGCCTGCTG
CAAGAGGCCTTTAGACGGTACTACAACTACGTGTTCGGCTTCTACAAGAGGCACCACGGACCTGCCAGATTCAGAGCCGAACCTC
AGCTGCAGAAGCTGCTCGTCAGCATCACCCTGGAATCCGAGTGCGAGAGCTTTCCCAGCCTGTCCAGCGACGAGACATACTCCC
TGCTGGTGCAAGAGCCTGTGGCTGTGCTGAAGGCCAACTCTGTGTGGGGTGCTCTGCGCGGACTGGAAACATTTTCCCAGCTGG
TGTACCAGGACAGCTTCGGCACCTTCACAATCAACGAGTCCTCTATCGCCGACTCTCCTAGATTCCCACACAGGGGCATCCTGAT
CGATACCTCCAGGCACTTCCTGCCTGTGAAAACCATCCTCAAGACCCTGGACGCCATGGCTTTCAACAAGTTTAATGTGCTGCATT
GGCACATCGTGGATGACCAAAGCTTCCCCTACCAGTCCACCACCTTTCCAGAGCTGTCCAACAAGGGCTCCTACAGCCTGTCTCA
CGTGTACACCCCTAACGACGTGCGGATGGTCCTGGAATACGCTAGGCTGCGGGGAATCAGAGTGATCCCTGAGTTCGATACTCC
CGGACACACACAGAGCTGGGGCAAGGGACAGAAGAACCTGCTGACCCCATGCTACAACCAGAAAACAAAGACCCAGGTGTTCGG
CCCAGTGGACCCAACCGTGAACACAACCTACGCTTTCTTCAACACATTCTTCAAAGAAATCAGCTCCGTGTTTCCCGACCAGTTCA
TCCATCTCGGCGGCGACGAAGTCGAGTTCCAGTGTTGGGCCAGCAATCCCAATATCCAAGGATTCATGAAGCGGAAAGGCTTCG
GCTCCGACTTCAGAAGGCTGGAATCTTTCTACATCAAGAAGATCCTCGAAATCATCAGCAGCCTGAAAAAGAACAGCATCGTCTG
GCAAGAAGTCTTTGACGACAAGGTCGAGCTGCAGCCAGGCACTGTGGTGGAAGTGTGGAAAAGCGAGCACTACAGCTACGAGCT
GAAGCAAGTGACAGGCAGCGGCTTCCCTGCTATCCTCTCTGCCCCTTGGTATCTGGACCTGATCAGCTACGGCCAGGATTGGAA
GAACTACTACAAGGTTGAGCCCCTCAACTTCGAGGGCAGCGAGAAGCAGAAACAGCTGGTCATTGGCGGCGAGGCTTGTCTCTG
GGGCGAGTTTGTGGATGCCACCAATCTGACCCCTAGGCTGTGGCCAAGGGCTTCTGCTGTGGGAGAGAGACTTTGGAGCCCCAA
GACCGTGACCGACCTGGAAAACGCCTATAAGAGACTGGCCGTGCACAGATGCAGAATGGTGTCCCGAGGAATCGCCGCTCAGCC
TCTGTACACCGGCTACTGCAACTACGAGAACAAGATCTGAGGATCCAACTTGTTTATTGCAGCTTATAATGGTTACAAATAAAGCAA
TAGCATCACAAATTTCACAAATAAAGCATTTTTTTCACTGCATTCTAGTTGTGGTTTGTCCAAACTCATCAATGTATOTTATGCGGCC
GCAGGAACCCCTAGTGATGGAGTTGGCCACTCCCTCTCTGCGCGCTCGCTCGCTCACTGAGGCCGGGCGACCAAAGGTCGCCC
GACGCCCGGGCTTTGCCCGGGGGGCCTCAGTGAGCGAGCGAGCGCGCAGCTGCCTGCAGG
L3 AB mouse gene construct
SEQ ID NO: 77
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGGGGGGGGGGGCGGGG
CGAGGGGGGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGCGGGGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGCGGGCGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGGCCGGCTGTAGACTGTGGGTTTCACTGCTGCTGGC
TGCCGCTCTGGCTTGTCTGGCTACAGCTCTTTGGCCCTGGCCTCAGTACATCCAGACCTACCACAGACGGTACACACTGTACCCC
AACAACTTCCAGTTCCGCTACCACGTGTCCTCTGCTGCTCAGGCTGGATGTGTGGTGCTGGACGAGGCCTTCAGAAGATACAGAA
ACCTGCTGTTCGGCAGCGGCAGCTGGCCTAGACCTAGCTTCTCTAACAAGCAGCAGACCCTGGGCAAGAACATCCTGGTGGTGT
CTGTGGTCACCGCCGAGTGCAACGAGTTCCCCAACCTGGAAAGCGTGGAAAACTACACCCTGACCATCAACGACGACCAGTGCC
TGCTGGCCTCTGAGACAGTTTGGGGAGCCCTGAGAGGCCTGGAAACCTTCTCTCAGCTCGTGTGGAAGTCTGCCGAGGGCACCT
TCTTCATCAACAAGACCAAGATCAAGGACTTCCCTAGGTTCCCTCACAGAGGCGTGCTGCTGGACACCAGCAGACACTACCTGCC
ACTGTCCTCCATCCTGGACACCCTGGATGTGATGGCCTACAACAAGTTCAACGTGTTCCACTGGCACCTGGTGGACGACAGCAGC
TTCCCTTACGAGAGCTTCACATTCCCCGAGCTGACCAGAAAGGGCAGCTTCAACCCCGTGACACACATCTACACAGCCCAGGACG
TGAAAGAAGTGATCGAGTACGCCAGACTGAGAGGCATCAGAGTGCTGGCCGAGTTCGACACACCTGGCCACACACTTTCTTGGG
GACCTGGTGCTCCTGGCCTGCTGACACCTTGTTACTCTGGCTCTCACCTGAGCGGCACATTCGGCCCTGTGAACCCTAGCCTGAA
CAGCACCTACGACTTTATGAGCACCCTGTTCCTCGAGATCAGCAGCGTGTTCCCCGACTTCTACCTGCACCTCGGCGGAGATGAG
GTGGACTTCACCTGTTGGAAGTCTAACCCCAACATCCAGGCTTTCATGAAGAAGAAGGGCTTCACCGACTTCAAGCAGCTGGAAA
GCTTCTACATTCAGACCCTGCTGGATATCGTGTCCGACTACGACAAGGGCTACGTCGTGTGGCAAGAGGTGTTCGACAACAAAGT
GAAAGTGCGGCCCGACACCATCATCCAAGTGTGGCGGGAAGAGATGCCCGTCGAGTACATGCTGGAAATGCAGGACATCACCAG
AGCCGGCTTCAGGGCTCTGCTTAGCGCTCCCTGGTATCTGAACAGAGTGAAGTACGGCCCCGACTGGAAGGACATGTACAAGGT
GGAACCCCTGGCCTTCCACGGCACCCCTGAACAGAAGGCTCTGGTTATCGGAGGCGAGGCCTGTATGTGGGGAGAGTACGTGG
ACAGCACCAACCTGGTGCCAAGACTGTGGCCTAGAGCTGGCGCTGTGGCTGAGAGACTGTGGTCCAGCAACCTGACCACCAACA
TCGACTTCGCCTTCAAGAGACTGAGCCACTTCAGATGCGAACTCGTGCGGAGAGGAATCCAGGCTCAGCCTATCTCTGTGGGCTA
CTGCGAGCAAGAGTTCGAGCAGACAGGCAGCGCCGGCAGCGCCGCCGGCAGCGGCGAGTTCCAGCCTGCTCTGTGGCCATTTC
CTAGAAGCGTGCAGATGTTCCCCAGACTGCTGTACATCAGCGCCGAGGACTTCAGCATCGATCACAGCCCCAACAGCACAGCTG
GCCCATCTTGTAGCCTGCTGCAAGAGGCCTTTAGACGGTACTACAACTACGTGTTCGGCTTCTACAAGAGGCACCACGGACCTGC
CAGATTCAGAGCCGAACCTCAGCTGCAGAAGCTGCTCGTCAGCATCACCCTGGAATCCGAGTGCGAGAGCTTTCCCAGCCTGTC
CAGCGACGAGACATACTCCCTGCTGGTGCAAGAGCCTGTGGCTGTGCTGAAGGCCAACTCTGTGTGGGGTGCTCTGCGCGGACT
GGAAACATTTTCCCAGCTGGTGTACCAGGACAGCTTCGGCACCTTCACAATCAACGAGTCCTCTATCGCCGACTCTCCTAGATTC
CCACACAGGGGCATCCTGATCGATACCTCCAGGCACTTCCTGCCTGTGAAAACCATCCTCAAGACCCTGGACGCCATGGCTTTCA
ACAAGTTTAATGTGCTGCATTGGCACATCGTGGATGACCAAAGCTTCCCCTACCAGTCCACCACCTTTCCAGAGCTGTCCAACAAG
GGCTCCTACAGCCTGTCTCACGTGTACACCCCTAACGACGTGCGGATGGTCCTGGAATACGCTAGGCTGCGGGGAATCAGAGTG
ATCCCTGAGTTCGATACTCCCGGACACACACAGAGCTGGGGCAAGGGACAGAAGAACCTGCTGACCCCATGCTACAACCAGAAA
ACAAAGACCCAGGTGTTCGGCCCAGTGGACCCAACCGTGAACACAACCTACGCTTTCTTCAACACATTCTTCAAAGAAATCAGCTC
CGTGTTTCCCGACCAGTTCATCCATCTCGGCGGCGACGAAGTCGAGTTCCAGTGTTGGGCCAGCAATCCCAATATCCAAGGATTC
ATGAAGCGGAAAGGCTTCGGCTCCGACTTCAGAAGGCTGGAATCTTTCTACATCAAGAAGATCCTCGAAATCATCAGCAGCCTGA
AAAAGAACAGCATCGTCTGGCAAGAAGTCTTTGACGACAAGGTCGAGCTGCAGCCAGGCACTGTGGTGGAAGTGTGGAAAAGCG
AGCACTACAGCTACGAGCTGAAGCAAGTGACAGGCAGCGGCTTCCCTGCTATCCTCTCTGCCCCTTGGTATCTGGACCTGATCAG
CTACGGCCAGGATTGGAAGAACTACTACAAGGTTGAGCCCCTCAACTTCGAGGGCAGCGAGAAGCAGAAACAGCTGGTCATTGG
CGGCGAGGCTTGTCTCTGGGGCGAGTTTGTGGATGCCACCAATCTGACCCCTAGGCTGTGGCCAAGGGCTTCTGCTGTGGGAGA
GAGACTTTGGAGCCCCAAGACCGTGACCGACCTGGAAAACGCCTATAAGAGACTGGCCGTGCACAGATGCAGAATGGTGTCCCG
AGGAATCGCCGCTCAGCCTCTGTACACCGGCTACTGCAACTACGAGAACAAGATCTGAGGATCCAACTTGTTTATTGCAGCTTATA
ATGGTTACAAATAAAGCAATAGCATCACAAATTTCACAAATAAAGCATTTTTTTCACTGCATTCTAGTTGTGGTTTGTCCAAACTCAT
CAATGTATOTTATGCGGCCGCAGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCGCGCAGCTGCCTGCAGG
L1 BA mouse gene construct
SEQ ID NO: 78
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGGGGG
CGAGGGGGGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGGGGGCGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGCGGGCGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGCCTCAGAGCCCTAGATCTGCTCCTGGACTGCTGCT
GCTCCAGGCTCTGGTGTCTCTTGTGTCTCTGGCACTGGTGGCCCCTGCTAGACTGCAACCTGCTCTGTGGCCATTTCCTAGAAGC
GTGCAGATGTTCCCCAGACTGCTGTACATCAGCGCCGAGGACTTCAGCATCGATCACAGCCCCAACAGCACAGCTGGCCCTAGT
TGTAGCCTGCTGCAAGAGGCCTTCAGACGGTACTACAACTACGTGTTCGGCTTCTACAAGAGGCACCACGGACCTGCCAGATTCA
GAGCTGAGCCCCAGCTGCAGAAACTGCTGGTGTCTATCACCCTGGAAAGCGAGTGCGAGAGCTTCCCTAGCCTGAGCAGCGACG
AGACATACAGCCTGCTGGTGCAAGAGCCTGTGGCCGTGCTGAAGGCTAACTCTGTTTGGGGCGCTCTGAGAGGCCTGGAAACCT
TCTCTCAGCTGGTGTACCAGGACAGCTTCGGCACCTTCACCATCAACGAGTCCTCTATCGCCGACTCTCCTAGATTCCCTCACAG
AGGCATCCTGATCGATACCAGCAGACACTTCCTGCCTGTGAAAACCATCCTGAAAACCCTGGACGCCATGGCCTTCAACAAGTTC
AACGTGCTGCACTGGCACATCGTGGACGACCAGAGCTTTCCCTACCAGTCCACCACCTTTCCAGAGCTGTCCAACAAGGGCAGC
TACAGCCTGAGCCACGTGTACACCCCTAACGACGTGCGGATGGTGCTGGAATACGCCAGACTGAGAGGCATCAGAGTGATCCCC
GAGTTCGACACCCCTGGCCACACACAGTCTTGGGGCAAGGGACAGAAGAACCTGCTGACCCCTTGCTACAACCAGAAAACAAAG
ACCCAGGTGTTCGGCCCTGTGGACCCTACCGTGAACACCACCTACGCTTTCTTCAACACCTTCTTCAAAGAAATCAGCAGCGTGT
TCCCCGACCAGTTCATCCACCTCGGCGGAGATGAGGTCGAGTTCCAGTGTTGGGCCAGCAATCCCAACATCCAGGGCTTTATGA
AGAGGAAAGGCTTCGGCAGCGACTTCAGAAGGCTGGAAAGCTTCTACATCAAGAAGATCCTCGAGATCATCAGCAGCCTGAAGAA
GAACAGCATCGTCTGGCAAGAGGTGTTCGACGACAAGGTGGAACTGCAGCCTGGCACCGTGGTGGAAGTGTGGAAGTCTGAGC
ACTACAGCTACGAGCTGAAGCAAGTGACCGGCTCTGGCTTCCCTGCCATCCTTTCTGCCCCTTGGTATCTGGACCTGATCAGCTA
CGGCCAGGACTGGAAGAACTACTACAAGGTCGAGCCCCTGAACTTCGAGGGCAGCGAGAAGCAGAAGCAGCTGGTTATCGGAG
GCGAGGCTTGTCTGTGGGGCGAGTTCGTGGATGCCACCAACCTGACACCTAGACTGTGGCCTAGAGCCTCTGCTGTGGGCGAG
AGACTGTGGTCCCCTAAGACAGTGACCGACCTGGAAAACGCCTACAAGAGACTGGCCGTGCACAGATGCAGAATGGTGTCCAGA
GGAATCGCCGCTCAGCCTCTGTACACCGGCTACTGCAACTACGAGAACAAGATCGGTGGTGGTGGATCTGGAGGCGGAGGATCA
GGCGGCGGAGGTTCTGGAGGTGGAGGTAGTACCCTGGGCAAGAACATCCTGGTGGTGTCTGTGGTCACCGCCGAGTGCAACGA
GTTCCCCAACCTGGAATCCGTGGAAAACTACACCCTGACAATCAACGACGACCAGTGCCTGCTGGCCTCCGAAACAGTTTGGGG
AGCACTGCGCGGACTGGAAACATTCAGCCAGCTCGTGTGGAAATCTGCCGAGGGCACATTCTTCATCAACAAGACCAAGATCAAG
GACTTCCCTAGGTTCCCACACAGGGGCGTGCTGCTGGACACCTCCAGACATTACCTGCCTCTGAGCAGCATCCTGGACACACTG
GACGTGATGGCTTATAACAAGTTTAATGTGTTCCATTGGCACCTGGTCGACGACAGCAGCTTCCCTTACGAGTCCTTCACATTCCC
CGAGCTGACAAGAAAGGGCTCTTTCAACCCCGTGACACACATCTACACAGCCCAGGACGTGAAAGAAGTGATCGAGTACGCTAG
GCTGCGGGGAATCAGAGTGCTGGCTGAGTTCGATACACCCGGACACACTCTGAGTTGGGGACCTGGTGCTCCTGGCCTCCTGAC
ACCATGTTACTCTGGCTCTCACCTGAGCGGCACATTCGGCCCAGTGAACCCAAGCCTGAATAGCACCTACGACTTTATGAGCACC
CTGTTTCTGGAAATCTCCTCCGTGTTTCCCGACTTCTACCTGCATCTCGGCGGCGACGAGGTGGACTTCACCTGTTGGAAGTCTA
ACCCCAATATCCAGGCATTCATGAAGAAGAAGGGGTTCACCGACTTCAAGCAGCTTGAGAGCTTCTATATCCAGACGCTGCTGGA
TATCGTGTCCGACTACGACAAGGGCTACGTTGTGTGGCAAGAAGTCTTTGACAACAAAGTGAAAGTGCGGCCCGACACCATCATC
CAAGTGTGGCGGGAAGAGATGCCCGTCGAGTACATGCTGGAAATGCAGGACATCACAAGAGCCGGCTTCAGGGCTCTGCTGAG
CGCTCCATGGTATCTGAACAGAGTGAAGTACGGCCCCGATTGGAAGGACATGTACAAAGTGGAACCTCTGGCCTTCCACGGCAC
CCCTGAACAGAAAGCTCTCGTGATTGGCGGCGAGGCCTGCATGTGGGGAGAATACGTGGACAGCACAAACCTGGTGCCTCGGCT
TTGGCCAAGAGCTGGTGCTGTGGCTGAAAGGCTGTGGTCTAGCAACCTGACCACCAACATCGACTTCGCCTTCAAGAGGCTGTC
CCACTTCAGATGCGAACTCGTGCGGAGAGGAATCCAGGCTCAGCCAATCTCTGTGGGCTACTGCGAGCAAGAGTTCGAGCAGAC
CTGAGGATCCAACTTGTTTATTGCAGCTTATAATGGTTACAAATAAAGCAATAGCATCACAAATTTCACAAATAAAGCATTTTTTTCA
CTGCATTCTAGTTGTGGTTTGTCCAAACTCATCAATGTATOTTATGCGGCCGCAGGAACCCCTAGTGATGGAGTTGGCCACTCCCT
CTCTGCGCGCTCGCTCGCTCACTGAGGCCGGGCGACCAAAGGTCGCCCGACGCCCGGGCTTTGCCCGGGGGCCTCAGTGAG
CGAGCGAGCGCGCAGCTGCCTGCAGG
L2 BA mouse gene construct
SEQ ID NO: 79
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGCGGGG
CGAGGGGCGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGGGGGGGGGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGCGGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGCCTCAGAGCCCTAGATCTGCTCCTGGACTGCTGCT
GCTCCAGGCTCTGGTGTCTCTTGTGTCTCTGGCACTGGTGGCCCCTGCTAGACTGCAACCTGCTCTGTGGCCATTTCCTAGAAGC
GTGCAGATGTTCCCCAGACTGCTGTACATCAGCGCCGAGGACTTCAGCATCGATCACAGCCCCAACAGCACAGCTGGCCCTAGT
TGTAGCCTGCTGCAAGAGGCCTTCAGACGGTACTACAACTACGTGTTCGGCTTCTACAAGAGGCACCACGGACCTGCCAGATTCA
GAGCTGAGCCCCAGCTGCAGAAACTGCTGGTGTCTATCACCCTGGAAAGCGAGTGCGAGAGCTTCCCTAGCCTGAGCAGCGACG
AGACATACAGCCTGCTGGTGCAAGAGCCTGTGGCCGTGCTGAAGGCTAACTCTGTTTGGGGCGCTCTGAGAGGCCTGGAAACCT
TCTCTCAGCTGGTGTACCAGGACAGCTTCGGCACCTTCACCATCAACGAGTCCTCTATCGCCGACTCTCCTAGATTCCCTCACAG
AGGCATCCTGATCGATACCAGCAGACACTTCCTGCCTGTGAAAACCATCCTGAAAACCCTGGACGCCATGGCCTTCAACAAGTTC
AACGTGCTGCACTGGCACATCGTGGACGACCAGAGCTTTCCCTACCAGTCCACCACCTTTCCAGAGCTGTCCAACAAGGGCAGC
TACAGCCTGAGCCACGTGTACACCCCTAACGACGTGOGGATGGTGCTGGAATACGCCAGACTGAGAGGCATCAGAGTGATCCCC
GAGTTCGACACCCCTGGCCACACACAGTCTTGGGGCAAGGGACAGAAGAACCTGCTGACCCCTTGCTACAACCAGAAAACAAAG
ACCCAGGTGTTCGGCCCTGTGGACCCTACCGTGAACACCACCTACGCTTTCTTCAACACCTTCTTCAAAGAAATCAGCAGCGTGT
TCCCCGACCAGTTCATCCACCTCGGCGGAGATGAGGTCGAGTTCCAGTGTTGGGCCAGCAATCCCAACATCCAGGGCTTTATGA
AGAGGAAAGGCTTCGGCAGCGACTTCAGAAGGCTGGAAAGCTTCTACATCAAGAAGATCCTCGAGATCATCAGCAGCCTGAAGAA
GAACAGCATCGTCTGGCAAGAGGTGTTCGACGACAAGGTGGAACTGCAGCCTGGCACCGTGGTGGAAGTGTGGAAGTCTGAGC
ACTACAGCTACGAGCTGAAGCAAGTGACCGGCTCTGGCTTCCCTGCCATCCTTTCTGCCCCTTGGTATCTGGACCTGATCAGCTA
CGGCCAGGACTGGAAGAACTACTACAAGGTCGAGCCCCTGAACTTCGAGGGCAGCGAGAAGCAGAAGCAGCTGGTTATCGGAG
GCGAGGCTTGTCTGTGGGGCGAGTTCGTGGATGCCACCAACCTGACACCTAGACTGTGGCCTAGAGCCTCTGCTGTGGGCGAG
AGACTGTGGTCCCCTAAGACAGTGACCGACCTGGAAAACGCCTACAAGAGACTGGCCGTGCACAGATGCAGAATGGTGTCCAGA
GGAATCGCCGCTCAGCCTCTGTACACCGGCTACTGCAACTACGAGAACAAGATCTCTGGAGGATCTAGCGGAGGATCCTCTGGC
AGCGAGACACCAGGAACAAGCGAGTCAGCAACACCAGAGAGCAGTGGCGGCAGCAGCGGCGGCAGCAGCACCCTGGGCAAGA
ACATCCTGGTGGTGTCTGTGGTCACCGCCGAGTGCAACGAGTTCCCCAACCTGGAATCCGTGGAAAACTACACCCTGACAATCAA
CGACGACCAGTGCCTGCTGGCCTCCGAAACAGTTTGGGGAGCACTGCGCGGACTGGAAACATTCAGCCAGCTCGTGTGGAAATC
TGCCGAGGGCACATTCTTCATCAACAAGACCAAGATCAAGGACTTCCCTAGGTTCCCACACAGGGGCGTGCTGCTGGACACCTC
CAGACATTACCTGCCTCTGAGCAGCATCCTGGACACACTGGACGTGATGGCTTATAACAAGTTTAATGTGTTCCATTGGCACCTGG
TCGACGACAGCAGCTTCCCTTACGAGTCCTTCACATTCCCCGAGCTGACAAGAAAGGGCTCTTTCAACCCCGTGACACACATCTA
CACAGCCCAGGACGTGAAAGAAGTGATCGAGTACGCTAGGCTGCGGGGAATCAGAGTGCTGGCTGAGTTCGATACACCCGGACA
CACTCTGAGTTGGGGACCTGGTGCTCCTGGCCTCCTGACACCATGTTACTCTGGCTCTCACCTGAGCGGCACATTCGGCCCAGT
GAACCCAAGCCTGAATAGCACCTACGACTTTATGAGCACCCTGTTTCTGGAAATCTCCTCCGTGTTTCCCGACTTCTACCTGCATC
TCGGCGGCGACGAGGTGGACTTCACCTGTTGGAAGTCTAACCCCAATATCCAGGCATTCATGAAGAAGAAGGGGTTCACCGACTT
CAAGCAGCTTGAGAGCTTCTATATCCAGACGCTGCTGGATATCGTGTCCGACTACGACAAGGGCTACGTTGTGTGGCAAGAAGTC
TTTGACAACAAAGTGAAAGTGCGGCCCGACACCATCATCCAAGTGTGGCGGGAAGAGATGCCCGTCGAGTACATGCTGGAAATG
CAGGACATCACAAGAGCCGGCTTCAGGGCTCTGCTGAGCGCTCCATGGTATCTGAACAGAGTGAAGTACGGCCCCGATTGGAAG
GACATGTACAAAGTGGAACCTCTGGCCTTCCACGGCACCCCTGAACAGAAAGCTCTCGTGATTGGCGGCGAGGCCTGCATGTGG
GGAGAATACGTGGACAGCACAAACCTGGTGCCTCGGCTTTGGCCAAGAGCTGGTGCTGTGGCTGAAAGGCTGTGGTCTAGCAAC
CTGACCACCAACATCGACTTCGCCTTCAAGAGGCTGTCCCACTTCAGATGCGAACTCGTGCGGAGAGGAATCCAGGCTCAGCCA
ATCTCTGTGGGCTACTGCGAGCAAGAGTTCGAGCAGACCTGAGGATCCAACTTGTTTATTGCAGCTTATAATGGTTACAAATAAAG
CAATAGCATCACAAATTTCACAAATAAAGCATTTTTTTCACTGCATTCTAGTTGTGGTTTGTCCAAACTCATCAATGTATOTTATGCG
GCCGCAGGAACCCCTAGTGATGGAGTTGGCCACTCCCTCTCTGCGCGCTCGCTCGCTCACTGAGGCCGGGCGACCAAAGGTCG
CCCGACGCCCGGGCTTTGCCCGGGGGGCCTCAGTGAGCGAGCGAGCGCGCAGCTGCCTGCAGG
L3 BA mouse gene construct
SEQ ID NO: 80
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGGGGG
CGAGGGGCGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGGGGGGGGGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGGGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGCCTCAGAGCCCTAGATCTGCTCCTGGACTGCTGCT
GCTCCAGGCTCTGGTGTCTCTTGTGTCTCTGGCACTGGTGGCCCCTGCTAGACTGCAACCTGCTCTGTGGCCATTTCCTAGAAGC
GTGCAGATGTTCCCCAGACTGCTGTACATCAGCGCCGAGGACTTCAGCATCGATCACAGCCCCAACAGCACAGCTGGCCCTAGT
TGTAGCCTGCTGCAAGAGGCCTTCAGACGGTACTACAACTACGTGTTCGGCTTCTACAAGAGGCACCACGGACCTGCCAGATTCA
GAGCTGAGCCCCAGCTGCAGAAACTGCTGGTGTCTATCACCCTGGAAAGCGAGTGCGAGAGCTTCCCTAGCCTGAGCAGCGACG
AGACATACAGCCTGCTGGTGCAAGAGCCTGTGGCCGTGCTGAAGGCTAACTCTGTTTGGGGCGCTCTGAGAGGCCTGGAAACCT
TCTCTCAGCTGGTGTACCAGGACAGCTTCGGCACCTTCACCATCAACGAGTCCTCTATCGCCGACTCTCCTAGATTCCCTCACAG
AGGCATCCTGATCGATACCAGCAGACACTTCCTGCCTGTGAAAACCATCCTGAAAACCCTGGACGCCATGGCCTTCAACAAGTTC
AACGTGCTGCACTGGCACATCGTGGACGACCAGAGCTTTCCCTACCAGTCCACCACCTTTCCAGAGCTGTCCAACAAGGGCAGC
TACAGCCTGAGCCACGTGTACACCCCTAACGACGTGCGGATGGTGCTGGAATACGCCAGACTGAGAGGCATCAGAGTGATCCCC
GAGTTCGACACCCCTGGCCACACACAGTCTTGGGGCAAGGGACAGAAGAACCTGCTGACCCCTTGCTACAACCAGAAAACAAAG
ACCCAGGTGTTCGGCCCTGTGGACCCTACCGTGAACACCACCTACGCTTTCTTCAACACCTTCTTCAAAGAAATCAGCAGCGTGT
TCCCCGACCAGTTCATCCACCTCGGCGGAGATGAGGTCGAGTTCCAGTGTTGGGCCAGCAATCCCAACATCCAGGGCTTTATGA
AGAGGAAAGGCTTCGGCAGCGACTTCAGAAGGCTGGAAAGCTTCTACATCAAGAAGATCCTCGAGATCATCAGCAGCCTGAAGAA
GAACAGCATCGTCTGGCAAGAGGTGTTCGACGACAAGGTGGAACTGCAGCCTGGCACCGTGGTGGAAGTGTGGAAGTCTGAGC
ACTACAGCTACGAGCTGAAGCAAGTGACCGGCTCTGGCTTCCCTGCCATCCTTTCTGCCCCTTGGTATCTGGACCTGATCAGCTA
CGGCCAGGACTGGAAGAACTACTACAAGGTCGAGCCCCTGAACTTCGAGGGCAGCGAGAAGCAGAAGCAGCTGGTTATCGGAG
GCGAGGCTTGTCTGTGGGGCGAGTTCGTGGATGCCACCAACCTGACACCTAGACTGTGGCCTAGAGCCTCTGCTGTGGGCGAG
AGACTGTGGTCCCCTAAGACAGTGACCGACCTGGAAAACGCCTACAAGAGACTGGCCGTGCACAGATGCAGAATGGTGTCCAGA
GGAATCGCCGCTCAGCCTCTGTACACCGGCTACTGCAACTACGAGAACAAGATCGGCAGCGCCGGCAGCGCCGCCGGCAGCGG
CGAGTTCACCCTGGGCAAGAACATCCTGGTGGTGTCTGTGGTCACCGCCGAGTGCAACGAGTTCCCCAACCTGGAATCCGTGGA
AAACTACACCCTGACAATCAACGACGACCAGTGCCTGCTGGCCTCCGAAACAGTTTGGGGAGCACTGCGCGGACTGGAAACATT
CAGCCAGCTCGTGTGGAAATCTGCCGAGGGCACATTCTTCATCAACAAGACCAAGATCAAGGACTTCCCTAGGTTCCCACACAGG
GGCGTGCTGCTGGACACCTCCAGACATTACCTGCCTCTGAGCAGCATCCTGGACACACTGGACGTGATGGCTTATAACAAGTTTA
ATGTGTTCCATTGGCACCTGGTCGACGACAGCAGCTTCCCTTACGAGTCCTTCACATTCCCCGAGCTGACAAGAAAGGGCTCTTT
CAACCCCGTGACACACATCTACACAGCCCAGGACGTGAAAGAAGTGATCGAGTACGCTAGGCTGCGGGGAATCAGAGTGCTGGC
TGAGTTCGATACACCCGGACACACTCTGAGTTGGGGACCTGGTGCTCCTGGCCTCCTGACACCATGTTACTCTGGCTCTCACCTG
AGCGGCACATTCGGCCCAGTGAACCCAAGCCTGAATAGCACCTACGACTTTATGAGCACCCTGTTTCTGGAAATCTCCTCCGTGT
TTCCCGACTTCTACCTGCATCTCGGCGGCGACGAGGTGGACTTCACCTGTTGGAAGTCTAACCCCAATATCCAGGCATTCATGAA
GAAGAAGGGGTTCACCGACTTCAAGCAGCTTGAGAGCTTCTATATCCAGACGCTGCTGGATATCGTGTCCGACTACGACAAGGGC
TACGTTGTGTGGCAAGAAGTCTTTGACAACAAAGTGAAAGTGCGGCCCGACACCATCATCCAAGTGTGGCGGGAAGAGATGCCC
GTCGAGTACATGCTGGAAATGCAGGACATCACAAGAGCCGGCTTCAGGGCTCTGCTGAGCGCTCCATGGTATCTGAACAGAGTG
AAGTACGGCCCCGATTGGAAGGACATGTACAAAGTGGAACCTCTGGCCTTCCACGGCACCCCTGAACAGAAAGCTCTCGTGATT
GGCGGCGAGGCCTGCATGTGGGGAGAATACGTGGACAGCACAAACCTGGTGCCTCGGCTTTGGCCAAGAGCTGGTGCTGTGGC
TGAAAGGCTGTGGTCTAGCAACCTGACCACCAACATCGACTTCGCCTTCAAGAGGCTGTCCCACTTCAGATGCGAACTCGTGCGG
AGAGGAATCCAGGCTCAGCCAATCTCTGTGGGCTACTGCGAGCAAGAGTTCGAGCAGACCTGAGGATCCAACTTGTTTATTGCAG
CTTATAATGGTTACAAATAAAGCAATAGCATCACAAATTTCACAAATAAAGCATTTTTTTCACTGCATTCTAGTTGTGGTTTGTCCAA
ACTCATCAATGTATOTTATGCGGCCGCAGGAACCCCTAGTGATGGAGTTGGCCACTCCCTCTCTGCGCGCTCGCTCGCTCACTGA
GGCCGGGCGACCAAAGGTCGCCCGACGCCCGGGCTTTGCCCGGGGGGCCTCAGTGAGCGAGCGAGCGCGCAGCTGCCTGCA
GG
L1 AB human noh gene construct
SEQ ID NO: 81
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGCGGGG
CGAGGGGCGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGGGGGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGCGGGCGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGACAAGCTCCAGGCTTTGGTTTTCGCTGCTGCTGGC
GGCAGCGTTCGCAGGACGGGCGACGGCCCTCTGGCCCTGGCCTCAGAACTTCCAAACCTCCGACCAGCGCTACGTCCTTTACC
CGAACAACTTTCAATTCCAGTACGATGTCAGCTCGGCCGCGCAGCCCGGCTGCTCAGTCCTCGACGAGGCCTTCCAGCGCTATC
GTGACCTGCTTTTCGGTTCCGGGTCTTGGCCCCGTCCTTACCTCACAGGGAAACGGCATACACTGGAGAAGAATGTGTTGGTTGT
CTCTGTAGTCACACCTGGATGTAACCAGCTTCCTACTTTGGAGTCAGTGGAGAATTATACCCTGACCATAAATGATGACCAGTGTT
TACTCCTCTCTGAGACTGTCTGGGGAGCTCTCCGAGGTCTGGAGACTTTTAGCCAGCTTGTTTGGAAATCTGCTGAGGGCACATT
CTTTATCAACAAGACTGAGATTGAGGACTTTCCCCGCTTTCCTCACCGGGGCTTGCTGTTGGATACATCTCGCCATTACCTGCCAC
TCTCTAGCATCCTGGACACTCTGGATGTCATGGCGTACAATAAATTGAACGTGTTCCACTGGCATCTGGTAGATGATCCTTCCTTC
CCATATGAGAGCTTCACTTTTCCAGAGCTCATGAGAAAGGGGTCCTACAACCCTGTCACCCACATCTACACAGCACAGGATGTGA
AGGAGGTCATTGAATACGCACGGCTCCGGGGTATCCGTGTGCTTGCAGAGTTTGACACTCCTGGCCACACTTTGTCCTGGGGAC
CAGGTATCCCTGGATTACTGACTCCTTGCTACTCTGGGTCTGAGCCCTCTGGCACCTTTGGACCAGTGAATCCCAGTCTCAATAAT
ACCTATGAGTTCATGAGCACATTCTTCTTAGAAGTCAGCTCTGTCTTCCCAGATTTTTATCTTCATCTTGGAGGAGATGAGGTTGAT
TTCACCTGCTGGAAGTCCAACCCAGAGATCCAGGACTTTATGAGGAAGAAAGGCTTCGGTGAGGACTTCAAGCAGCTGGAGTCCT
TCTACATCCAGACGCTGCTGGACATCGTCTCTTCTTATGGCAAGGGCTATGTGGTGTGGCAGGAGGTGTTTGATAATAAAGTAAA
GATTCAGCCAGACACAATCATACAGGTGTGGCGAGAGGATATTCCAGTGAACTATATGAAGGAGCTGGAACTGGTCACCAAGGCC
GGCTTCCGGGCCCTTCTCTCTGCCCCCTGGTACCTGAACCGTATATCCTATGGCCCTGACTGGAAGGATTTCTACATAGTGGAAC
CCCTGGCATTTGAAGGTACCCCTGAGCAGAAGGCTCTGGTGATTGGTGGAGAGGCTTGTATGTGGGGAGAATATGTGGACAACA
CAAACCTGGTCCCCAGGCTCTGGCCCAGAGCAGGGGCTGTTGCCGAAAGGCTGTGGAGCAACAAGTTGACATCTGACCTGACAT
TTGCCTATGAACGTTTGTCACACTTCCGCTGTGAATTGCTGAGGCGAGGTGTCCAGGCCCAACCCCTCAATGTAGGCTTCTGTGA
GCAGGAGTTTGAACAGACCGGTGGTGGTGGATCTGGAGGCGGAGGATCAGGCGGCGGAGGTTCTGGAGGTGGAGGTAGTGCT
CGGGCCCCGAGCGTCTCGGCCAAGCCGGGGCCGGCGCTGTGGCCCCTGCCGCTCTTGGTGAAGATGACCCCGAACCTGCTGC
ATCTCGCCCCGGAGAACTTCTACATCAGCCACAGCCCCAATTCCACGGCGGGCCCCTCCTGCACCCTGCTGGAGGAAGCGTTTC
GACGATATCATGGCTATATTTTTGGTTTCTACAAGTGGCATCATGAACCTGCTGAATTCCAGGCTAAAACCCAGGTTCAGCAACTT
CTTGTCTCAATCACCCTTCAGTCAGAGTGTGATGCTTTCCCCAACATATCTTCAGATGAGTCTTATACTTTACTTGTGAAAGAACCA
GTGGCTGTCCTTAAGGCCAACAGAGTTTGGGGAGCATTACGAGGTTTAGAGACCTTTAGCCAGTTAGTTTATCAAGATTCTTATGG
AACTTTCACCATCAATGAATCCACCATTATTGATTCTCCAAGGTTTTCTCACAGAGGAATTTTGATTGATACATCCAGACATTATCTG
CCAGTTAAGATTATTCTTAAAACTCTGGATGCCATGGCTTTTAATAAGTTTAATGTTCTTCACTGGCACATAGTTGATGACCAGTCTT
TCCCATATCAGAGCATCACTTTTCCTGAGTTAAGCAATAAAGGAAGCTATTCTTTGTCTCATGTTTATACACCAAATGATGTCCGTAT
GGTGATTGAATATGCCAGATTACGAGGAATTCGAGTCCTGCCAGAATTTGATACCCCTGGGCATACACTATCTTGGGGAAAAGGT
CAGAAAGACCTCCTGACTCCATGTTACAGTAGACAAAACAAGTTGGACTCTTTTGGACCTATAAACCCTACTCTGAATACAACATAC
AGCTTCCTTACTACATTTTTCAAAGAAATTAGTGAGGTGTTTCCAGATCAATTCATTCATTTGGGAGGAGATGAAGTGGAATTTAAA
TGTTGGGAATCAAATCCAAAAATTCAAGATTTCATGAGGCAAAAAGGCTTTGGCACAGATTTTAAGAAACTAGAATCTTTCTACATT
CAAAAGGTTTTGGATATTATTGCAACCATAAACAAGGGATCCATTGTCTGGCAGGAGGTTTTTGATGATAAAGCAAAGCTTGCGCC
GGGCACAATAGTTGAAGTATGGAAAGACAGCGCATATCCTGAGGAACTCAGTAGAGTCACAGCATCTGGCTTCCCTGTAATCCTT
TCTGCTCCTTGGTACTTAGATTTGATTAGCTATGGACAAGATTGGAGGAAATACTATAAAGTGGAACCTCTTGATTTTGGCGGTACT
CAGAAACAGAAACAACTTTTCATTGGTGGAGAAGCTTGTCTATGGGGAGAATATGTGGATGCAACTAACCTCACTCCAAGATTATG
GCCTCGGGCAAGTGCTGTTGGTGAGAGACTCTGGAGTTCCAAAGATGTCAGAGATATGGATGACGCCTATGACAGACTGACAAG
GCACCGCTGCAGGATGGTCGAACGTGGAATAGCTGCACAACCTCTTTATGCTGGATATTGTAACCATGAGAACATGTAAGGATCC
AACTTGTTTATTGCAGCTTATAATGGTTACAAATAAAGCAATAGCATCACAAATTTCACAAATAAAGCATTTTTTTCACTGCATTCTA
GTTGTGGTTTGTCCAAACTCATCAATGTATOTTATGCGGCCGCAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGGGGGCCTCAGTGAGCGAGCGAGC
GCGCAGCTGCCTGCAGG
L1 AB human GA gene construct
SEQ ID NO: 82
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGGGGG
CGAGGGGCGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGGGGGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGGGGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGACATCTAGCAGACTGTGGTTCAGCCTGCTGCTGGC
CGCTGCTTTTGCTGGCAGAGCTACAGCTCTTTGGCCCTGGCCTCAGAACTTCCAGACCAGCGACCAGAGATACGTGCTGTACCC
CAACAACTTCCAGTTCCAGTACGACGTGTCCAGCGCCGCTCAGCCTGGATGTTCTGTGCTGGATGAGGCCTTCCAGCGGTACAG
GGATCTGCTGTTTGGCAGCGGCTCTTGGCCCAGACCTTACCTGACAGGCAAGCGGCACACCCTGGAAAAGAACGTGCTGGTGGT
GTCCGTGGTCACCCCTGGCTGTAATCAGCTGCCCACACTGGAAAGCGTGGAAAACTACACCCTGACCATCAACGACGACCAGTG
TCTGCTGCTGAGCGAGACAGTTTGGGGAGCCCTGAGAGGCCTGGAAACCTTTTCTCAGCTCGTGTGGAAGTCCGCCGAGGGCAC
CTTCTTCATCAACAAGACCGAGATCGAGGACTTCCCCAGATTTCCCCACAGAGGACTGCTGCTCGACACCAGCAGACATTACCTG
CCTCTGAGCAGCATCCTGGATACCCTGGACGTGATGGCCTACAACAAGCTGAACGTGTTCCACTGGCACCTGGTGGACGACCCT
AGCTTCCCTTACGAGAGCTTCACATTCCCCGAGCTGATGCGGAAGGGCAGCTACAACCCTGTGACACACATCTACACAGCCCAG
GACGTGAAAGAAGTGATCGAGTACGCCCGGCTGCGGGGCATTAGAGTGCTGGCCGAATTTGACACCCCTGGACACACCCTGTCT
TGGGGCCCTGGAATTCCTGGACTGCTGACCCCTTGTTACAGCGGCAGCGAGCCTTCTGGCACATTCGGCCCTGTGAACCCCAGC
CTGAACAACACCTACGAGTTTATGAGCACATTCTTTCTGGAAGTCTCCAGCGTGTTCCCCGACTTCTATCTGCATCTCGGAGGCGA
CGAGGTGGACTTCACCTGTTGGAAGTCTAACCCCGAGATCCAGGATTTCATGCGGAAGAAAGGCTTCGGCGAGGATTTCAAGCA
GCTGGAAAGCTTCTACATCCAGACACTGCTGGACATCGTGTCTAGCTACGGCAAGGGCTACGTCGTGTGGCAAGAGGTGTTCGA
CAACAAAGTGAAGATCCAGCCTGACACCATCATCCAAGTGTGGCGCGAGGACATCCCCGTGAACTACATGAAGGAACTCGAGCT
GGTCACCAAGGCCGGCTTTAGAGCACTGCTGTCTGCCCCTTGGTATCTGAACCGGATCAGCTACGGCCCCGACTGGAAGGATTT
CTACATCGTGGAACCCCTGGCCTTCGAGGGCACACCTGAACAGAAGGCCCTGGTTATTGGAGGCGAGGCCTGTATGTGGGGCG
AGTACGTGGACAACACCAACCTGGTGCCTAGACTGTGGCCTAGAGCTGGCGCTGTGGCCGAAAGACTCTGGTCCAACAAACTGA
CCTCCGACCTGACCTTCGCCTACGAGAGACTGAGCCACTTCAGATGCGAGCTGCTGAGAAGAGGCGTGCAGGCCCAGCCTCTGA
ATGTGGGCTTCTGCGAGCAAGAGTTCGAGCAGACAGGTGGCGGAGGATCTGGCGGAGGTGGAAGCGGCGGAGGCGGTTCTGG
TGGTGGTGGATCTGCTAGAGCCCCTAGCGTGTCAGCCAAACCTGGACCTGCTTTGTGGCCTCTGCCTCTGCTGGTCAAGATGAC
CCCTAACCTGCTGCATCTGGCCCCTGAGAACTTTTACATCTCTCACAGCCCCAACAGCACCGCCGGACCTAGCTGTACACTGCTC
GAGGAAGCCTTCAGAAGATACCACGGCTACATCTTCGGCTTCTACAAGTGGCACCACGAGCCTGCCGAGTTCCAGGCCAAAACA
CAGGTGCAGCAGCTCCTCGTGTCTATCACCCTGCAGAGCGAGTGCGACGCCTTTCCTAACATCAGCAGCGACGAGAGCTATACC
CTGCTTGTGAAAGAACCCGTGGCCGTGCTGAAGGCCAACAGAGTGTGGGGAGCACTGCGGGGACTCGAGACATTTTCCCAGCTG
GTGTACCAGGACTCCTACGGCACCTTCACCATCAATGAGAGCACAATCATCGACAGCCCCAGATTCAGCCACCGGGGCATCCTG
ATCGATACCAGCAGGCACTATCTGCCCGTGAAGATCATCCTGAAAACACTGGACGCCATGGCCTTTAACAAGTTCAATGTGCTGC
ACTGGCATATCGTGGACGATCAGAGCTTCCCCTACCAGTCCATCACCTTTCCAGAGCTGAGCAACAAGGGCTCCTACAGCCTGAG
CCACGTGTACACCCCTAACGACGTGCGGATGGTCATCGAGTATGCCAGACTGAGAGGCATCAGGGTGCTGCCCGAGTTCGATAC
ACCAGGCCATACACTGAGCTGGGGCAAGGGCCAGAAAGACCTGCTGACACCCTGCTACTCCCGGCAGAACAAGCTGGACAGCTT
TGGCCCCATCAATCCCACACTGAATACCACCTACAGCTTTCTGACCACCTTTTTCAAAGAAATCAGCGAGGTGTTCCCGGACCAGT
TCATCCATCTCGGCGGAGATGAAGTGGAATTCAAGTGCTGGGAGAGCAACCCCAAGATTCAGGACTTTATGAGACAGAAAGGATT
CGGGACCGACTTCAAGAAACTTGAGAGCTTCTATATTCAGAAGGTCCTGGACATCATTGCCACCATTAACAAGGGCAGCATTGTCT
GGCAAGAAGTCTTTGACGACAAGGCCAAGCTGGCCCCAGGCACAATCGTGGAAGTGTGGAAGGACAGCGCTTACCCCGAGGAA
CTGAGCAGAGTGACAGCCAGCGGCTTCCCTGTGATTCTGAGCGCTCCCTGGTATCTGGATCTGATCTCTTACGGCCAGGACTGG
CGGAAGTACTACAAGGTGGAACCTCTGGATTTCGGCGGCACCCAGAAGCAGAAGCAGCTGTTTATTGGCGGAGAAGCCTGCCTG
TGGGGAGAATATGTGGACGCCACCAATCTGACCCCTCGGTTGTGGCCCAGAGCCTCTGCAGTGGGAGAGAGACTTTGGAGCAGC
AAGGACGTGCGCGACATGGACGACGCCTATGACAGACTGACCCGGCACAGATGCCGGATGGTGGAAAGAGGAATTGCCGCACA
GCCTCTGTACGCCGGCTACTGCAACCACGAGAATATGTAGGGATCCAACTTGTTTATTGCAGCTTATAATGGTTACAAATAAAGCA
ATAGCATCACAAATTTCACAAATAAAGCATTTTTTTCACTGCATTCTAGTTGTGGTTTGTCCAAACTCATCAATGTATOTTATGCGGC
CGCAGGAACCCCTAGTGATGGAGTTGGCCACTCCCTCTCTGCGCGCTCGCTCGCTCACTGAGGCCGGGCGACCAAAGGTCGCC
CGACGCCCGGGCTTTGCCCGGGGGGCCTCAGTGAGCGAGCGAGCGCGCAGCTGCCTGCAGG
L1 AB human IDT gene construct
SEQ ID NO: 83
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGGGGGGGGGGGCGGGG
CGAGGGGGGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGGGGGGGGGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGGGGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGACAAGCAGCAGACTGTGGTTCTCCCTGCTTTTGGC
GGCGGCTTTTGCCGGACGGGCCACAGCCCTGTGGCCGTGGCCGCAAAATTTTCAGACGAGCGACCAACGGTATGTTCTTTATCC
AAATAACTTTCAATTTCAGTATGATGTGTCCTCCGCTGCACAGCCTGGCTGCAGCGTCCTGGACGAGGCATTTCAGCGCTATAGA
GATCTCCTCTTTGGGTCTGGAAGCTGGCCCAGACCATACTTGACTGGCAAGAGGCACACCCTCGAGAAGAATGTTTTGGTGGTTT
CTGTAGTAACCCCTGGGTGCAATCAACTTCCGACCCTTGAGTCAGTAGAGAATTATACGTTGACAATAAATGACGATCAATGCCTT
CTTCTTAGTGAGACGGTTTGGGGAGCGCTGCGAGGGCTTGAGACATTTAGCCAGCTTGTATGGAAGAGTGCGGAGGGTACCTTC
TTCATAAATAAAACGGAGATCGAAGATTTTCCGCGATTCCCTCACAGGGGTCTGCTTCTGGATACCAGCAGGCATTATCTGCCTCT
TAGCAGCATACTCGACACACTGGACGTGATGGCGTACAACAAACTCAACGTGTTCCACTGGCATCTGGTGGACGACCCAAGCTTT
CCATACGAGAGTTTTACGTTTCCAGAGCTCATGAGAAAGGGTAGTTATAATCCCGTGACCCATATATATACCGCGCAGGACGTGAA
AGAAGTCATTGAGTACGCGCGACTCCGAGGAATTAGAGTACTGGCAGAGTTCGACACTCCTGGGCACACGCTCAGTTGGGGGCC
TGGAATACCTGGATTGTTGACTCCCTGCTATAGTGGCAGTGAGCCCTCAGGGACCTTTGGTCCGGTTAATCCCAGTCTTAACAAC
ACATACGAGTTCATGTCTACGTTTTTTCTGGAGGTGTCTTCAGTATTCCCTGACTTCTACCTGCACCTTGGGGGAGATGAGGTTGA
CTTCACTTGTTGGAAGTCTAACCCTGAAATACAGGACTTCATGCGGAAAAAAGGATTCGGGGAGGATTTCAAACAACTGGAGTCCT
TTTATATTCAGACCCTTTTGGACATTGTTTCCAGCTATGGCAAAGGGTATGTGGTGTGGCAGGAAGTTTTCGACAACAAAGTCAAG
ATCCAACCGGATACCATTATACAGGTCTGGAGGGAGGACATCCCCGTAAACTACATGAAGGAACTTGAACTGGTCACCAAAGCTG
GCTTTAGAGCGCTGCTCTCTGCGCCTTGGTATTTGAATAGGATTAGTTATGGGCCCGATTGGAAGGACTTTTATATCGTCGAGCCG
CTTGCCTTTGAGGGAACCCCTGAGCAAAAAGCACTTGTGATTGGGGGAGAGGCTTGTATGTGGGGAGAATATGTCGATAATACGA
ACCTGGTACCCCGGCTGTGGCCTCGCGCTGGGGCCGTGGCGGAGCGCCTCTGGTCCAATAAGTTGACAAGTGATTTGACCTTCG
CTTACGAACGGTTGTCTCATTTCAGATGTGAGCTGCTGAGACGCGGCGTACAAGCACAACCCCTGAACGTTGGTTTTTGCGAACA
AGAATTCGAACAGACCGGGGGGGGAGGCTCCGGAGGGGGGGGAGTGGAGGCGGGGGATCAGGAGGCGGTGGCAGCGCCAG
GGCGCCTTCAGTAAGCGCCAAACCCGGCCCAGCCCTGTGGCCTTTGCCACTCCTGGTAAAAATGACTCCTAATCTGCTGCATCTG
GCGCCTGAAAATTTTTATATAAGCCACTCTCCTAATAGTACTGCAGGTCCCTCTTGCACTTTGCTCGAAGAAGCTTTTCGAAGGTAT
CACGGTTATATTTTTGGATTTTATAAGTGGCACCATGAACCTGCGGAATTCCAAGCGAAAACTCAAGTCCAACAGTTGCTCGTAAG
TATTACACTTCAGAGCGAGTGTGATGCTTTCCCTAATATCAGTTCTGACGAAAGTTACACGCTCCTGGTAAAGGAGCCTGTCGCTG
TACTGAAGGCGAATCGCGTTTGGGGCGCCTTGCGAGGCCTGGAAACTTTCAGTCAGTTGGTGTATCAGGATTCCTATGGGACATT
CACGATCAATGAATCCACTATTATAGACAGCCCTAGGTTTAGTCATAGGGGCATACTGATTGACACTTCTAGACATTACCTGCCAG
TCAAAATCATCCTGAAAACACTTGATGCCATGGCATTCAATAAGTTTAATGTTCTCCATTGGCACATCGTGGACGATCAAAGCTTTC
CATACCAGTCTATAACATTTCCGGAGCTTAGTAACAAGGGGAGCTATTCACTCTCACACGTTTACACCCCGAACGACGTGCGCATG
GTTATTGAATACGCCAGACTTCGAGGCATTAGAGTTCTTCCAGAATTTGATACACCAGGCCACACGCTCTCCTGGGGCAAAGGCC
AGAAGGACCTGOTTACCCCATGTTACTCCCGACAAAACAAACTCGATTCTTTTGGGCCAATAAACCCAACGTTGAACACTACGTAC
TCCTTCCTGACTACGTTTTTTAAGGAAATAAGTGAGGTATTCCCTGACCAGTTTATTCACTTGGGTGGTGATGAAGTCGAATTTAAG
TGTTGGGAGTCAAACCCCAAAATACAAGACTTCATGCGCCAGAAGGGGTTTGGGACCGACTTTAAAAAACTCGAGTCTTTCTACAT
TCAAAAGGTTCTCGATATTATCGCAACAATTAACAAAGGGAGCATTGTGTGGCAAGAGGTATTTGACGATAAAGCCAAACTGGCGC
CCGGAACGATAGTCGAGGTCTGGAAAGACAGCGCCTATCCCGAGGAGCTTAGTCGAGTCACGGCTTCAGGTTTTCCAGTTATCCT
TAGTGCCCCGTGGTACTTGGATTTGATTTCCTACGGTCAAGACTGGCGCAAGTATTATAAGGTTGAACCTCTTGATTTTGGTGGGA
CCCAAAAACAAAAGCAGTTGTTCATCGGTGGGGAAGCTTGCTTGTGGGGTGAGTACGTAGACGCGACTAATCTTACGCCTCGATT
GTGGCCTCGGGCATCTGCAGTTGGGGAGCGCTTGTGGAGCTCAAAGGACGTGCGAGACATGGATGACGCTTATGATAGACTGAC
CCGACATAGGTGTCGAATGGTGGAGAGAGGTATAGCGGCCCAACCTCTTTATGCCGGATACTGCAACCATGAAAACATGTAAGGA
TCCAACTTGTTTATTGCAGCTTATAATGGTTACAAATAAAGCAATAGCATCACAAATTTCACAAATAAAGCATTTTTTTCACTGCATT
CTAGTTGTGGTTTGTCCAAACTCATCAATGTATOTTATGCGGCCGCAGGAACCCCTAGTGATGGAGTTGGCCACTCCCTCTCTGC
GCGCTCGCTCGCTCACTGAGGCCGGGCGACCAAAGGTCGCCCGACGCCCGGGCTTTGCCCGGGGGGCCTCAGTGAGCGAGCG
AGCGCGCAGCTGCCTGCAGG
L1 AB human NV gene construct
SEQ ID NO: 84
CCTGCAGGCAGCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAACTCCATCACTAGGGGTTCCTGCGGCCGCGGTACCCGTTACATAA
CTTACGGTAAATGGCCCGCCTGGCTGACCGCCCAACGACCCCCGCCCATTGACGTCAATAGTAACGCCAATAGGGACTTTCCATT
GACGTCAATGGGTGGAGTATTTACGGTAAACTGCCCACTTGGCAGTACATCAAGTGTATCATATGCCAAGTACGCCCCCTATTGAC
GTCAATGACGGTAAATGGCCCGCCTGGCATTGTGCCCAGTACATGACCTTATGGGACTTTCCTACTTGGCAGTACATCTACGTATT
AGTCATCGCTATTACCATGGTCGAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGGGGGGGGGGGGGGGGGGGGCGCGCCAGGCGGGGGGGGGGGGGG
CGAGGGGGGGGGGGGGGCGAGGCGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGGGGGGGGGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTGAGCGGGGGGGACGGCCCT
TCTCCTCCGGGCTGTAATTAGCTGAGCAAGAGGTAAGGGTTTAAGGGATGGTTGGTTGGTGGGGTATTAATGTTTAATTACCTGG
AGCACCTGCCTGAAATCACTTTTTTTCAGGTTGGAGCTAGCGCCACCATGACCTCATCCCGCTTGTGGTTCTCTCTGTTGTTGGCG
GCCGCCTTTGCAGGAAGGGCTACTGCCTTGTGGCCCTGGCCACAAAATTTCCAGACCAGTGATCAGCGATATGTACTCTATCCAA
ATAACTTCCAGTTTCAATACGACGTATCCAGCGCCGCTCAGCCAGGCTGTAGCGTGCTGGATGAAGCGTTTCAACGCTATCGAGA
TCTGTTGTTCGGGTCAGGTTCTTGGCCCAGGCCGTATCTCACTGGGAAAAGGCACACCCTGGAAAAGAATGTGCTGGTTGTCAGT
GTGGTTACACCAGGATGCAATCAGCTCCCAACACTTGAGAGCGTGGAAAACTATACTCTCACAATCAACGACGATCAGTGTCTCCT
GCTCTCCGAAACAGTCTGGGGCGCTCTGCGGGGGTTGGAAACGTTCTCACAGCTGGTATGGAAGTCCGCCGAGGGAACTTTCTT
CATCAACAAAACGGAGATCGAAGACTTTCCCAGGTTCCCACACAGAGGTCTGCTTCTCGACACTAGCCGGCATTACCTTCCCCTG
AGCTCTATTCTTGACACCCTCGACGTAATGGCCTATAACAAGCTGAACGTGTTTCATTGGCATCTCGTGGACGATCCAAGCTTCCC
ATACGAGTCCTTCACATTCCCAGAGCTGATGCGCAAAGGGTCATACAATCCAGTGACACATATTTACACGGCCCAGGATGTGAAA
GAGGTTATTGAGTACGCCAGACTCAGGGGCATTAGGGTGCTGGCCGAGTTTGATACACCAGGGCATACCCTCTCATGGGGCCCT
GGTATCCCTGGGCTGCTGACTCCCTGCTATTCAGGAAGTGAGCCGAGTGGGACTTTCGGGCCAGTGAACCCCTCTCTGAACAAC
ACATACGAGTTCATGTCCACATTTTTCCTCGAGGTGAGTTCAGTGTTCCCCGATTTTTATCTTCACCTTGGTGGAGACGAGGTTGA
CTTCACCTGTTGGAAGAGCAACCCCGAGATTCAGGACTTCATGCGCAAAAAGGGGTTTGGAGAGGATTTTAAGCAGCTTGAGAGC
TTTTATATACAGACACTGCTCGACATCGTGTCTTCATATGGAAAAGGCTATGTGGTTTGGCAAGAGGTCTTTGACAACAAAGTCAA
GATCCAGCCTGACACGATTATCCAAGTATGGAGAGAGGACATACCCGTCAACTACATGAAGGAGCTGGAGCTGGTGACCAAGGC
TGGGTTTAGGGCCCTGCTCTCAGCTCCCTGGTACCTGAACCGAATATCCTATGGGCCGGATTGGAAAGACTTCTACATAGTGGAA
CCACTGGCCTTCGAAGGGACTCCTGAGCAGAAGGCATTGGTGATCGGCGGTGAGGCTTGTATGTGGGGCGAGTACGTAGACAAT
ACGAACCTCGTGCCAAGGCTTTGGCCAAGAGCTGGTGCAGTTGCAGAGCGACTGTGGAGCAATAAGCTGACAAGTGACCTCACA
TTTGCCTACGAGCGGCTGTCCCACTTTCGCTGTGAACTCCTGAGACGGGGAGTTCAAGCCCAGCCTCTGAATGTTGGCTTTTGCG
AGCAGGAGTTCGAACAGACAGGGGGAGGAGGTAGCGGGGGAGGAGGAAGTGGGGGTGGAGGTAGCGGAGGTGGTGGAAGCG
CTAGAGCACCATCAGTTAGTGCCAAACCAGGGCCTGCATTGTGGCCACTGCCCCTCCTGGTCAAAATGACACCTAATCTGCTCCA
TCTTGCCCCGGAGAATTTCTATATCAGCCACTCCCCCAATTCAACCGCCGGGCCCTCATGCACCCTCCTCGAAGAGGCCTTTCGG
AGATACCATGGGTATATATTCGGCTTCTACAAATGGCATCACGAACCTGCTGAGTTCCAGGCCAAGACCCAAGTCCAACAGTTGCT
GGTGTCAATCACACTGCAGAGCGAATGCGATGCTTTTCCCAACATTTCTTCCGACGAGTCTTACACGCTCCTTGTGAAGGAGCCA
GTAGCTGTCTTGAAGGCCAATCGCGTGTGGGGTGCCTTGAGGGGCTTGGAAACATTTTCCCAGCTGGTCTACCAGGATTCCTATG
GGACCTTTACCATCAACGAAAGTACGATAATTGACAGTCCTCGGTTCTCACACAGGGGCATCTTGATCGATACAAGCAGGCATTAC
TTGCCAGTCAAGATCATCCTTAAGACACTGGATGCCATGGCTTTCAATAAGTTCAACGTGCTTCATTGGCACATTGTGGATGATCA
GAGCTTCCCATACCAGAGCATTACCTTCCCAGAGCTTTCCAATAAGGGTAGTTATTCTCTCAGCCACGTGTACACACCCAATGATG
TGCGCATGGTCATCGAGTATGCCCGGCTGAGAGGGATTAGGGTCCTGCCCGAATTTGACACGCCAGGCCACACACTCTCCTGGG
GCAAGGGCCAGAAGGATCTTTTGACTCCATGTTATTCAAGACAAAATAAGTTGGACAGCTTTGGACCCATCAACCCCACCCTTAAC
ACAACTTACTCATTCCTGACCACATTCTTTAAGGAGATCAGTGAGGTCTTCCCCGACCAGTTTATTCATCTCGGTGGCGATGAGGT
CGAGTTTAAATGCTGGGAAAGCAACCCCAAGATCCAGGACTTCATGCGGCAAAAGGGATTCGGAACTGATTTCAAAAAGCTCGAG
TCCTTCTATATTCAGAAGGTCTTGGACATCATCGCTACCATAAACAAGGGGAGCATCGTTTGGCAGGAGGTGTTCGACGATAAAG
CCAAGCTGGCTCCAGGGACTATTGTCGAGGTGTGGAAAGATAGTGCCTACCCCGAGGAACTTTCACGGGTGACCGCCTCTGGTT
TTCCTGTGATCCTCTCAGCACCGTGGTATOTTGATCTCATCTCTTACGGACAGGACTGGCGGAAGTACTACAAAGTTGAACCACTG
GATTTTGGGGGAACGCAGAAACAAAAGCAGCTGTTTATCGGAGGCGAAGCATGTCTGTGGGGAGAGTATGTAGATGCAACCAAC
CTTACCCCACGGCTGTGGCCACGGGCTTCTGCTGTAGGTGAGAGGCTCTGGAGCAGCAAAGACGTTCGCGACATGGACGACGC
ATATGACCGGCTTACTAGACACCGCTGCAGAATGGTTGAGAGGGGAATTGCCGCCCAGCCCCTTTATGCCGGATATTGCAATCAT
GAAAATATGTAAGGATCCAACTTGTTTATTGCAGCTTATAATGGTTACAAATAAAGCAATAGCATCACAAATTTCACAAATAAAGCAT
TTTTTTCACTGCATTCTAGTTGTGGTTTGTCCAAACTCATCAATGTATOTTATGCGGCCGCAGGAACCCCTAGTGATGGAGTTGGC
CACTCCCTCTCTGCGCGCTCGCTCGCTCACTGAGGCCGGGCGACCAAAGGTCGCCCGACGCCCGGGCTTTGCCCGGGGGGCCT
CAGTGAGCGAGCGAGCGCGCAGCTGCCTGCAGG

1. A gene construct for expressing a covalently linked alpha beta dimer of beta-hexosaminidase comprising:
a. a nucleotide sequence encoding an alpha subunit of a beta-hexosaminidase;
b. a nucleotide sequence encoding a peptide linker; and
c. a nucleotide sequence encoding a beta subunit of a beta-hexosaminidase.

2. A gene construct according to claim 1, wherein the nucleotide sequence encoding an alpha subunit of a beta-hexosaminidase is positioned upstream of the nucleotide sequence encoding a beta subunit of a beta-hexosaminidase.

3. A gene construct according to claim 1, wherein the peptide linker is a flexible peptide linker.

4. A gene construct according to claim 1, wherein the peptide linker is a non-cleavable peptide.

5. A gene construct according to claim 1, wherein the peptide linker is not a self-cleavable peptide.

6. A gene construct according to claim 1, wherein at least 30% of the amino acid residues of the peptide linker are glycine residues.

7. A gene construct according to claim 1, wherein the nucleotide sequence encoding the peptide linker is selected from the group consisting of:
a. a nucleotide sequence encoding a polypeptide comprising an amino acid sequence that has at least 70% sequence identity with the amino acid sequence of SEQ ID NOs: 11, 12 or 13;
b. a nucleotide sequence comprising a sequence that has at least 70% sequence identity with the nucleotide sequence of SEQ ID NOs: 14, 15 or 16; and
c. a nucleotide sequence which differs from the sequence of a nucleotide sequence of (b) due to the degeneracy of the genetic code.

8. A gene construct according to claim 1, wherein the gene construct further comprises a promoter, preferably wherein said promotor is a constitutive promoter, more preferably a CBA promoter or a derivative thereof, even more preferably a Cbh promoter.

9. A gene construct according to claim 1, wherein the gene construct is flanked by adeno-associated viral ITRs, preferably AAV2 ITRs.

10. A gene construct according to claim 1, wherein the nucleotide sequence encoding an alpha subunit of a beta-hexosaminidase and/or the nucleotide sequence encoding a beta subunit of a beta-hexosaminidase are optimized, for example codon-optimized, preferably for expression in a human cell.

11. A gene construct according to claim 1, wherein the nucleotide sequence encoding an alpha subunit of a beta-hexosaminidase is selected from the group consisting of:
a. a nucleotide sequence encoding a polypeptide comprising an amino acid sequence that has at least 70% sequence identity with the amino acid sequence of SEQ ID NOs: 1 or 2;
b. a nucleotide sequence that has at least 70% sequence identity with the nucleotide sequence of SEQ ID NOs: 3, 4, 5, 64, 66, or 68; and
c. a nucleotide sequence which differs from the sequence of a nucleotide sequence of (b) due to the degeneracy of the genetic code;
and/or wherein the nucleotide sequence encoding a beta subunit of a beta-hexosaminidase is selected from the group consisting of:
a. a nucleotide sequence encoding a polypeptide comprising an amino acid sequence that has at least 70% sequence identity with the amino acid sequence of SEQ ID NOs: 6 or 7;
b. a nucleotide sequence that has at least 70% sequence identity with the nucleotide sequence of SEQ ID NOs: 8, 9, 10, 65, 67, or 69; and
c. a nucleotide sequence which differs from the sequence of a nucleotide sequence of (b) due to the degeneracy of the genetic code.

12. An expression vector comprising a gene construct according to claim 1.

13. An expression vector according to claim 12, wherein the expression vector is a viral vector, preferably an adeno-associated viral vector, more preferably an adeno-associated viral vector of serotype 1, 2, BR1, rh8, rh10, PHP.B, TT or 9, most preferably an adeno-associated viral vector of serotype 9.

14.-17. (canceled)

18. A method for treating GM2 gangliosidoses in a subject in need thereof, the method comprising administering to the subject the gene construct as defined in claim 1.

19. The method according to claim 18, wherein the GM2 gangliosidosis is selected from the group consisting of Sandhoff disease and Tay-Sachs disease.

20. The method according to claim 18, wherein the gene construct administered by intra-CSF administration.


SEQUENCE LISTING (88 sequences)

SEQ ID NO: 1 - protein, 529 aa, Homo sapiens
  source 1..529: note = alfa subunit beta hexosaminidase
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 2 - protein, 528 aa, Mus musculus
  source 1..528: note = alfa subunit beta hexosaminidase
MAGCRLWVSLLLAAALACLATALWPWPQYIQTYHRRYTLYPNNFQFRYHVSSAAQAGCVVLDEAFRRYRNLLFGSGSWPRPSFSNKQQTLGKNILVVSVVTAECNEFPNLESVENYTLTINDDQCLLASETVWGALRGLETFSQLVWKSAEGTFFINKTKIKDFPRFPHRGVLLDTSRHYLPLSSILDTLDVMAYNKFNVFHWHLVDDSSFPYESFTFPELTRKGSFNPVTHIYTAQDVKEVIEYARLRGIRVLAEFDTPGHTLSWGPGAPGLLTPCYSGSHLSGTFGPVNPSLNSTYDFMSTLFLEISSVFPDFYLHLGGDEVDFTCWKSNPNIQAFMKKKGFTDFKQLESFYIQTLLDIVSDYDKGYVVWQEVFDNKVKVRPDTIIQVWREEMPVEYMLEMQDITRAGFRALLSAPWYLNRVKYGPDWKDMYKVEPLAFHGTPEQKALVIGGEACMWGEYVDSTNLVPRLWPRAGAVAERLWSSNLTTNIDFAFKRLSHFRCELVRRGIQAQPISVGYCEQEFEQT

SEQ ID NO: 3 - DNA, 1590 bp, Homo sapiens
  source 1..1590: mol_type = other DNA; note = alfa subunit of beta hexosaminidase
atgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagacctga

SEQ ID NO: 4 - DNA, 1587 bp, Mus musculus
  source 1..1587: mol_type = other DNA; note = alfa subunit of beta hexosaminidase
atggccggctgcaggctctgggtttcgctgctgctggcggcggcgttggcttgcttggccacggcactgtggccgtggccccagtacatccaaacctaccaccggcgctacaccctgtaccccaacaacttccagttccggtaccatgtcagttcggccgcgcaggcgggctgcgtcgtcctcgacgaggcctttcgacgctaccgtaacctgctcttcggttccggctcttggccccgacccagcttctcaaataaacagcaaacgttggggaagaacattctggtggtctccgtcgtcacagctgaatgtaatgaatttcctaatttggagtcggtagaaaattacaccctaaccattaatgatgaccagtgtttactcgcctctgagactgtctggggcgctctccgaggtctggagactttcagtcagcttgtttggaaatcagctgagggcacgttctttatcaacaagacaaagattaaagactttcctcgattccctcaccggggcgtactgctggatacatctcgccattacctgccattgtctagcatcctggatacactggatgtcatggcatacaataaattcaacgtgttccactggcacttggtggacgactcttccttcccatatgagagcttcactttcccagagctcaccagaaaggggtccttcaaccctgtcactcacatctacacagcacaggatgtgaaggaggtcattgaatacgcaaggcttcggggtatccgtgtgctggcagaatttgacactcctggccacactttgtcctgggggccaggtgcccctgggttattaacaccttgctactctgggtctcatctctctggcacatttggaccggtgaaccccagtctcaacagcacctatgacttcatgagcacactcttcctggagatcagctcagtcttcccggacttttatctccacctgggaggggatgaagtcgacttcacctgctggaagtccaaccccaacatccaggccttcatgaagaaaaagggctttactgacttcaagcagctggagtccttctacatccagacgctgctggacatcgtctctgattatgacaagggctatgtggtgtggcaggaggtatttgataataaagtgaaggttcggccagatacaatcatacaggtgtggcgggaagaaatgccagtagagtacatgttggagatgcaagatatcaccagggctggcttccgggccctgctgtctgctccctggtacctgaaccgtgtaaagtatggccctgactggaaggacatgtacaaagtggagcccctggcgtttcatggtacgcctgaacagaaggctctggtcattggaggggaggcctgtatgtggggagagtatgtggacagcaccaacctggtccccagactctggcccagagcgggtgccgtcgctgagagactgtggagcagtaacctgacaactaatatagactttgcctttaaacgtttgtcgcatttccgttgtgagctggtgaggagaggaatccaggcccagcccatcagtgtaggctactgtgagcaggagtttgagcagacttga

SEQ ID NO: 5 - DNA, 1584 bp, synthetic construct
  source 1..1584: mol_type = other DNA; note = Optimized nucleotide sequence of alfa subunit of beta hexosaminidase
atggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagaca

SEQ ID NO: 6 - protein, 556 aa, Homo sapiens
  source 1..556: note = beta subunit beta hexosaminidase
MELCGLGLPRPPMLLALLLATLLAAMLALLTQVALVVQVAEAARAPSVSAKPGPALWPLPLLVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 7 - protein, 536 aa, Mus musculus
  source 1..536: note = beta subunit beta hexosaminidase
MPQSPRSAPGLLLLQALVSLVSLALVAPARLQPALWPFPRSVQMFPRLLYISAEDFSIDHSPNSTAGPSCSLLQEAFRRYYNYVFGFYKRHHGPARFRAEPQLQKLLVSITLESECESFPSLSSDETYSLLVQEPVAVLKANSVWGALRGLETFSQLVYQDSFGTFTINESSIADSPRFPHRGILIDTSRHFLPVKTILKTLDAMAFNKFNVLHWHIVDDQSFPYQSTTFPELSNKGSYSLSHVYTPNDVRMVLEYARLRGIRVIPEFDTPGHTQSWGKGQKNLLTPCYNQKTKTQVFGPVDPTVNTTYAFFNTFFKEISSVFPDQFIHLGGDEVEFQCWASNPNIQGFMKRKGFGSDFRRLESFYIKKILEIISSLKKNSIVWQEVFDDKVELQPGTVVEVWKSEHYSYELKQVTGSGFPAILSAPWYLDLISYGQDWKNYYKVEPLNFEGSEKQKQLVIGGEACLWGEFVDATNLTPRLWPRASAVGERLWSPKTVTDLENAYKRLAVHRCRMVSRGIAAQPLYTGYCNYENKI

SEQ ID NO: 8 - DNA, 1671 bp, Homo sapiens
  source 1..1671: mol_type = other DNA; note = beta subunit beta hexosaminidase
atggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctcttggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaa

SEQ ID NO: 9 - DNA, 1611 bp, Mus musculus
  source 1..1611: mol_type = other DNA; note = beta subunit beta hexosaminidase
atgccgcagtccccgcgtagcgcccccgggctgctgctgctgcaggcgctggtgtcgctagtgtcgctggccctagtggccccggcccgactgcaacctgcgctatggcccttcccgcgctcggtgcagatgttcccgcggctgttgtacatctccgcggaggacttcagcatcgaccacagtcccaattccacagcgggcccttcctgctcgctgctacaggaggcgtttcggcgatattacaactatgtttttggtttctacaagagacatcatggccctgctagatttcgagctgagccacagttgcagaagctcctggtctccattaccctcgagtcagagtgcgagtccttccctagtctgtcttcagatgaaacctattctctgcttgtacaagaaccagtagccgtcctcaaggccaacagcgtttggggagcgttacgaggtttagagacgtttagccagttagtttaccaagactctttcgggactttcaccatcaatgaatccagtatagctgattctccaagattccctcatagaggaattttaattgatacatctagacacttcctgcctgtgaagacaattttaaaaactctggatgccatggcttttaataagtttaatgttcttcactggcacatagtggacgaccagtctttcccttatcagagtaccacttttcctgagctaagcaataagggaagctactctttgtctcatgtctatacaccaaacgatgtccggatggtgctggagtacgcccggctccgagggattcgagtcataccagaatttgatacccctggccatacacagtcttggggcaaaggacagaaaaaccttctaactccatgttacaatcaaaaaactaaaactcaagtgtttgggcctgtagacccaactgtaaacacaacgtatgcattctttaacacatttttcaaagaaatcagcagtgtgtttccagatcagttcatccacttgggaggagatgaagtagaatttcaatgttgggcatcaaatccaaacatccaaggtttcatgaagagaaagggctttggcagcgattttagaagactagaatccttttatattaaaaagattttggaaattatttcatccttaaagaagaactccattgtttggcaagaagtttttgatgataaggtggagcttcagccgggcacagtagtcgaagtgtggaagagtgagcattattcatatgagctaaagcaagtcacaggctctggcttccctgccatcctttctgctccttggtacttagacctgatcagctatgggcaagactggaaaaactactacaaagttgagccccttaattttgaaggctctgagaagcagaaacaacttgttattggtggagaagcttgcctgtggggagaatttgtggatgcaactaaccttactccaagattatggcctcgagcaagcgctgttggtgagagactctggagccctaaaactgtcactgacctagaaaatgcctacaaacgactggccgtgcaccgctgcagaatggtcagccgtggaatagctgcacaacctctctatactggatactgtaactatgagaataaaatatag

SEQ ID NO: 10 - DNA, 1518 bp, synthetic construct
  source 1..1518: mol_type = other DNA; note = Optimized nucleotide sequence of beta subunit beta hexosaminidase
cagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctga

SEQ ID NO: 11 - protein, 20 aa, synthetic construct
  source 1..20: note = peptide linker L1
GGGGSGGGGSGGGGSGGGGS

SEQ ID NO: 12 - protein, 33 aa, synthetic construct
  source 1..33: note = peptide linker L2
SGGSSGGSSGSETPGTSESATPESSGGSSGGSS

SEQ ID NO: 13 - protein, 12 aa, synthetic construct
  source 1..12: note = peptide linker L3
GSAGSAAGSGEF

SEQ ID NO: 14 - DNA, 60 bp, synthetic construct
  source 1..60: mol_type = other DNA; note = peptide linker L1
ggtggtggtggatctggaggcggaggatcaggcggcggaggttctggaggtggaggtagt

SEQ ID NO: 15 - DNA, 99 bp, synthetic construct
  source 1..99: mol_type = other DNA; note = peptide linker L2
tctggaggatctagcggaggatcctctggcagcgagacaccaggaacaagcgagtcagcaacaccagagagcagtggcggcagcagcggcggcagcagc

SEQ ID NO: 16 - DNA, 36 bp, synthetic construct
  source 1..36: mol_type = other DNA; note = peptide linker L3
ggcagcgccggcagcgccgccggcagcggcgagttc

SEQ ID NO: 17 - protein, 1053 aa, synthetic construct
  source 1..1053: note = covalently linked alfa beta dimer of beta hexosaminidase L1
MAGCRLWVSLLLAAALACLATALWPWPQYIQTYHRRYTLYPNNFQFRYHVSSAAQAGCVVLDEAFRRYRNLLFGSGSWPRPSFSNKQQTLGKNILVVSVVTAECNEFPNLESVENYTLTINDDQCLLASETVWGALRGLETFSQLVWKSAEGTFFINKTKIKDFPRFPHRGVLLDTSRHYLPLSSILDTLDVMAYNKFNVFHWHLVDDSSFPYESFTFPELTRKGSFNPVTHIYTAQDVKEVIEYARLRGIRVLAEFDTPGHTLSWGPGAPGLLTPCYSGSHLSGTFGPVNPSLNSTYDFMSTLFLEISSVFPDFYLHLGGDEVDFTCWKSNPNIQAFMKKKGFTDFKQLESFYIQTLLDIVSDYDKGYVVWQEVFDNKVKVRPDTIIQVWREEMPVEYMLEMQDITRAGFRALLSAPWYLNRVKYGPDWKDMYKVEPLAFHGTPEQKALVIGGEACMWGEYVDSTNLVPRLWPRAGAVAERLWSSNLTTNIDFAFKRLSHFRCELVRRGIQAQPISVGYCEQEFEQTGGGGSGGGGSGGGGSGGGGSQPALWPFPRSVQMFPRLLYISAEDFSIDHSPNSTAGPSCSLLQEAFRRYYNYVFGFYKRHHGPARFRAEPQLQKLLVSITLESECESFPSLSSDETYSLLVQEPVAVLKANSVWGALRGLETFSQLVYQDSFGTFTINESSIADSPRFPHRGILIDTSRHFLPVKTILKTLDAMAFNKFNVLHWHIVDDQSFPYQSTTFPELSNKGSYSLSHVYTPNDVRMVLEYARLRGIRVIPEFDTPGHTQSWGKGQKNLLTPCYNQKTKTQVFGPVDPTVNTTYAFFNTFFKEISSVFPDQFIHLGGDEVEFQCWASNPNIQGFMKRKGFGSDFRRLESFYIKKILEIISSLKKNSIVWQEVFDDKVELQPGTVVEVWKSEHYSYELKQVTGSGFPAILSAPWYLDLISYGQDWKNYYKVEPLNFEGSEKQKQLVIGGEACLWGEFVDATNLTPRLWPRASAVGERLWSPKTVTDLENAYKRLAVHRCRMVSRGIAAQPLYTGYCNYENKI

SEQ ID NO: 18 - protein, 1066 aa, synthetic construct
  source 1..1066: note = covalently linked alfa beta dimer of beta hexosaminidase L2
MAGCRLWVSLLLAAALACLATALWPWPQYIQTYHRRYTLYPNNFQFRYHVSSAAQAGCVVLDEAFRRYRNLLFGSGSWPRPSFSNKQQTLGKNILVVSVVTAECNEFPNLESVENYTLTINDDQCLLASETVWGALRGLETFSQLVWKSAEGTFFINKTKIKDFPRFPHRGVLLDTSRHYLPLSSILDTLDVMAYNKFNVFHWHLVDDSSFPYESFTFPELTRKGSFNPVTHIYTAQDVKEVIEYARLRGIRVLAEFDTPGHTLSWGPGAPGLLTPCYSGSHLSGTFGPVNPSLNSTYDFMSTLFLEISSVFPDFYLHLGGDEVDFTCWKSNPNIQAFMKKKGFTDFKQLESFYIQTLLDIVSDYDKGYVVWQEVFDNKVKVRPDTIIQVWREEMPVEYMLEMQDITRAGFRALLSAPWYLNRVKYGPDWKDMYKVEPLAFHGTPEQKALVIGGEACMWGEYVDSTNLVPRLWPRAGAVAERLWSSNLTTNIDFAFKRLSHFRCELVRRGIQAQPISVGYCEQEFEQTSGGSSGGSSGSETPGTSESATPESSGGSSGGSSQPALWPFPRSVQMFPRLLYISAEDFSIDHSPNSTAGPSCSLLQEAFRRYYNYVFGFYKRHHGPARFRAEPQLQKLLVSITLESECESFPSLSSDETYSLLVQEPVAVLKANSVWGALRGLETFSQLVYQDSFGTFTINESSIADSPRFPHRGILIDTSRHFLPVKTILKTLDAMAFNKFNVLHWHIVDDQSFPYQSTTFPELSNKGSYSLSHVYTPNDVRMVLEYARLRGIRVIPEFDTPGHTQSWGKGQKNLLTPCYNQKTKTQVFGPVDPTVNTTYAFFNTFFKEISSVFPDQFIHLGGDEVEFQCWASNPNIQGFMKRKGFGSDFRRLESFYIKKILEIISSLKKNSIVWQEVFDDKVELQPGTVVEVWKSEHYSYELKQVTGSGFPAILSAPWYLDLISYGQDWKNYYKVEPLNFEGSEKQKQLVIGGEACLWGEFVDATNLTPRLWPRASAVGERLWSPKTVTDLENAYKRLAVHRCRMVSRGIAAQPLYTGYCNYENKI

SEQ ID NO: 19 - protein, 1045 aa, synthetic construct
  source 1..1045: note = covalently linked alfa beta dimer of beta hexosaminidase L3
MAGCRLWVSLLLAAALACLATALWPWPQYIQTYHRRYTLYPNNFQFRYHVSSAAQAGCVVLDEAFRRYRNLLFGSGSWPRPSFSNKQQTLGKNILVVSVVTAECNEFPNLESVENYTLTINDDQCLLASETVWGALRGLETFSQLVWKSAEGTFFINKTKIKDFPRFPHRGVLLDTSRHYLPLSSILDTLDVMAYNKFNVFHWHLVDDSSFPYESFTFPELTRKGSFNPVTHIYTAQDVKEVIEYARLRGIRVLAEFDTPGHTLSWGPGAPGLLTPCYSGSHLSGTFGPVNPSLNSTYDFMSTLFLEISSVFPDFYLHLGGDEVDFTCWKSNPNIQAFMKKKGFTDFKQLESFYIQTLLDIVSDYDKGYVVWQEVFDNKVKVRPDTIIQVWREEMPVEYMLEMQDITRAGFRALLSAPWYLNRVKYGPDWKDMYKVEPLAFHGTPEQKALVIGGEACMWGEYVDSTNLVPRLWPRAGAVAERLWSSNLTTNIDFAFKRLSHFRCELVRRGIQAQPISVGYCEQEFEQTGSAGSAAGSGEFQPALWPFPRSVQMFPRLLYISAEDFSIDHSPNSTAGPSCSLLQEAFRRYYNYVFGFYKRHHGPARFRAEPQLQKLLVSITLESECESFPSLSSDETYSLLVQEPVAVLKANSVWGALRGLETFSQLVYQDSFGTFTINESSIADSPRFPHRGILIDTSRHFLPVKTILKTLDAMAFNKFNVLHWHIVDDQSFPYQSTTFPELSNKGSYSLSHVYTPNDVRMVLEYARLRGIRVIPEFDTPGHTQSWGKGQKNLLTPCYNQKTKTQVFGPVDPTVNTTYAFFNTFFKEISSVFPDQFIHLGGDEVEFQCWASNPNIQGFMKRKGFGSDFRRLESFYIKKILEIISSLKKNSIVWQEVFDDKVELQPGTVVEVWKSEHYSYELKQVTGSGFPAILSAPWYLDLISYGQDWKNYYKVEPLNFEGSEKQKQLVIGGEACLWGEFVDATNLTPRLWPRASAVGERLWSPKTVTDLENAYKRLAVHRCRMVSRGIAAQPLYTGYCNYENKI

SEQ ID NO: 20 - DNA, 3162 bp, synthetic construct
  source 1..3162: mol_type = other DNA; note = covalently linked alfa beta dimer of beta hexosaminidase L1
atggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacaggtggtggtggatctggaggcggaggatcaggcggcggaggttctggaggtggaggtagtcagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctga

SEQ ID NO: 21 - DNA, 3201 bp, synthetic construct
  source 1..3201: mol_type = other DNA; note = covalently linked alfa beta dimer of beta hexosaminidase L2
atggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacatctggaggatctagcggaggatcctctggcagcgagacaccaggaacaagcgagtcagcaacaccagagagcagtggcggcagcagcggcggcagcagccagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctga

SEQ ID NO: 22 - DNA, 3138 bp, synthetic construct
  source 1..3138: mol_type = other DNA; note = covalently linked alfa beta dimer of beta hexosaminidase L3
atggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacaggcagcgccggcagcgccgccggcagcggcgagttccagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctga

SEQ ID NO: 23 - DNA, 278 bp, synthetic construct
  source 1..278: mol_type = other DNA; note = CBA promoter
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 24 - DNA, 801 bp, synthetic construct
  source 1..801: mol_type = other DNA; note = Cbh promoter
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggag

SEQ ID NO: 25 - DNA, 141 bp, synthetic construct
  source 1..141: mol_type = other DNA; note = AAV2 5' ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 26 - DNA, 141 bp, synthetic construct
  source 1..141: mol_type = other DNA; note = AAV2 3' ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 27 - DNA, 122 bp, synthetic construct
  source 1..122: mol_type = other DNA; note = SV40 polyadenylation signal
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 28 - DNA, 7004 bp, synthetic construct
  source 1..7004: mol_type = other DNA; note = pAAV-Cbh-omHexa-L1-omHexb-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacaggtggtggtggatctggaggcggaggatcaggcggcggaggttctggaggtggaggtagtcagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 29 - DNA, 7043 bp, synthetic construct
  source 1..7043: mol_type = other DNA; note = pAAV-Cbh-omHexa-L2-omHexb-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacatctggaggatctagcggaggatcctctggcagcgagacaccaggaacaagcgagtcagcaacaccagagagcagtggcggcagcagcggcggcagcagccagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 30 - DNA, 6980 bp, synthetic construct
  source 1..6980: mol_type = other DNA; note = pAAV-Cbh-omHexa-L3-omHexb-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacaggcagcgccggcagcgccgccggcagcggcgagttccagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 31 - DNA, 6833 bp, synthetic construct
  source 1..6833: mol_type = other DNA; note = pAAV-Cbh-omHexb-L1-omHexa-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgcctcagagccctagatctgctcctggactgctgctgctccaggctctggtgtctcttgtgtctctggcactggtggcccctgctagactgcaacctgctctgtggccatttcctagaagcgtgcagatgttccccagactgctgtacatcagcgccgaggacttcagcatcgatcacagccccaacagcacagctggccctagttgtagcctgctgcaagaggccttcagacggtactacaactacgtgttcggcttctacaagaggcaccacggacctgccagattcagagctgagccccagctgcagaaactgctggtgtctatcaccctggaaagcgagtgcgagagcttccctagcctgagcagcgacgagacatacagcctgctggtgcaagagcctgtggccgtgctgaaggctaactctgtttggggcgctctgagaggcctggaaaccttctctcagctggtgtaccaggacagcttcggcaccttcaccatcaacgagtcctctatcgccgactctcctagattccctcacagaggcatcctgatcgataccagcagacacttcctgcctgtgaaaaccatcctgaaaaccctggacgccatggccttcaacaagttcaacgtgctgcactggcacatcgtggacgaccagagctttccctaccagtccaccacctttccagagctgtccaacaagggcagctacagcctgagccacgtgtacacccctaacgacgtgcggatggtgctggaatacgccagactgagaggcatcagagtgatccccgagttcgacacccctggccacacacagtcttggggcaagggacagaagaacctgctgaccccttgctacaaccagaaaacaaagacccaggtgttcggccctgtggaccctaccgtgaacaccacctacgctttcttcaacaccttcttcaaagaaatcagcagcgtgttccccgaccagttcatccacctcggcggagatgaggtcgagttccagtgttgggccagcaatcccaacatccagggctttatgaagaggaaaggcttcggcagcgacttcagaaggctggaaagcttctacatcaagaagatcctcgagatcatcagcagcctgaagaagaacagcatcgtctggcaagaggtgttcgacgacaaggtggaactgcagcctggcaccgtggtggaagtgtggaagtctgagcactacagctacgagctgaagcaagtgaccggctctggcttccctgccatcctttctgccccttggtatctggacctgatcagctacggccaggactggaagaactactacaaggtcgagcccctgaacttcgagggcagcgagaagcagaagcagctggttatcggaggcgaggcttgtctgtggggcgagttcgtggatgccaccaacctgacacctagactgtggcctagagcctctgctgtgggcgagagactgtggtcccctaagacagtgaccgacctggaaaacgcctacaagagactggccgtgcacagatgcagaatggtgtccagaggaatcgccgctcagcctctgtacaccggctactgcaactacgagaacaagatcggtggtggtggatctggaggcggaggatcaggcggcggaggttctggaggtggaggtagtaccctgggcaagaacatcctggtggtgtctgtggtcaccgccgagtgcaacgagttccccaacctggaatccgtggaaaactacaccctgacaatcaacgacgaccagtgcctgctggcctccgaaacagtttggggagcactgcgcggactggaaacattcagccagctcgtgtggaaatctgccgagggcacattcttcatcaacaagaccaagatcaaggacttccctaggttcccacacaggggcgtgctgctggacacctccagacattacctgcctctgagcagcatcctggacacactggacgtgatggcttataacaagtttaatgtgttccattggcacctggtcgacgacagcagcttcccttacgagtccttcacattccccgagctgacaagaaagggctctttcaaccccgtgacacacatctacacagcccaggacgtgaaagaagtgatcgagtacgctaggctgcggggaatcagagtgctggctgagttcgatacacccggacacactctgagttggggacctggtgctcctggcctcctgacaccatgttactctggctctcacctgagcggcacattcggcccagtgaacccaagcctgaatagcacctacgactttatgagcaccctgtttctggaaatctcctccgtgtttcccgacttctacctgcatctcggcggcgacgaggtggacttcacctgttggaagtctaaccccaatatccaggcattcatgaagaagaaggggttcaccgacttcaagcagcttgagagcttctatatccagacgctgctggatatcgtgtccgactacgacaagggctacgttgtgtggcaagaagtctttgacaacaaagtgaaagtgcggcccgacaccatcatccaagtgtggcgggaagagatgcccgtcgagtacatgctggaaatgcaggacatcacaagagccggcttcagggctctgctgagcgctccatggtatctgaacagagtgaagtacggccccgattggaaggacatgtacaaagtggaacctctggccttccacggcacccctgaacagaaagctctcgtgattggcggcgaggcctgcatgtggggagaatacgtggacagcacaaacctggtgcctcggctttggccaagagctggtgctgtggctgaaaggctgtggtctagcaacctgaccaccaacatcgacttcgccttcaagaggctgtcccacttcagatgcgaactcgtgcggagaggaatccaggctcagccaatctctgtgggctactgcgagcaagagttcgagcagacctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 32 - DNA, 6872 bp, synthetic construct
  source 1..6872: mol_type = other DNA; note = pAAV-Cbh-omHexb-L2-omHexa-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgcctcagagccctagatctgctcctggactgctgctgctccaggctctggtgtctcttgtgtctctggcactggtggcccctgctagactgcaacctgctctgtggccatttcctagaagcgtgcagatgttccccagactgctgtacatcagcgccgaggacttcagcatcgatcacagccccaacagcacagctggccctagttgtagcctgctgcaagaggccttcagacggtactacaactacgtgttcggcttctacaagaggcaccacggacctgccagattcagagctgagccccagctgcagaaactgctggtgtctatcaccctggaaagcgagtgcgagagcttccctagcctgagcagcgacgagacatacagcctgctggtgcaagagcctgtggccgtgctgaaggctaactctgtttggggcgctctgagaggcctggaaaccttctctcagctggtgtaccaggacagcttcggcaccttcaccatcaacgagtcctctatcgccgactctcctagattccctcacagaggcatcctgatcgataccagcagacacttcctgcctgtgaaaaccatcctgaaaaccctggacgccatggccttcaacaagttcaacgtgctgcactggcacatcgtggacgaccagagctttccctaccagtccaccacctttccagagctgtccaacaagggcagctacagcctgagccacgtgtacacccctaacgacgtgcggatggtgctggaatacgccagactgagaggcatcagagtgatccccgagttcgacacccctggccacacacagtcttggggcaagggacagaagaacctgctgaccccttgctacaaccagaaaacaaagacccaggtgttcggccctgtggaccctaccgtgaacaccacctacgctttcttcaacaccttcttcaaagaaatcagcagcgtgttccccgaccagttcatccacctcggcggagatgaggtcgagttccagtgttgggccagcaatcccaacatccagggctttatgaagaggaaaggcttcggcagcgacttcagaaggctggaaagcttctacatcaagaagatcctcgagatcatcagcagcctgaagaagaacagcatcgtctggcaagaggtgttcgacgacaaggtggaactgcagcctggcaccgtggtggaagtgtggaagtctgagcactacagctacgagctgaagcaagtgaccggctctggcttccctgccatcctttctgccccttggtatctggacctgatcagctacggccaggactggaagaactactacaaggtcgagcccctgaacttcgagggcagcgagaagcagaagcagctggttatcggaggcgaggcttgtctgtggggcgagttcgtggatgccaccaacctgacacctagactgtggcctagagcctctgctgtgggcgagagactgtggtcccctaagacagtgaccgacctggaaaacgcctacaagagactggccgtgcacagatgcagaatggtgtccagaggaatcgccgctcagcctctgtacaccggctactgcaactacgagaacaagatctctggaggatctagcggaggatcctctggcagcgagacaccaggaacaagcgagtcagcaacaccagagagcagtggcggcagcagcggcggcagcagcaccctgggcaagaacatcctggtggtgtctgtggtcaccgccgagtgcaacgagttccccaacctggaatccgtggaaaactacaccctgacaatcaacgacgaccagtgcctgctggcctccgaaacagtttggggagcactgcgcggactggaaacattcagccagctcgtgtggaaatctgccgagggcacattcttcatcaacaagaccaagatcaaggacttccctaggttcccacacaggggcgtgctgctggacacctccagacattacctgcctctgagcagcatcctggacacactggacgtgatggcttataacaagtttaatgtgttccattggcacctggtcgacgacagcagcttcccttacgagtccttcacattccccgagctgacaagaaagggctctttcaaccccgtgacacacatctacacagcccaggacgtgaaagaagtgatcgagtacgctaggctgcggggaatcagagtgctggctgagttcgatacacccggacacactctgagttggggacctggtgctcctggcctcctgacaccatgttactctggctctcacctgagcggcacattcggcccagtgaacccaagcctgaatagcacctacgactttatgagcaccctgtttctggaaatctcctccgtgtttcccgacttctacctgcatctcggcggcgacgaggtggacttcacctgttggaagtctaaccccaatatccaggcattcatgaagaagaaggggttcaccgacttcaagcagcttgagagcttctatatccagacgctgctggatatcgtgtccgactacgacaagggctacgttgtgtggcaagaagtctttgacaacaaagtgaaagtgcggcccgacaccatcatccaagtgtggcgggaagagatgcccgtcgagtacatgctggaaatgcaggacatcacaagagccggcttcagggctctgctgagcgctccatggtatctgaacagagtgaagtacggccccgattggaaggacatgtacaaagtggaacctctggccttccacggcacccctgaacagaaagctctcgtgattggcggcgaggcctgcatgtggggagaatacgtggacagcacaaacctggtgcctcggctttggccaagagctggtgctgtggctgaaaggctgtggtctagcaacctgaccaccaacatcgacttcgccttcaagaggctgtcccacttcagatgcgaactcgtgcggagaggaatccaggctcagccaatctctgtgggctactgcgagcaagagttcgagcagacctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 33 - DNA, 6809 bp, synthetic construct
  source 1..6809: mol_type = other DNA; note = pAAV-Cbh-omHexb-L3-omHexa-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgcctcagagccctagatctgctcctggactgctgctgctccaggctctggtgtctcttgtgtctctggcactggtggcccctgctagactgcaacctgctctgtggccatttcctagaagcgtgcagatgttccccagactgctgtacatcagcgccgaggacttcagcatcgatcacagccccaacagcacagctggccctagttgtagcctgctgcaagaggccttcagacggtactacaactacgtgttcggcttctacaagaggcaccacggacctgccagattcagagctgagccccagctgcagaaactgctggtgtctatcaccctggaaagcgagtgcgagagcttccctagcctgagcagcgacgagacatacagcctgctggtgcaagagcctgtggccgtgctgaaggctaactctgtttggggcgctctgagaggcctggaaaccttctctcagctggtgtaccaggacagcttcggcaccttcaccatcaacgagtcctctatcgccgactctcctagattccctcacagaggcatcctgatcgataccagcagacacttcctgcctgtgaaaaccatcctgaaaaccctggacgccatggccttcaacaagttcaacgtgctgcactggcacatcgtggacgaccagagctttccctaccagtccaccacctttccagagctgtccaacaagggcagctacagcctgagccacgtgtacacccctaacgacgtgcggatggtgctggaatacgccagactgagaggcatcagagtgatccccgagttcgacacccctggccacacacagtcttggggcaagggacagaagaacctgctgaccccttgctacaaccagaaaacaaagacccaggtgttcggccctgtggaccctaccgtgaacaccacctacgctttcttcaacaccttcttcaaagaaatcagcagcgtgttccccgaccagttcatccacctcggcggagatgaggtcgagttccagtgttgggccagcaatcccaacatccagggctttatgaagaggaaaggcttcggcagcgacttcagaaggctggaaagcttctacatcaagaagatcctcgagatcatcagcagcctgaagaagaacagcatcgtctggcaagaggtgttcgacgacaaggtggaactgcagcctggcaccgtggtggaagtgtggaagtctgagcactacagctacgagctgaagcaagtgaccggctctggcttccctgccatcctttctgccccttggtatctggacctgatcagctacggccaggactggaagaactactacaaggtcgagcccctgaacttcgagggcagcgagaagcagaagcagctggttatcggaggcgaggcttgtctgtggggcgagttcgtggatgccaccaacctgacacctagactgtggcctagagcctctgctgtgggcgagagactgtggtcccctaagacagtgaccgacctggaaaacgcctacaagagactggccgtgcacagatgcagaatggtgtccagaggaatcgccgctcagcctctgtacaccggctactgcaactacgagaacaagatcggcagcgccggcagcgccgccggcagcggcgagttcaccctgggcaagaacatcctggtggtgtctgtggtcaccgccgagtgcaacgagttccccaacctggaatccgtggaaaactacaccctgacaatcaacgacgaccagtgcctgctggcctccgaaacagtttggggagcactgcgcggactggaaacattcagccagctcgtgtggaaatctgccgagggcacattcttcatcaacaagaccaagatcaaggacttccctaggttcccacacaggggcgtgctgctggacacctccagacattacctgcctctgagcagcatcctggacacactggacgtgatggcttataacaagtttaatgtgttccattggcacctggtcgacgacagcagcttcccttacgagtccttcacattccccgagctgacaagaaagggctctttcaaccccgtgacacacatctacacagcccaggacgtgaaagaagtgatcgagtacgctaggctgcggggaatcagagtgctggctgagttcgatacacccggacacactctgagttggggacctggtgctcctggcctcctgacaccatgttactctggctctcacctgagcggcacattcggcccagtgaacccaagcctgaatagcacctacgactttatgagcaccctgtttctggaaatctcctccgtgtttcccgacttctacctgcatctcggcggcgacgaggtggacttcacctgttggaagtctaaccccaatatccaggcattcatgaagaagaaggggttcaccgacttcaagcagcttgagagcttctatatccagacgctgctggatatcgtgtccgactacgacaagggctacgttgtgtggcaagaagtctttgacaacaaagtgaaagtgcggcccgacaccatcatccaagtgtggcgggaagagatgcccgtcgagtacatgctggaaatgcaggacatcacaagagccggcttcagggctctgctgagcgctccatggtatctgaacagagtgaagtacggccccgattggaaggacatgtacaaagtggaacctctggccttccacggcacccctgaacagaaagctctcgtgattggcggcgaggcctgcatgtggggagaatacgtggacagcacaaacctggtgcctcggctttggccaagagctggtgctgtggctgaaaggctgtggtctagcaacctgaccaccaacatcgacttcgccttcaagaggctgtcccacttcagatgcgaactcgtgcggagaggaatccaggctcagccaatctctgtgggctactgcgagcaagagttcgagcagacctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 34 - DNA, 5429 bp, synthetic construct
  source 1..5429: mol_type = other DNA; note = pAAV-Cbh-omHexa-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggctggatgtagactgtgggtttccctgctgctggctgccgctcttgcttgtctggctacagctctttggccctggcctcagtacatccagacctaccacagacggtacacactgtaccccaacaacttccagttccgctaccacgtgtcctctgctgctcaggcaggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 35 - DNA, 5453 bp, synthetic construct
  source 1..5453: mol_type = other DNA; note = pAAV-Cbh-omHexb-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgcctcaatctccaagatctgctcctggcctgctgctgctccaggctctggtgtctctggtttcactggcactggtggcccctgctagactgcaacctgctctgtggccatttcctagaagcgtgcagatgttccccagactgctgtacatcagcgccgaggacttcagcatcgatcacagccccaacagcacagctggccctagttgtagcctgctgcaagaggccttcagacggtactacaactacgtgttcggcttctacaagaggcaccacggacctgccagattcagagctgagccccagctgcagaaactgctggtgtctatcaccctggaaagcgagtgcgagagcttccctagcctgagcagcgacgagacatacagcctgctggtgcaagagcctgtggccgtgctgaaggctaactctgtttggggcgctctgagaggcctggaaaccttctctcagctggtgtaccaggacagcttcggcaccttcaccatcaacgagtcctctatcgccgactctcctagattccctcacagaggcatcctgatcgataccagcagacacttcctgcctgtgaaaaccatcctgaaaaccctggacgccatggccttcaacaagttcaacgtgctgcactggcacatcgtggacgaccagagctttccctaccagtccaccacctttccagagctgtccaacaagggcagctacagcctgagccacgtgtacacccctaacgacgtgcggatggtgctggaatacgccagactgagaggcatcagagtgatccccgagttcgacacccctggccacacacagtcttggggcaagggacagaagaacctgctgaccccttgctacaaccagaaaacaaagacccaggtgttcggccctgtggaccctaccgtgaacaccacctacgctttcttcaacaccttcttcaaagaaatcagcagcgtgttccccgaccagttcatccacctcggcggagatgaggtcgagttccagtgttgggccagcaatcccaacatccagggctttatgaagaggaaaggcttcggcagcgacttcagaaggctggaaagcttctacatcaagaagatcctcgagatcatcagcagcctgaagaagaacagcatcgtctggcaagaggtgttcgacgacaaggtggaactgcagcctggcaccgtggtggaagtgtggaagtctgagcactacagctacgagctgaagcaagtgaccggctctggcttccctgccatcctttctgccccttggtatctggacctgatcagctacggccaggactggaagaactactacaaggtcgagcccctgaacttcgagggcagcgagaagcagaagcagctggttatcggaggcgaggcttgtctgtggggcgagttcgtggatgccaccaacctgacacctagactgtggcctagagcctctgctgtgggcgagagactgtggtcccctaagacagtgaccgacctggaaaacgcctacaagagactggccgtgcacagatgcagaatggtgtccagaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 36 - DNA, 7103 bp, synthetic construct
  source 1..7103: mol_type = other DNA; note = pAAV-Cbh-omHexa-P2A-omHexb-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacaggatctggcgccaccaacttcagcctgctgaaacaggctggcgacgtggaagagaaccccggacctatgcctcagagccctagatctgctcccgggctgctgcttctgcaagctctggtgtctctcgtgtctctggccctggttgctccagctagactgcagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 37 - DNA, 20 bp, synthetic construct
  source 1..20: mol_type = other DNA; note = CD68 forward primer
tggcggtggaatacaatgtg

SEQ ID NO: 38 - DNA, 20 bp, synthetic construct
  source 1..20: mol_type = other DNA; note = CD68 reverse primer
tgcttgcatttccacagcag

SEQ ID NO: 39 - DNA, 20 bp, synthetic construct
  source 1..20: mol_type = other DNA; note = Plp1 forward primer
ttccagaggccaacatcaag

SEQ ID NO: 40 - DNA, 20 bp, synthetic construct
  source 1..20: mol_type = other DNA; note = Plp1 reverse primer
aggagccatacaacagtcag

SEQ ID NO: 41 - DNA, 21 bp, synthetic construct
  source 1..21: mol_type = other DNA; note = Ugt8 forward primer
agtttccaagaccaacgctgc

SEQ ID NO: 42 - DNA, 21 bp, synthetic construct
  source 1..21: mol_type = other DNA; note = Ugt8 reverse primer
tgttcctgagcaccacttacc

SEQ ID NO: 43 - DNA, 20 bp, synthetic construct
  source 1..20: mol_type = other DNA; note = Tbp forward primer
tgactcctggaattcccatc

SEQ ID NO: 44 - DNA, 20 bp, synthetic construct
  source 1..20: mol_type = other DNA; note = Tbp reverse primer
tgctgctgtctttgttgctc

SEQ ID NO: 45 - DNA, 21 bp, synthetic construct
  source 1..21: mol_type = other DNA; note = Forward common primer Sandhoff
attttaaaattcaggcctcga

SEQ ID NO: 46 - DNA, 21 bp, synthetic construct
  source 1..21: mol_type = other DNA; note = Reverse WT primer Sandhoff
cattctgcagcggtgcacggc

SEQ ID NO: 47 - DNA, 21 bp, synthetic construct
  source 1..21: mol_type = other DNA; note = Reverse Mutant primer Sandhoff
catagcgttggctacccgtga

SEQ ID NO: 48 - DNA, 22 bp, synthetic construct
  source 1..22: mol_type = other DNA; note = Forward WT primer Tay-Sachs
ggtgtatgtgtaacactcgtgg

SEQ ID NO: 49 - DNA, 22 bp, synthetic construct
  source 1..22: mol_type = other DNA; note = Reverse WT primer Tay-Sachs
cagttctaggctcagaatgagg

SEQ ID NO: 50 - DNA, 20 bp, synthetic construct
  source 1..20: mol_type = other DNA; note = Forward Mutant primer Tay-Sachs
cttgggtggagaggctattc

SEQ ID NO: 51 - DNA, 20 bp, synthetic construct
  source 1..20: mol_type = other DNA; note = Reverse Mutant primer Tay-Sachs
aggtgagatgacaggagatc

SEQ ID NO: 52 - protein, 5 aa, synthetic construct
  source 1..5: note = (GGGGS)n peptide linker
GGGGS

SEQ ID NO: 53 - protein, 6 aa, synthetic construct
  source 1..6: note = Gly6 peptide linker
GGGGGG

SEQ ID NO: 54 - protein, 8 aa, synthetic construct
  source 1..8: note = Gly8 peptide linker
GGGGGGGG

SEQ ID NO: 55 - protein, 18 aa, synthetic construct
  source 1..18: note = KESGSVSSEQLAQFRSLD peptide linker
KESGSVSSEQLAQFRSLD

SEQ ID NO: 56 - protein, 14 aa, synthetic construct
  source 1..14: note = EGKSSGSGSESKST peptide linker
EGKSSGSGSESKST

SEQ ID NO: 57 - protein, 22 aa, synthetic construct
  source 1..22: note = Signal peptide (mouse)
MAGCRLWVSLLLAAALACLATA

SEQ ID NO: 58 - protein, 22 aa, synthetic construct
  source 1..22: note = Signal peptide (human)
MTSSRLWFSLLLAAAFAGRATA

SEQ ID NO: 59 - DNA, 7102 bp, synthetic construct
  source 1..7102: mol_type = other DNA; note = pAAV-Cbh-omHexb-P2A-omHexa-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctgaggatctggcgccaccaacttcagcctgctgaaacaggctggcgacgtggaagagaaccccggacctatgcctcagagccctagatctgctcccgggctgctgcttctgcaagctctggtgtctctcgtgtctctggccctggttgctccagctagactgatggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 60 - DNA, 6683 bp, synthetic construct
  source 1..6683: mol_type = other DNA; note = pAAV-Cbh-nohHEXA-L1-nohHEXB-SV40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagacctgaggtggtggtggatctggaggcggaggatcaggcggcggaggttctggaggtggaggtagtatggagctgtgcgggctggggctgccccggccgcccatgctgctggcgctgctgttggcgacactgctggcggcgatgttggcgctgctgactcaggtggcgctggtggtgcaggtggcggaggcggctcgggccccgagcgtctcggccaagccggggccggcgctgtggcccctgccgctcttggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaacgggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 61 - DNA, 7034 bp, synthetic construct
  source 1..7034: mol_type = other DNA; note = pAAV-Cbh-ohHEXA-L1-ohHEXB-SV40 GA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgacatctagcagactgtggttcagcctgctgctggccgctgcttttgctggcagagctacagctctttggccctggcctcagaacttccagaccagcgaccagagatacgtgctgtaccccaacaacttccagttccagtacgacgtgtccagcgccgctcagcctggatgttctgtgctggatgaggccttccagcggtacagggatctgctgtttggcagcggctcttggcccagaccttacctgacaggcaagcggcacaccctggaaaagaacgtgctggtggtgtccgtggtcacccctggctgtaatcagctgcccacactggaaagcgtggaaaactacaccctgaccatcaacgacgaccagtgtctgctgctgagcgagacagtttggggagccctgagaggcctggaaaccttttctcagctcgtgtggaagtccgccgagggcaccttcttcatcaacaagaccgagatcgaggacttccccagatttccccacagaggactgctgctcgacaccagcagacattacctgcctctgagcagcatcctggataccctggacgtgatggcctacaacaagctgaacgtgttccactggcacctggtggacgaccctagcttcccttacgagagcttcacattccccgagctgatgcggaagggcagctacaaccctgtgacacacatctacacagcccaggacgtgaaagaagtgatcgagtacgcccggctgcggggcattagagtgctggccgaatttgacacccctggacacaccctgtcttggggccctggaattcctggactgctgaccccttgttacagcggcagcgagccttctggcacattcggccctgtgaaccccagcctgaacaacacctacgagtttatgagcacattctttctggaagtctccagcgtgttccccgacttctatctgcatctcggaggcgacgaggtggacttcacctgttggaagtctaaccccgagatccaggatttcatgcggaagaaaggcttcggcgaggatttcaagcagctggaaagcttctacatccagacactgctggacatcgtgtctagctacggcaagggctacgtcgtgtggcaagaggtgttcgacaacaaagtgaagatccagcctgacaccatcatccaagtgtggcgcgaggacatccccgtgaactacatgaaggaactcgagctggtcaccaaggccggctttagagcactgctgtctgccccttggtatctgaaccggatcagctacggccccgactggaaggatttctacatcgtggaacccctggccttcgagggcacacctgaacagaaggccctggttattggaggcgaggcctgtatgtggggcgagtacgtggacaacaccaacctggtgcctagactgtggcctagagctggcgctgtggccgaaagactctggtccaacaaactgacctccgacctgaccttcgcctacgagagactgagccacttcagatgcgagctgctgagaagaggcgtgcaggcccagcctctgaatgtgggcttctgcgagcaagagttcgagcagacaggtggcggaggatctggcggaggtggaagcggcggaggcggttctggtggtggtggatctgctagagcccctagcgtgtcagccaaacctggacctgctttgtggcctctgcctctgctggtcaagatgacccctaacctgctgcatctggcccctgagaacttttacatctctcacagccccaacagcaccgccggacctagctgtacactgctcgaggaagccttcagaagataccacggctacatcttcggcttctacaagtggcaccacgagcctgccgagttccaggccaaaacacaggtgcagcagctcctcgtgtctatcaccctgcagagcgagtgcgacgcctttcctaacatcagcagcgacgagagctataccctgcttgtgaaagaacccgtggccgtgctgaaggccaacagagtgtggggagcactgcggggactcgagacattttcccagctggtgtaccaggactcctacggcaccttcaccatcaatgagagcacaatcatcgacagccccagattcagccaccggggcatcctgatcgataccagcaggcactatctgcccgtgaagatcatcctgaaaacactggacgccatggcctttaacaagttcaatgtgctgcactggcatatcgtggacgatcagagcttcccctaccagtccatcacctttccagagctgagcaacaagggctcctacagcctgagccacgtgtacacccctaacgacgtgcggatggtcatcgagtatgccagactgagaggcatcagggtgctgcccgagttcgatacaccaggccatacactgagctggggcaagggccagaaagacctgctgacaccctgctactcccggcagaacaagctggacagctttggccccatcaatcccacactgaataccacctacagctttctgaccacctttttcaaagaaatcagcgaggtgttcccggaccagttcatccatctcggcggagatgaagtggaattcaagtgctgggagagcaaccccaagattcaggactttatgagacagaaaggattcgggaccgacttcaagaaacttgagagcttctatattcagaaggtcctggacatcattgccaccattaacaagggcagcattgtctggcaagaagtctttgacgacaaggccaagctggccccaggcacaatcgtggaagtgtggaaggacagcgcttaccccgaggaactgagcagagtgacagccagcggcttccctgtgattctgagcgctccctggtatctggatctgatctcttacggccaggactggcggaagtactacaaggtggaacctctggatttcggcggcacccagaagcagaagcagctgtttattggcggagaagcctgcctgtggggagaatatgtggacgccaccaatctgacccctcggttgtggcccagagcctctgcagtgggagagagactttggagcagcaaggacgtgcgcgacatggacgacgcctatgacagactgacccggcacagatgccggatggtggaaagaggaattgccgcacagcctctgtacgccggctactgcaaccacgagaatatgtagggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 62 - DNA, 7034 bp, synthetic construct
  source 1..7034: mol_type = other DNA; note = pAAV-Cbh-ohHEXA-L1-ohHEXB-SV40 IDT
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgacaagcagcagactgtggttctccctgcttttggcggcggcttttgccggacgggccacagccctgtggccgtggccgcaaaattttcagacgagcgaccaacggtatgttctttatccaaataactttcaatttcagtatgatgtgtcctccgctgcacagcctggctgcagcgtcctggacgaggcatttcagcgctatagagatctcctctttgggtctggaagctggcccagaccatacttgactggcaagaggcacaccctcgagaagaatgttttggtggtttctgtagtaacccctgggtgcaatcaacttccgacccttgagtcagtagagaattatacgttgacaataaatgacgatcaatgccttcttcttagtgagacggtttggggagcgctgcgagggcttgagacatttagccagcttgtatggaagagtgcggagggtaccttcttcataaataaaacggagatcgaagattttccgcgattccctcacaggggtctgcttctggataccagcaggcattatctgcctcttagcagcatactcgacacactggacgtgatggcgtacaacaaactcaacgtgttccactggcatctggtggacgacccaagctttccatacgagagttttacgtttccagagctcatgagaaagggtagttataatcccgtgacccatatatataccgcgcaggacgtgaaagaagtcattgagtacgcgcgactccgaggaattagagtactggcagagttcgacactcctgggcacacgctcagttgggggcctggaatacctggattgttgactccctgctatagtggcagtgagccctcagggacctttggtccggttaatcccagtcttaacaacacatacgagttcatgtctacgttttttctggaggtgtcttcagtattccctgacttctacctgcaccttgggggagatgaggttgacttcacttgttggaagtctaaccctgaaatacaggacttcatgcggaaaaaaggattcggggaggatttcaaacaactggagtccttttatattcagacccttttggacattgtttccagctatggcaaagggtatgtggtgtggcaggaagttttcgacaacaaagtcaagatccaaccggataccattatacaggtctggagggaggacatccccgtaaactacatgaaggaacttgaactggtcaccaaagctggctttagagcgctgctctctgcgccttggtatttgaataggattagttatgggcccgattggaaggacttttatatcgtcgagccgcttgcctttgagggaacccctgagcaaaaagcacttgtgattgggggagaggcttgtatgtggggagaatatgtcgataatacgaacctggtaccccggctgtggcctcgcgctggggccgtggcggagcgcctctggtccaataagttgacaagtgatttgaccttcgcttacgaacggttgtctcatttcagatgtgagctgctgagacgcggcgtacaagcacaacccctgaacgttggtttttgcgaacaagaattcgaacagaccggcgggggaggctccggaggcggggggagtggaggcgggggatcaggaggcggtggcagcgccagggcgccttcagtaagcgccaaacccggcccagccctgtggcctttgccactcctggtaaaaatgactcctaatctgctgcatctggcgcctgaaaatttttatataagccactctcctaatagtactgcaggtccctcttgcactttgctcgaagaagcttttcgaaggtatcacggttatatttttggattttataagtggcaccatgaacctgcggaattccaagcgaaaactcaagtccaacagttgctcgtaagtattacacttcagagcgagtgtgatgctttccctaatatcagttctgacgaaagttacacgctcctggtaaaggagcctgtcgctgtactgaaggcgaatcgcgtttggggcgccttgcgaggcctggaaactttcagtcagttggtgtatcaggattcctatgggacattcacgatcaatgaatccactattatagacagccctaggtttagtcataggggcatactgattgacacttctagacattacctgccagtcaaaatcatcctgaaaacacttgatgccatggcattcaataagtttaatgttctccattggcacatcgtggacgatcaaagctttccataccagtctataacatttccggagcttagtaacaaggggagctattcactctcacacgtttacaccccgaacgacgtgcgcatggttattgaatacgccagacttcgaggcattagagttcttccagaatttgatacaccaggccacacgctctcctggggcaaaggccagaaggacctgcttaccccatgttactcccgacaaaacaaactcgattcttttgggccaataaacccaacgttgaacactacgtactccttcctgactacgttttttaaggaaataagtgaggtattccctgaccagtttattcacttgggtggtgatgaagtcgaatttaagtgttgggagtcaaaccccaaaatacaagacttcatgcgccagaaggggtttgggaccgactttaaaaaactcgagtctttctacattcaaaaggttctcgatattatcgcaacaattaacaaagggagcattgtgtggcaagaggtatttgacgataaagccaaactggcgcccggaacgatagtcgaggtctggaaagacagcgcctatcccgaggagcttagtcgagtcacggcttcaggttttccagttatccttagtgccccgtggtacttggatttgatttcctacggtcaagactggcgcaagtattataaggttgaacctcttgattttggtgggacccaaaaacaaaagcagttgttcatcggtggggaagcttgcttgtggggtgagtacgtagacgcgactaatcttacgcctcgattgtggcctcgggcatctgcagttggggagcgcttgtggagctcaaaggacgtgcgagacatggatgacgcttatgatagactgacccgacataggtgtcgaatggtggagagaggtatagcggcccaacctctttatgccggatactgcaaccatgaaaacatgtaaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 63 - DNA, 7034 bp, synthetic construct
  source 1..7034: mol_type = other DNA; note = pAAV-Cbh-ohHEXA-L1-ohHEXB-SV40 NV
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgacctcatcccgcttgtggttctctctgttgttggcggccgcctttgcaggaagggctactgccttgtggccctggccacaaaatttccagaccagtgatcagcgatatgtactctatccaaataacttccagtttcaatacgacgtatccagcgccgctcagccaggctgtagcgtgctggatgaagcgtttcaacgctatcgagatctgttgttcgggtcaggttcttggcccaggccgtatctcactgggaaaaggcacaccctggaaaagaatgtgctggttgtcagtgtggttacaccaggatgcaatcagctcccaacacttgagagcgtggaaaactatactctcacaatcaacgacgatcagtgtctcctgctctccgaaacagtctggggcgctctgcgggggttggaaacgttctcacagctggtatggaagtccgccgagggaactttcttcatcaacaaaacggagatcgaagactttcccaggttcccacacagaggtctgcttctcgacactagccggcattaccttcccctgagctctattcttgacaccctcgacgtaatggcctataacaagctgaacgtgtttcattggcatctcgtggacgatccaagcttcccatacgagtccttcacattcccagagctgatgcgcaaagggtcatacaatccagtgacacatatttacacggcccaggatgtgaaagaggttattgagtacgccagactcaggggcattagggtgctggccgagtttgatacaccagggcataccctctcatggggccctggtatccctgggctgctgactccctgctattcaggaagtgagccgagtgggactttcgggccagtgaacccctctctgaacaacacatacgagttcatgtccacatttttcctcgaggtgagttcagtgttccccgatttttatcttcaccttggtggagacgaggttgacttcacctgttggaagagcaaccccgagattcaggacttcatgcgcaaaaaggggtttggagaggattttaagcagcttgagagcttttatatacagacactgctcgacatcgtgtcttcatatggaaaaggctatgtggtttggcaagaggtctttgacaacaaagtcaagatccagcctgacacgattatccaagtatggagagaggacatacccgtcaactacatgaaggagctggagctggtgaccaaggctgggtttagggccctgctctcagctccctggtacctgaaccgaatatcctatgggccggattggaaagacttctacatagtggaaccactggccttcgaagggactcctgagcagaaggcattggtgatcggcggtgaggcttgtatgtggggcgagtacgtagacaatacgaacctcgtgccaaggctttggccaagagctggtgcagttgcagagcgactgtggagcaataagctgacaagtgacctcacatttgcctacgagcggctgtcccactttcgctgtgaactcctgagacggggagttcaagcccagcctctgaatgttggcttttgcgagcaggagttcgaacagacagggggaggaggtagcgggggaggaggaagtgggggtggaggtagcggaggtggtggaagcgctagagcaccatcagttagtgccaaaccagggcctgcattgtggccactgcccctcctggtcaaaatgacacctaatctgctccatcttgccccggagaatttctatatcagccactcccccaattcaaccgccgggccctcatgcaccctcctcgaagaggcctttcggagataccatgggtatatattcggcttctacaaatggcatcacgaacctgctgagttccaggccaagacccaagtccaacagttgctggtgtcaatcacactgcagagcgaatgcgatgcttttcccaacatttcttccgacgagtcttacacgctccttgtgaaggagccagtagctgtcttgaaggccaatcgcgtgtggggtgccttgaggggcttggaaacattttcccagctggtctaccaggattcctatgggacctttaccatcaacgaaagtacgataattgacagtcctcggttctcacacaggggcatcttgatcgatacaagcaggcattacttgccagtcaagatcatccttaagacactggatgccatggctttcaataagttcaacgtgcttcattggcacattgtggatgatcagagcttcccataccagagcattaccttcccagagctttccaataagggtagttattctctcagccacgtgtacacacccaatgatgtgcgcatggtcatcgagtatgcccggctgagagggattagggtcctgcccgaatttgacacgccaggccacacactctcctggggcaagggccagaaggatcttttgactccatgttattcaagacaaaataagttggacagctttggacccatcaaccccacccttaacacaacttactcattcctgaccacattctttaaggagatcagtgaggtcttccccgaccagtttattcatctcggtggcgatgaggtcgagtttaaatgctgggaaagcaaccccaagatccaggacttcatgcggcaaaagggattcggaactgatttcaaaaagctcgagtccttctatattcagaaggtcttggacatcatcgctaccataaacaaggggagcatcgtttggcaggaggtgttcgacgataaagccaagctggctccagggactattgtcgaggtgtggaaagatagtgcctaccccgaggaactttcacgggtgaccgcctctggttttcctgtgatcctctcagcaccgtggtatcttgatctcatctcttacggacaggactggcggaagtactacaaagttgaaccactggattttgggggaacgcagaaacaaaagcagctgtttatcggaggcgaagcatgtctgtggggagagtatgtagatgcaaccaaccttaccccacggctgtggccacgggcttctgctgtaggtgagaggctctggagcagcaaagacgttcgcgacatggacgacgcatatgaccggcttactagacaccgctgcagaatggttgagaggggaattgccgcccagcccctttatgccggatattgcaatcatgaaaatatgtaaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 64 - DNA, 1586 bp, synthetic construct
  source 1..1586: mol_type = other DNA; note = GA optimized nucleotide sequence of alfa subunit of beta hexosaminidase
tgacatctagcagactgtggttcagcctgctgctggccgctgcttttgctggcagagctacagctctttggccctggcctcagaacttccagaccagcgaccagagatacgtgctgtaccccaacaacttccagttccagtacgacgtgtccagcgccgctcagcctggatgttctgtgctggatgaggccttccagcggtacagggatctgctgtttggcagcggctcttggcccagaccttacctgacaggcaagcggcacaccctggaaaagaacgtgctggtggtgtccgtggtcacccctggctgtaatcagctgcccacactggaaagcgtggaaaactacaccctgaccatcaacgacgaccagtgtctgctgctgagcgagacagtttggggagccctgagaggcctggaaaccttttctcagctcgtgtggaagtccgccgagggcaccttcttcatcaacaagaccgagatcgaggacttccccagatttccccacagaggactgctgctcgacaccagcagacattacctgcctctgagcagcatcctggataccctggacgtgatggcctacaacaagctgaacgtgttccactggcacctggtggacgaccctagcttcccttacgagagcttcacattccccgagctgatgcggaagggcagctacaaccctgtgacacacatctacacagcccaggacgtgaaagaagtgatcgagtacgcccggctgcggggcattagagtgctggccgaatttgacacccctggacacaccctgtcttggggccctggaattcctggactgctgaccccttgttacagcggcagcgagccttctggcacattcggccctgtgaaccccagcctgaacaacacctacgagtttatgagcacattctttctggaagtctccagcgtgttccccgacttctatctgcatctcggaggcgacgaggtggacttcacctgttggaagtctaaccccgagatccaggatttcatgcggaagaaaggcttcggcgaggatttcaagcagctggaaagcttctacatccagacactgctggacatcgtgtctagctacggcaagggctacgtcgtgtggcaagaggtgttcgacaacaaagtgaagatccagcctgacaccatcatccaagtgtggcgcgaggacatccccgtgaactacatgaaggaactcgagctggtcaccaaggccggctttagagcactgctgtctgccccttggtatctgaaccggatcagctacggccccgactggaaggatttctacatcgtggaacccctggccttcgagggcacacctgaacagaaggccctggttattggaggcgaggcctgtatgtggggcgagtacgtggacaacaccaacctggtgcctagactgtggcctagagctggcgctgtggccgaaagactctggtccaacaaactgacctccgacctgaccttcgcctacgagagactgagccacttcagatgcgagctgctgagaagaggcgtgcaggcccagcctctgaatgtgggcttctgcgagcaagagttcgagcagaca

SEQ ID NO: 65 - DNA, 1545 bp, synthetic construct
  source 1..1545: mol_type = other DNA; note = GA optimized nucleotide sequence of beta subunit of beta hexosaminidase
gctagagcccctagcgtgtcagccaaacctggacctgctttgtggcctctgcctctgctggtcaagatgacccctaacctgctgcatctggcccctgagaacttttacatctctcacagccccaacagcaccgccggacctagctgtacactgctcgaggaagccttcagaagataccacggctacatcttcggcttctacaagtggcaccacgagcctgccgagttccaggccaaaacacaggtgcagcagctcctcgtgtctatcaccctgcagagcgagtgcgacgcctttcctaacatcagcagcgacgagagctataccctgcttgtgaaagaacccgtggccgtgctgaaggccaacagagtgtggggagcactgcggggactcgagacattttcccagctggtgtaccaggactcctacggcaccttcaccatcaatgagagcacaatcatcgacagccccagattcagccaccggggcatcctgatcgataccagcaggcactatctgcccgtgaagatcatcctgaaaacactggacgccatggcctttaacaagttcaatgtgctgcactggcatatcgtggacgatcagagcttcccctaccagtccatcacctttccagagctgagcaacaagggctcctacagcctgagccacgtgtacacccctaacgacgtgcggatggtcatcgagtatgccagactgagaggcatcagggtgctgcccgagttcgatacaccaggccatacactgagctggggcaagggccagaaagacctgctgacaccctgctactcccggcagaacaagctggacagctttggccccatcaatcccacactgaataccacctacagctttctgaccacctttttcaaagaaatcagcgaggtgttcccggaccagttcatccatctcggcggagatgaagtggaattcaagtgctgggagagcaaccccaagattcaggactttatgagacagaaaggattcgggaccgacttcaagaaacttgagagcttctatattcagaaggtcctggacatcattgccaccattaacaagggcagcattgtctggcaagaagtctttgacgacaaggccaagctggccccaggcacaatcgtggaagtgtggaaggacagcgcttaccccgaggaactgagcagagtgacagccagcggcttccctgtgattctgagcgctccctggtatctggatctgatctcttacggccaggactggcggaagtactacaaggtggaacctctggatttcggcggcacccagaagcagaagcagctgtttattggcggagaagcctgcctgtggggagaatatgtggacgccaccaatctgacccctcggttgtggcccagagcctctgcagtgggagagagactttggagcagcaaggacgtgcgcgacatggacgacgcctatgacagactgacccggcacagatgccggatggtggaaagaggaattgccgcacagcctctgtacgccggctactgcaaccacgagaatatgtag

SEQ ID NO: 66 - DNA, 1587 bp, synthetic construct
  source 1..1587: mol_type = other DNA; note = IDT optimized nucleotide sequence of alfa subunit of beta hexosaminidase
atgacaagcagcagactgtggttctccctgcttttggcggcggcttttgccggacgggccacagccctgtggccgtggccgcaaaattttcagacgagcgaccaacggtatgttctttatccaaataactttcaatttcagtatgatgtgtcctccgctgcacagcctggctgcagcgtcctggacgaggcatttcagcgctatagagatctcctctttgggtctggaagctggcccagaccatacttgactggcaagaggcacaccctcgagaagaatgttttggtggtttctgtagtaacccctgggtgcaatcaacttccgacccttgagtcagtagagaattatacgttgacaataaatgacgatcaatgccttcttcttagtgagacggtttggggagcgctgcgagggcttgagacatttagccagcttgtatggaagagtgcggagggtaccttcttcataaataaaacggagatcgaagattttccgcgattccctcacaggggtctgcttctggataccagcaggcattatctgcctcttagcagcatactcgacacactggacgtgatggcgtacaacaaactcaacgtgttccactggcatctggtggacgacccaagctttccatacgagagttttacgtttccagagctcatgagaaagggtagttataatcccgtgacccatatatataccgcgcaggacgtgaaagaagtcattgagtacgcgcgactccgaggaattagagtactggcagagttcgacactcctgggcacacgctcagttgggggcctggaatacctggattgttgactccctgctatagtggcagtgagccctcagggacctttggtccggttaatcccagtcttaacaacacatacgagttcatgtctacgttttttctggaggtgtcttcagtattccctgacttctacctgcaccttgggggagatgaggttgacttcacttgttggaagtctaaccctgaaatacaggacttcatgcggaaaaaaggattcggggaggatttcaaacaactggagtccttttatattcagacccttttggacattgtttccagctatggcaaagggtatgtggtgtggcaggaagttttcgacaacaaagtcaagatccaaccggataccattatacaggtctggagggaggacatccccgtaaactacatgaaggaacttgaactggtcaccaaagctggctttagagcgctgctctctgcgccttggtatttgaataggattagttatgggcccgattggaaggacttttatatcgtcgagccgcttgcctttgagggaacccctgagcaaaaagcacttgtgattgggggagaggcttgtatgtggggagaatatgtcgataatacgaacctggtaccccggctgtggcctcgcgctggggccgtggcggagcgcctctggtccaataagttgacaagtgatttgaccttcgcttacgaacggttgtctcatttcagatgtgagctgctgagacgcggcgtacaagcacaacccctgaacgttggtttttgcgaacaagaattcgaacagacc

SEQ ID NO: 67 - DNA, 1544 bp, synthetic construct
  source 1..1544: mol_type = other DNA; note = IDT optimized nucleotide sequence of beta subunit of beta hexosaminidase
ccagggcgccttcagtaagcgccaaacccggcccagccctgtggcctttgccactcctggtaaaaatgactcctaatctgctgcatctggcgcctgaaaatttttatataagccactctcctaatagtactgcaggtccctcttgcactttgctcgaagaagcttttcgaaggtatcacggttatatttttggattttataagtggcaccatgaacctgcggaattccaagcgaaaactcaagtccaacagttgctcgtaagtattacacttcagagcgagtgtgatgctttccctaatatcagttctgacgaaagttacacgctcctggtaaaggagcctgtcgctgtactgaaggcgaatcgcgtttggggcgccttgcgaggcctggaaactttcagtcagttggtgtatcaggattcctatgggacattcacgatcaatgaatccactattatagacagccctaggtttagtcataggggcatactgattgacacttctagacattacctgccagtcaaaatcatcctgaaaacacttgatgccatggcattcaataagtttaatgttctccattggcacatcgtggacgatcaaagctttccataccagtctataacatttccggagcttagtaacaaggggagctattcactctcacacgtttacaccccgaacgacgtgcgcatggttattgaatacgccagacttcgaggcattagagttcttccagaatttgatacaccaggccacacgctctcctggggcaaaggccagaaggacctgcttaccccatgttactcccgacaaaacaaactcgattcttttgggccaataaacccaacgttgaacactacgtactccttcctgactacgttttttaaggaaataagtgaggtattccctgaccagtttattcacttgggtggtgatgaagtcgaatttaagtgttgggagtcaaaccccaaaatacaagacttcatgcgccagaaggggtttgggaccgactttaaaaaactcgagtctttctacattcaaaaggttctcgatattatcgcaacaattaacaaagggagcattgtgtggcaagaggtatttgacgataaagccaaactggcgcccggaacgatagtcgaggtctggaaagacagcgcctatcccgaggagcttagtcgagtcacggcttcaggttttccagttatccttagtgccccgtggtacttggatttgatttcctacggtcaagactggcgcaagtattataaggttgaacctcttgattttggtgggacccaaaaacaaaagcagttgttcatcggtggggaagcttgcttgtggggtgagtacgtagacgcgactaatcttacgcctcgattgtggcctcgggcatctgcagttggggagcgcttgtggagctcaaaggacgtgcgagacatggatgacgcttatgatagactgacccgacataggtgtcgaatggtggagagaggtatagcggcccaacctctttatgccggatactgcaaccatgaaaacatgtaa

SEQ ID NO: 68 - DNA, 1587 bp, synthetic construct
  source 1..1587: mol_type = other DNA; note = NV optimized nucleotide sequence of alfa subunit of beta hexosaminidase
atgacctcatcccgcttgtggttctctctgttgttggcggccgcctttgcaggaagggctactgccttgtggccctggccacaaaatttccagaccagtgatcagcgatatgtactctatccaaataacttccagtttcaatacgacgtatccagcgccgctcagccaggctgtagcgtgctggatgaagcgtttcaacgctatcgagatctgttgttcgggtcaggttcttggcccaggccgtatctcactgggaaaaggcacaccctggaaaagaatgtgctggttgtcagtgtggttacaccaggatgcaatcagctcccaacacttgagagcgtggaaaactatactctcacaatcaacgacgatcagtgtctcctgctctccgaaacagtctggggcgctctgcgggggttggaaacgttctcacagctggtatggaagtccgccgagggaactttcttcatcaacaaaacggagatcgaagactttcccaggttcccacacagaggtctgcttctcgacactagccggcattaccttcccctgagctctattcttgacaccctcgacgtaatggcctataacaagctgaacgtgtttcattggcatctcgtggacgatccaagcttcccatacgagtccttcacattcccagagctgatgcgcaaagggtcatacaatccagtgacacatatttacacggcccaggatgtgaaagaggttattgagtacgccagactcaggggcattagggtgctggccgagtttgatacaccagggcataccctctcatggggccctggtatccctgggctgctgactccctgctattcaggaagtgagccgagtgggactttcgggccagtgaacccctctctgaacaacacatacgagttcatgtccacatttttcctcgaggtgagttcagtgttccccgatttttatcttcaccttggtggagacgaggttgacttcacctgttggaagagcaaccccgagattcaggacttcatgcgcaaaaaggggtttggagaggattttaagcagcttgagagcttttatatacagacactgctcgacatcgtgtcttcatatggaaaaggctatgtggtttggcaagaggtctttgacaacaaagtcaagatccagcctgacacgattatccaagtatggagagaggacatacccgtcaactacatgaaggagctggagctggtgaccaaggctgggtttagggccctgctctcagctccctggtacctgaaccgaatatcctatgggccggattggaaagacttctacatagtggaaccactggccttcgaagggactcctgagcagaaggcattggtgatcggcggtgaggcttgtatgtggggcgagtacgtagacaatacgaacctcgtgccaaggctttggccaagagctggtgcagttgcagagcgactgtggagcaataagctgacaagtgacctcacatttgcctacgagcggctgtcccactttcgctgtgaactcctgagacggggagttcaagcccagcctctgaatgttggcttttgcgagcaggagttcgaacagaca

SEQ ID NO: 69 - DNA, 1545 bp, synthetic construct
  source 1..1545: mol_type = other DNA; note = NV optimized nucleotide sequence of beta subunit of beta hexosaminidase
gctagagcaccatcagttagtgccaaaccagggcctgcattgtggccactgcccctcctggtcaaaatgacacctaatctgctccatcttgccccggagaatttctatatcagccactcccccaattcaaccgccgggccctcatgcaccctcctcgaagaggcctttcggagataccatgggtatatattcggcttctacaaatggcatcacgaacctgctgagttccaggccaagacccaagtccaacagttgctggtgtcaatcacactgcagagcgaatgcgatgcttttcccaacatttcttccgacgagtcttacacgctccttgtgaaggagccagtagctgtcttgaaggccaatcgcgtgtggggtgccttgaggggcttggaaacattttcccagctggtctaccaggattcctatgggacctttaccatcaacgaaagtacgataattgacagtcctcggttctcacacaggggcatcttgatcgatacaagcaggcattacttgccagtcaagatcatccttaagacactggatgccatggctttcaataagttcaacgtgcttcattggcacattgtggatgatcagagcttcccataccagagcattaccttcccagagctttccaataagggtagttattctctcagccacgtgtacacacccaatgatgtgcgcatggtcatcgagtatgcccggctgagagggattagggtcctgcccgaatttgacacgccaggccacacactctcctggggcaagggccagaaggatcttttgactccatgttattcaagacaaaataagttggacagctttggacccatcaaccccacccttaacacaacttactcattcctgaccacattctttaaggagatcagtgaggtcttccccgaccagtttattcatctcggtggcgatgaggtcgagtttaaatgctgggaaagcaaccccaagatccaggacttcatgcggcaaaagggattcggaactgatttcaaaaagctcgagtccttctatattcagaaggtcttggacatcatcgctaccataaacaaggggagcatcgtttggcaggaggtgttcgacgataaagccaagctggctccagggactattgtcgaggtgtggaaagatagtgcctaccccgaggaactttcacgggtgaccgcctctggttttcctgtgatcctctcagcaccgtggtatcttgatctcatctcttacggacaggactggcggaagtactacaaagttgaaccactggattttgggggaacgcagaaacaaaagcagctgtttatcggaggcgaagcatgtctgtggggagagtatgtagatgcaaccaaccttaccccacggctgtggccacgggcttctgctgtaggtgagaggctctggagcagcaaagacgttcgcgacatggacgacgcatatgaccggcttactagacaccgctgcagaatggttgagaggggaattgccgcccagcccctttatgccggatattgcaatcatgaaaatatgtaa

SEQ ID NO: 70 - protein, 1063 aa, synthetic construct
  source 1..1063: note = covalently linked human alfa beta dimer of beta hexosaminidase L1
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQTGGGGSGGGGSGGGGSGGGGSARAPSVSAKPGPALWPLPLLVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 71 - DNA, 3192 bp, synthetic construct
  source 1..3192: mol_type = other DNA; note = covalently linked human alfa beta dimer of beta hexosaminidase L1
atgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagaccggtggtggtggatctggaggcggaggatcaggcggcggaggttctggaggtggaggtagtgctcgggccccgagcgtctcggccaagccggggccggcgctgtggcccctgccgctcttggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaa

SEQ ID NO: 72 - DNA, 3192 bp, synthetic construct
  source 1..3192: mol_type = other DNA; note = covalently linked GA optimized alfa beta dimer of beta hexosaminidase L1
atgacatctagcagactgtggttcagcctgctgctggccgctgcttttgctggcagagctacagctctttggccctggcctcagaacttccagaccagcgaccagagatacgtgctgtaccccaacaacttccagttccagtacgacgtgtccagcgccgctcagcctggatgttctgtgctggatgaggccttccagcggtacagggatctgctgtttggcagcggctcttggcccagaccttacctgacaggcaagcggcacaccctggaaaagaacgtgctggtggtgtccgtggtcacccctggctgtaatcagctgcccacactggaaagcgtggaaaactacaccctgaccatcaacgacgaccagtgtctgctgctgagcgagacagtttggggagccctgagaggcctggaaaccttttctcagctcgtgtggaagtccgccgagggcaccttcttcatcaacaagaccgagatcgaggacttccccagatttccccacagaggactgctgctcgacaccagcagacattacctgcctctgagcagcatcctggataccctggacgtgatggcctacaacaagctgaacgtgttccactggcacctggtggacgaccctagcttcccttacgagagcttcacattccccgagctgatgcggaagggcagctacaaccctgtgacacacatctacacagcccaggacgtgaaagaagtgatcgagtacgcccggctgcggggcattagagtgctggccgaatttgacacccctggacacaccctgtcttggggccctggaattcctggactgctgaccccttgttacagcggcagcgagccttctggcacattcggccctgtgaaccccagcctgaacaacacctacgagtttatgagcacattctttctggaagtctccagcgtgttccccgacttctatctgcatctcggaggcgacgaggtggacttcacctgttggaagtctaaccccgagatccaggatttcatgcggaagaaaggcttcggcgaggatttcaagcagctggaaagcttctacatccagacactgctggacatcgtgtctagctacggcaagggctacgtcgtgtggcaagaggtgttcgacaacaaagtgaagatccagcctgacaccatcatccaagtgtggcgcgaggacatccccgtgaactacatgaaggaactcgagctggtcaccaaggccggctttagagcactgctgtctgccccttggtatctgaaccggatcagctacggccccgactggaaggatttctacatcgtggaacccctggccttcgagggcacacctgaacagaaggccctggttattggaggcgaggcctgtatgtggggcgagtacgtggacaacaccaacctggtgcctagactgtggcctagagctggcgctgtggccgaaagactctggtccaacaaactgacctccgacctgaccttcgcctacgagagactgagccacttcagatgcgagctgctgagaagaggcgtgcaggcccagcctctgaatgtgggcttctgcgagcaagagttcgagcagacaggtggcggaggatctggcggaggtggaagcggcggaggcggttctggtggtggtggatctgctagagcccctagcgtgtcagccaaacctggacctgctttgtggcctctgcctctgctggtcaagatgacccctaacctgctgcatctggcccctgagaacttttacatctctcacagccccaacagcaccgccggacctagctgtacactgctcgaggaagccttcagaagataccacggctacatcttcggcttctacaagtggcaccacgagcctgccgagttccaggccaaaacacaggtgcagcagctcctcgtgtctatcaccctgcagagcgagtgcgacgcctttcctaacatcagcagcgacgagagctataccctgcttgtgaaagaacccgtggccgtgctgaaggccaacagagtgtggggagcactgcggggactcgagacattttcccagctggtgtaccaggactcctacggcaccttcaccatcaatgagagcacaatcatcgacagccccagattcagccaccggggcatcctgatcgataccagcaggcactatctgcccgtgaagatcatcctgaaaacactggacgccatggcctttaacaagttcaatgtgctgcactggcatatcgtggacgatcagagcttcccctaccagtccatcacctttccagagctgagcaacaagggctcctacagcctgagccacgtgtacacccctaacgacgtgcggatggtcatcgagtatgccagactgagaggcatcagggtgctgcccgagttcgatacaccaggccatacactgagctggggcaagggccagaaagacctgctgacaccctgctactcccggcagaacaagctggacagctttggccccatcaatcccacactgaataccacctacagctttctgaccacctttttcaaagaaatcagcgaggtgttcccggaccagttcatccatctcggcggagatgaagtggaattcaagtgctgggagagcaaccccaagattcaggactttatgagacagaaaggattcgggaccgacttcaagaaacttgagagcttctatattcagaaggtcctggacatcattgccaccattaacaagggcagcattgtctggcaagaagtctttgacgacaaggccaagctggccccaggcacaatcgtggaagtgtggaaggacagcgcttaccccgaggaactgagcagagtgacagccagcggcttccctgtgattctgagcgctccctggtatctggatctgatctcttacggccaggactggcggaagtactacaaggtggaacctctggatttcggcggcacccagaagcagaagcagctgtttattggcggagaagcctgcctgtggggagaatatgtggacgccaccaatctgacccctcggttgtggcccagagcctctgcagtgggagagagactttggagcagcaaggacgtgcgcgacatggacgacgcctatgacagactgacccggcacagatgccggatggtggaaagaggaattgccgcacagcctctgtacgccggctactgcaaccacgagaatatgtag

SEQ ID NO: 73 - DNA, 3192 bp, synthetic construct
  source 1..3192: mol_type = other DNA; note = covalently linked IDT optimized alfa beta dimer of beta hexosaminidase L1
atgacaagcagcagactgtggttctccctgcttttggcggcggcttttgccggacgggccacagccctgtggccgtggccgcaaaattttcagacgagcgaccaacggtatgttctttatccaaataactttcaatttcagtatgatgtgtcctccgctgcacagcctggctgcagcgtcctggacgaggcatttcagcgctatagagatctcctctttgggtctggaagctggcccagaccatacttgactggcaagaggcacaccctcgagaagaatgttttggtggtttctgtagtaacccctgggtgcaatcaacttccgacccttgagtcagtagagaattatacgttgacaataaatgacgatcaatgccttcttcttagtgagacggtttggggagcgctgcgagggcttgagacatttagccagcttgtatggaagagtgcggagggtaccttcttcataaataaaacggagatcgaagattttccgcgattccctcacaggggtctgcttctggataccagcaggcattatctgcctcttagcagcatactcgacacactggacgtgatggcgtacaacaaactcaacgtgttccactggcatctggtggacgacccaagctttccatacgagagttttacgtttccagagctcatgagaaagggtagttataatcccgtgacccatatatataccgcgcaggacgtgaaagaagtcattgagtacgcgcgactccgaggaattagagtactggcagagttcgacactcctgggcacacgctcagttgggggcctggaatacctggattgttgactccctgctatagtggcagtgagccctcagggacctttggtccggttaatcccagtcttaacaacacatacgagttcatgtctacgttttttctggaggtgtcttcagtattccctgacttctacctgcaccttgggggagatgaggttgacttcacttgttggaagtctaaccctgaaatacaggacttcatgcggaaaaaaggattcggggaggatttcaaacaactggagtccttttatattcagacccttttggacattgtttccagctatggcaaagggtatgtggtgtggcaggaagttttcgacaacaaagtcaagatccaaccggataccattatacaggtctggagggaggacatccccgtaaactacatgaaggaacttgaactggtcaccaaagctggctttagagcgctgctctctgcgccttggtatttgaataggattagttatgggcccgattggaaggacttttatatcgtcgagccgcttgcctttgagggaacccctgagcaaaaagcacttgtgattgggggagaggcttgtatgtggggagaatatgtcgataatacgaacctggtaccccggctgtggcctcgcgctggggccgtggcggagcgcctctggtccaataagttgacaagtgatttgaccttcgcttacgaacggttgtctcatttcagatgtgagctgctgagacgcggcgtacaagcacaacccctgaacgttggtttttgcgaacaagaattcgaacagaccggcgggggaggctccggaggcggggggagtggaggcgggggatcaggaggcggtggcagcgccagggcgccttcagtaagcgccaaacccggcccagccctgtggcctttgccactcctggtaaaaatgactcctaatctgctgcatctggcgcctgaaaatttttatataagccactctcctaatagtactgcaggtccctcttgcactttgctcgaagaagcttttcgaaggtatcacggttatatttttggattttataagtggcaccatgaacctgcggaattccaagcgaaaactcaagtccaacagttgctcgtaagtattacacttcagagcgagtgtgatgctttccctaatatcagttctgacgaaagttacacgctcctggtaaaggagcctgtcgctgtactgaaggcgaatcgcgtttggggcgccttgcgaggcctggaaactttcagtcagttggtgtatcaggattcctatgggacattcacgatcaatgaatccactattatagacagccctaggtttagtcataggggcatactgattgacacttctagacattacctgccagtcaaaatcatcctgaaaacacttgatgccatggcattcaataagtttaatgttctccattggcacatcgtggacgatcaaagctttccataccagtctataacatttccggagcttagtaacaaggggagctattcactctcacacgtttacaccccgaacgacgtgcgcatggttattgaatacgccagacttcgaggcattagagttcttccagaatttgatacaccaggccacacgctctcctggggcaaaggccagaaggacctgcttaccccatgttactcccgacaaaacaaactcgattcttttgggccaataaacccaacgttgaacactacgtactccttcctgactacgttttttaaggaaataagtgaggtattccctgaccagtttattcacttgggtggtgatgaagtcgaatttaagtgttgggagtcaaaccccaaaatacaagacttcatgcgccagaaggggtttgggaccgactttaaaaaactcgagtctttctacattcaaaaggttctcgatattatcgcaacaattaacaaagggagcattgtgtggcaagaggtatttgacgataaagccaaactggcgcccggaacgatagtcgaggtctggaaagacagcgcctatcccgaggagcttagtcgagtcacggcttcaggttttccagttatccttagtgccccgtggtacttggatttgatttcctacggtcaagactggcgcaagtattataaggttgaacctcttgattttggtgggacccaaaaacaaaagcagttgttcatcggtggggaagcttgcttgtggggtgagtacgtagacgcgactaatcttacgcctcgattgtggcctcgggcatctgcagttggggagcgcttgtggagctcaaaggacgtgcgagacatggatgacgcttatgatagactgacccgacataggtgtcgaatggtggagagaggtatagcggcccaacctctttatgccggatactgcaaccatgaaaacatgtaa

SEQ ID NO: 74 - DNA, 3192 bp, synthetic construct
  source 1..3192: mol_type = other DNA; note = covalently linked NV optimized alfa beta dimer of beta hexosaminidase L1
atgacctcatcccgcttgtggttctctctgttgttggcggccgcctttgcaggaagggctactgccttgtggccctggccacaaaatttccagaccagtgatcagcgatatgtactctatccaaataacttccagtttcaatacgacgtatccagcgccgctcagccaggctgtagcgtgctggatgaagcgtttcaacgctatcgagatctgttgttcgggtcaggttcttggcccaggccgtatctcactgggaaaaggcacaccctggaaaagaatgtgctggttgtcagtgtggttacaccaggatgcaatcagctcccaacacttgagagcgtggaaaactatactctcacaatcaacgacgatcagtgtctcctgctctccgaaacagtctggggcgctctgcgggggttggaaacgttctcacagctggtatggaagtccgccgagggaactttcttcatcaacaaaacggagatcgaagactttcccaggttcccacacagaggtctgcttctcgacactagccggcattaccttcccctgagctctattcttgacaccctcgacgtaatggcctataacaagctgaacgtgtttcattggcatctcgtggacgatccaagcttcccatacgagtccttcacattcccagagctgatgcgcaaagggtcatacaatccagtgacacatatttacacggcccaggatgtgaaagaggttattgagtacgccagactcaggggcattagggtgctggccgagtttgatacaccagggcataccctctcatggggccctggtatccctgggctgctgactccctgctattcaggaagtgagccgagtgggactttcgggccagtgaacccctctctgaacaacacatacgagttcatgtccacatttttcctcgaggtgagttcagtgttccccgatttttatcttcaccttggtggagacgaggttgacttcacctgttggaagagcaaccccgagattcaggacttcatgcgcaaaaaggggtttggagaggattttaagcagcttgagagcttttatatacagacactgctcgacatcgtgtcttcatatggaaaaggctatgtggtttggcaagaggtctttgacaacaaagtcaagatccagcctgacacgattatccaagtatggagagaggacatacccgtcaactacatgaaggagctggagctggtgaccaaggctgggtttagggccctgctctcagctccctggtacctgaaccgaatatcctatgggccggattggaaagacttctacatagtggaaccactggccttcgaagggactcctgagcagaaggcattggtgatcggcggtgaggcttgtatgtggggcgagtacgtagacaatacgaacctcgtgccaaggctttggccaagagctggtgcagttgcagagcgactgtggagcaataagctgacaagtgacctcacatttgcctacgagcggctgtcccactttcgctgtgaactcctgagacggggagttcaagcccagcctctgaatgttggcttttgcgagcaggagttcgaacagacagggggaggaggtagcgggggaggaggaagtgggggtggaggtagcggaggtggtggaagcgctagagcaccatcagttagtgccaaaccagggcctgcattgtggccactgcccctcctggtcaaaatgacacctaatctgctccatcttgccccggagaatttctatatcagccactcccccaattcaaccgccgggccctcatgcaccctcctcgaagaggcctttcggagataccatgggtatatattcggcttctacaaatggcatcacgaacctgctgagttccaggccaagacccaagtccaacagttgctggtgtcaatcacactgcagagcgaatgcgatgcttttcccaacatttcttccgacgagtcttacacgctccttgtgaaggagccagtagctgtcttgaaggccaatcgcgtgtggggtgccttgaggggcttggaaacattttcccagctggtctaccaggattcctatgggacctttaccatcaacgaaagtacgataattgacagtcctcggttctcacacaggggcatcttgatcgatacaagcaggcattacttgccagtcaagatcatccttaagacactggatgccatggctttcaataagttcaacgtgcttcattggcacattgtggatgatcagagcttcccataccagagcattaccttcccagagctttccaataagggtagttattctctcagccacgtgtacacacccaatgatgtgcgcatggtcatcgagtatgcccggctgagagggattagggtcctgcccgaatttgacacgccaggccacacactctcctggggcaagggccagaaggatcttttgactccatgttattcaagacaaaataagttggacagctttggacccatcaaccccacccttaacacaacttactcattcctgaccacattctttaaggagatcagtgaggtcttccccgaccagtttattcatctcggtggcgatgaggtcgagtttaaatgctgggaaagcaaccccaagatccaggacttcatgcggcaaaagggattcggaactgatttcaaaaagctcgagtccttctatattcagaaggtcttggacatcatcgctaccataaacaaggggagcatcgtttggcaggaggtgttcgacgataaagccaagctggctccagggactattgtcgaggtgtggaaagatagtgcctaccccgaggaactttcacgggtgaccgcctctggttttcctgtgatcctctcagcaccgtggtatcttgatctcatctcttacggacaggactggcggaagtactacaaagttgaaccactggattttgggggaacgcagaaacaaaagcagctgtttatcggaggcgaagcatgtctgtggggagagtatgtagatgcaaccaaccttaccccacggctgtggccacgggcttctgctgtaggtgagaggctctggagcagcaaagacgttcgcgacatggacgacgcatatgaccggcttactagacaccgctgcagaatggttgagaggggaattgccgcccagcccctttatgccggatattgcaatcatgaaaatatgtaa

SEQ ID NO: 75 - DNA, 4407 bp, synthetic construct
  source 1..4407: mol_type = other DNA; note = L1 AB mouse gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacaggtggtggtggatctggaggcggaggatcaggcggcggaggttctggaggtggaggtagtcagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 76 - DNA, 4446 bp, synthetic construct
  source 1..4446: mol_type = other DNA; note = L2 AB mouse gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacatctggaggatctagcggaggatcctctggcagcgagacaccaggaacaagcgagtcagcaacaccagagagcagtggcggcagcagcggcggcagcagccagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 77 - DNA, 4383 bp, synthetic construct
  source 1..4383: mol_type = other DNA; note = L3 AB mouse gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacaggcagcgccggcagcgccgccggcagcggcgagttccagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 78 - DNA, 4236 bp, synthetic construct
  source 1..4236: mol_type = other DNA; note = L1 BA mouse gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgcctcagagccctagatctgctcctggactgctgctgctccaggctctggtgtctcttgtgtctctggcactggtggcccctgctagactgcaacctgctctgtggccatttcctagaagcgtgcagatgttccccagactgctgtacatcagcgccgaggacttcagcatcgatcacagccccaacagcacagctggccctagttgtagcctgctgcaagaggccttcagacggtactacaactacgtgttcggcttctacaagaggcaccacggacctgccagattcagagctgagccccagctgcagaaactgctggtgtctatcaccctggaaagcgagtgcgagagcttccctagcctgagcagcgacgagacatacagcctgctggtgcaagagcctgtggccgtgctgaaggctaactctgtttggggcgctctgagaggcctggaaaccttctctcagctggtgtaccaggacagcttcggcaccttcaccatcaacgagtcctctatcgccgactctcctagattccctcacagaggcatcctgatcgataccagcagacacttcctgcctgtgaaaaccatcctgaaaaccctggacgccatggccttcaacaagttcaacgtgctgcactggcacatcgtggacgaccagagctttccctaccagtccaccacctttccagagctgtccaacaagggcagctacagcctgagccacgtgtacacccctaacgacgtgcggatggtgctggaatacgccagactgagaggcatcagagtgatccccgagttcgacacccctggccacacacagtcttggggcaagggacagaagaacctgctgaccccttgctacaaccagaaaacaaagacccaggtgttcggccctgtggaccctaccgtgaacaccacctacgctttcttcaacaccttcttcaaagaaatcagcagcgtgttccccgaccagttcatccacctcggcggagatgaggtcgagttccagtgttgggccagcaatcccaacatccagggctttatgaagaggaaaggcttcggcagcgacttcagaaggctggaaagcttctacatcaagaagatcctcgagatcatcagcagcctgaagaagaacagcatcgtctggcaagaggtgttcgacgacaaggtggaactgcagcctggcaccgtggtggaagtgtggaagtctgagcactacagctacgagctgaagcaagtgaccggctctggcttccctgccatcctttctgccccttggtatctggacctgatcagctacggccaggactggaagaactactacaaggtcgagcccctgaacttcgagggcagcgagaagcagaagcagctggttatcggaggcgaggcttgtctgtggggcgagttcgtggatgccaccaacctgacacctagactgtggcctagagcctctgctgtgggcgagagactgtggtcccctaagacagtgaccgacctggaaaacgcctacaagagactggccgtgcacagatgcagaatggtgtccagaggaatcgccgctcagcctctgtacaccggctactgcaactacgagaacaagatcggtggtggtggatctggaggcggaggatcaggcggcggaggttctggaggtggaggtagtaccctgggcaagaacatcctggtggtgtctgtggtcaccgccgagtgcaacgagttccccaacctggaatccgtggaaaactacaccctgacaatcaacgacgaccagtgcctgctggcctccgaaacagtttggggagcactgcgcggactggaaacattcagccagctcgtgtggaaatctgccgagggcacattcttcatcaacaagaccaagatcaaggacttccctaggttcccacacaggggcgtgctgctggacacctccagacattacctgcctctgagcagcatcctggacacactggacgtgatggcttataacaagtttaatgtgttccattggcacctggtcgacgacagcagcttcccttacgagtccttcacattccccgagctgacaagaaagggctctttcaaccccgtgacacacatctacacagcccaggacgtgaaagaagtgatcgagtacgctaggctgcggggaatcagagtgctggctgagttcgatacacccggacacactctgagttggggacctggtgctcctggcctcctgacaccatgttactctggctctcacctgagcggcacattcggcccagtgaacccaagcctgaatagcacctacgactttatgagcaccctgtttctggaaatctcctccgtgtttcccgacttctacctgcatctcggcggcgacgaggtggacttcacctgttggaagtctaaccccaatatccaggcattcatgaagaagaaggggttcaccgacttcaagcagcttgagagcttctatatccagacgctgctggatatcgtgtccgactacgacaagggctacgttgtgtggcaagaagtctttgacaacaaagtgaaagtgcggcccgacaccatcatccaagtgtggcgggaagagatgcccgtcgagtacatgctggaaatgcaggacatcacaagagccggcttcagggctctgctgagcgctccatggtatctgaacagagtgaagtacggccccgattggaaggacatgtacaaagtggaacctctggccttccacggcacccctgaacagaaagctctcgtgattggcggcgaggcctgcatgtggggagaatacgtggacagcacaaacctggtgcctcggctttggccaagagctggtgctgtggctgaaaggctgtggtctagcaacctgaccaccaacatcgacttcgccttcaagaggctgtcccacttcagatgcgaactcgtgcggagaggaatccaggctcagccaatctctgtgggctactgcgagcaagagttcgagcagacctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 79 - DNA, 4275 bp, synthetic construct
  source 1..4275: mol_type = other DNA; note = L2 BA mouse gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgcctcagagccctagatctgctcctggactgctgctgctccaggctctggtgtctcttgtgtctctggcactggtggcccctgctagactgcaacctgctctgtggccatttcctagaagcgtgcagatgttccccagactgctgtacatcagcgccgaggacttcagcatcgatcacagccccaacagcacagctggccctagttgtagcctgctgcaagaggccttcagacggtactacaactacgtgttcggcttctacaagaggcaccacggacctgccagattcagagctgagccccagctgcagaaactgctggtgtctatcaccctggaaagcgagtgcgagagcttccctagcctgagcagcgacgagacatacagcctgctggtgcaagagcctgtggccgtgctgaaggctaactctgtttggggcgctctgagaggcctggaaaccttctctcagctggtgtaccaggacagcttcggcaccttcaccatcaacgagtcctctatcgccgactctcctagattccctcacagaggcatcctgatcgataccagcagacacttcctgcctgtgaaaaccatcctgaaaaccctggacgccatggccttcaacaagttcaacgtgctgcactggcacatcgtggacgaccagagctttccctaccagtccaccacctttccagagctgtccaacaagggcagctacagcctgagccacgtgtacacccctaacgacgtgcggatggtgctggaatacgccagactgagaggcatcagagtgatccccgagttcgacacccctggccacacacagtcttggggcaagggacagaagaacctgctgaccccttgctacaaccagaaaacaaagacccaggtgttcggccctgtggaccctaccgtgaacaccacctacgctttcttcaacaccttcttcaaagaaatcagcagcgtgttccccgaccagttcatccacctcggcggagatgaggtcgagttccagtgttgggccagcaatcccaacatccagggctttatgaagaggaaaggcttcggcagcgacttcagaaggctggaaagcttctacatcaagaagatcctcgagatcatcagcagcctgaagaagaacagcatcgtctggcaagaggtgttcgacgacaaggtggaactgcagcctggcaccgtggtggaagtgtggaagtctgagcactacagctacgagctgaagcaagtgaccggctctggcttccctgccatcctttctgccccttggtatctggacctgatcagctacggccaggactggaagaactactacaaggtcgagcccctgaacttcgagggcagcgagaagcagaagcagctggttatcggaggcgaggcttgtctgtggggcgagttcgtggatgccaccaacctgacacctagactgtggcctagagcctctgctgtgggcgagagactgtggtcccctaagacagtgaccgacctggaaaacgcctacaagagactggccgtgcacagatgcagaatggtgtccagaggaatcgccgctcagcctctgtacaccggctactgcaactacgagaacaagatctctggaggatctagcggaggatcctctggcagcgagacaccaggaacaagcgagtcagcaacaccagagagcagtggcggcagcagcggcggcagcagcaccctgggcaagaacatcctggtggtgtctgtggtcaccgccgagtgcaacgagttccccaacctggaatccgtggaaaactacaccctgacaatcaacgacgaccagtgcctgctggcctccgaaacagtttggggagcactgcgcggactggaaacattcagccagctcgtgtggaaatctgccgagggcacattcttcatcaacaagaccaagatcaaggacttccctaggttcccacacaggggcgtgctgctggacacctccagacattacctgcctctgagcagcatcctggacacactggacgtgatggcttataacaagtttaatgtgttccattggcacctggtcgacgacagcagcttcccttacgagtccttcacattccccgagctgacaagaaagggctctttcaaccccgtgacacacatctacacagcccaggacgtgaaagaagtgatcgagtacgctaggctgcggggaatcagagtgctggctgagttcgatacacccggacacactctgagttggggacctggtgctcctggcctcctgacaccatgttactctggctctcacctgagcggcacattcggcccagtgaacccaagcctgaatagcacctacgactttatgagcaccctgtttctggaaatctcctccgtgtttcccgacttctacctgcatctcggcggcgacgaggtggacttcacctgttggaagtctaaccccaatatccaggcattcatgaagaagaaggggttcaccgacttcaagcagcttgagagcttctatatccagacgctgctggatatcgtgtccgactacgacaagggctacgttgtgtggcaagaagtctttgacaacaaagtgaaagtgcggcccgacaccatcatccaagtgtggcgggaagagatgcccgtcgagtacatgctggaaatgcaggacatcacaagagccggcttcagggctctgctgagcgctccatggtatctgaacagagtgaagtacggccccgattggaaggacatgtacaaagtggaacctctggccttccacggcacccctgaacagaaagctctcgtgattggcggcgaggcctgcatgtggggagaatacgtggacagcacaaacctggtgcctcggctttggccaagagctggtgctgtggctgaaaggctgtggtctagcaacctgaccaccaacatcgacttcgccttcaagaggctgtcccacttcagatgcgaactcgtgcggagaggaatccaggctcagccaatctctgtgggctactgcgagcaagagttcgagcagacctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 80 - DNA, 4212 bp, synthetic construct
  source 1..4212: mol_type = other DNA; note = L3 BA mouse gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgcctcagagccctagatctgctcctggactgctgctgctccaggctctggtgtctcttgtgtctctggcactggtggcccctgctagactgcaacctgctctgtggccatttcctagaagcgtgcagatgttccccagactgctgtacatcagcgccgaggacttcagcatcgatcacagccccaacagcacagctggccctagttgtagcctgctgcaagaggccttcagacggtactacaactacgtgttcggcttctacaagaggcaccacggacctgccagattcagagctgagccccagctgcagaaactgctggtgtctatcaccctggaaagcgagtgcgagagcttccctagcctgagcagcgacgagacatacagcctgctggtgcaagagcctgtggccgtgctgaaggctaactctgtttggggcgctctgagaggcctggaaaccttctctcagctggtgtaccaggacagcttcggcaccttcaccatcaacgagtcctctatcgccgactctcctagattccctcacagaggcatcctgatcgataccagcagacacttcctgcctgtgaaaaccatcctgaaaaccctggacgccatggccttcaacaagttcaacgtgctgcactggcacatcgtggacgaccagagctttccctaccagtccaccacctttccagagctgtccaacaagggcagctacagcctgagccacgtgtacacccctaacgacgtgcggatggtgctggaatacgccagactgagaggcatcagagtgatccccgagttcgacacccctggccacacacagtcttggggcaagggacagaagaacctgctgaccccttgctacaaccagaaaacaaagacccaggtgttcggccctgtggaccctaccgtgaacaccacctacgctttcttcaacaccttcttcaaagaaatcagcagcgtgttccccgaccagttcatccacctcggcggagatgaggtcgagttccagtgttgggccagcaatcccaacatccagggctttatgaagaggaaaggcttcggcagcgacttcagaaggctggaaagcttctacatcaagaagatcctcgagatcatcagcagcctgaagaagaacagcatcgtctggcaagaggtgttcgacgacaaggtggaactgcagcctggcaccgtggtggaagtgtggaagtctgagcactacagctacgagctgaagcaagtgaccggctctggcttccctgccatcctttctgccccttggtatctggacctgatcagctacggccaggactggaagaactactacaaggtcgagcccctgaacttcgagggcagcgagaagcagaagcagctggttatcggaggcgaggcttgtctgtggggcgagttcgtggatgccaccaacctgacacctagactgtggcctagagcctctgctgtgggcgagagactgtggtcccctaagacagtgaccgacctggaaaacgcctacaagagactggccgtgcacagatgcagaatggtgtccagaggaatcgccgctcagcctctgtacaccggctactgcaactacgagaacaagatcggcagcgccggcagcgccgccggcagcggcgagttcaccctgggcaagaacatcctggtggtgtctgtggtcaccgccgagtgcaacgagttccccaacctggaatccgtggaaaactacaccctgacaatcaacgacgaccagtgcctgctggcctccgaaacagtttggggagcactgcgcggactggaaacattcagccagctcgtgtggaaatctgccgagggcacattcttcatcaacaagaccaagatcaaggacttccctaggttcccacacaggggcgtgctgctggacacctccagacattacctgcctctgagcagcatcctggacacactggacgtgatggcttataacaagtttaatgtgttccattggcacctggtcgacgacagcagcttcccttacgagtccttcacattccccgagctgacaagaaagggctctttcaaccccgtgacacacatctacacagcccaggacgtgaaagaagtgatcgagtacgctaggctgcggggaatcagagtgctggctgagttcgatacacccggacacactctgagttggggacctggtgctcctggcctcctgacaccatgttactctggctctcacctgagcggcacattcggcccagtgaacccaagcctgaatagcacctacgactttatgagcaccctgtttctggaaatctcctccgtgtttcccgacttctacctgcatctcggcggcgacgaggtggacttcacctgttggaagtctaaccccaatatccaggcattcatgaagaagaaggggttcaccgacttcaagcagcttgagagcttctatatccagacgctgctggatatcgtgtccgactacgacaagggctacgttgtgtggcaagaagtctttgacaacaaagtgaaagtgcggcccgacaccatcatccaagtgtggcgggaagagatgcccgtcgagtacatgctggaaatgcaggacatcacaagagccggcttcagggctctgctgagcgctccatggtatctgaacagagtgaagtacggccccgattggaaggacatgtacaaagtggaacctctggccttccacggcacccctgaacagaaagctctcgtgattggcggcgaggcctgcatgtggggagaatacgtggacagcacaaacctggtgcctcggctttggccaagagctggtgctgtggctgaaaggctgtggtctagcaacctgaccaccaacatcgacttcgccttcaagaggctgtcccacttcagatgcgaactcgtgcggagaggaatccaggctcagccaatctctgtgggctactgcgagcaagagttcgagcagacctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 81 - DNA, 4437 bp, synthetic construct
  source 1..4437: mol_type = other DNA; note = L1 AB human noh gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagaccggtggtggtggatctggaggcggaggatcaggcggcggaggttctggaggtggaggtagtgctcgggccccgagcgtctcggccaagccggggccggcgctgtggcccctgccgctcttggtgaagatgaccccgaacctgctgcatctcgccccggagaacttctacatcagccacagccccaattccacggcgggcccctcctgcaccctgctggaggaagcgtttcgacgatatcatggctatatttttggtttctacaagtggcatcatgaacctgctgaattccaggctaaaacccaggttcagcaacttcttgtctcaatcacccttcagtcagagtgtgatgctttccccaacatatcttcagatgagtcttatactttacttgtgaaagaaccagtggctgtccttaaggccaacagagtttggggagcattacgaggtttagagacctttagccagttagtttatcaagattcttatggaactttcaccatcaatgaatccaccattattgattctccaaggttttctcacagaggaattttgattgatacatccagacattatctgccagttaagattattcttaaaactctggatgccatggcttttaataagtttaatgttcttcactggcacatagttgatgaccagtctttcccatatcagagcatcacttttcctgagttaagcaataaaggaagctattctttgtctcatgtttatacaccaaatgatgtccgtatggtgattgaatatgccagattacgaggaattcgagtcctgccagaatttgatacccctgggcatacactatcttggggaaaaggtcagaaagacctcctgactccatgttacagtagacaaaacaagttggactcttttggacctataaaccctactctgaatacaacatacagcttccttactacatttttcaaagaaattagtgaggtgtttccagatcaattcattcatttgggaggagatgaagtggaatttaaatgttgggaatcaaatccaaaaattcaagatttcatgaggcaaaaaggctttggcacagattttaagaaactagaatctttctacattcaaaaggttttggatattattgcaaccataaacaagggatccattgtctggcaggaggtttttgatgataaagcaaagcttgcgccgggcacaatagttgaagtatggaaagacagcgcatatcctgaggaactcagtagagtcacagcatctggcttccctgtaatcctttctgctccttggtacttagatttgattagctatggacaagattggaggaaatactataaagtggaacctcttgattttggcggtactcagaaacagaaacaacttttcattggtggagaagcttgtctatggggagaatatgtggatgcaactaacctcactccaagattatggcctcgggcaagtgctgttggtgagagactctggagttccaaagatgtcagagatatggatgacgcctatgacagactgacaaggcaccgctgcaggatggtcgaacgtggaatagctgcacaacctctttatgctggatattgtaaccatgagaacatgtaaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 82 - DNA, 4437 bp, synthetic construct
  source 1..4437: mol_type = other DNA; note = L1 AB human GA gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgacatctagcagactgtggttcagcctgctgctggccgctgcttttgctggcagagctacagctctttggccctggcctcagaacttccagaccagcgaccagagatacgtgctgtaccccaacaacttccagttccagtacgacgtgtccagcgccgctcagcctggatgttctgtgctggatgaggccttccagcggtacagggatctgctgtttggcagcggctcttggcccagaccttacctgacaggcaagcggcacaccctggaaaagaacgtgctggtggtgtccgtggtcacccctggctgtaatcagctgcccacactggaaagcgtggaaaactacaccctgaccatcaacgacgaccagtgtctgctgctgagcgagacagtttggggagccctgagaggcctggaaaccttttctcagctcgtgtggaagtccgccgagggcaccttcttcatcaacaagaccgagatcgaggacttccccagatttccccacagaggactgctgctcgacaccagcagacattacctgcctctgagcagcatcctggataccctggacgtgatggcctacaacaagctgaacgtgttccactggcacctggtggacgaccctagcttcccttacgagagcttcacattccccgagctgatgcggaagggcagctacaaccctgtgacacacatctacacagcccaggacgtgaaagaagtgatcgagtacgcccggctgcggggcattagagtgctggccgaatttgacacccctggacacaccctgtcttggggccctggaattcctggactgctgaccccttgttacagcggcagcgagccttctggcacattcggccctgtgaaccccagcctgaacaacacctacgagtttatgagcacattctttctggaagtctccagcgtgttccccgacttctatctgcatctcggaggcgacgaggtggacttcacctgttggaagtctaaccccgagatccaggatttcatgcggaagaaaggcttcggcgaggatttcaagcagctggaaagcttctacatccagacactgctggacatcgtgtctagctacggcaagggctacgtcgtgtggcaagaggtgttcgacaacaaagtgaagatccagcctgacaccatcatccaagtgtggcgcgaggacatccccgtgaactacatgaaggaactcgagctggtcaccaaggccggctttagagcactgctgtctgccccttggtatctgaaccggatcagctacggccccgactggaaggatttctacatcgtggaacccctggccttcgagggcacacctgaacagaaggccctggttattggaggcgaggcctgtatgtggggcgagtacgtggacaacaccaacctggtgcctagactgtggcctagagctggcgctgtggccgaaagactctggtccaacaaactgacctccgacctgaccttcgcctacgagagactgagccacttcagatgcgagctgctgagaagaggcgtgcaggcccagcctctgaatgtgggcttctgcgagcaagagttcgagcagacaggtggcggaggatctggcggaggtggaagcggcggaggcggttctggtggtggtggatctgctagagcccctagcgtgtcagccaaacctggacctgctttgtggcctctgcctctgctggtcaagatgacccctaacctgctgcatctggcccctgagaacttttacatctctcacagccccaacagcaccgccggacctagctgtacactgctcgaggaagccttcagaagataccacggctacatcttcggcttctacaagtggcaccacgagcctgccgagttccaggccaaaacacaggtgcagcagctcctcgtgtctatcaccctgcagagcgagtgcgacgcctttcctaacatcagcagcgacgagagctataccctgcttgtgaaagaacccgtggccgtgctgaaggccaacagagtgtggggagcactgcggggactcgagacattttcccagctggtgtaccaggactcctacggcaccttcaccatcaatgagagcacaatcatcgacagccccagattcagccaccggggcatcctgatcgataccagcaggcactatctgcccgtgaagatcatcctgaaaacactggacgccatggcctttaacaagttcaatgtgctgcactggcatatcgtggacgatcagagcttcccctaccagtccatcacctttccagagctgagcaacaagggctcctacagcctgagccacgtgtacacccctaacgacgtgcggatggtcatcgagtatgccagactgagaggcatcagggtgctgcccgagttcgatacaccaggccatacactgagctggggcaagggccagaaagacctgctgacaccctgctactcccggcagaacaagctggacagctttggccccatcaatcccacactgaataccacctacagctttctgaccacctttttcaaagaaatcagcgaggtgttcccggaccagttcatccatctcggcggagatgaagtggaattcaagtgctgggagagcaaccccaagattcaggactttatgagacagaaaggattcgggaccgacttcaagaaacttgagagcttctatattcagaaggtcctggacatcattgccaccattaacaagggcagcattgtctggcaagaagtctttgacgacaaggccaagctggccccaggcacaatcgtggaagtgtggaaggacagcgcttaccccgaggaactgagcagagtgacagccagcggcttccctgtgattctgagcgctccctggtatctggatctgatctcttacggccaggactggcggaagtactacaaggtggaacctctggatttcggcggcacccagaagcagaagcagctgtttattggcggagaagcctgcctgtggggagaatatgtggacgccaccaatctgacccctcggttgtggcccagagcctctgcagtgggagagagactttggagcagcaaggacgtgcgcgacatggacgacgcctatgacagactgacccggcacagatgccggatggtggaaagaggaattgccgcacagcctctgtacgccggctactgcaaccacgagaatatgtagggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 83 - DNA, 4437 bp, synthetic construct
  source 1..4437: mol_type = other DNA; note = L1 AB human IDT gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgacaagcagcagactgtggttctccctgcttttggcggcggcttttgccggacgggccacagccctgtggccgtggccgcaaaattttcagacgagcgaccaacggtatgttctttatccaaataactttcaatttcagtatgatgtgtcctccgctgcacagcctggctgcagcgtcctggacgaggcatttcagcgctatagagatctcctctttgggtctggaagctggcccagaccatacttgactggcaagaggcacaccctcgagaagaatgttttggtggtttctgtagtaacccctgggtgcaatcaacttccgacccttgagtcagtagagaattatacgttgacaataaatgacgatcaatgccttcttcttagtgagacggtttggggagcgctgcgagggcttgagacatttagccagcttgtatggaagagtgcggagggtaccttcttcataaataaaacggagatcgaagattttccgcgattccctcacaggggtctgcttctggataccagcaggcattatctgcctcttagcagcatactcgacacactggacgtgatggcgtacaacaaactcaacgtgttccactggcatctggtggacgacccaagctttccatacgagagttttacgtttccagagctcatgagaaagggtagttataatcccgtgacccatatatataccgcgcaggacgtgaaagaagtcattgagtacgcgcgactccgaggaattagagtactggcagagttcgacactcctgggcacacgctcagttgggggcctggaatacctggattgttgactccctgctatagtggcagtgagccctcagggacctttggtccggttaatcccagtcttaacaacacatacgagttcatgtctacgttttttctggaggtgtcttcagtattccctgacttctacctgcaccttgggggagatgaggttgacttcacttgttggaagtctaaccctgaaatacaggacttcatgcggaaaaaaggattcggggaggatttcaaacaactggagtccttttatattcagacccttttggacattgtttccagctatggcaaagggtatgtggtgtggcaggaagttttcgacaacaaagtcaagatccaaccggataccattatacaggtctggagggaggacatccccgtaaactacatgaaggaacttgaactggtcaccaaagctggctttagagcgctgctctctgcgccttggtatttgaataggattagttatgggcccgattggaaggacttttatatcgtcgagccgcttgcctttgagggaacccctgagcaaaaagcacttgtgattgggggagaggcttgtatgtggggagaatatgtcgataatacgaacctggtaccccggctgtggcctcgcgctggggccgtggcggagcgcctctggtccaataagttgacaagtgatttgaccttcgcttacgaacggttgtctcatttcagatgtgagctgctgagacgcggcgtacaagcacaacccctgaacgttggtttttgcgaacaagaattcgaacagaccggcgggggaggctccggaggcggggggagtggaggcgggggatcaggaggcggtggcagcgccagggcgccttcagtaagcgccaaacccggcccagccctgtggcctttgccactcctggtaaaaatgactcctaatctgctgcatctggcgcctgaaaatttttatataagccactctcctaatagtactgcaggtccctcttgcactttgctcgaagaagcttttcgaaggtatcacggttatatttttggattttataagtggcaccatgaacctgcggaattccaagcgaaaactcaagtccaacagttgctcgtaagtattacacttcagagcgagtgtgatgctttccctaatatcagttctgacgaaagttacacgctcctggtaaaggagcctgtcgctgtactgaaggcgaatcgcgtttggggcgccttgcgaggcctggaaactttcagtcagttggtgtatcaggattcctatgggacattcacgatcaatgaatccactattatagacagccctaggtttagtcataggggcatactgattgacacttctagacattacctgccagtcaaaatcatcctgaaaacacttgatgccatggcattcaataagtttaatgttctccattggcacatcgtggacgatcaaagctttccataccagtctataacatttccggagcttagtaacaaggggagctattcactctcacacgtttacaccccgaacgacgtgcgcatggttattgaatacgccagacttcgaggcattagagttcttccagaatttgatacaccaggccacacgctctcctggggcaaaggccagaaggacctgcttaccccatgttactcccgacaaaacaaactcgattcttttgggccaataaacccaacgttgaacactacgtactccttcctgactacgttttttaaggaaataagtgaggtattccctgaccagtttattcacttgggtggtgatgaagtcgaatttaagtgttgggagtcaaaccccaaaatacaagacttcatgcgccagaaggggtttgggaccgactttaaaaaactcgagtctttctacattcaaaaggttctcgatattatcgcaacaattaacaaagggagcattgtgtggcaagaggtatttgacgataaagccaaactggcgcccggaacgatagtcgaggtctggaaagacagcgcctatcccgaggagcttagtcgagtcacggcttcaggttttccagttatccttagtgccccgtggtacttggatttgatttcctacggtcaagactggcgcaagtattataaggttgaacctcttgattttggtgggacccaaaaacaaaagcagttgttcatcggtggggaagcttgcttgtggggtgagtacgtagacgcgactaatcttacgcctcgattgtggcctcgggcatctgcagttggggagcgcttgtggagctcaaaggacgtgcgagacatggatgacgcttatgatagactgacccgacataggtgtcgaatggtggagagaggtatagcggcccaacctctttatgccggatactgcaaccatgaaaacatgtaaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 84 - DNA, 4437 bp, synthetic construct
  source 1..4437: mol_type = other DNA; note = L1 AB human NV gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgacctcatcccgcttgtggttctctctgttgttggcggccgcctttgcaggaagggctactgccttgtggccctggccacaaaatttccagaccagtgatcagcgatatgtactctatccaaataacttccagtttcaatacgacgtatccagcgccgctcagccaggctgtagcgtgctggatgaagcgtttcaacgctatcgagatctgttgttcgggtcaggttcttggcccaggccgtatctcactgggaaaaggcacaccctggaaaagaatgtgctggttgtcagtgtggttacaccaggatgcaatcagctcccaacacttgagagcgtggaaaactatactctcacaatcaacgacgatcagtgtctcctgctctccgaaacagtctggggcgctctgcgggggttggaaacgttctcacagctggtatggaagtccgccgagggaactttcttcatcaacaaaacggagatcgaagactttcccaggttcccacacagaggtctgcttctcgacactagccggcattaccttcccctgagctctattcttgacaccctcgacgtaatggcctataacaagctgaacgtgtttcattggcatctcgtggacgatccaagcttcccatacgagtccttcacattcccagagctgatgcgcaaagggtcatacaatccagtgacacatatttacacggcccaggatgtgaaagaggttattgagtacgccagactcaggggcattagggtgctggccgagtttgatacaccagggcataccctctcatggggccctggtatccctgggctgctgactccctgctattcaggaagtgagccgagtgggactttcgggccagtgaacccctctctgaacaacacatacgagttcatgtccacatttttcctcgaggtgagttcagtgttccccgatttttatcttcaccttggtggagacgaggttgacttcacctgttggaagagcaaccccgagattcaggacttcatgcgcaaaaaggggtttggagaggattttaagcagcttgagagcttttatatacagacactgctcgacatcgtgtcttcatatggaaaaggctatgtggtttggcaagaggtctttgacaacaaagtcaagatccagcctgacacgattatccaagtatggagagaggacatacccgtcaactacatgaaggagctggagctggtgaccaaggctgggtttagggccctgctctcagctccctggtacctgaaccgaatatcctatgggccggattggaaagacttctacatagtggaaccactggccttcgaagggactcctgagcagaaggcattggtgatcggcggtgaggcttgtatgtggggcgagtacgtagacaatacgaacctcgtgccaaggctttggccaagagctggtgcagttgcagagcgactgtggagcaataagctgacaagtgacctcacatttgcctacgagcggctgtcccactttcgctgtgaactcctgagacggggagttcaagcccagcctctgaatgttggcttttgcgagcaggagttcgaacagacagggggaggaggtagcgggggaggaggaagtgggggtggaggtagcggaggtggtggaagcgctagagcaccatcagttagtgccaaaccagggcctgcattgtggccactgcccctcctggtcaaaatgacacctaatctgctccatcttgccccggagaatttctatatcagccactcccccaattcaaccgccgggccctcatgcaccctcctcgaagaggcctttcggagataccatgggtatatattcggcttctacaaatggcatcacgaacctgctgagttccaggccaagacccaagtccaacagttgctggtgtcaatcacactgcagagcgaatgcgatgcttttcccaacatttcttccgacgagtcttacacgctccttgtgaaggagccagtagctgtcttgaaggccaatcgcgtgtggggtgccttgaggggcttggaaacattttcccagctggtctaccaggattcctatgggacctttaccatcaacgaaagtacgataattgacagtcctcggttctcacacaggggcatcttgatcgatacaagcaggcattacttgccagtcaagatcatccttaagacactggatgccatggctttcaataagttcaacgtgcttcattggcacattgtggatgatcagagcttcccataccagagcattaccttcccagagctttccaataagggtagttattctctcagccacgtgtacacacccaatgatgtgcgcatggtcatcgagtatgcccggctgagagggattagggtcctgcccgaatttgacacgccaggccacacactctcctggggcaagggccagaaggatcttttgactccatgttattcaagacaaaataagttggacagctttggacccatcaaccccacccttaacacaacttactcattcctgaccacattctttaaggagatcagtgaggtcttccccgaccagtttattcatctcggtggcgatgaggtcgagtttaaatgctgggaaagcaaccccaagatccaggacttcatgcggcaaaagggattcggaactgatttcaaaaagctcgagtccttctatattcagaaggtcttggacatcatcgctaccataaacaaggggagcatcgtttggcaggaggtgttcgacgataaagccaagctggctccagggactattgtcgaggtgtggaaagatagtgcctaccccgaggaactttcacgggtgaccgcctctggttttcctgtgatcctctcagcaccgtggtatcttgatctcatctcttacggacaggactggcggaagtactacaaagttgaaccactggattttgggggaacgcagaaacaaaagcagctgtttatcggaggcgaagcatgtctgtggggagagtatgtagatgcaaccaaccttaccccacggctgtggccacgggcttctgctgtaggtgagaggctctggagcagcaaagacgttcgcgacatggacgacgcatatgaccggcttactagacaccgctgcagaatggttgagaggggaattgccgcccagcccctttatgccggatattgcaatcatgaaaatatgtaaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 85 - DNA, 2832 bp, synthetic construct
  source 1..2832: mol_type = other DNA; note = Hexa single gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggctggatgtagactgtgggtttccctgctgctggctgccgctcttgcttgtctggctacagctctttggccctggcctcagtacatccagacctaccacagacggtacacactgtaccccaacaacttccagttccgctaccacgtgtcctctgctgctcaggcaggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 86 - DNA, 2856 bp, synthetic construct
  source 1..2856: mol_type = other DNA; note = Hexb single gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgcctcaatctccaagatctgctcctggcctgctgctgctccaggctctggtgtctctggtttcactggcactggtggcccctgctagactgcaacctgctctgtggccatttcctagaagcgtgcagatgttccccagactgctgtacatcagcgccgaggacttcagcatcgatcacagccccaacagcacagctggccctagttgtagcctgctgcaagaggccttcagacggtactacaactacgtgttcggcttctacaagaggcaccacggacctgccagattcagagctgagccccagctgcagaaactgctggtgtctatcaccctggaaagcgagtgcgagagcttccctagcctgagcagcgacgagacatacagcctgctggtgcaagagcctgtggccgtgctgaaggctaactctgtttggggcgctctgagaggcctggaaaccttctctcagctggtgtaccaggacagcttcggcaccttcaccatcaacgagtcctctatcgccgactctcctagattccctcacagaggcatcctgatcgataccagcagacacttcctgcctgtgaaaaccatcctgaaaaccctggacgccatggccttcaacaagttcaacgtgctgcactggcacatcgtggacgaccagagctttccctaccagtccaccacctttccagagctgtccaacaagggcagctacagcctgagccacgtgtacacccctaacgacgtgcggatggtgctggaatacgccagactgagaggcatcagagtgatccccgagttcgacacccctggccacacacagtcttggggcaagggacagaagaacctgctgaccccttgctacaaccagaaaacaaagacccaggtgttcggccctgtggaccctaccgtgaacaccacctacgctttcttcaacaccttcttcaaagaaatcagcagcgtgttccccgaccagttcatccacctcggcggagatgaggtcgagttccagtgttgggccagcaatcccaacatccagggctttatgaagaggaaaggcttcggcagcgacttcagaaggctggaaagcttctacatcaagaagatcctcgagatcatcagcagcctgaagaagaacagcatcgtctggcaagaggtgttcgacgacaaggtggaactgcagcctggcaccgtggtggaagtgtggaagtctgagcactacagctacgagctgaagcaagtgaccggctctggcttccctgccatcctttctgccccttggtatctggacctgatcagctacggccaggactggaagaactactacaaggtcgagcccctgaacttcgagggcagcgagaagcagaagcagctggttatcggaggcgaggcttgtctgtggggcgagttcgtggatgccaccaacctgacacctagactgtggcctagagcctctgctgtgggcgagagactgtggtcccctaagacagtgaccgacctggaaaacgcctacaagagactggccgtgcacagatgcagaatggtgtccagaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 87 - DNA, 4506 bp, synthetic construct
  source 1..4506: mol_type = other DNA; note = P2A AB mouse gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatggccggctgtagactgtgggtttcactgctgctggctgccgctctggcttgtctggctacagctctttggccctggcctcagtacatccagacctaccacagacggtacacactgtaccccaacaacttccagttccgctaccacgtgtcctctgctgctcaggctggatgtgtggtgctggacgaggccttcagaagatacagaaacctgctgttcggcagcggcagctggcctagacctagcttctctaacaagcagcagaccctgggcaagaacatcctggtggtgtctgtggtcaccgccgagtgcaacgagttccccaacctggaaagcgtggaaaactacaccctgaccatcaacgacgaccagtgcctgctggcctctgagacagtttggggagccctgagaggcctggaaaccttctctcagctcgtgtggaagtctgccgagggcaccttcttcatcaacaagaccaagatcaaggacttccctaggttccctcacagaggcgtgctgctggacaccagcagacactacctgccactgtcctccatcctggacaccctggatgtgatggcctacaacaagttcaacgtgttccactggcacctggtggacgacagcagcttcccttacgagagcttcacattccccgagctgaccagaaagggcagcttcaaccccgtgacacacatctacacagcccaggacgtgaaagaagtgatcgagtacgccagactgagaggcatcagagtgctggccgagttcgacacacctggccacacactttcttggggacctggtgctcctggcctgctgacaccttgttactctggctctcacctgagcggcacattcggccctgtgaaccctagcctgaacagcacctacgactttatgagcaccctgttcctcgagatcagcagcgtgttccccgacttctacctgcacctcggcggagatgaggtggacttcacctgttggaagtctaaccccaacatccaggctttcatgaagaagaagggcttcaccgacttcaagcagctggaaagcttctacattcagaccctgctggatatcgtgtccgactacgacaagggctacgtcgtgtggcaagaggtgttcgacaacaaagtgaaagtgcggcccgacaccatcatccaagtgtggcgggaagagatgcccgtcgagtacatgctggaaatgcaggacatcaccagagccggcttcagggctctgcttagcgctccctggtatctgaacagagtgaagtacggccccgactggaaggacatgtacaaggtggaacccctggccttccacggcacccctgaacagaaggctctggttatcggaggcgaggcctgtatgtggggagagtacgtggacagcaccaacctggtgccaagactgtggcctagagctggcgctgtggctgagagactgtggtccagcaacctgaccaccaacatcgacttcgccttcaagagactgagccacttcagatgcgaactcgtgcggagaggaatccaggctcagcctatctctgtgggctactgcgagcaagagttcgagcagacaggatctggcgccaccaacttcagcctgctgaaacaggctggcgacgtggaagagaaccccggacctatgcctcagagccctagatctgctcccgggctgctgcttctgcaagctctggtgtctctcgtgtctctggccctggttgctccagctagactgcagcctgctctgtggccatttcctagaagcgtgcagatgttccccagactgctgtacatcagcgccgaggacttcagcatcgatcacagccccaacagcacagctggcccatcttgtagcctgctgcaagaggcctttagacggtactacaactacgtgttcggcttctacaagaggcaccacggacctgccagattcagagccgaacctcagctgcagaagctgctcgtcagcatcaccctggaatccgagtgcgagagctttcccagcctgtccagcgacgagacatactccctgctggtgcaagagcctgtggctgtgctgaaggccaactctgtgtggggtgctctgcgcggactggaaacattttcccagctggtgtaccaggacagcttcggcaccttcacaatcaacgagtcctctatcgccgactctcctagattcccacacaggggcatcctgatcgatacctccaggcacttcctgcctgtgaaaaccatcctcaagaccctggacgccatggctttcaacaagtttaatgtgctgcattggcacatcgtggatgaccaaagcttcccctaccagtccaccacctttccagagctgtccaacaagggctcctacagcctgtctcacgtgtacacccctaacgacgtgcggatggtcctggaatacgctaggctgcggggaatcagagtgatccctgagttcgatactcccggacacacacagagctggggcaagggacagaagaacctgctgaccccatgctacaaccagaaaacaaagacccaggtgttcggcccagtggacccaaccgtgaacacaacctacgctttcttcaacacattcttcaaagaaatcagctccgtgtttcccgaccagttcatccatctcggcggcgacgaagtcgagttccagtgttgggccagcaatcccaatatccaaggattcatgaagcggaaaggcttcggctccgacttcagaaggctggaatctttctacatcaagaagatcctcgaaatcatcagcagcctgaaaaagaacagcatcgtctggcaagaagtctttgacgacaaggtcgagctgcagccaggcactgtggtggaagtgtggaaaagcgagcactacagctacgagctgaagcaagtgacaggcagcggcttccctgctatcctctctgccccttggtatctggacctgatcagctacggccaggattggaagaactactacaaggttgagcccctcaacttcgagggcagcgagaagcagaaacagctggtcattggcggcgaggcttgtctctggggcgagtttgtggatgccaccaatctgacccctaggctgtggccaagggcttctgctgtgggagagagactttggagccccaagaccgtgaccgacctggaaaacgcctataagagactggccgtgcacagatgcagaatggtgtcccgaggaatcgccgctcagcctctgtacaccggctactgcaactacgagaacaagatctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 88 - DNA, 4506 bp, synthetic construct
  source 1..4506: mol_type = other DNA; note = P2A BA mouse gene construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagctagcgccaccatgcctcagagccctagatctgctcctggactgctgctgctccaggctctggtgtctcttgtgtctctggcactggtggcccctgctagactgcaacctgctctgtggccatttcctagaagcgtgcagatgttccccagactgctgtacatcagcgccgaggacttcagcatcgatcacagccccaacagcacagctggccctagttgtagcctgctgcaagaggccttcagacggtactacaactacgtgttcggcttctacaagaggcaccacggacctgccagattcagagctgagccccagctgcagaaactgctggtgtctatcaccctggaaagcgagtgcgagagcttccctagcctgagcagcgacgagacatacagcctgctggtgcaagagcctgtggccgtgctgaaggctaactctgtttggggcgctctgagaggcctggaaaccttctctcagctggtgtaccaggacagcttcggcaccttcaccatcaacgagtcctctatcgccgactctcctagattccctcacagaggcatcctgatcgataccagcagacacttcctgcctgtgaaaaccatcctgaaaaccctggacgccatggccttcaacaagttcaacgtgctgcactggcacatcgtggacgaccagagctttccctaccagtccaccacctttccagagctgtccaacaagggcagctacagcctgagccacgtgtacacccctaacgacgtgcggatggtgctggaatacgccagactgagaggcatcagagtgatccccgagttcgacacccctggccacacacagtcttggggcaagggacagaagaacctgctgaccccttgctacaaccagaaaacaaagacccaggtgttcggccctgtggaccctaccgtgaacaccacctacgctttcttcaacaccttcttcaaagaaatcagcagcgtgttccccgaccagttcatccacctcggcggagatgaggtcgagttccagtgttgggccagcaatcccaacatccagggctttatgaagaggaaaggcttcggcagcgacttcagaaggctggaaagcttctacatcaagaagatcctcgagatcatcagcagcctgaagaagaacagcatcgtctggcaagaggtgttcgacgacaaggtggaactgcagcctggcaccgtggtggaagtgtggaagtctgagcactacagctacgagctgaagcaagtgaccggctctggcttccctgccatcctttctgccccttggtatctggacctgatcagctacggccaggactggaagaactactacaaggtcgagcccctgaacttcgagggcagcgagaagcagaagcagctggttatcggaggcgaggcttgtctgtggggcgagttcgtggatgccaccaacctgacacctagactgtggcctagagcctctgctgtgggcgagagactgtggtcccctaagacagtgaccgacctggaaaacgcctacaagagactggccgtgcacagatgcagaatggtgtccagaggaatcgccgctcagcctctgtacaccggctactgcaactacgagaacaagatcggcagcggcgccaccaacttctcactgctgaaacaggctggcgacgtggaagagaacccaggacctatggctggctgcagactgtgggtttccctgttgctggctgctgccctggcatgtcttgctacagcactttggccctggcctcagtacatccagacctaccacagacgctacacactgtaccccaacaacttccagttccgctaccacgtgtcctctgccgctcaagctggatgtgtggtcctggacgaggctttcagaagatacagaaacctgctgttcggctccggcagctggcctagacctagcttctctaacaagcagcagaccctgggcaagaacatcctggtggtgtctgtggtcaccgccgagtgcaacgagttccccaacctggaatccgtggaaaactacaccctgacaatcaacgacgaccagtgcctgctggcctccgaaacagtttggggagcactgcgcggactggaaacattcagccagctcgtgtggaaatctgccgagggcacattcttcatcaacaagaccaagatcaaggacttccctaggttcccacacaggggcgtgctgctggacacctccagacattacctgcctctgagcagcatcctggacacactggacgtgatggcttataacaagtttaatgtgttccattggcacctggtcgacgacagcagcttcccttacgagtccttcacattccccgagctgacaagaaagggctctttcaaccccgtgacacacatctacacagcccaggacgtgaaagaagtgatcgagtacgctaggctgcggggaatcagagtgctggctgagttcgatacacccggacacactctgagttggggacctggtgctcctggcctcctgacaccatgttactctggctctcacctgagcggcacattcggcccagtgaacccaagcctgaatagcacctacgactttatgagcaccctgtttctggaaatctcctccgtgtttcccgacttctacctgcatctcggcggcgacgaggtggacttcacctgttggaagtctaaccccaatatccaggcattcatgaagaagaaggggttcaccgacttcaagcagcttgagagcttctatatccagacgctgctggatatcgtgtccgactacgacaagggctacgttgtgtggcaagaagtctttgacaacaaagtgaaagtgcggcccgacaccatcatccaagtgtggcgggaagagatgcccgtcgagtacatgctggaaatgcaggacatcacaagagccggcttcagggctctgctgagcgctccatggtatctgaacagagtgaagtacggccccgattggaaggacatgtacaaagtggaacctctggccttccacggcacccctgaacagaaagctctcgtgattggcggcgaggcctgcatgtggggagaatacgtggacagcacaaacctggtgcctcggctttggccaagagctggtgctgtggctgaaaggctgtggtctagcaacctgaccaccaacatcgacttcgccttcaagaggctgtcccacttcagatgcgaactcgtgcggagaggaatccaggctcagccaatctctgtgggctactgcgagcaagagttcgagcagacctgaggatccaacttgtttattgcagcttataatggttacaaataaagcaatagcatcacaaatttcacaaataaagcatttttttcactgcattctagttgtggtttgtccaaactcatcaatgtatcttatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg